(19) United States
(12) Patent Application Publication
(10) Pub. No.: US 2026/0055409 A1
(43) Pub. Date: Feb. 26, 2026

(54) COMPOUNDS AND METHODS FOR TREATING HUMAN SUBJECTS

(71) Applicant: Stoke Therapeutics, Inc., Bedford, MA (US)

(72) Inventors: Isabel Aznarez, Jamaica Plain, MA (US); Sethumadhavan Divakaramenon, Lexington, MA (US); Juergen Scharner, Arlington, MA (US); Hyun-Yong Jeon, Arlington, MA (US)

(21) Appl. No.: 19/372,895

(22) Filed: Oct. 29, 2025

Related U.S. Application Data
(63) Continuation of application No. PCT/US2024/027823, filed on May 3, 2024.
(60) Provisional application No. 63/499,779, filed on May 3, 2023.

(51) Int. Cl. C12N 15/113

(52) U.S. Cl. CPC C12N 15/113; C12N 2310/14; C12N 2310/315; C12N 2310/321; C12N 2320/33

(57) ABSTRACT

Described herein are compounds and methods that can be used to treat a disease or condition by increasing SYNGAP1 protein.

### CROSS-REFERENCE

This application is a continuation of International Application No. PCT/US2024/027823, filed May 3, 2024, which claims benefit of U.S. Provisional Patent Application No. 63/499,779 filed May 3, 2023, each of which is incorporated herein by reference in its entirety.

### SEQUENCE LISTING

The instant application contains a Sequence Listing which has been submitted electronically in XML format and is hereby incorporated by reference in its entirety. Said XML copy, created on Oct. 29, 2025, is named 47991-740_301_SL.xml and is 2,793,768 bytes in size.

### BACKGROUND

Deficient SYNGAP1 protein or activity has been observed in subjects with mental retardation, or intellectual disability, epileptic encephalopathy, or autism. There is a need for therapeutic agents that can be used to treat such conditions or diseases by increasing SYNGAP1 protein or activity.

### SUMMARY

Provided herein are therapeutic agents that can be used to increase SYNGAP1 protein or activity and treat conditions or diseases.

Provided herein is a compound of Formula (I):

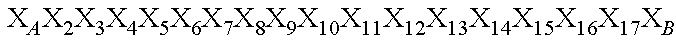

wherein
X_{A} is

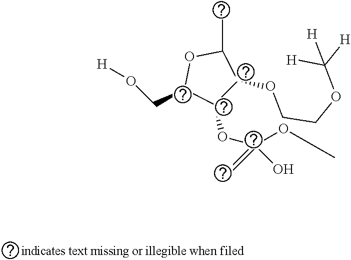

### and B_{A} is

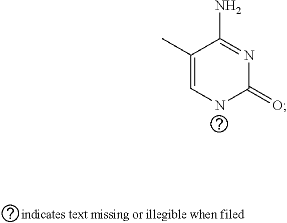

### X₂ is

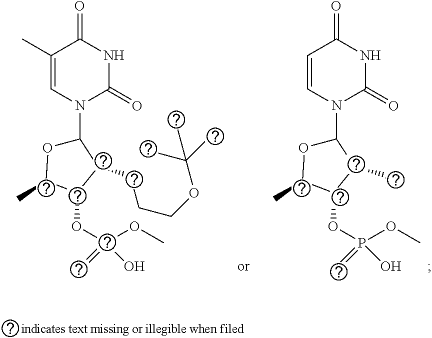

X₃ is

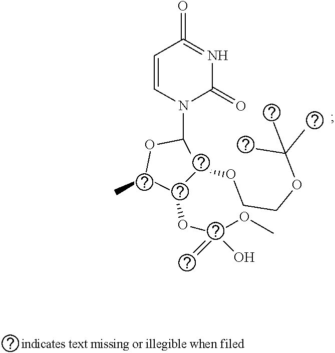

### X₄ is

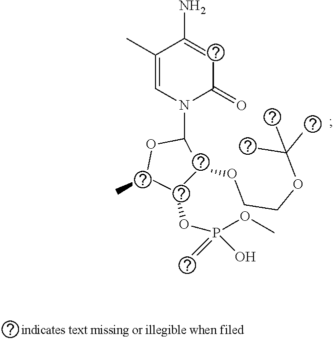 [SMILES: C=P(O)(OC)O[C@@H]1[C@@H](C)OC(n2cc(C)c(N)cc2=O)[C@@H]1OCCOC(C)(C)C]

### X₅ is

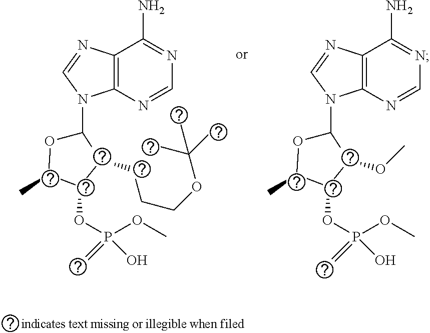 [SMILES: C=P(O)(OC)O[C@@H]1[C@@H](C)OC(n2cnc3c(N)ncnc32)[C@@H]1CCCOC(C)(C)C ; "or" ; C=P(O)(OC)O[C@@H]1[C@@H](C)OC(n2cnc3c(N)ncnc32)[C@@H]1OC]

X₆ is

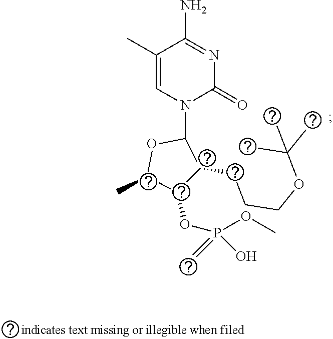 [SMILES: C=P(O)(OC)O[C@@H]1[C@@H](C)OC(n2cc(C)c(N)nc2=O)[C@@H]1CCCOC(C)(C)C]

### X₇ is

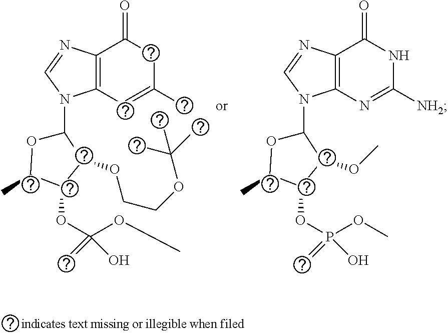

### X₈ is

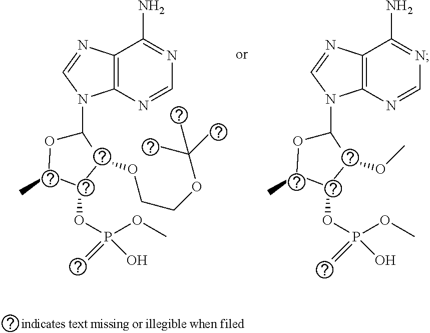 [SMILES: C=P(O)(OC)O[C@@H]1[C@@H](C)OC(n2cnc3c(N)ncnc32)[C@@H]1OCCOC(C)(C)C ; "or" ; C=P(O)(OC)O[C@@H]1[C@@H](C)OC(n2cnc3c(N)ncnc32)[C@@H]1OC]

X₉ is

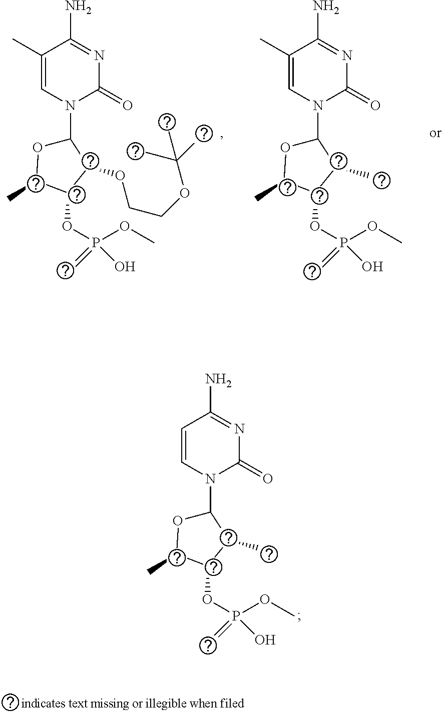

### X₁₀ is

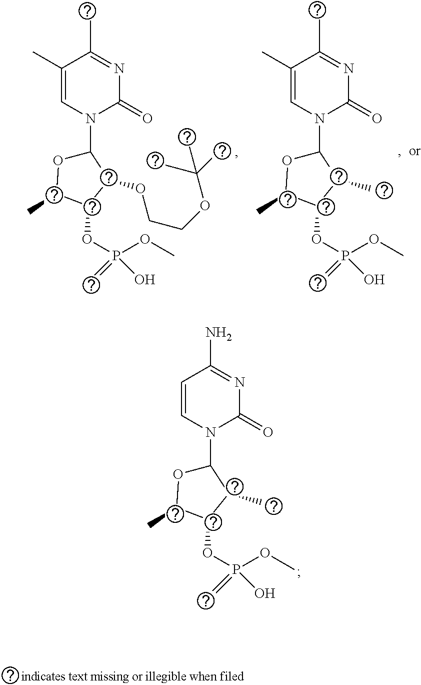

### X₁₁ is

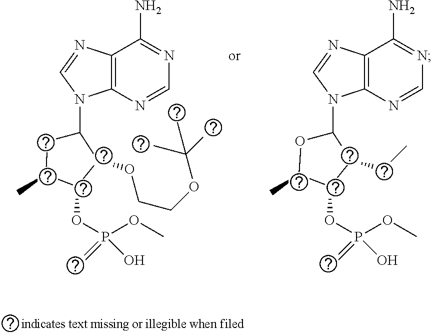 [SMILES: C=P(O)(OC)O[C@@H]1[C@@H](C)CC(n2cnc3c(N)ncnc32)[C@@H]1OCCOC(C)(C)C ; C=P(O)(OC)O[C@@H]1[C@@H](C)OC(n2cnc3c(N)ncnc32)[C@@H]1CC]

### X₁₂ is

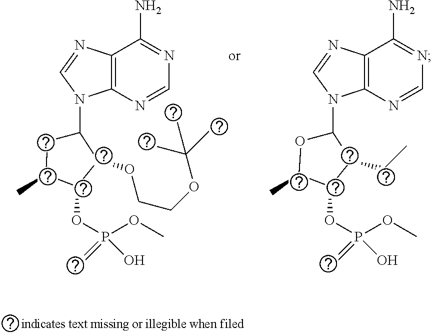 [SMILES: C=P(O)(OC)O[C@@H]1[C@@H](C)CC(n2cnc3c(N)ncnc32)[C@@H]1OCCOC(C)(C)C ; C=P(O)(OC)O[C@@H]1[C@@H](C)OC(n2cnc3c(N)ncnc32)[C@@H]1CC]

X₁₃ is

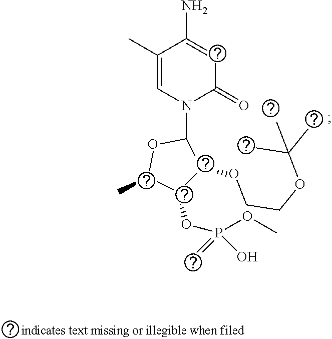 [SMILES: C=P(O)(OC)O[C@@H]1[C@@H](C)OC(n2cc(C)c(N)cc2=O)[C@@H]1OCCOC(C)(C)C]

### X₁₄ is

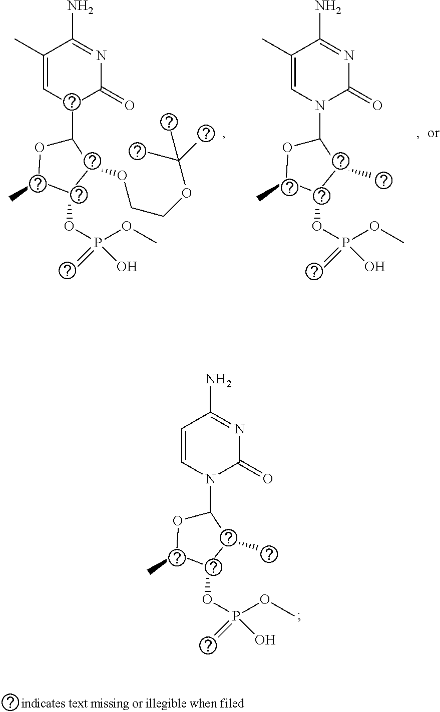

X₁₅ is

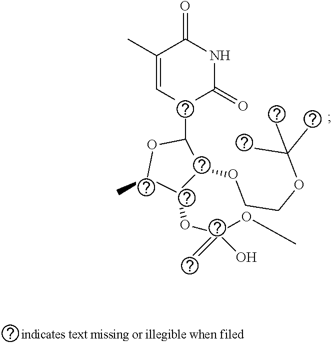

### X₁₆ is

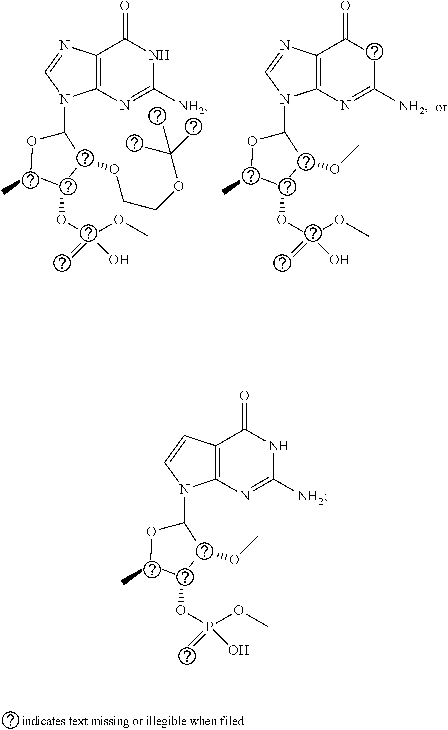

### X₁₇ is

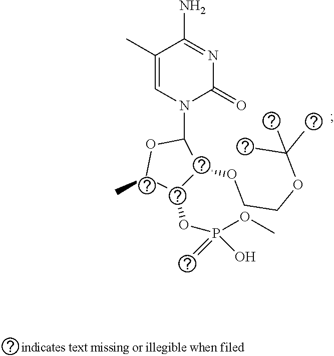 [SMILES: C=P(O)(OC)O[C@@H]1[C@@H](C)OC(n2cc(C)c(N)nc2=O)[C@@H]1OCCOC(C)(C)C]

### X_{B} is

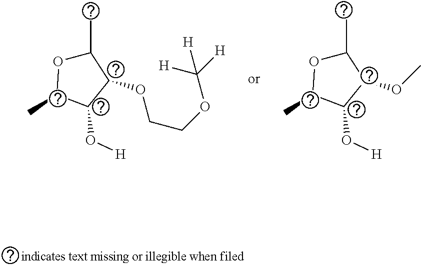

### and B_{B} is

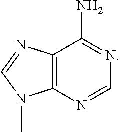

In some embodiments,
X₂ is

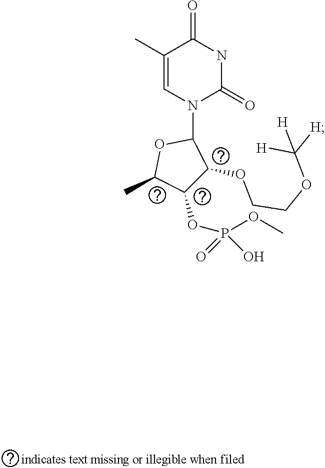

### X₅ is

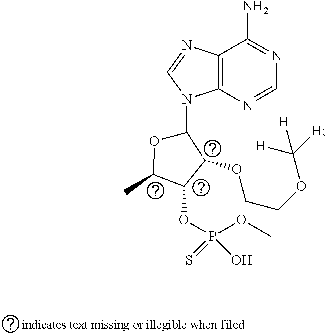

### X₇ is

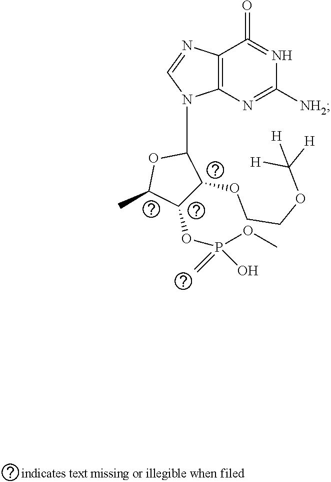

### X₈ is

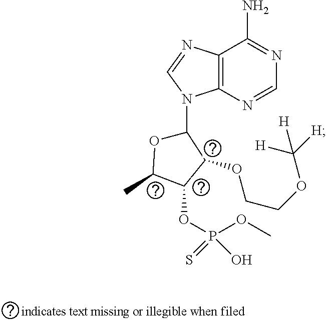

X₉ is

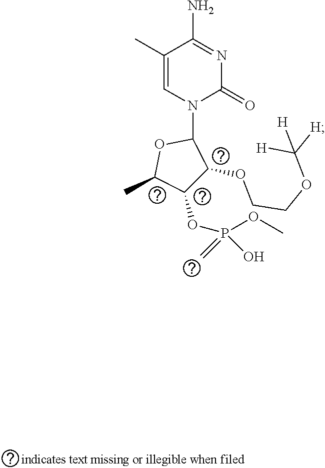

### X₁₀ is

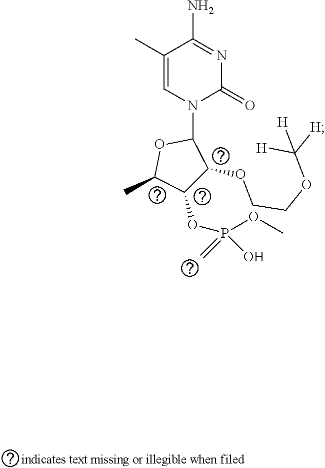

### X₁₁ is

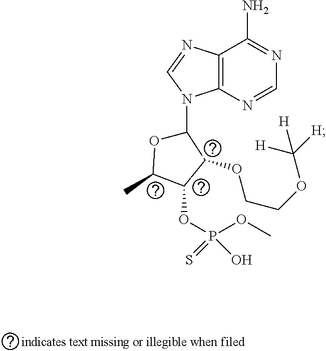

### X₁₂ is

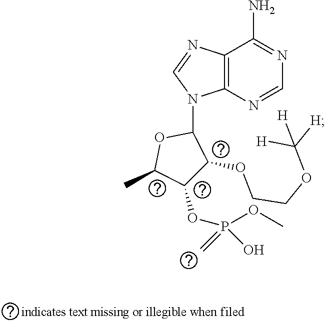

X₁₄ is

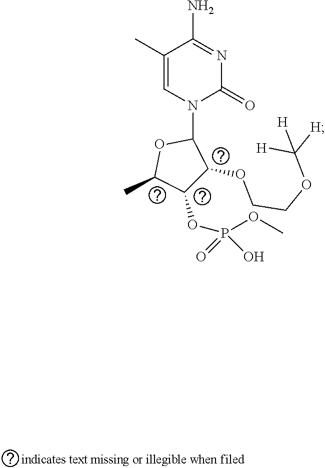

### X₁₆ is

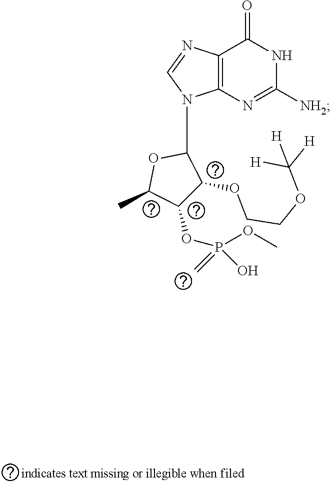 [SMILES: C=P(O)(OC)O[C@@H]1[C@@H](C)OC(n2cnc3c(=O)[nH]c(N)nc32)[C@@H]1OCCOC]

### and X_{B} is

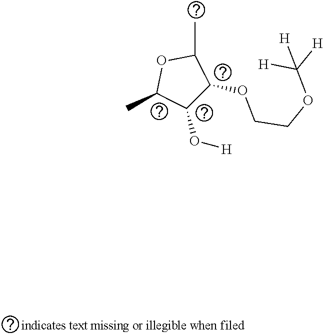

### and B_{B} is

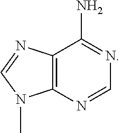

In some embodiments,
X₂ is

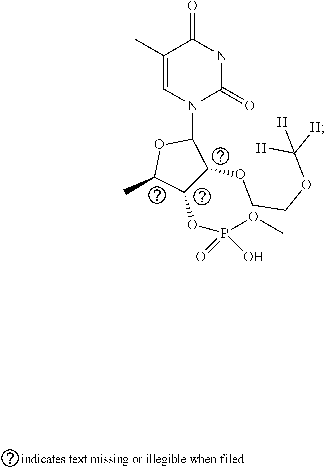

### X₅ is

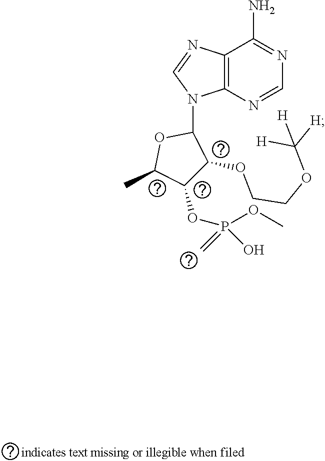

### X₇ is

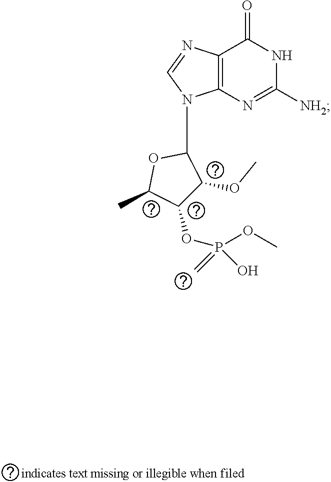 [SMILES: C=P(O)(OC)O[C@@H]1[C@@H](C)OC(n2cnc3c(=O)[nH]c(N)nc32)[C@@H]1OC]

### X₈ is

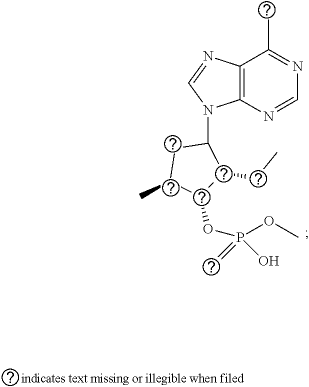

X₉ is

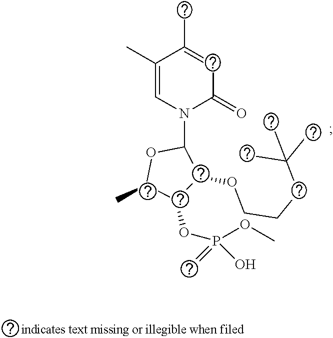 [SMILES: C=P(O)(OC)O[C@@H]1[C@@H](C)OC(n2cc(C)c(C)cc2=O)[C@@H]1OCCCC(C)(C)C]

### X₁₀ is

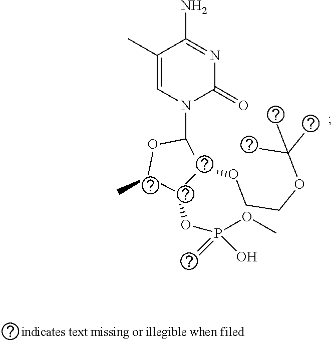 [SMILES: C=P(O)(OC)O[C@@H]1[C@@H](C)OC(n2cc(C)c(N)nc2=O)[C@@H]1OCCOC(C)(C)C]

### X₁₁ is

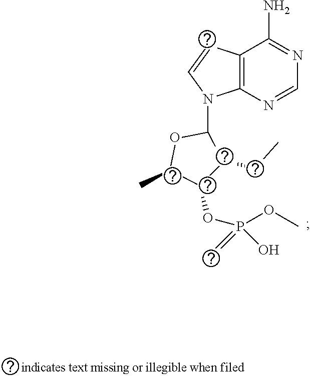

### X₁₂ is

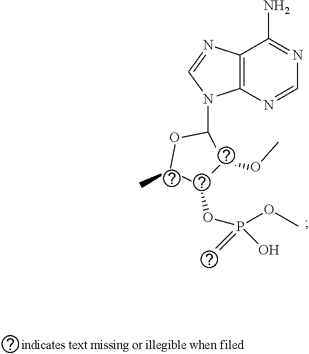

X₁₄ is

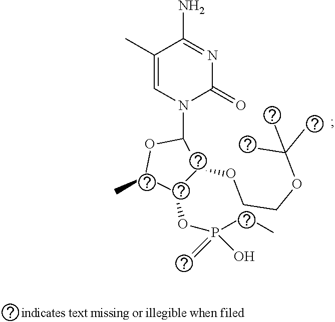 [SMILES: C=P(O)(CC)O[C@@H]1[C@@H](C)OC(n2cc(C)c(N)nc2=O)[C@@H]1OCCOC(C)(C)C]

### X₁₆ is

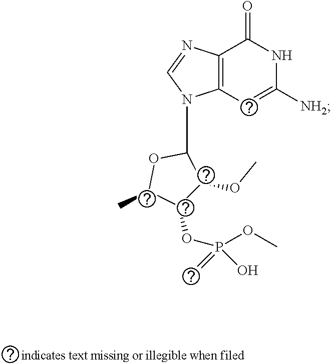 [SMILES: C=P(O)(OC)O[C@@H]1[C@@H](C)OC(n2cnc3c(=O)[nH]c(N)cc32)[C@@H]1OC]

### and X_{B} is

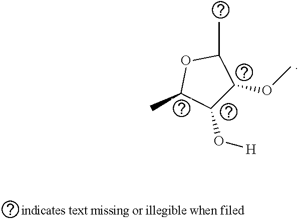

In some embodiments,
X₂ is

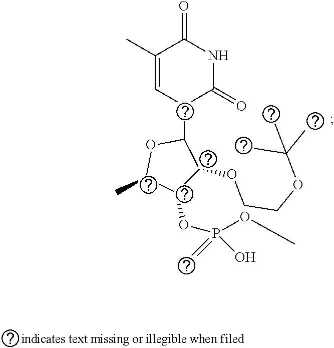 [SMILES: C=P(O)(OC)O[C@@H]1[C@@H](C)OC(C2C=C(C)C(=O)NC2=O)[C@@H]1OCCOC(C)(C)C]

### X₅ is

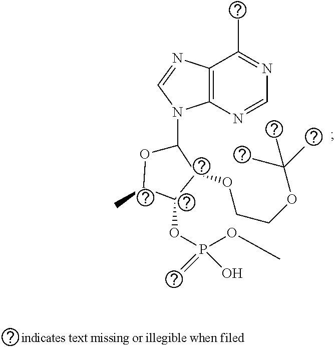 [SMILES: C=P(O)(OC)O[C@@H]1[C@@H](C)OC(n2cnc3c(C)ncnc32)[C@@H]1OCCOC(C)(C)C]

### X₇ is

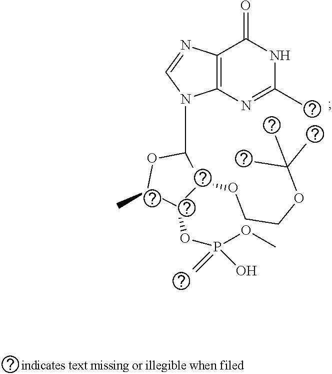 [SMILES: C=P(O)(OC)O[C@@H]1[C@@H](C)OC(n2cnc3c(=O)[nH]c(C)nc32)[C@@H]1OCCOC(C)(C)C]

### X₈ is

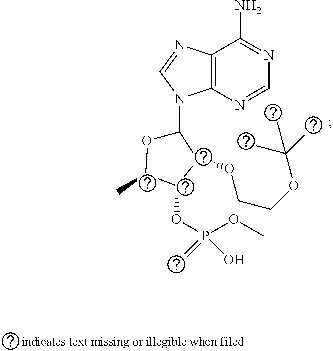 [SMILES: C=P(O)(OC)O[C@@H]1[C@@H](C)OC(n2cnc3c(N)ncnc32)[C@@H]1OCCOC(C)(C)C]

X₉ is

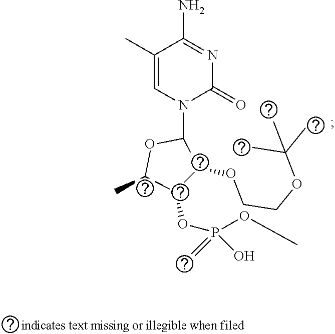 [SMILES: C=P(O)(OC)O[C@@H]1[C@@H](C)OC(n2cc(C)c(N)nc2=O)[C@@H]1OCCOC(C)(C)C]

### X₁₀ is

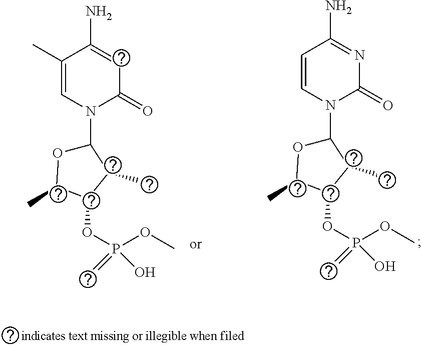

### X₁₁ is

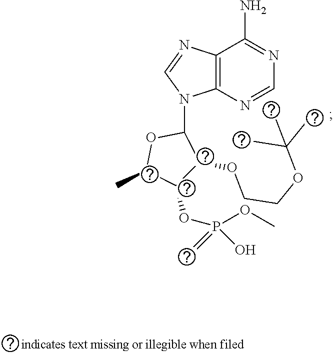 [SMILES: C=P(O)(OC)O[C@@H]1[C@@H](C)OC(n2cnc3c(N)ncnc32)[C@@H]1OCCOC(C)(C)C]

X₁₂ is

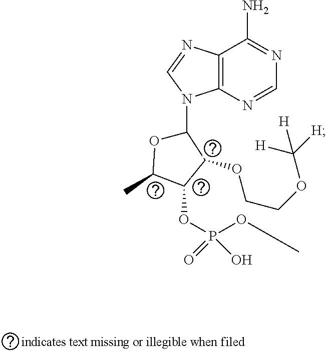

### X₁₄ is

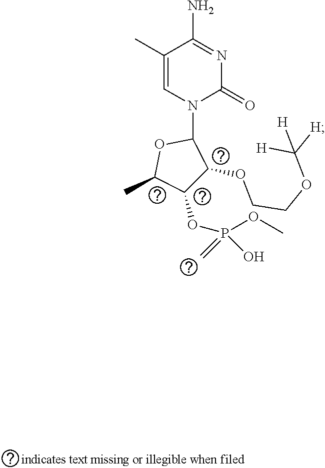

### X₁₆ is

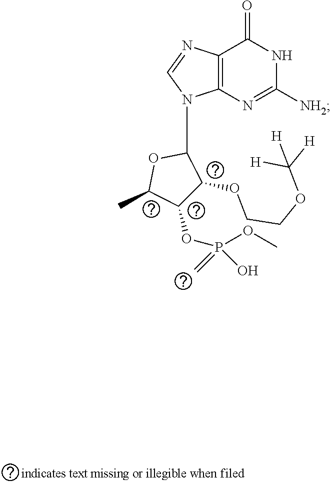 [SMILES: C=P(O)(OC)O[C@@H]1[C@@H](C)OC(n2cnc3c(=O)[nH]c(N)nc32)[C@@H]1OCCOC]

### and X_{B} is

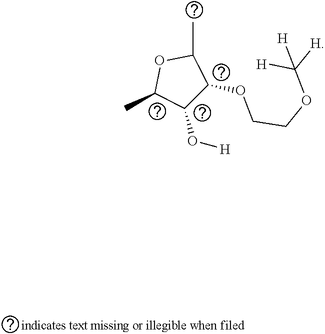

In some embodiments,
X₂ is

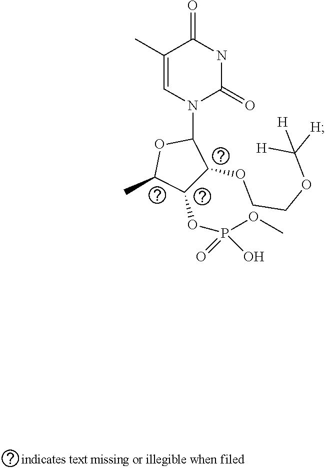

### X₅ is

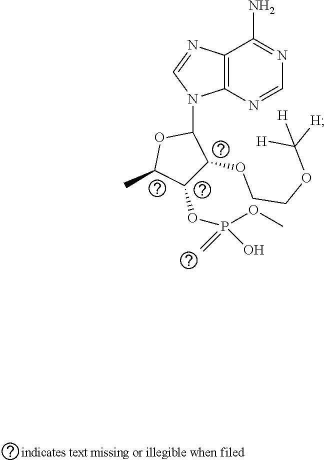

### X₇ is

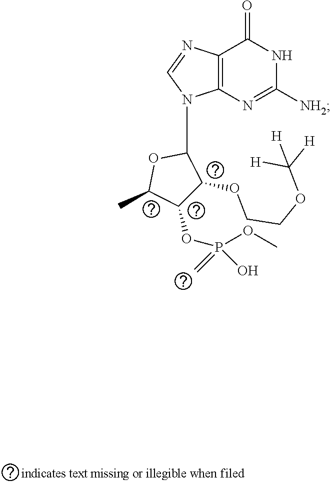 [SMILES: C=P(O)(OC)O[C@@H]1[C@@H](C)OC(n2cnc3c(=O)[nH]c(N)nc32)[C@@H]1OCCOC]

### X₈ is

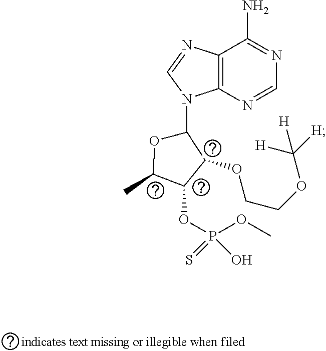

X₉ is

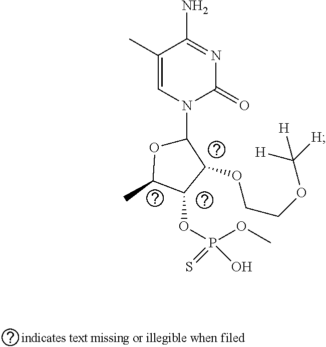

### X₁₀ is

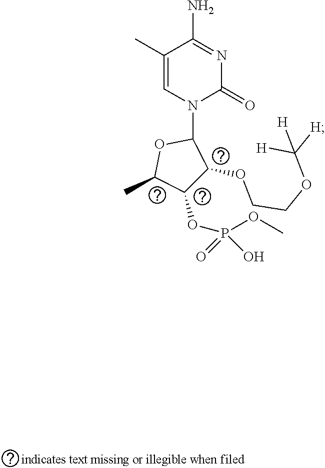

### X₁₁ is

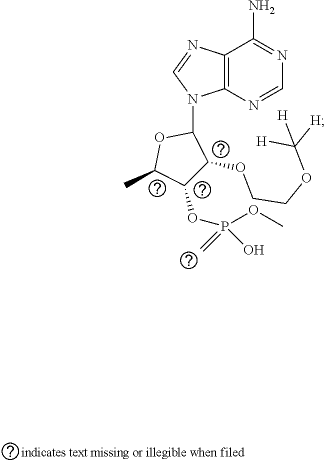

### X₁₂ is

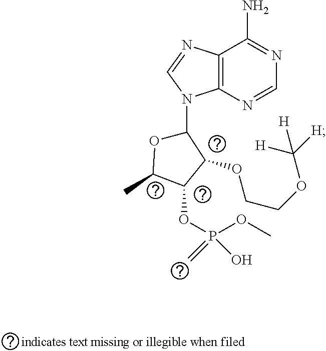

X₁₄ is

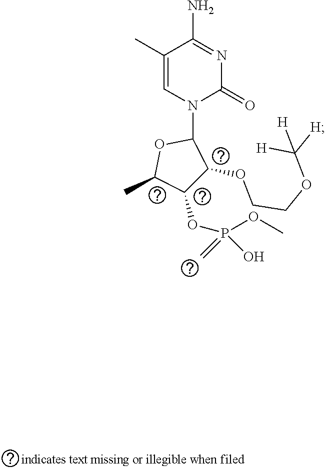

### X₁₆ is

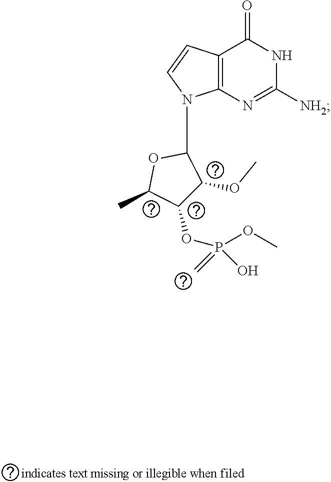 [SMILES: C=P(O)(OC)O[C@@H]1[C@@H](C)OC(n2ccc3c(=O)[nH]c(N)nc32)[C@@H]1OC]

### and X_{B} is

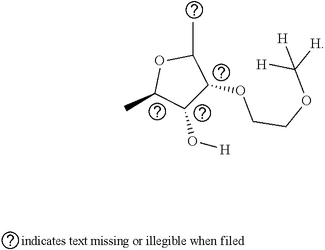

In some embodiments,
X₂ is

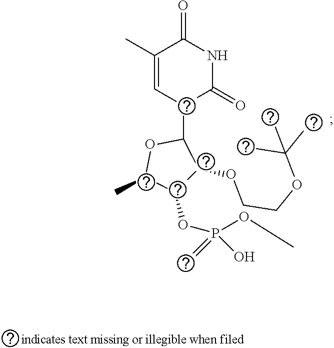 [SMILES: C=P(O)(OC)O[C@@H]1[C@@H](C)OC(C2C=C(C)C(=O)NC2=O)[C@@H]1OCCOC(C)(C)C]

### X₅ is

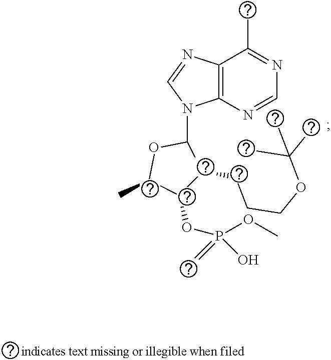 [SMILES: C=P(O)(OC)O[C@@H]1[C@@H](C)OC(n2cnc3c(C)ncnc32)[C@@H]1CCCOC(C)(C)C]

### X₇ is

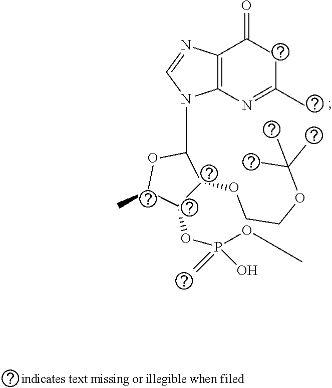 [SMILES: C=P(O)(OC)O[C@@H]1[C@@H](C)OC(n2cnc3c2N=C(C)CC3=O)[C@@H]1OCCOC(C)(C)C]

### X₈ is

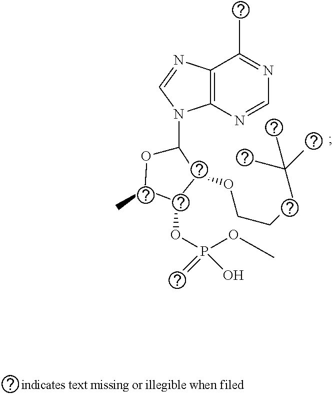 [SMILES: C=P(O)(OC)O[C@@H]1[C@@H](C)OC(n2cnc3c(C)ncnc32)[C@@H]1OCCCC(C)(C)C]

X₉ is

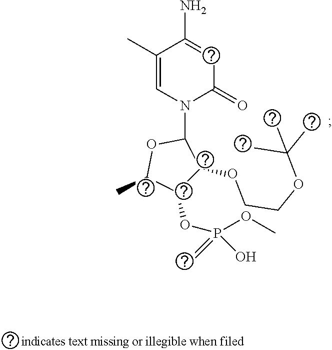 [SMILES: C=P(O)(OC)O[C@@H]1[C@@H](C)OC(n2cc(C)c(N)cc2=O)[C@@H]1OCCOC(C)(C)C]

### X₁₀ is

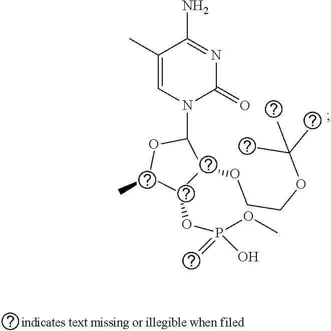 [SMILES: C=P(O)(OC)O[C@@H]1[C@@H](C)OC(n2cc(C)c(N)nc2=O)[C@@H]1OCCOC(C)(C)C]

### X₁₁ is

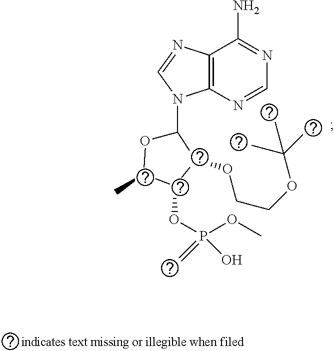 [SMILES: C=P(O)(OC)O[C@@H]1[C@@H](C)OC(n2cnc3c(N)ncnc32)[C@@H]1OCCOC(C)(C)C]

### X₁₂ is

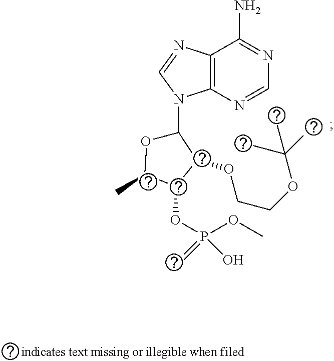 [SMILES: C=P(O)(OC)O[C@@H]1[C@@H](C)OC(n2cnc3c(N)ncnc32)[C@@H]1OCCOC(C)(C)C]

X₁₄ is

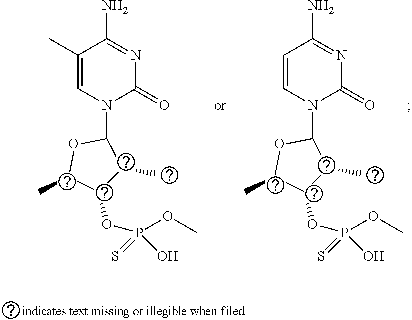

### X₁₆ is

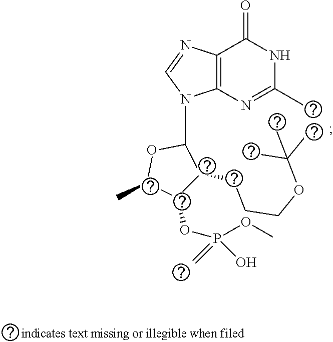 [SMILES: C=P(O)(OC)O[C@@H]1[C@@H](C)OC(n2cnc3c(=O)[nH]c(C)nc32)[C@@H]1CCCOC(C)(C)C]

### and X_{B} is

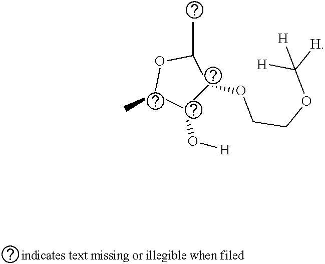

In some embodiments,
X₂ is

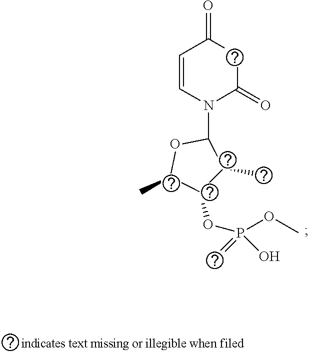

### X₅ is

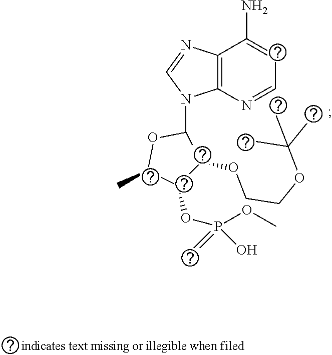 [SMILES: C=P(O)(OC)O[C@@H]1[C@@H](C)OC(n2cnc3c(N)ccnc32)[C@@H]1OCCOC(C)(C)C]

### X₇ is

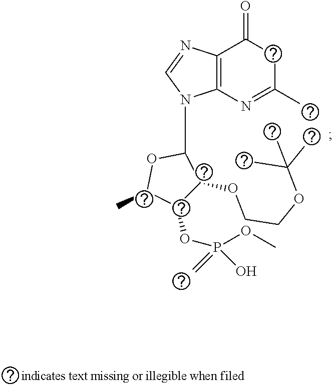 [SMILES: C=P(O)(OC)O[C@@H]1[C@@H](C)OC(n2cnc3c2N=C(C)CC3=O)[C@@H]1OCCOC(C)(C)C]

### X₈ is

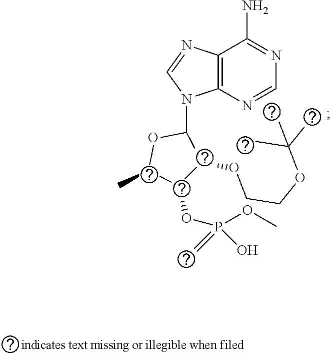 [SMILES: C=P(O)(OC)O[C@@H]1[C@@H](C)OC(n2cnc3c(N)ncnc32)[C@@H]1OCCOC(C)(C)C]

X₉ is

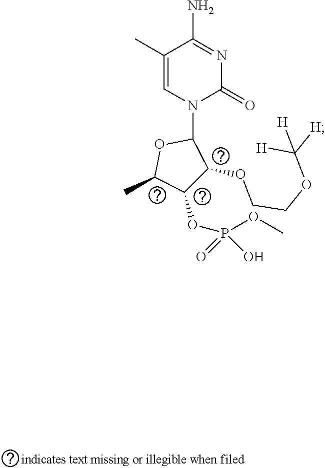

### X₁₀ is

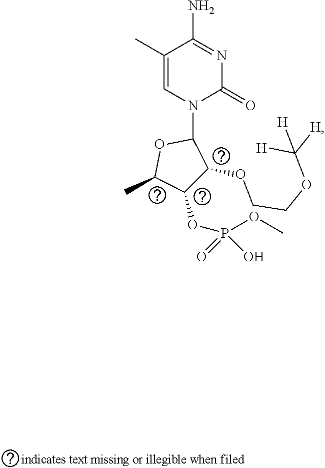

### X₁₁ is

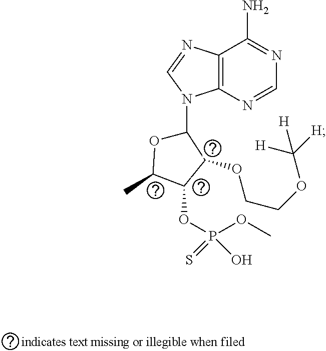

### X₁₂ is

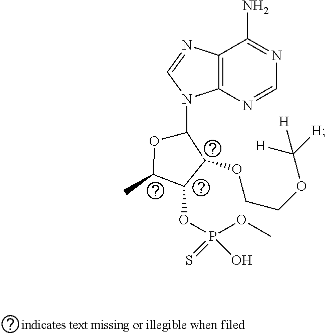

X₁₄ is

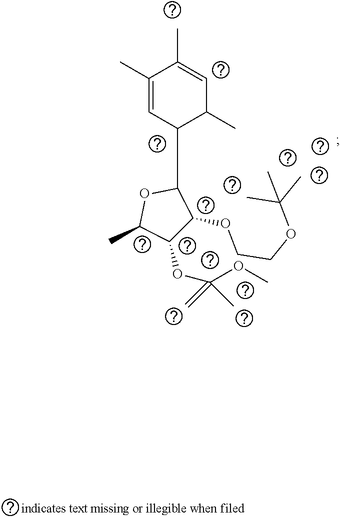

### X₁₆ is

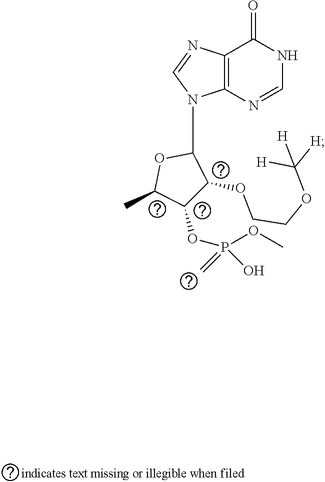

### and X_{B} is

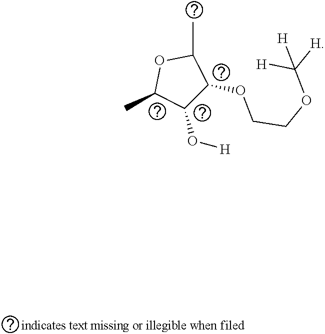

In some embodiments,
X₂ is

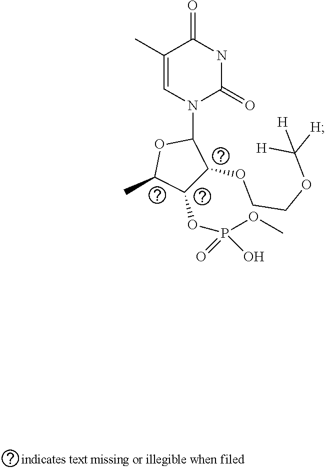

### X₅ is

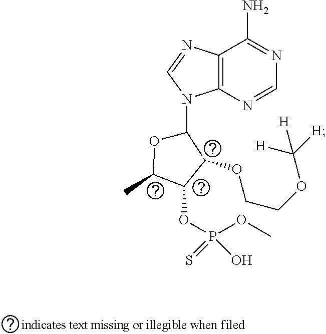

### X₇ is

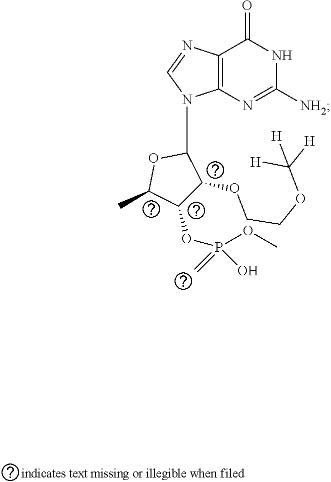 [SMILES: C=P(O)(OC)O[C@@H]1[C@@H](C)OC(n2cnc3c(=O)[nH]c(N)nc32)[C@@H]1OCCOC]

### X₈ is

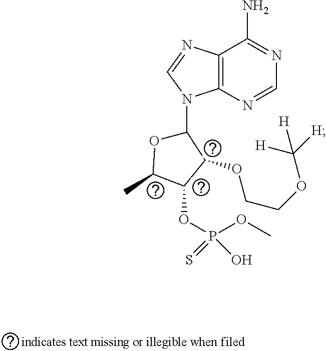

X₉ is

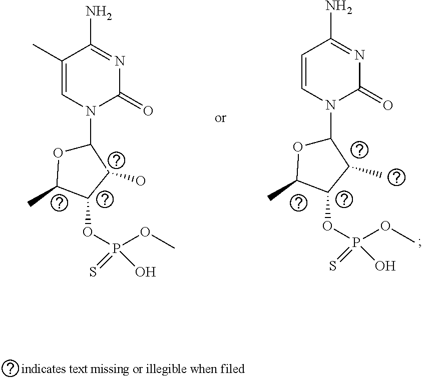

### X₁₀ is

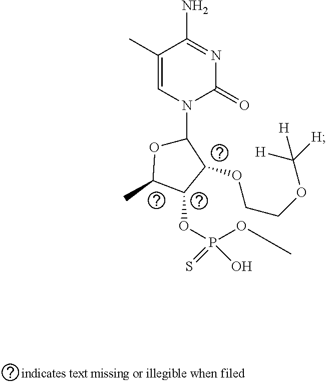

### X₁₁ is

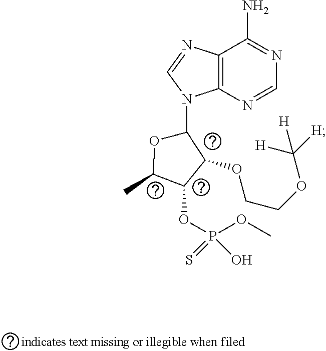

X₁₂ is

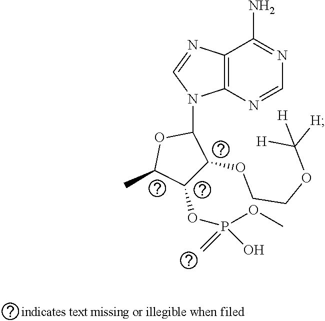

### X₁₄ is

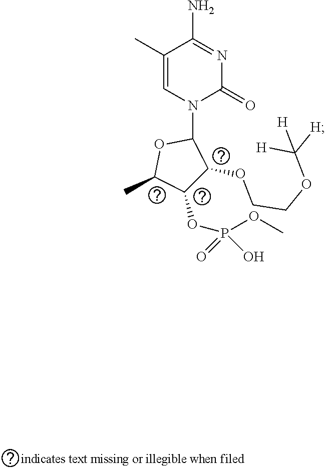

### X₁₆ is

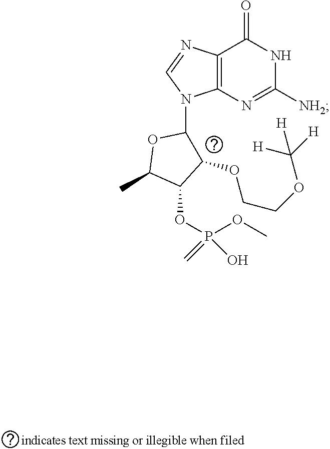 [SMILES: C=P(O)(OC)O[C@@H]1[C@@H](C)OC(n2cnc3c(=O)[nH]c(N)nc32)[C@@H]1OCCOC]

### and X_{B} is

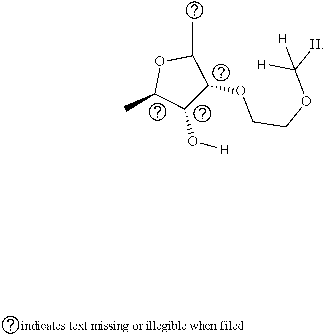

Provided herein is a compound of Formula (I):

(SEQ ID NO: 1)
X_{A}X₂X₃X₄X₅X₆X₇X₈X₉X₁₀X₁₁X₁₂X₁₃X₁₄X₁₅X₁₆X₁₇X_{B}

wherein
X_{A} is

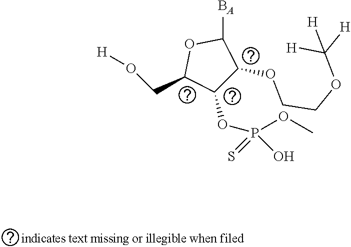

### and B_{A} is

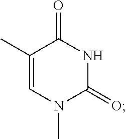

### X₂ is

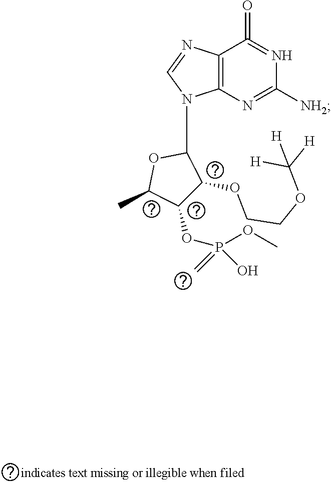

### X₃ is

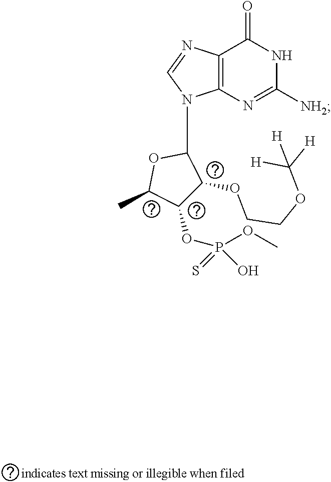 [SMILES: COCCO[C@H]1C(n2cnc3c(=O)[nH]c(N)nc32)O[C@H](C)[C@H]1OP(O)(=S)OC]

X₄ is

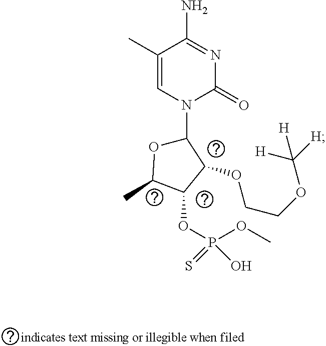

### X₅ is

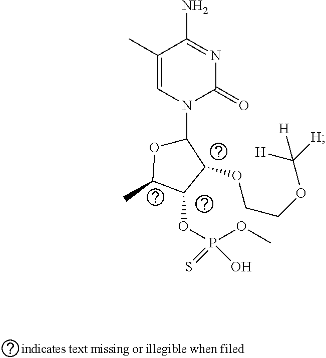

### X₆ is

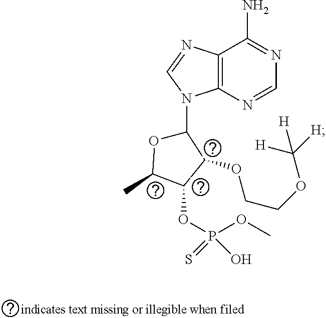

### X₇ is

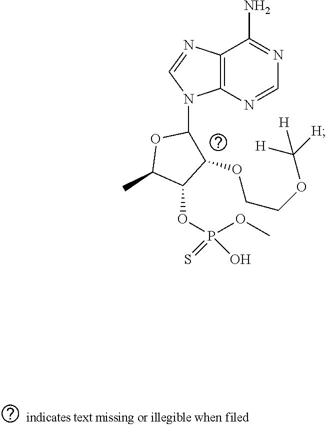

X₈ is

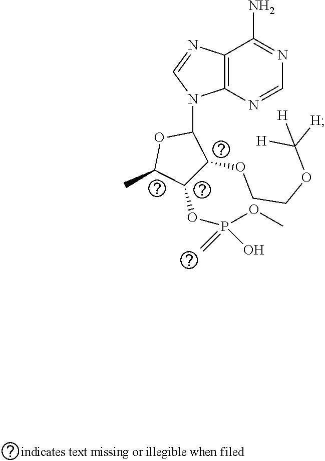

### X₉ is

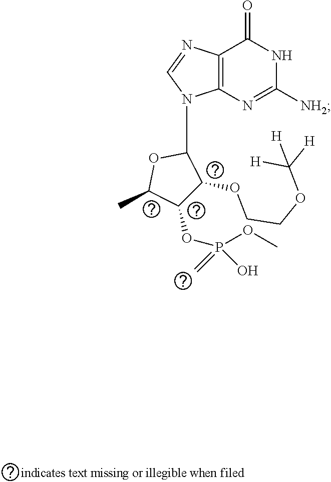 [SMILES: C=P(O)(OC)O[C@@H]1[C@@H](C)OC(n2cnc3c(=O)[nH]c(N)nc32)[C@@H]1OCCOC]

### X₁₀ is

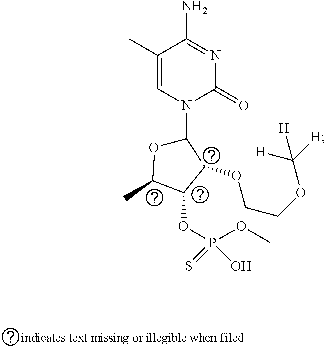

### X₁₁ is

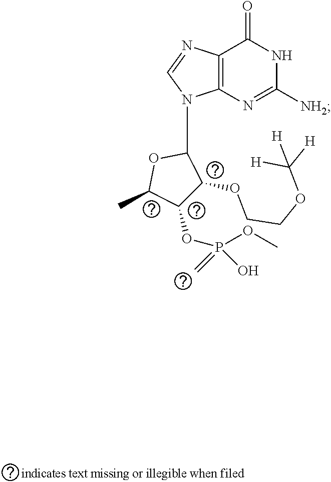 [SMILES: C=P(O)(OC)O[C@@H]1[C@@H](C)OC(n2cnc3c(=O)[nH]c(N)nc32)[C@@H]1OCCOC]

X₁₂ is

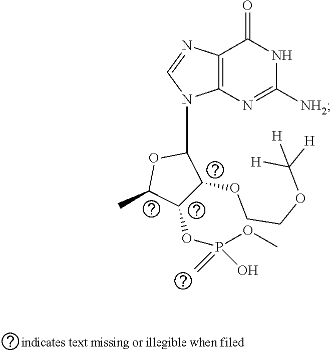 [SMILES: C=P(O)(OC)O[C@@H]1[C@@H](C)OC(n2cnc3c(=O)[nH]c(N)nc32)[C@@H]1OCCOC]

### X₁₃ is

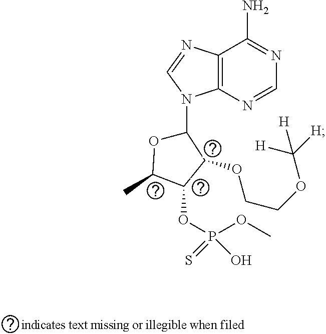

### X₁₄ is

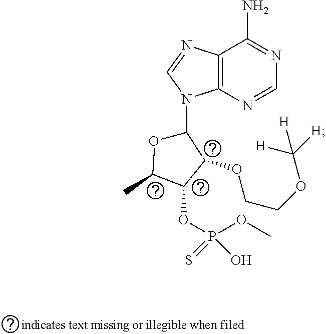

### X₁₅ is

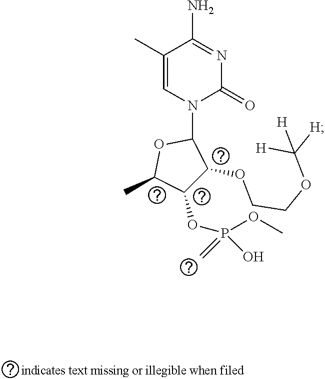

X₁₆ is

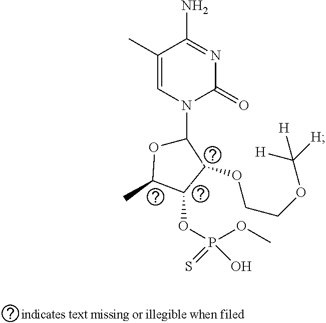

### X₁₇ is

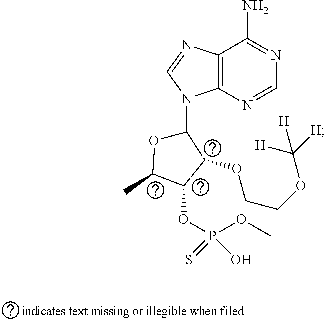

### and X_{B} is

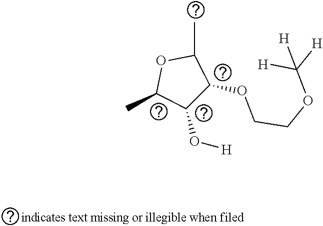

### and B_{B} is

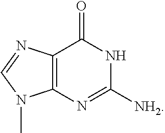

Provided herein is a compound of Formula (I):

(SEQ ID NO: 2)
X_{A}X₂X₃X₄X₅X₆X₇X₈X₉X₁₀X₁₁X₁₂X₁₃X₁₄X₁₅X_{B}

wherein
X_{A} is

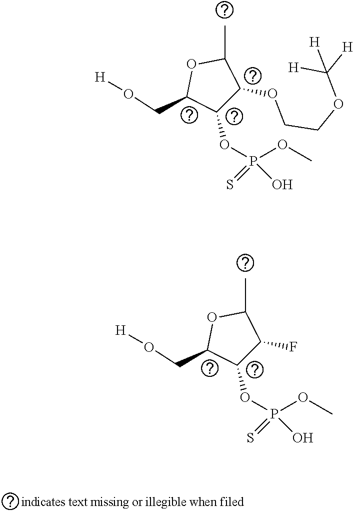 [SMILES: COCCO[C@H]1C(C)O[C@H](CO)[C@H]1OP(O)(=S)OC ; COP(O)(=S)O[C@@H]1[C@@H](CO)OC(C)[C@@H]1F]

### and B_{A} is

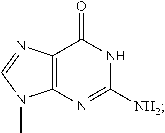

X₂ is

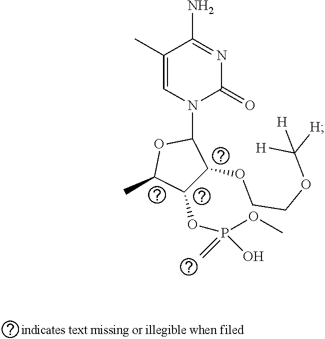

### X₃ is

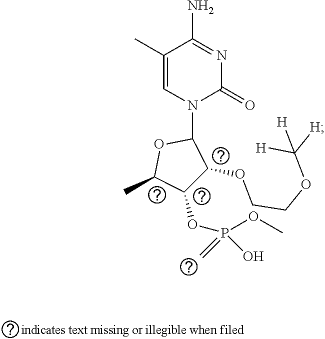

### X₄ is

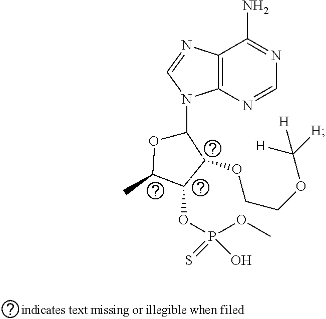

### X₅ is

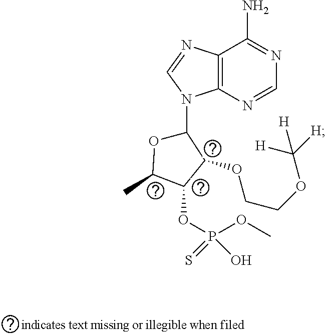

X₆ is

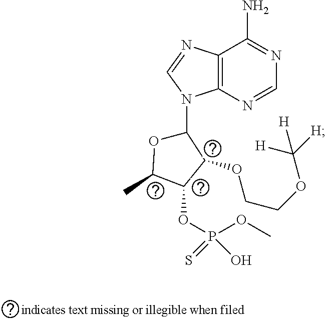

### X₇ is

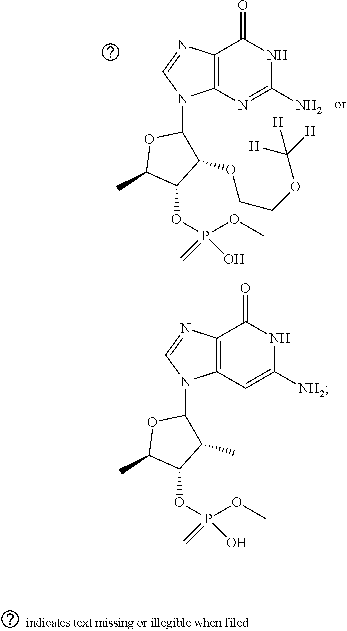

X₈ is

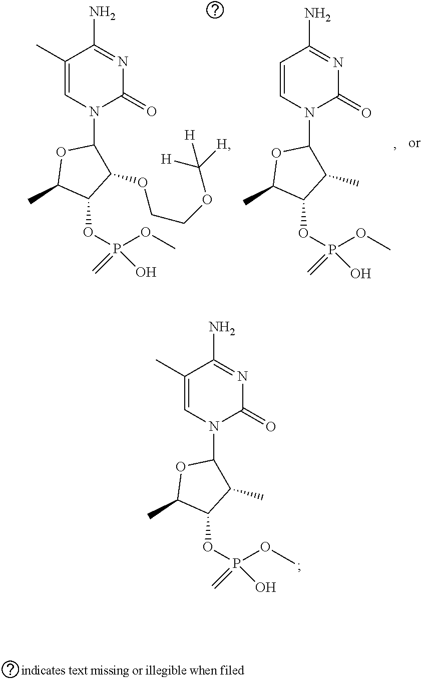

### X₉ is

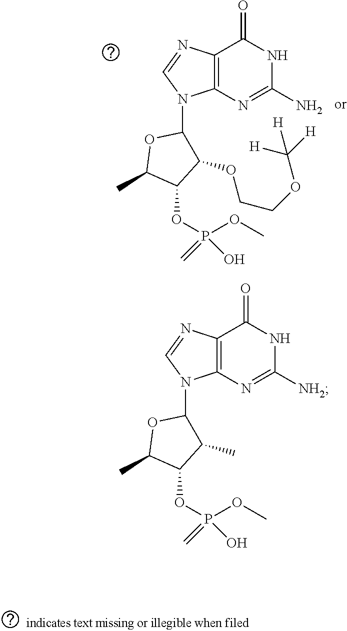

### X₁₀ is

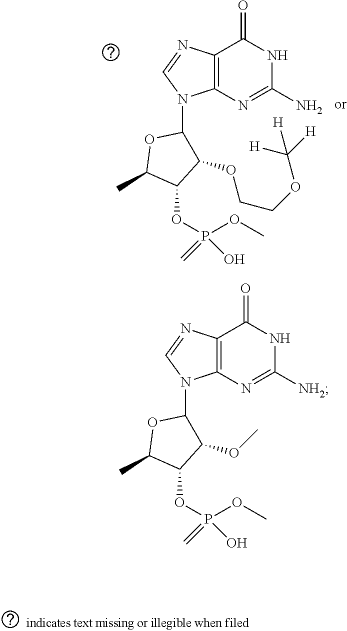

### X₁₁ is

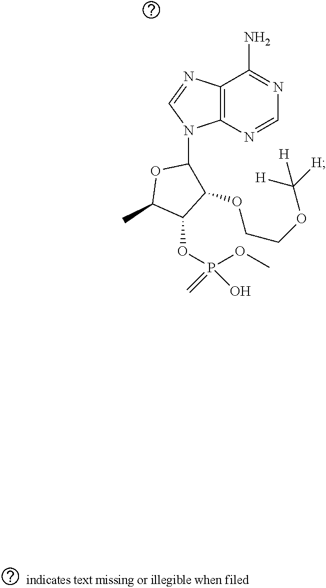

### X₁₂ is

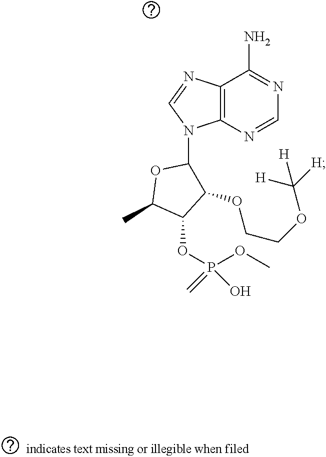

### X₁₃ is

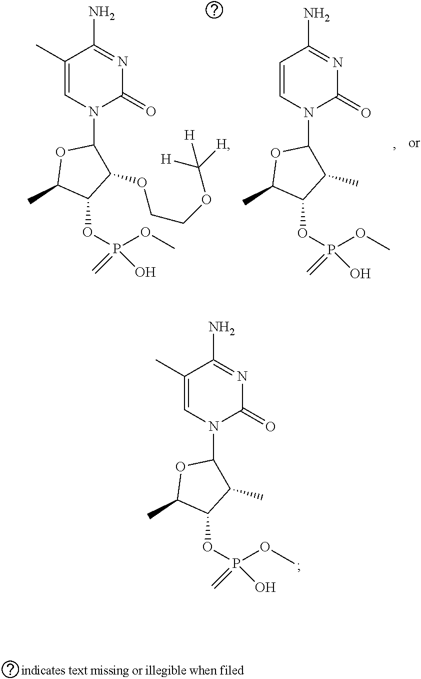

### X₁₄ is

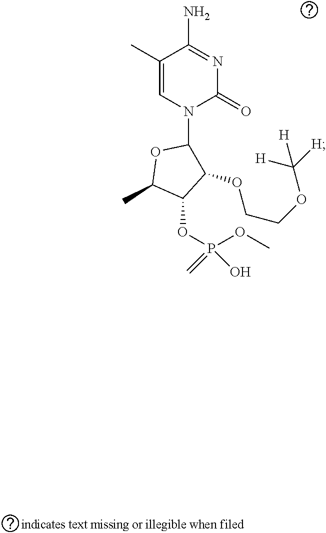

### X₁₅ is

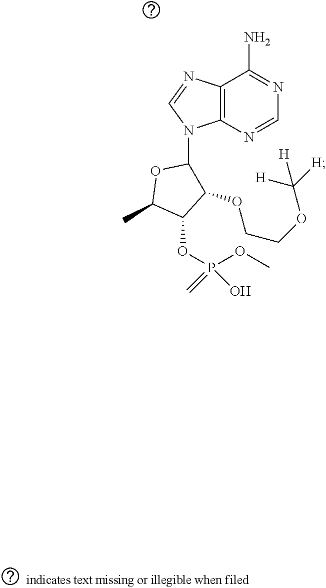

### and X_{B} is

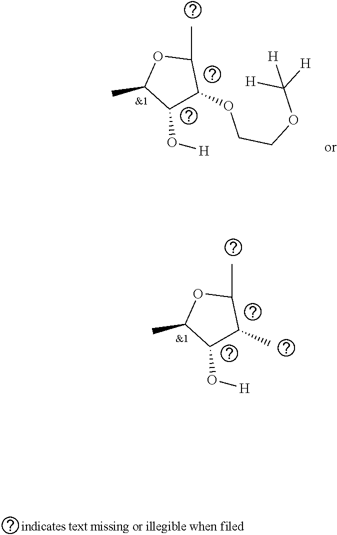

### and B_{B} is

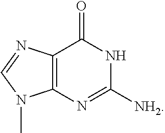

In some embodiments,
X_{A} is

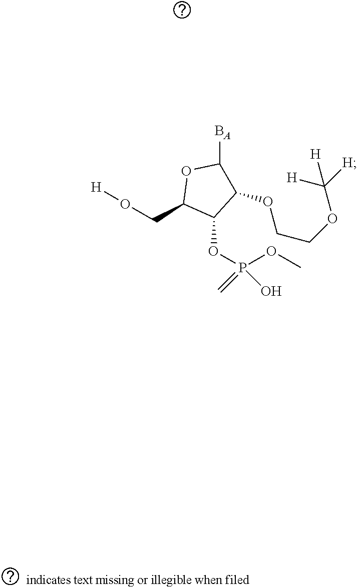

### X₇ is

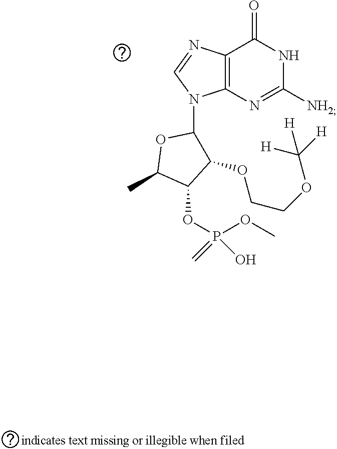 [SMILES: C=P(O)(OC)O[C@@H]1[C@@H](C)OC(n2cnc3c(=O)[nH]c(N)nc32)[C@@H]1OCCOC]

### X₈ is

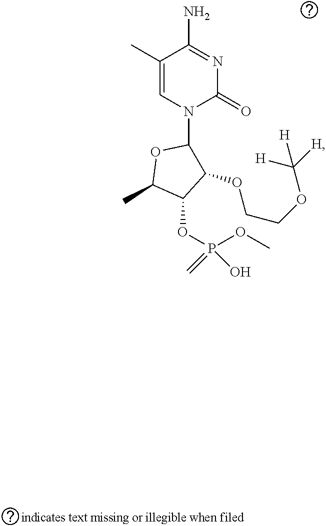

### X₉ is

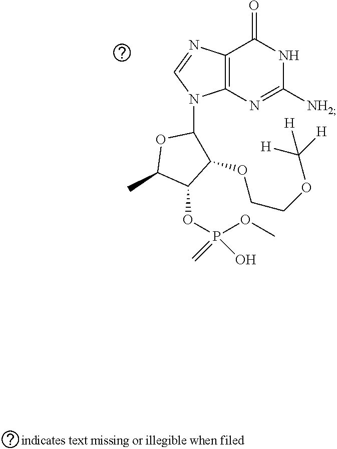 [SMILES: C=P(O)(OC)O[C@@H]1[C@@H](C)OC(n2cnc3c(=O)[nH]c(N)nc32)[C@@H]1OCCOC]

X₁₀ is

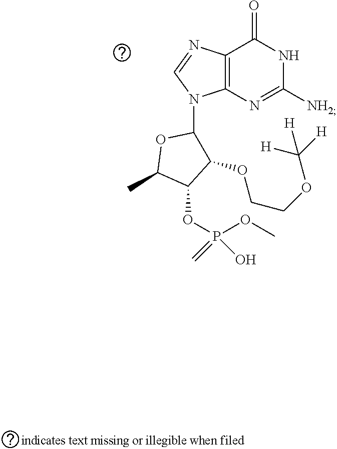 [SMILES: C=P(O)(OC)O[C@@H]1[C@@H](C)OC(n2cnc3c(=O)[nH]c(N)nc32)[C@@H]1OCCOC]

### X₁₃ is

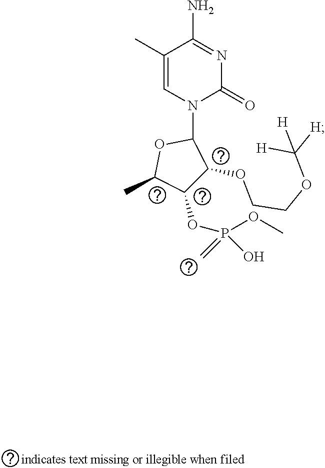

### and X_{B} is

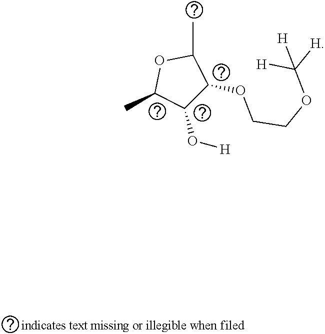

In some embodiments,
X_{A} is

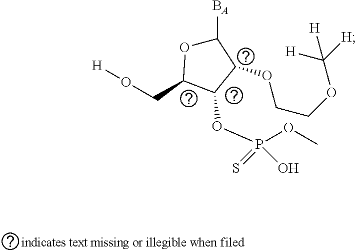

### X₇ is

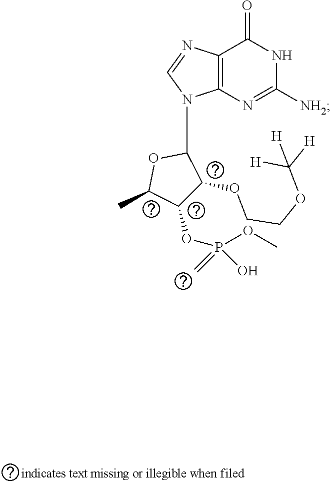 [SMILES: C=P(O)(OC)O[C@@H]1[C@@H](C)OC(n2cnc3c(=O)[nH]c(N)nc32)[C@@H]1OCCOC]

### X₈ is

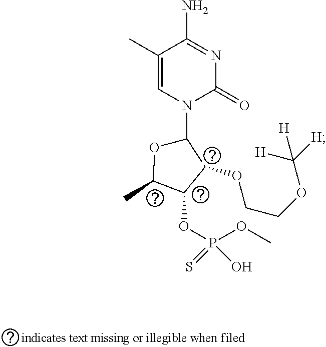

### X₉ is

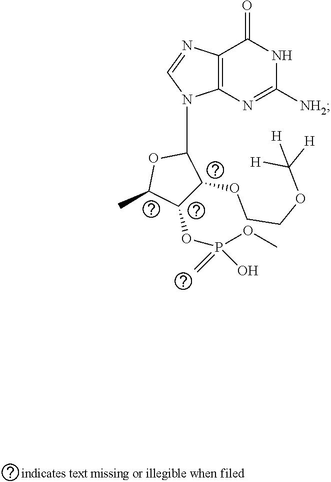 [SMILES: C=P(O)(OC)O[C@@H]1[C@@H](C)OC(n2cnc3c(=O)[nH]c(N)nc32)[C@@H]1OCCOC]

X₁₀ is

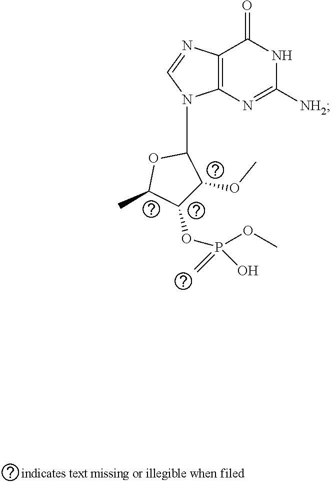 [SMILES: C=P(O)(OC)O[C@@H]1[C@@H](C)OC(n2cnc3c(=O)[nH]c(N)nc32)[C@@H]1OC]

### X₁₃ is

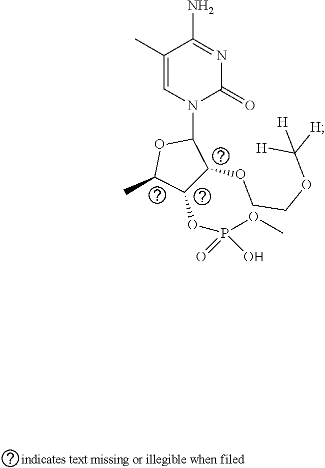

### and X_{B} is

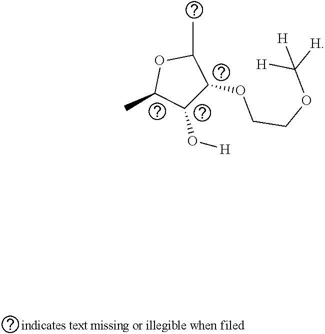

In some embodiments,
X_{A} is

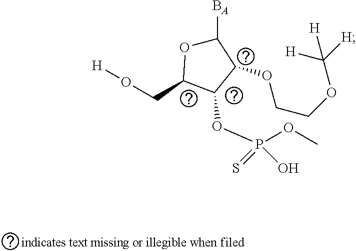

### X₇ is

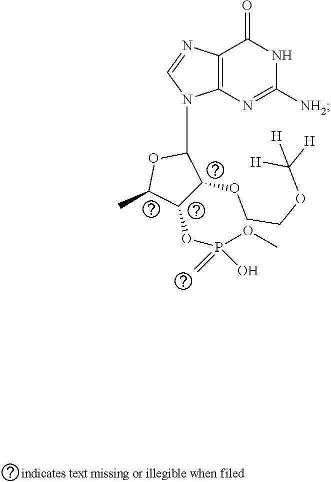 [SMILES: C=P(O)(OC)O[C@@H]1[C@@H](C)OC(n2cnc3c(=O)[nH]c(N)nc32)[C@@H]1OCCOC]

### X₈ is

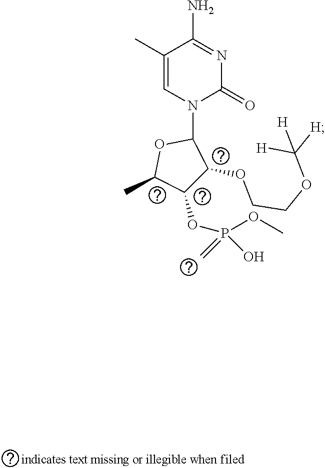

### X₉ is

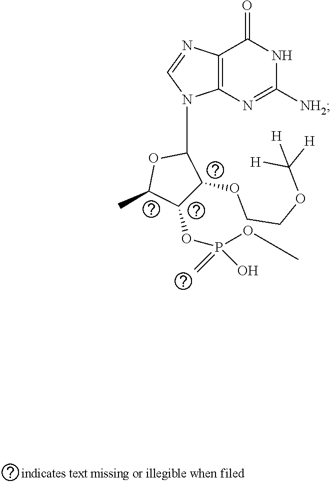 [SMILES: C=P(O)(OC)O[C@@H]1[C@@H](C)OC(n2cnc3c(=O)[nH]c(N)nc32)[C@@H]1OCCOC]

X₁₀ is

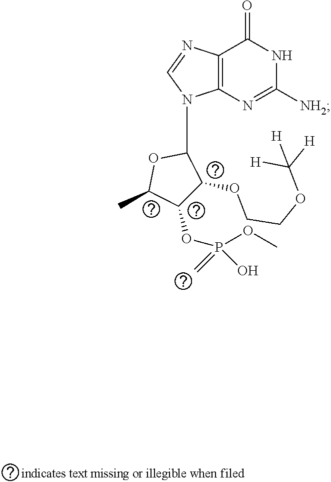 [SMILES: C=P(O)(OC)O[C@@H]1[C@@H](C)OC(n2cnc3c(=O)[nH]c(N)nc32)[C@@H]1OCCOC]

### X₁₃ is

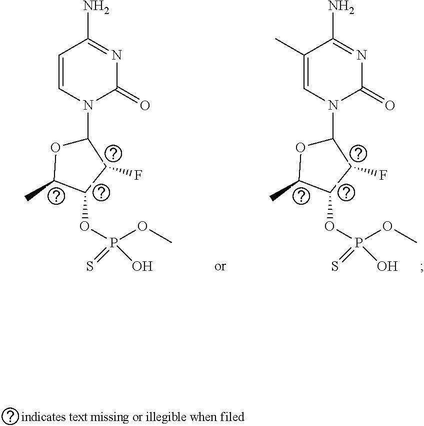

### and X_{B} is

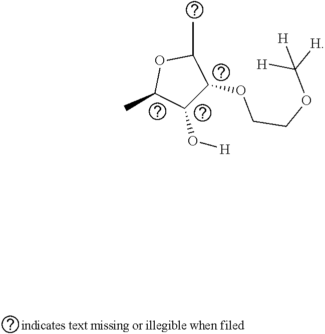

In some embodiments,
X_{A} is

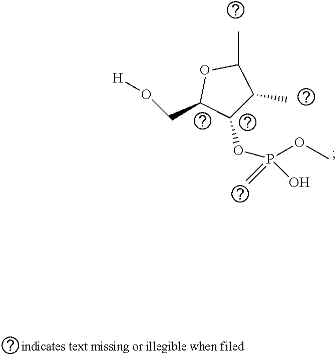

### X₇ is

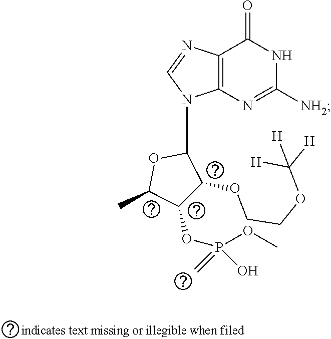 [SMILES: C=P(O)(OC)O[C@@H]1[C@@H](C)OC(n2cnc3c(=O)[nH]c(N)nc32)[C@@H]1OCCOC]

### X₈ is

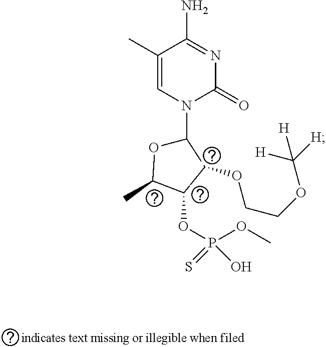

### X₉ is

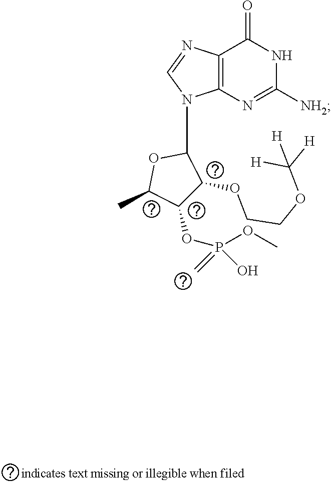 [SMILES: C=P(O)(OC)O[C@@H]1[C@@H](C)OC(n2cnc3c(=O)[nH]c(N)nc32)[C@@H]1OCCOC]

### X₁₀ is

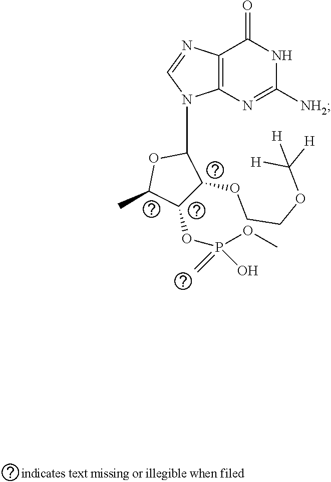 [SMILES: C=P(O)(OC)O[C@@H]1[C@@H](C)OC(n2cnc3c(=O)[nH]c(N)nc32)[C@@H]1OCCOC]

### X₁₃ is

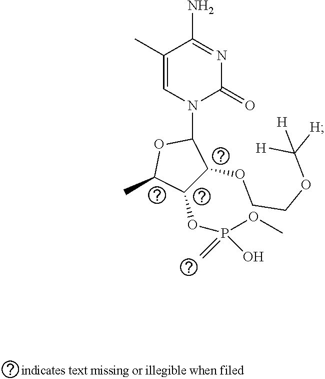

### and X_{B} is

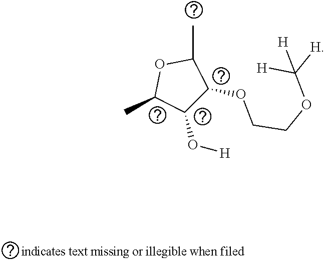

In some embodiments,
X_{A} is

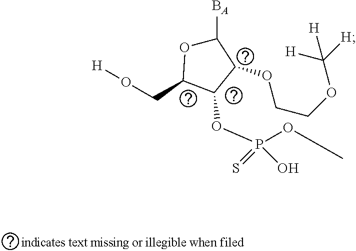

### X₇ is

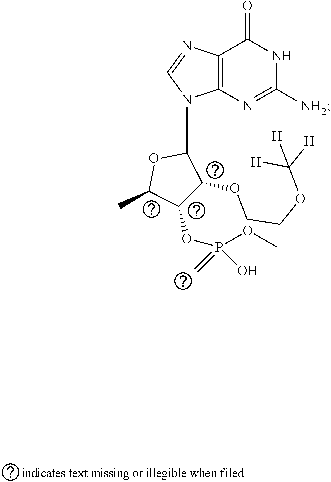 [SMILES: C=P(O)(OC)O[C@@H]1[C@@H](C)OC(n2cnc3c(=O)[nH]c(N)nc32)[C@@H]1OCCOC]

### X₈ is

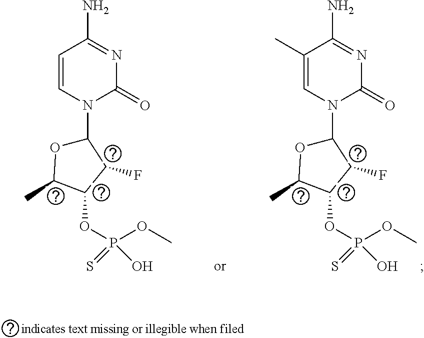

X₉ is

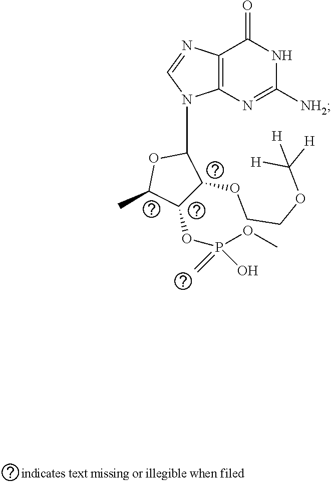 [SMILES: C=P(O)(OC)O[C@@H]1[C@@H](C)OC(n2cnc3c(=O)[nH]c(N)nc32)[C@@H]1OCCOC]

### X₁₀ is

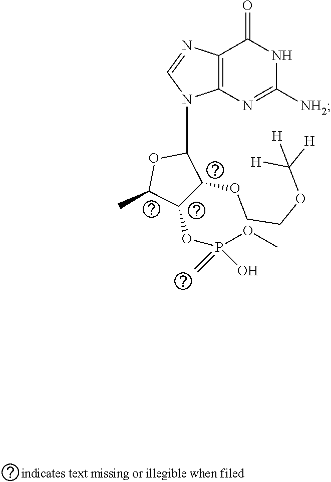 [SMILES: C=P(O)(OC)O[C@@H]1[C@@H](C)OC(n2cnc3c(=O)[nH]c(N)nc32)[C@@H]1OCCOC]

### X₁₃ is

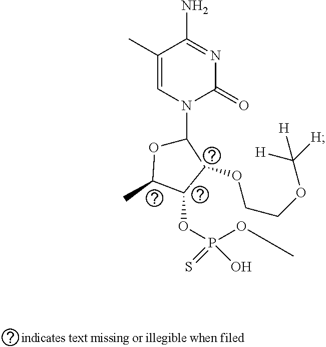

### and X_{B} is

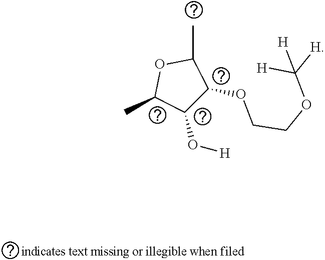

In some embodiments,
X_{A} is

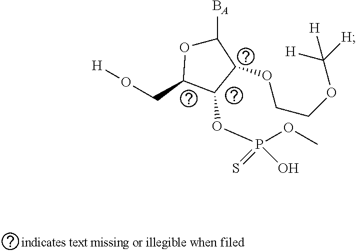

### X₇ is

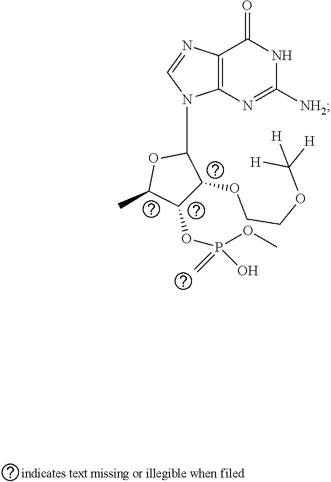 [SMILES: C=P(O)(OC)O[C@@H]1[C@@H](C)OC(n2cnc3c(=O)[nH]c(N)nc32)[C@@H]1OCCOC]

### X₈ is

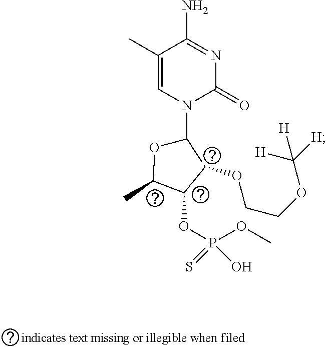

### X₉ is

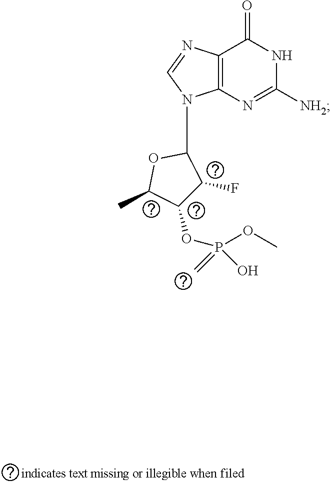 [SMILES: C=P(O)(OC)O[C@@H]1[C@@H](C)OC(n2cnc3c(=O)[nH]c(N)nc32)[C@@H]1F]

X₁₀ is

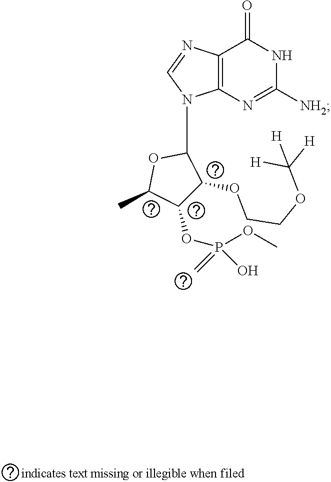 [SMILES: C=P(O)(OC)O[C@@H]1[C@@H](C)OC(n2cnc3c(=O)[nH]c(N)nc32)[C@@H]1OCCOC]

### X₁₃ is

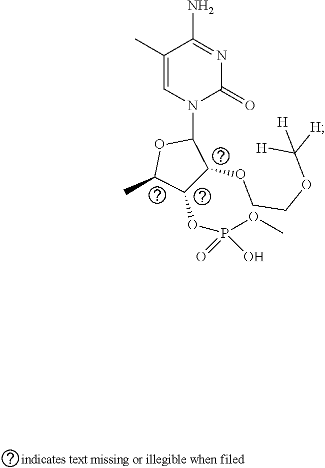

### and X_{B} is

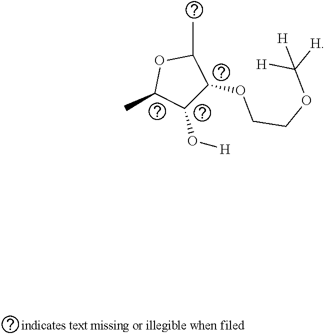

In some embodiments,
X_{A} is

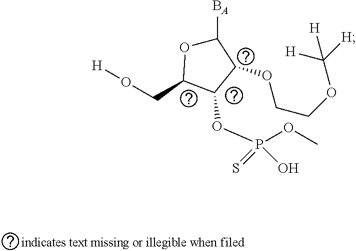

### X₇ is

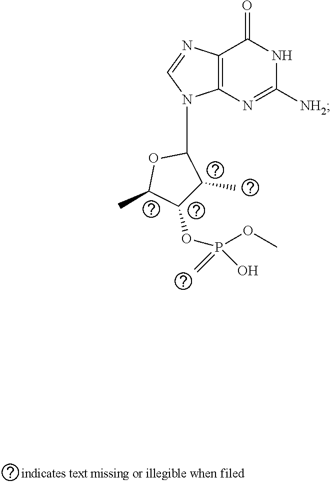 [SMILES: C=P(O)(OC)O[C@@H]1[C@@H](C)OC(n2cnc3c(=O)[nH]c(N)nc32)[C@@H]1C]

### X₈ is

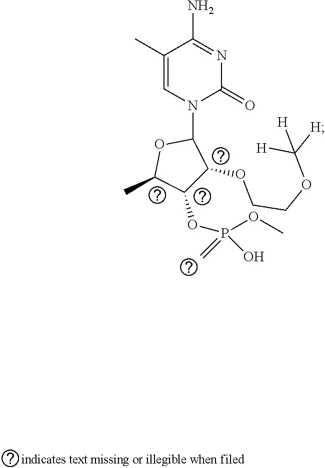

### X₉ is

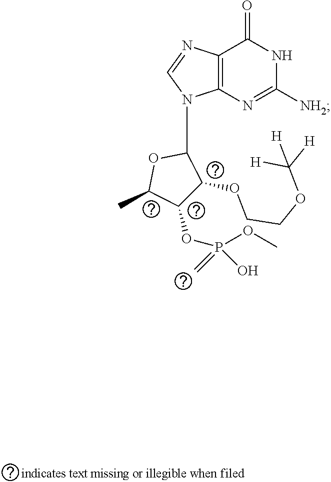 [SMILES: C=P(O)(OC)O[C@@H]1[C@@H](C)OC(n2cnc3c(=O)[nH]c(N)nc32)[C@@H]1OCCOC]

X₁₀ is

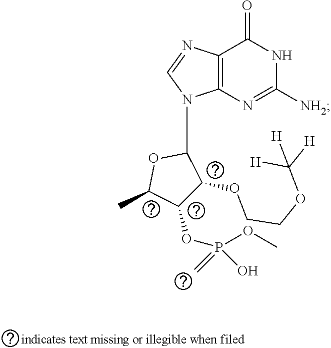 [SMILES: C=P(O)(OC)O[C@@H]1[C@@H](C)OC(n2cnc3c(=O)[nH]c(N)nc32)[C@@H]1OCCOC]

### X₁₃ is

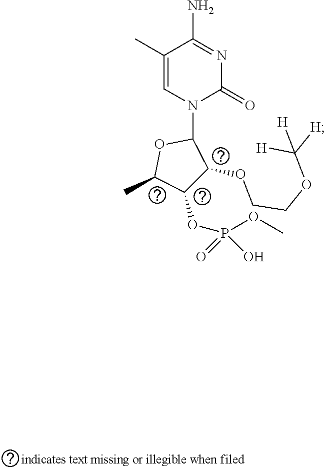

### and X_{B} is

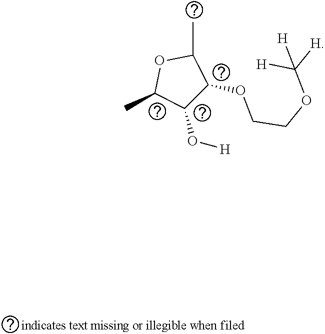

In some embodiments,
X_{A} is

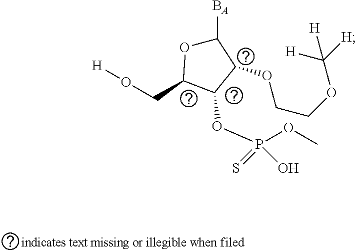

### X₇ is

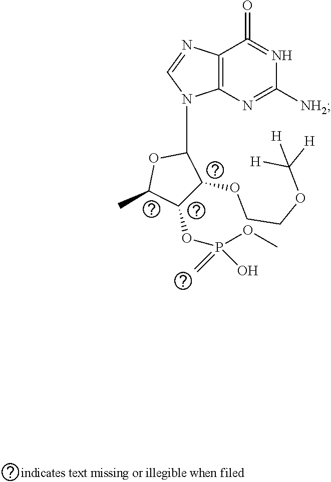

### X₈ is

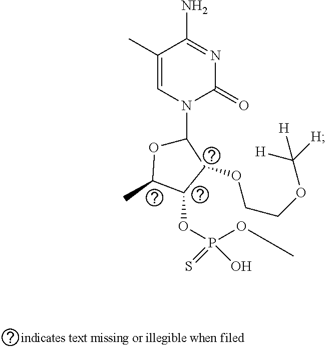

### X₉ is

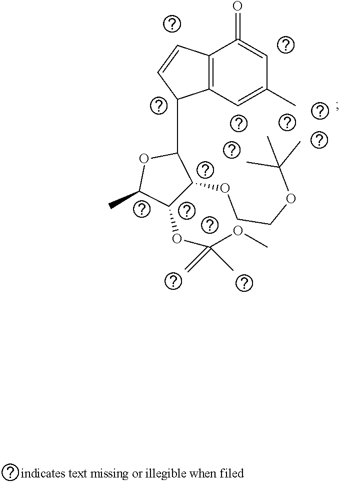

X₁₀ is

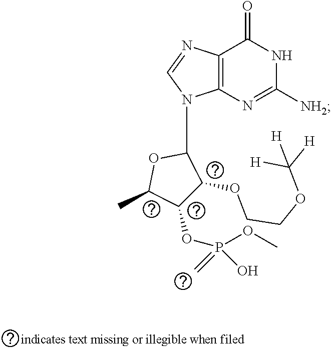 [SMILES: C=P(O)(OC)O[C@@H]1[C@@H](C)OC(n2cnc3c(=O)[nH]c(N)nc32)[C@@H]1OCCOC]

### X₁₃ is

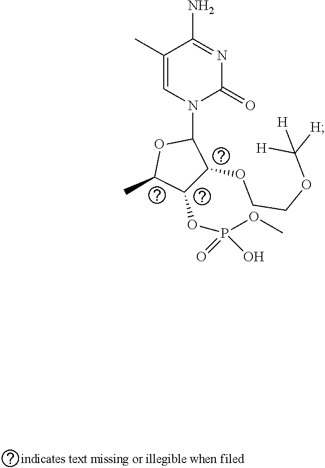

### and X_{B} is

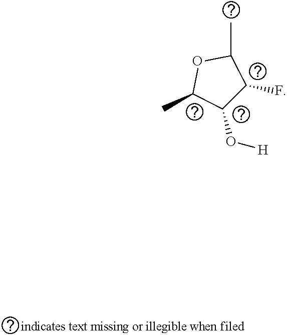

Provided herein is a compound of Formula (I):

(SEQ ID NO: 3)
X_{A}X₂X₃X₄X₅X₆X₇X₈X₉X₁₀X₁₁X₁₂X₁₃X₁₄X₁₅X_{B}

wherein
X_{A} is

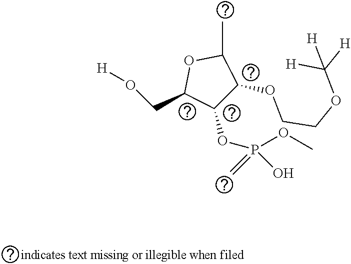 [SMILES: C=P(O)(OC)O[C@@H]1[C@@H](CO)OC(C)[C@@H]1OCCOC]

### and B_{A} is

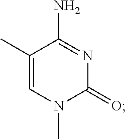

### X₂ is

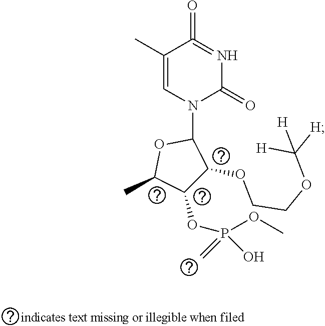

### X₃ is

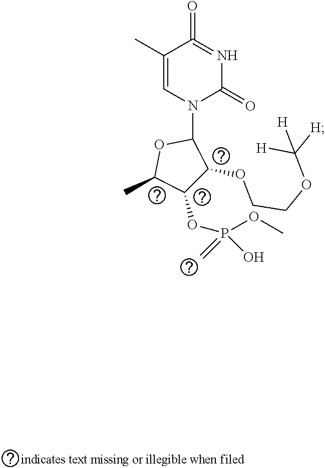

X₄ is

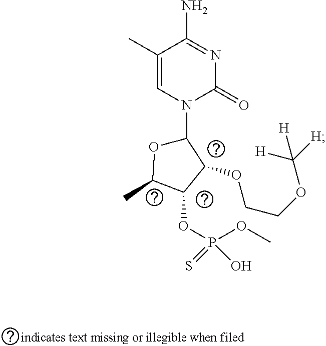

### X₅ is

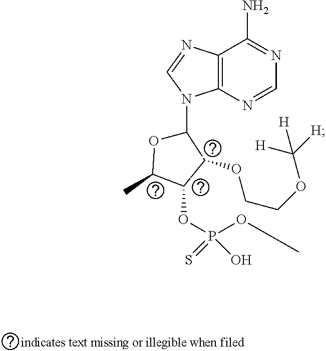

### X₆ is

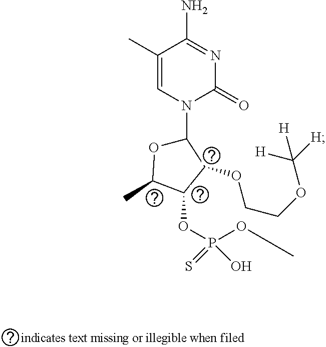

### X₇ is

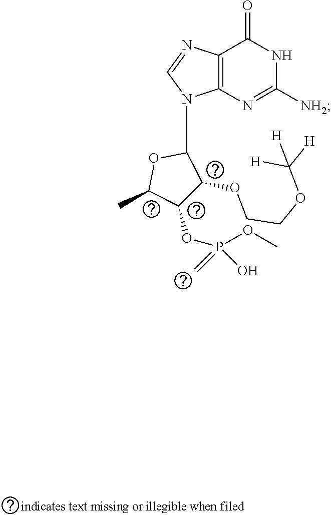 [SMILES: C=P(O)(OC)O[C@@H]1[C@@H](C)OC(n2cnc3c(=O)[nH]c(N)nc32)[C@@H]1OCCOC]

X₈ is

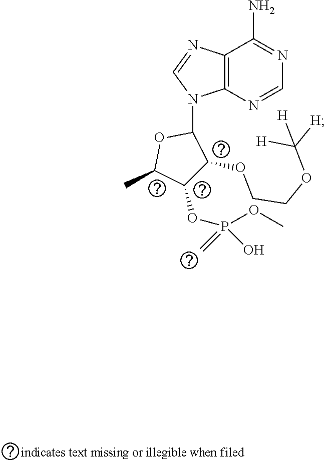

### X₉ is

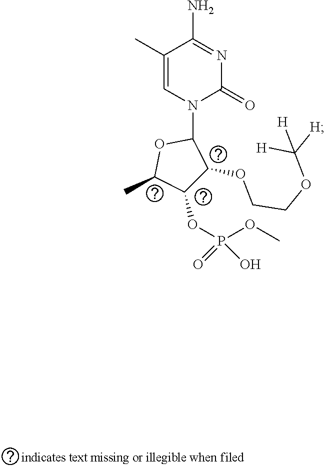

### X₁₀ is

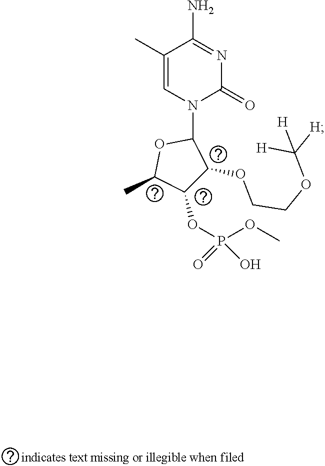

### X₁₁ is

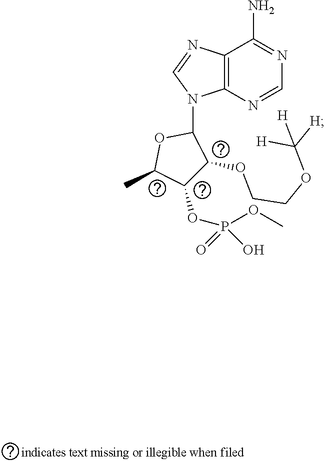

X₁₂ is

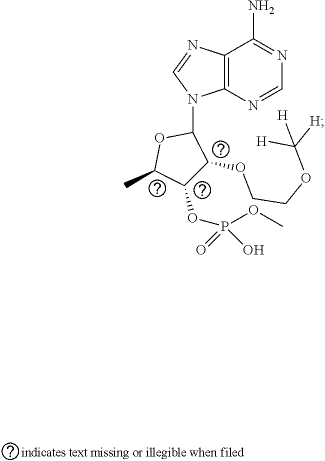

### X₁₃ is

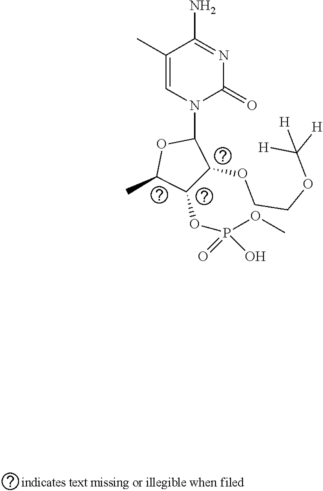 [SMILES: COCCO[C@H]1C(n2cc(C)c(N)nc2=O)O[C@H](C)[C@H]1OP(=O)(O)OC]

or absent, X₁₄ is

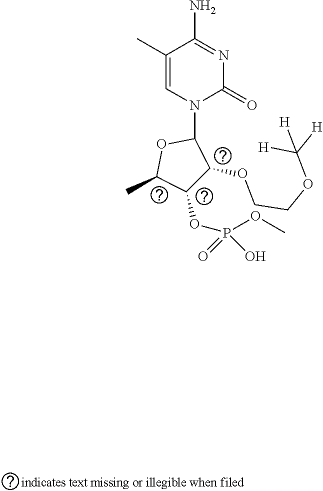 [SMILES: COCCO[C@H]1C(n2cc(C)c(N)nc2=O)O[C@H](C)[C@H]1OP(=O)(O)OC]

or absent;
X₁₅ is

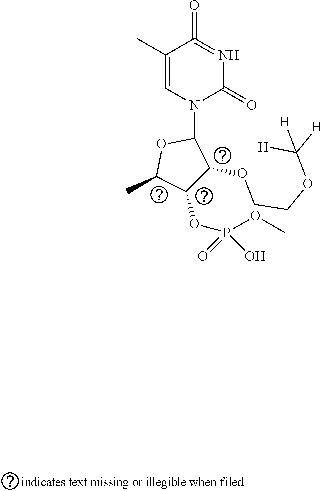

or absent; and X_{B} is

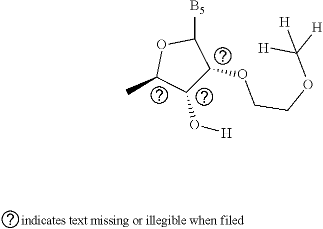

### and B_{B} is

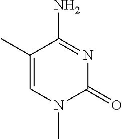

when each of X₁₃ X₁₄ and X₁₅ is absent,

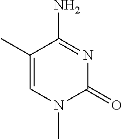

when X₁₃ is

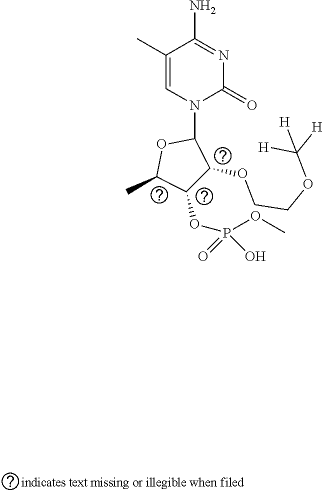 [SMILES: COCCO[C@H]1C(n2cc(C)c(N)nc2=O)O[C@H](C)[C@H]1OP(=O)(O)OC]

and each of X₁₄ and X₁₅ is absent,

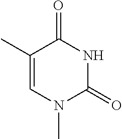

when X₁₃ is

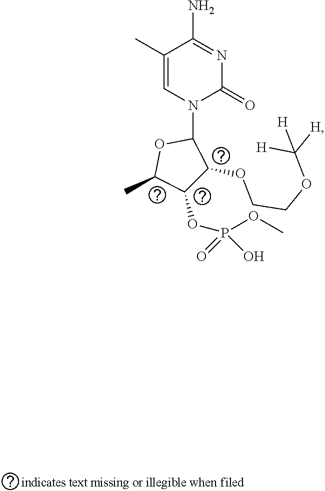

### X₁₄ is

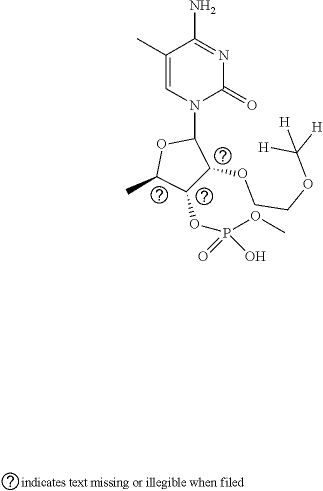 [SMILES: COCCO[C@H]1C(n2cc(C)c(N)nc2=O)O[C@H](C)[C@H]1OP(=O)(O)OC]

and X₁₅ is absent, or

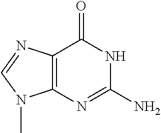

when X₁₃ is

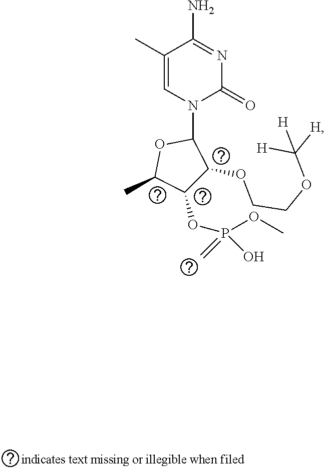

### X₁₄ is

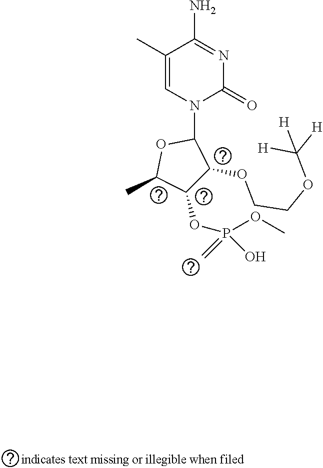 [SMILES: C=P(O)(OC)O[C@@H]1[C@@H](C)OC(n2cc(C)c(N)nc2=O)[C@@H]1OCCOC]

### and X₁₅ is

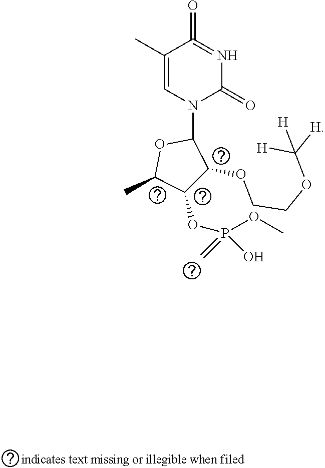

In some embodiments,
X₁₃ is

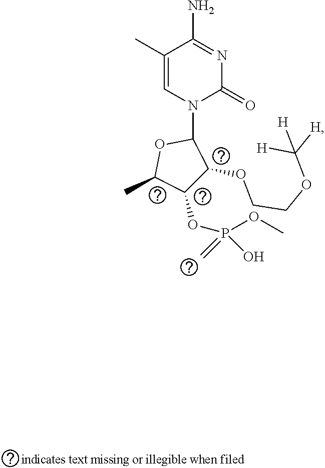

### X₁₄ is

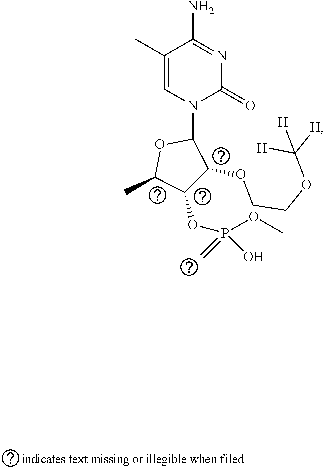

### and X₁₅ is

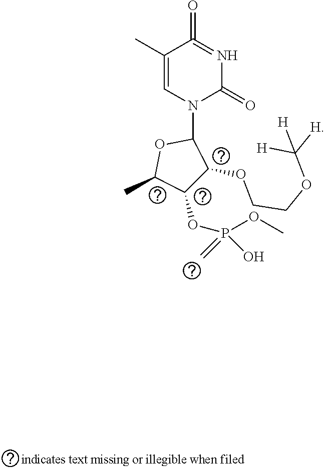

In some embodiments,
X₁₃ is

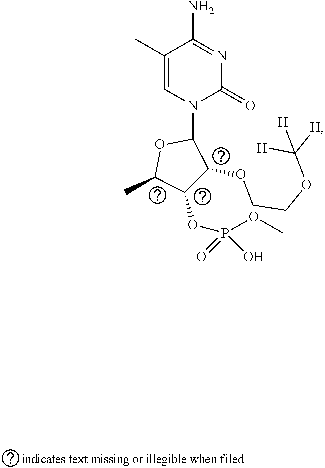

### X₁₄ is

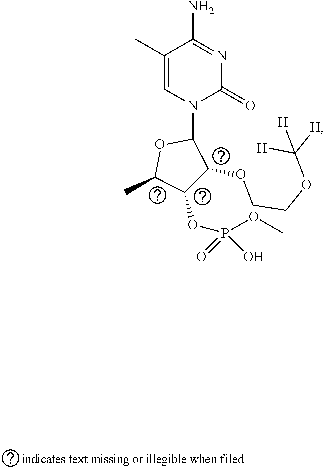

and X₁₅ is absent.

In some embodiments,
X₁₃ is

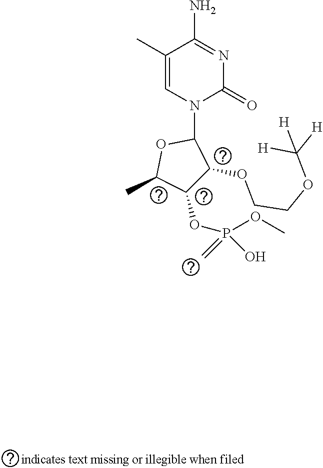 [SMILES: C=P(O)(OC)O[C@@H]1[C@@H](C)OC(n2cc(C)c(N)nc2=O)[C@@H]1OCCOC]

and each of X₁₄ and X₁₅ is absent.

In some embodiments, each of X₁₃, X₁₄, and X₁₅ is absent.

Provided herein is a compound of Formula (I):

(SEQ ID NO: 4)
X_{A}X₂X₃X₄X₅X₆X₇X₈X₉X₁₀X₁₁X₁₂X₁₃X₁₄X₁₅X_{B}

wherein
X_{A} is

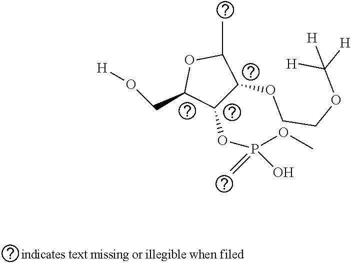 [SMILES: C=P(O)(OC)O[C@@H]1[C@@H](CO)OC(C)[C@@H]1OCCOC]

### and B_{A} is

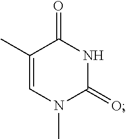

### X₂ is

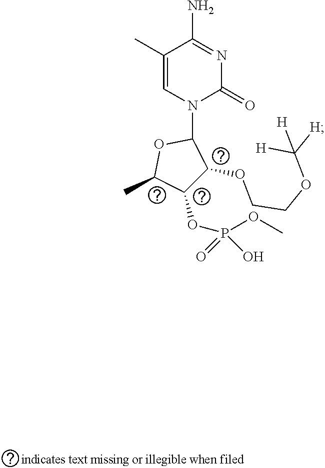

### X₃ is

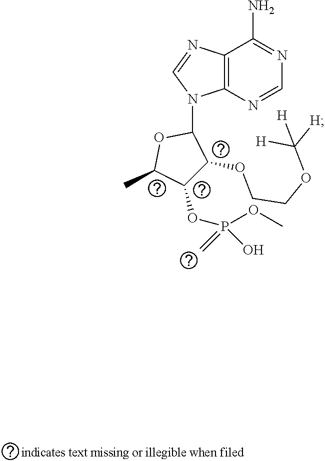

X₄ is

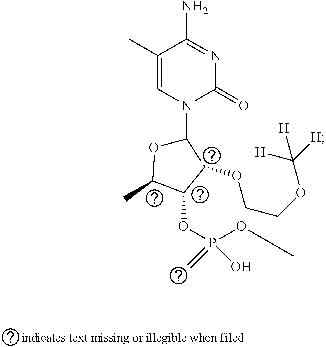

### X₅ is

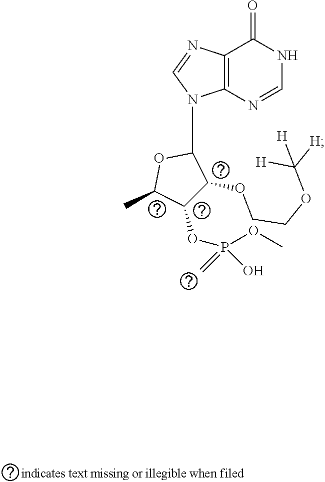

### X₆ is

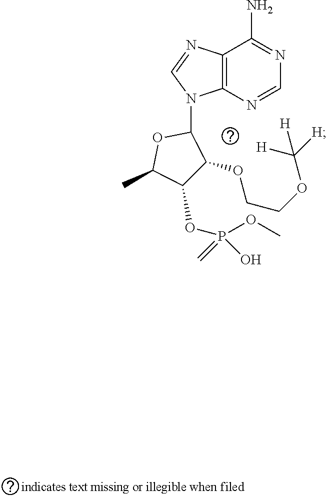

### X₇ is

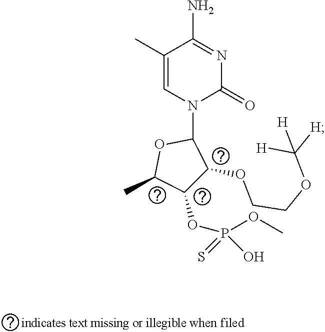

X₈ is

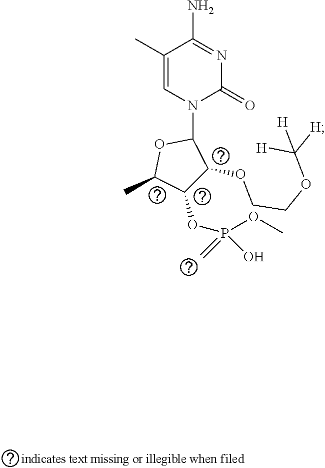

### X₉ is

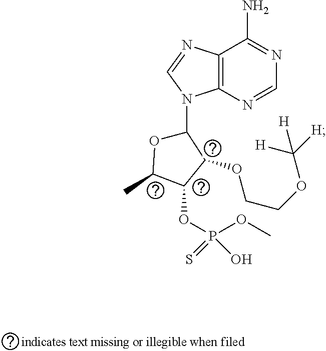

### X₁₀ is

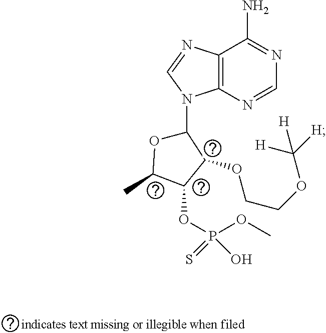

### X₁₁ is

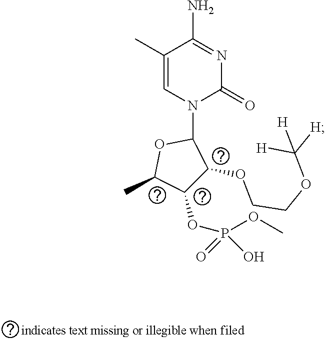

X₁₂ is

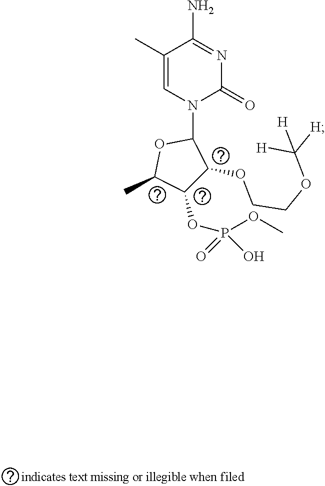

### X₁₃ is

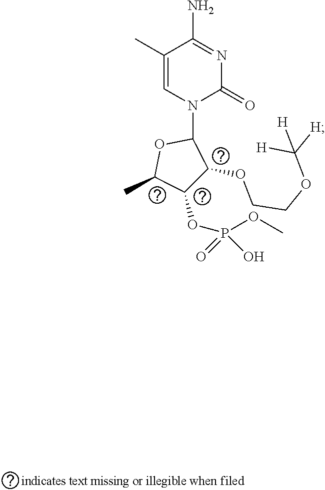

### and X₁₄ is

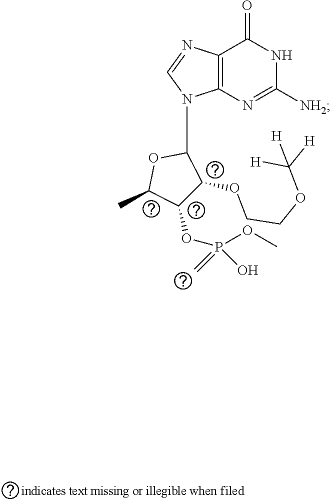 [SMILES: C=P(O)(OC)O[C@@H]1[C@@H](C)OC(n2cnc3c(=O)[nH]c(N)nc32)[C@@H]1OCCOC]

### X₁₅ is

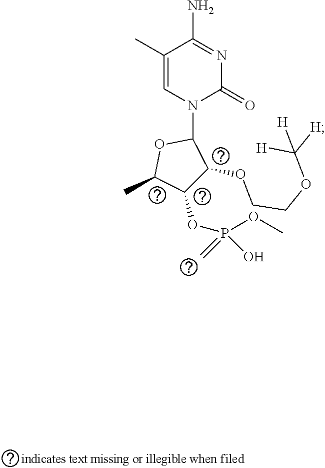

X_{B} is

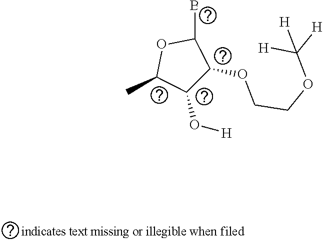

### and B_{B} is

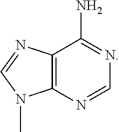

Provided herein is a compound of Formula (I):

(SEQ ID NO: 5)
X_{A}X₂X₃X₄X₅X₆X₇X₈X₉X₁₀X₁₁X₁₂X₁₃X₁₄X_{B}

wherein:
X_{A} is

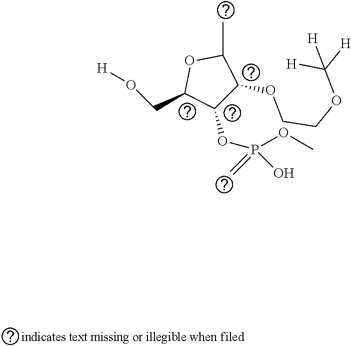 [SMILES: C=P(O)(OC)O[C@@H]1[C@@H](CO)OC(C)[C@@H]1OCCOC]

### and B_{A} is

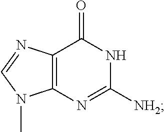

### X₂ is

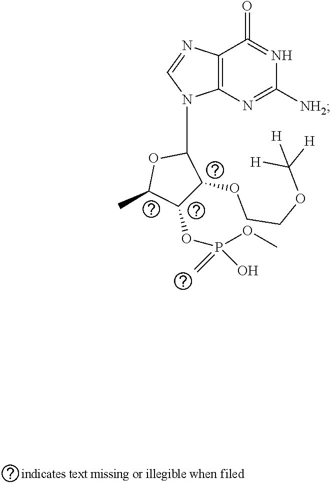

X₃ is

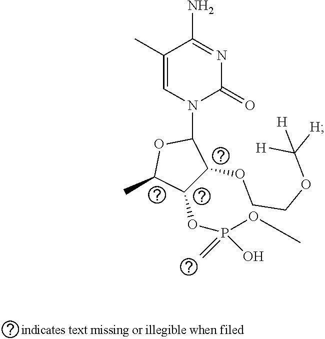

### X₄ is

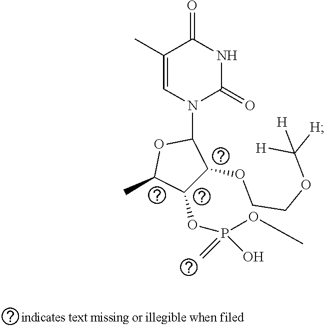

### X₅ is

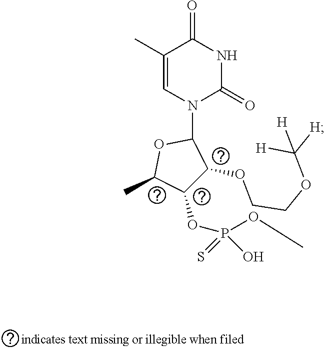

### X₆ is

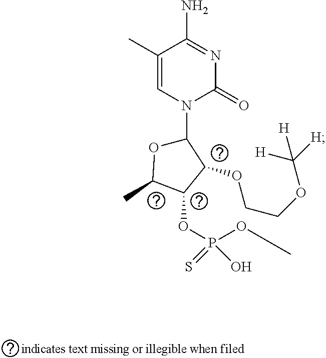

X₇ is

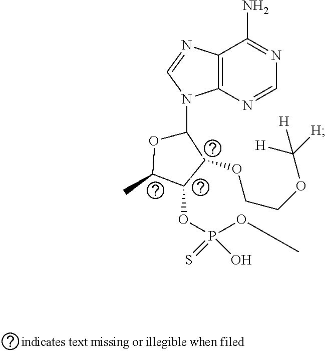

### X₈ is

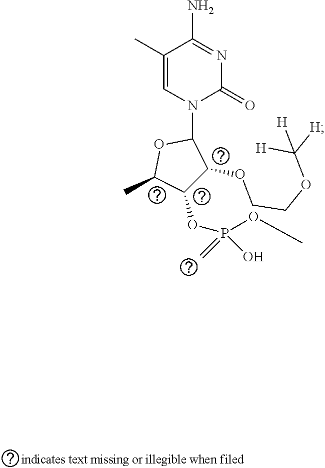

### X₉ is

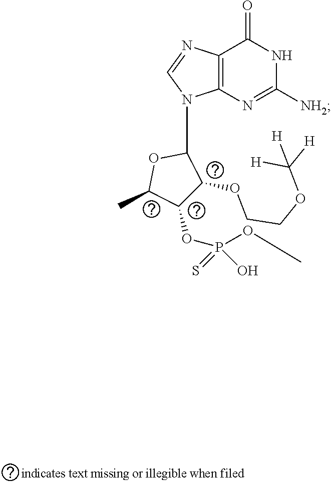 [SMILES: COCCO[C@H]1C(n2cnc3c(=O)[nH]c(N)nc32)O[C@H](C)[C@H]1OP(O)(=S)OC]

### X₁₀ is

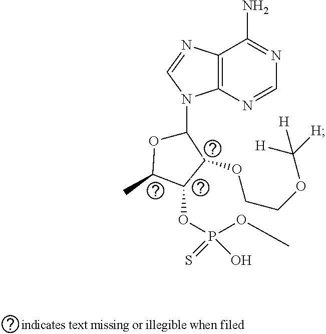

X₁₁ is

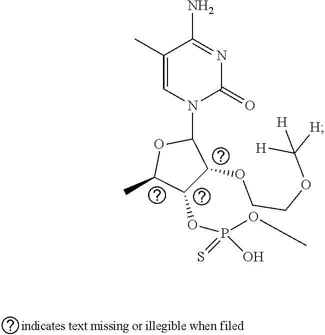

### X₁₂ is

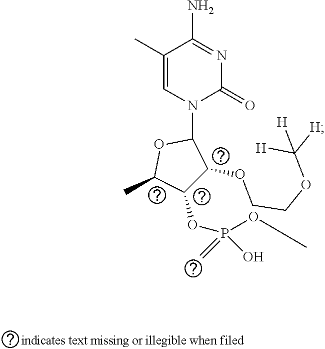

### X₁₃ is

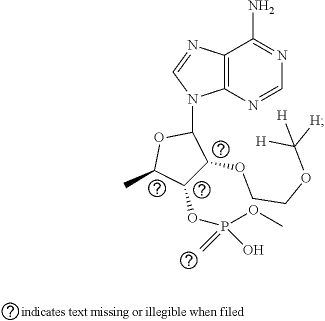

### X₁₄ is

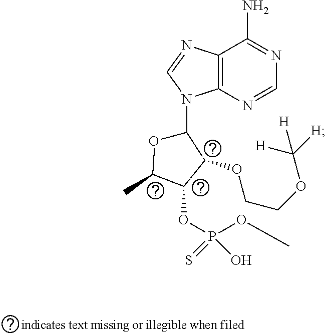

and
X_{B} is

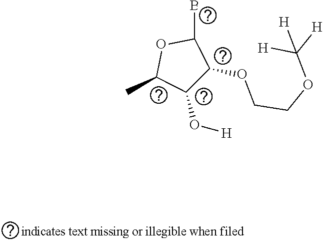

### and B_{B} is

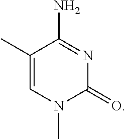

Provided herein is a compound of Formula (I):

(SEQ ID NO: 6)
X_{A}X₂X₃X₄X₅X₆X₇X₈X₉X₁₀X₁₁X₁₂X₁₃X_{B}

wherein
X_{A} is

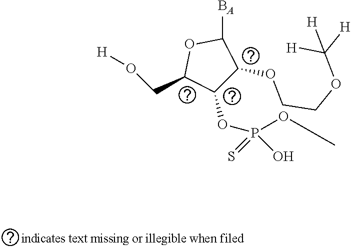

### and B_{A} is

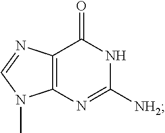

### X₂ is

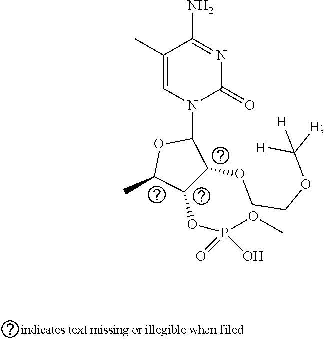

X₃ is

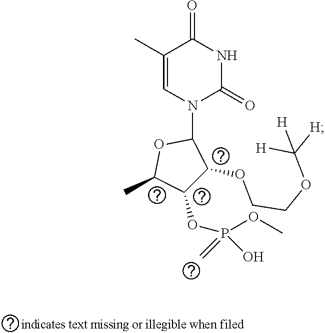

### X₄ is

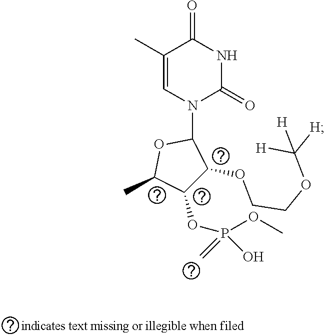

### X₅ is

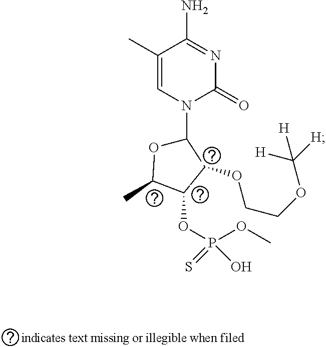

### X₆ is

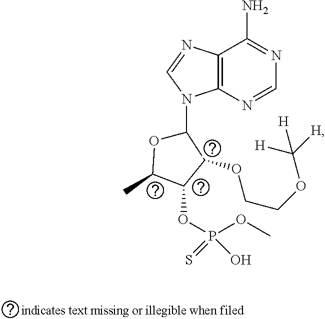

X₇ is

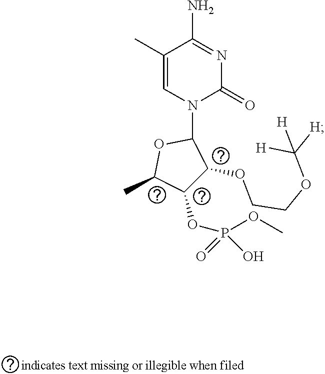

### X₈ is

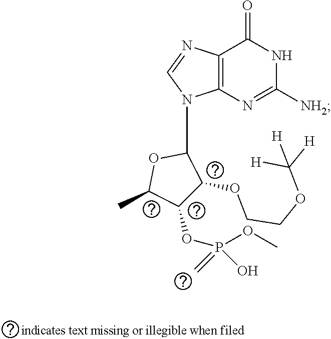 [SMILES: C=P(O)(OC)O[C@@H]1[C@@H](C)OC(n2cnc3c(=O)[nH]c(N)nc32)[C@@H]1OCCOC]

### X₉ is

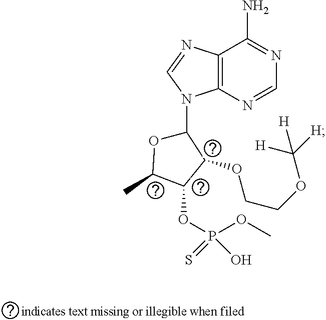

### X₁₀ is

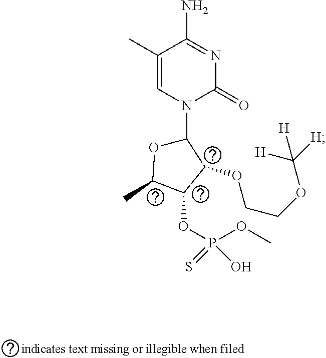

X₁₁ is

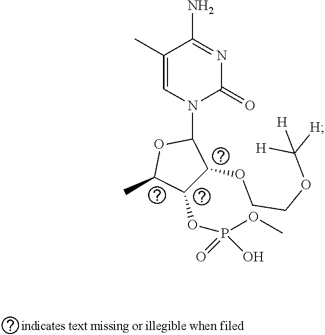

### X₁₂ is

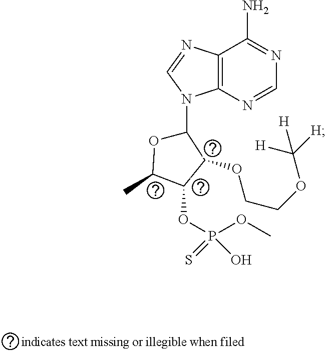

### X₁₃ is

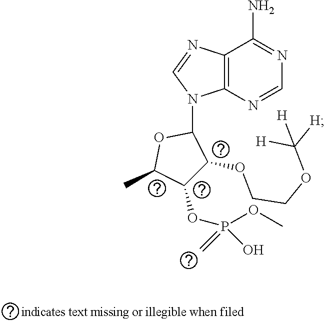

### and X_{B} is

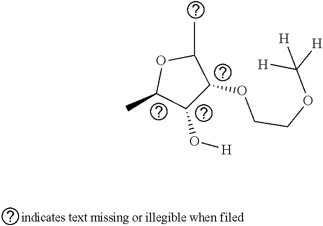

and B_{B} is

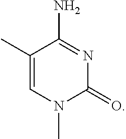

Provided herein is a compound of Formula (I):

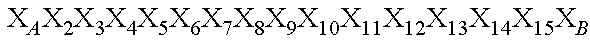

wherein
X_{A} is

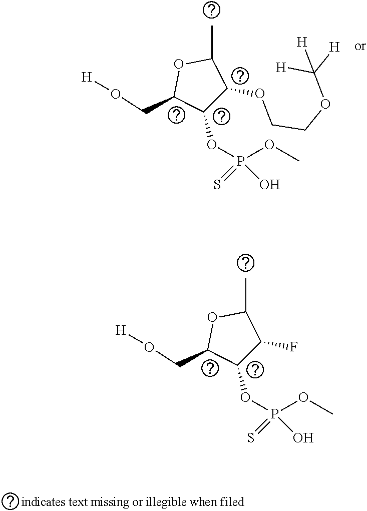 [SMILES: COCCO[C@H]1C(C)O[C@H](CO)[C@H]1OP(O)(=S)OC ; "or" ; COP(O)(=S)O[C@@H]1[C@@H](CO)OC(C)[C@@H]1F]

### and B_{A} is

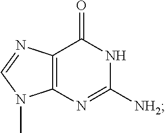

X₂ is

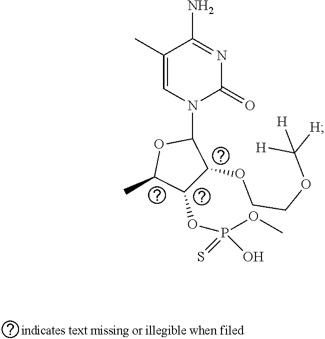

### X₃ is

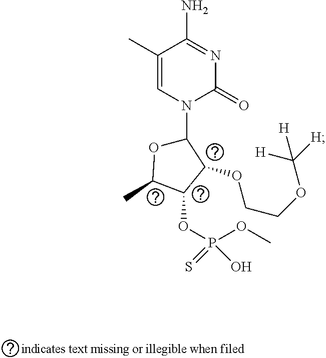

### X₄ is

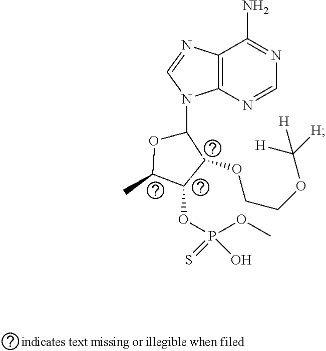

### X₅ is

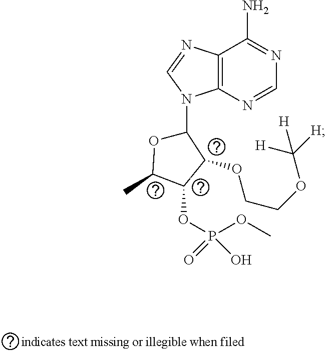

X₆ is

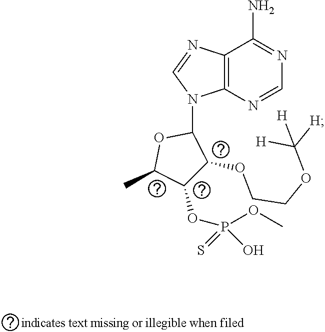

### X₇ is

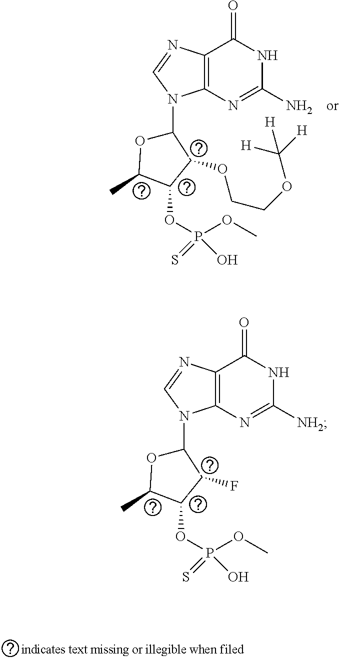 [SMILES: COCCO[C@H]1C(n2cnc3c(=O)[nH]c(N)nc32)O[C@H](C)[C@H]1OP(O)(=S)OC ; COP(O)(=S)O[C@@H]1[C@@H](C)OC(n2cnc3c(=O)[nH]c(N)nc32)[C@@H]1F]

### X₈ is

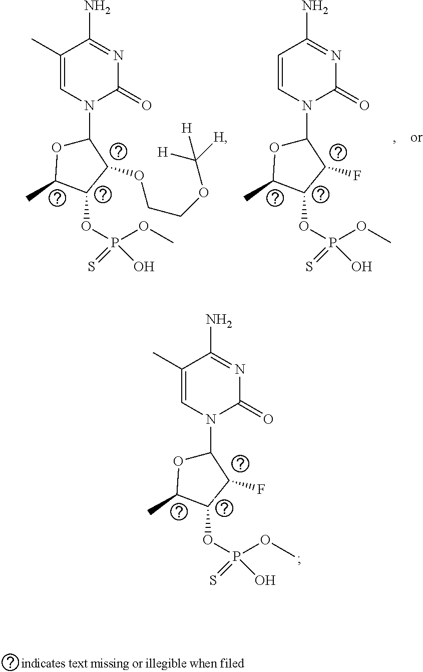

### X₉ is

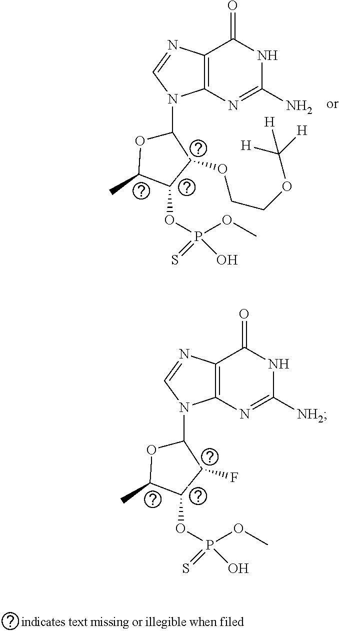 [SMILES: COCCO[C@H]1C(n2cnc3c(=O)[nH]c(N)nc32)O[C@H](C)[C@H]1OP(O)(=S)OC ; COP(O)(=S)O[C@@H]1[C@@H](C)OC(n2cnc3c(=O)[nH]c(N)nc32)[C@@H]1F]

### X₁₀ is

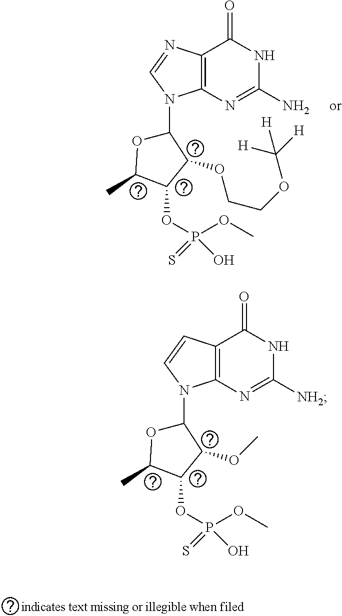

X₁₁ is

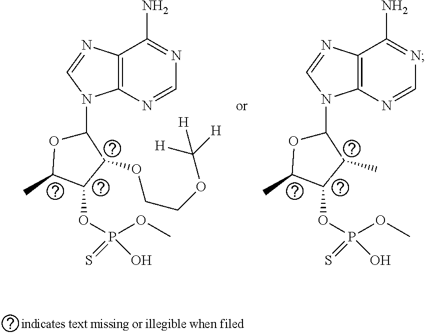 [SMILES: COCCO[C@H]1C(n2cnc3c(N)ncnc32)O[C@H](C)[C@H]1OP(O)(=S)OC ; COP(O)(=S)O[C@@H]1[C@@H](C)OC(n2cnc3c(N)ncnc32)[C@@H]1C]

### X₁₂ is

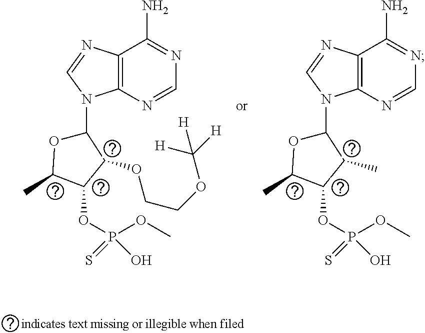 [SMILES: COCCO[C@H]1C(n2cnc3c(N)ncnc32)O[C@H](C)[C@H]1OP(O)(=S)OC ; COP(O)(=S)O[C@@H]1[C@@H](C)OC(n2cnc3c(N)ncnc32)[C@@H]1C]

### X₁₃ is

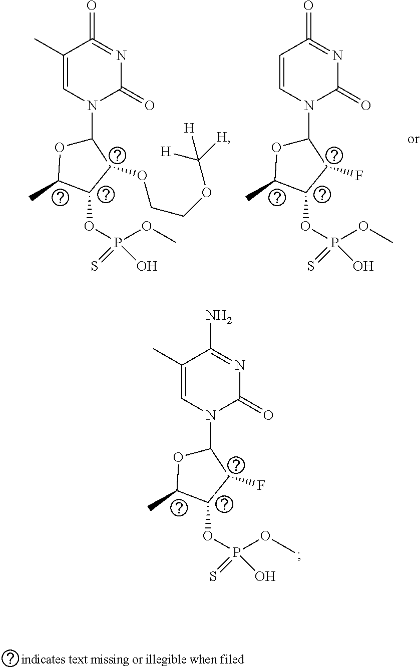

### X₁₄ is

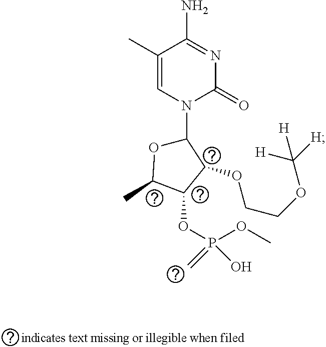

### X₁₅ is

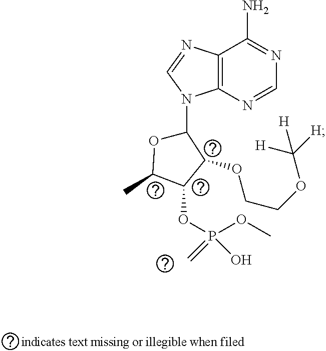

and
X_{B} is

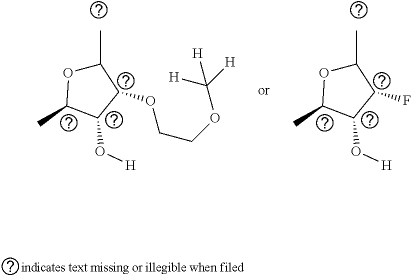

### and B_{B} is

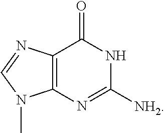

In some embodiments,
X_{A} is

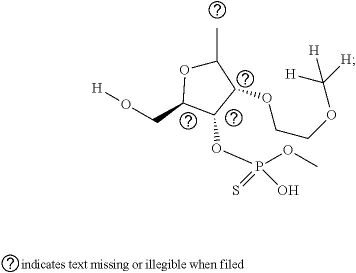

### X₇ is

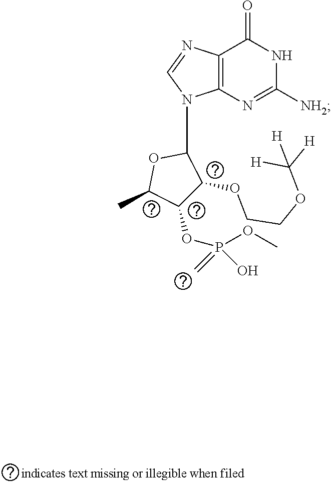 [SMILES: C=P(O)(OC)O[C@@H]1[C@@H](C)OC(n2cnc3c(=O)[nH]c(N)nc32)[C@@H]1OCCOC]

### X₈ is

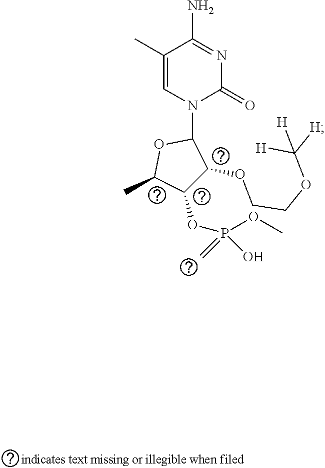

### X₉ is

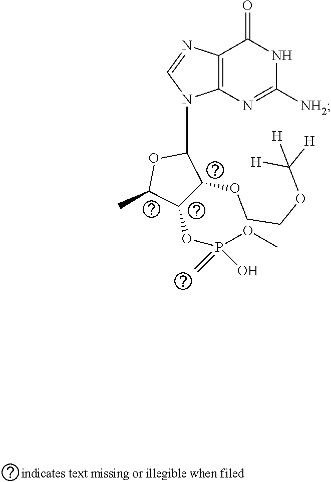 [SMILES: C=P(O)(OC)O[C@@H]1[C@@H](C)OC(n2cnc3c(=O)[nH]c(N)nc32)[C@@H]1OCCOC]

X₁₀ is

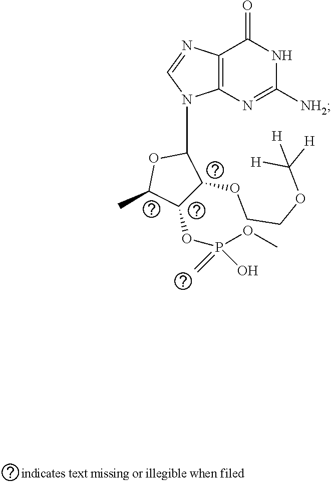 [SMILES: C=P(O)(OC)O[C@@H]1[C@@H](C)OC(n2cnc3c(=O)[nH]c(N)nc32)[C@@H]1OCCOC]

### X₁₂ is

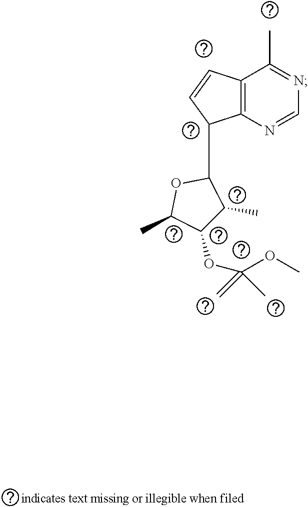

### X₁₃ is

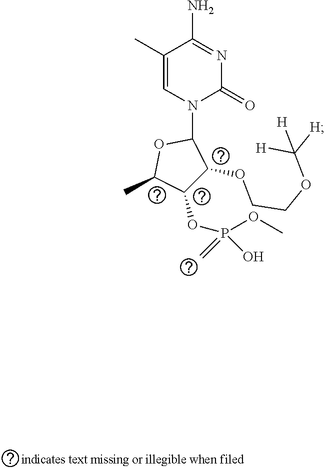

### and X_{B} is

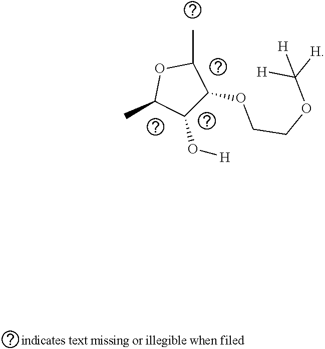

In some embodiments,
X_{A} is

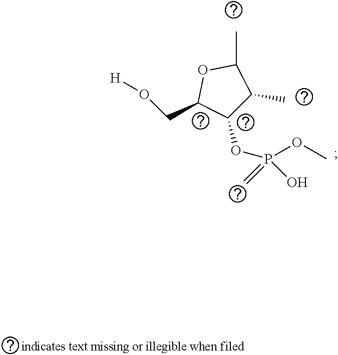

### X₇ is

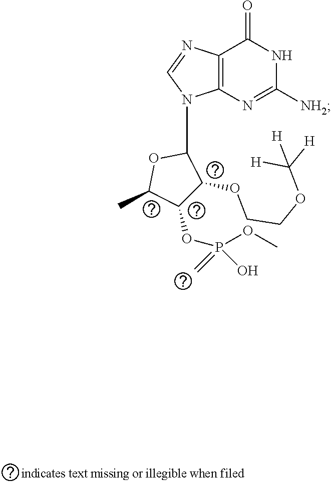 [SMILES: C=P(O)(OC)O[C@@H]1[C@@H](C)OC(n2cnc3c(=O)[nH]c(N)nc32)[C@@H]1OCCOC]

### X₈ is

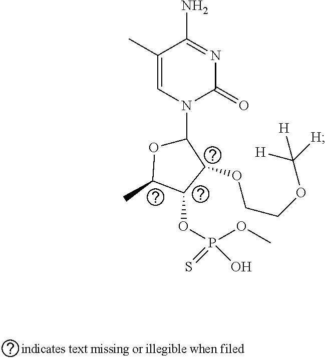

### X₉ is

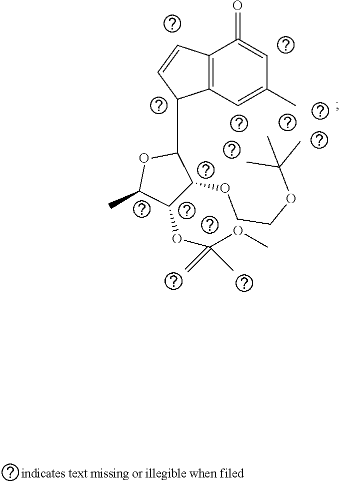

X₁₀ is

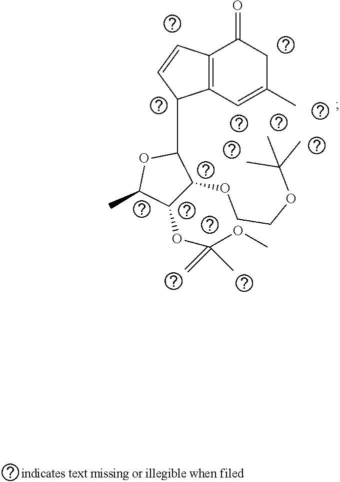

### X₁₂ is

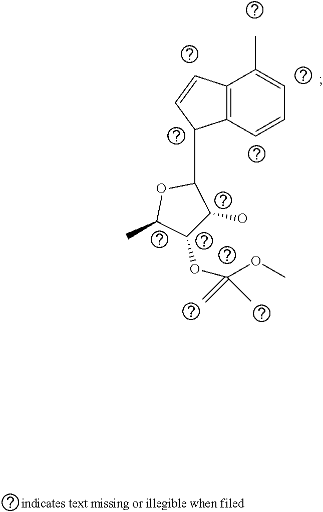

### X₁₃ is

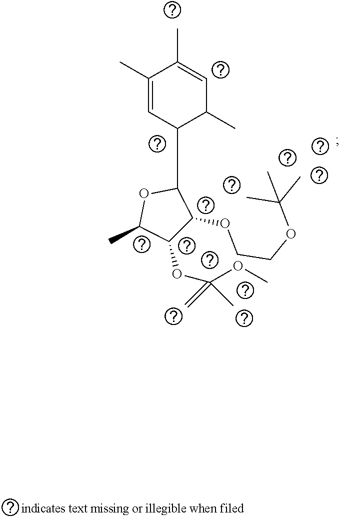

### and X_{B} is

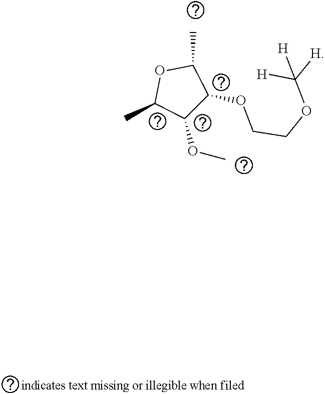

Also provided herein is a compound selected from the group consisting of (SEQ ID NOs: 7-16, 1, 18-25, and 25-29 disclosed below, respectively, in order of appearance):

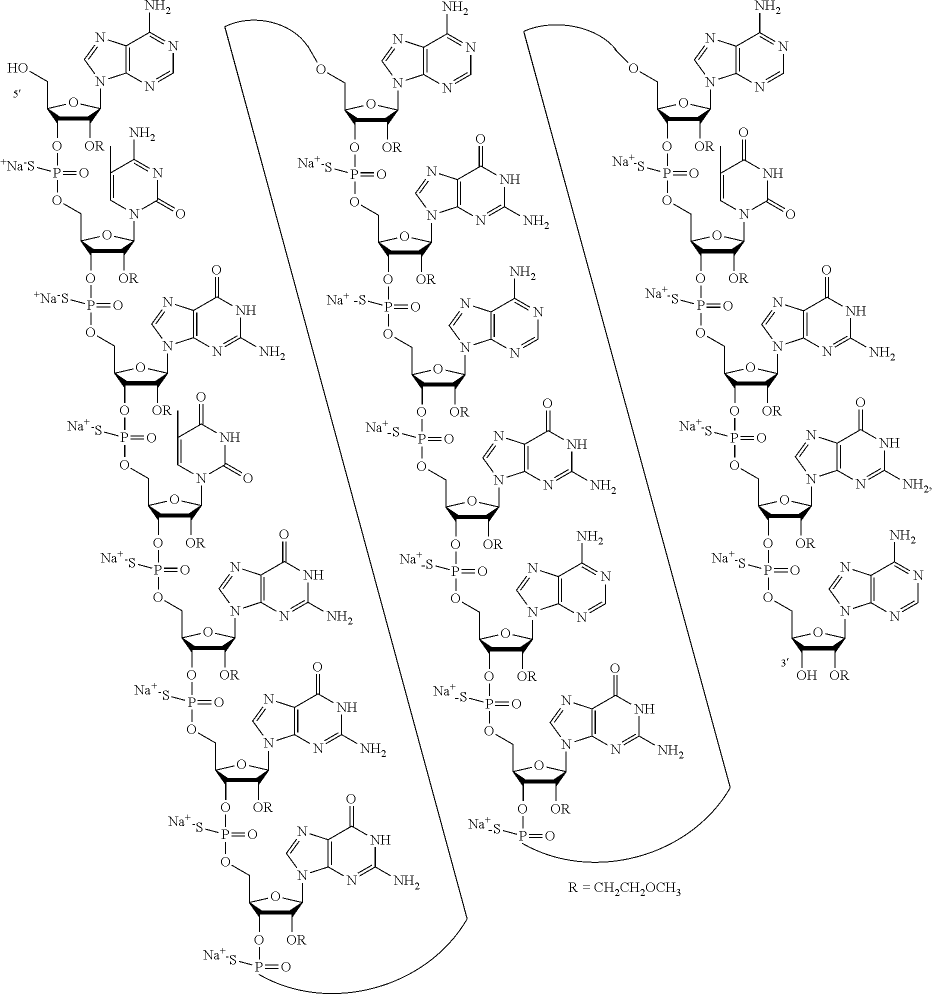

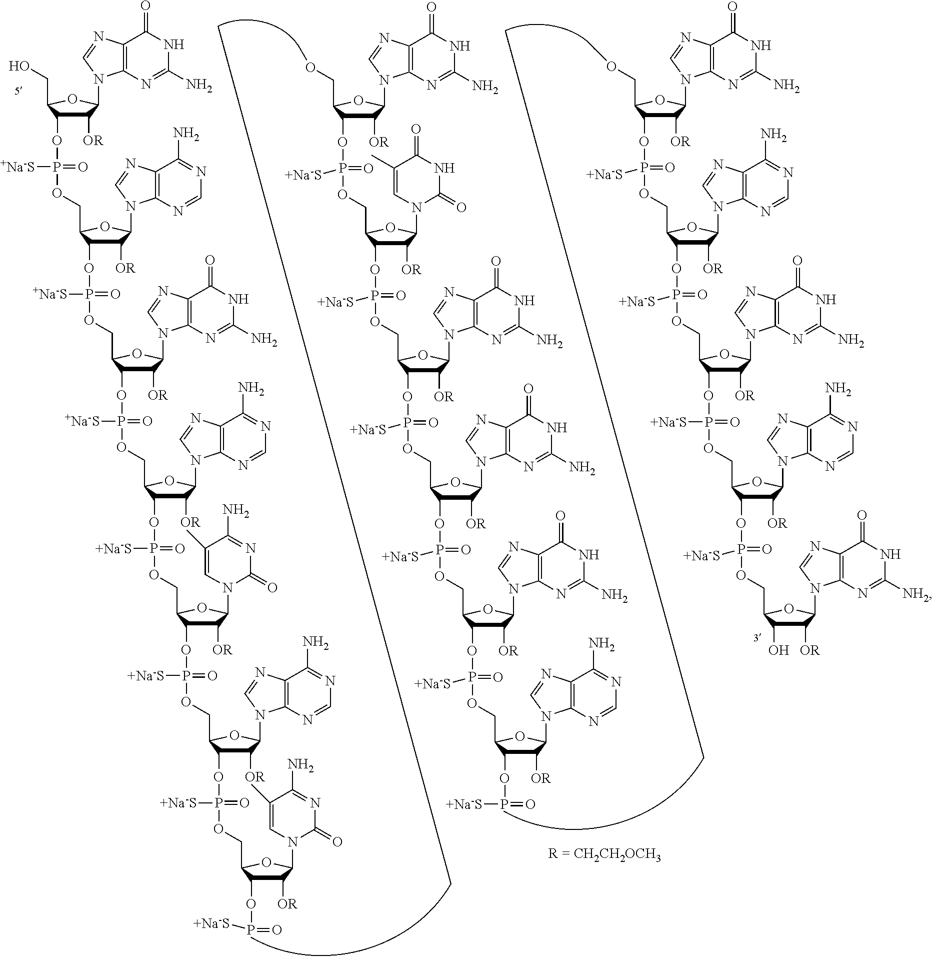

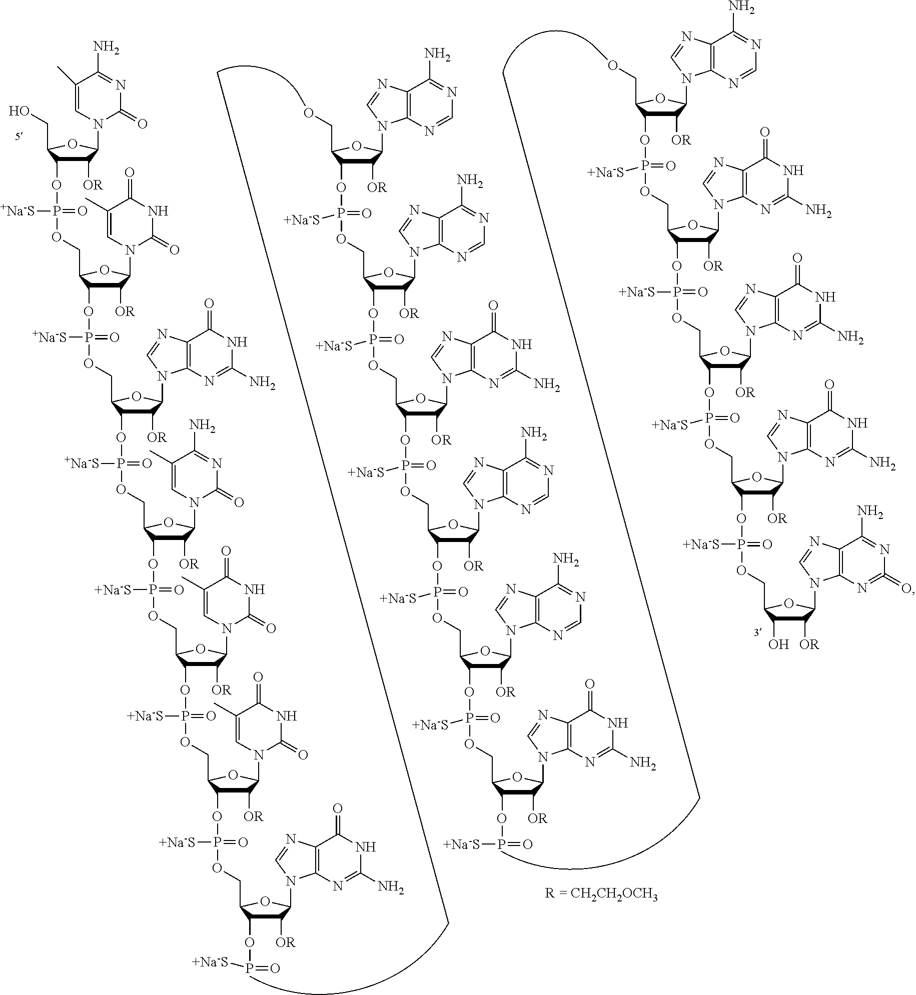

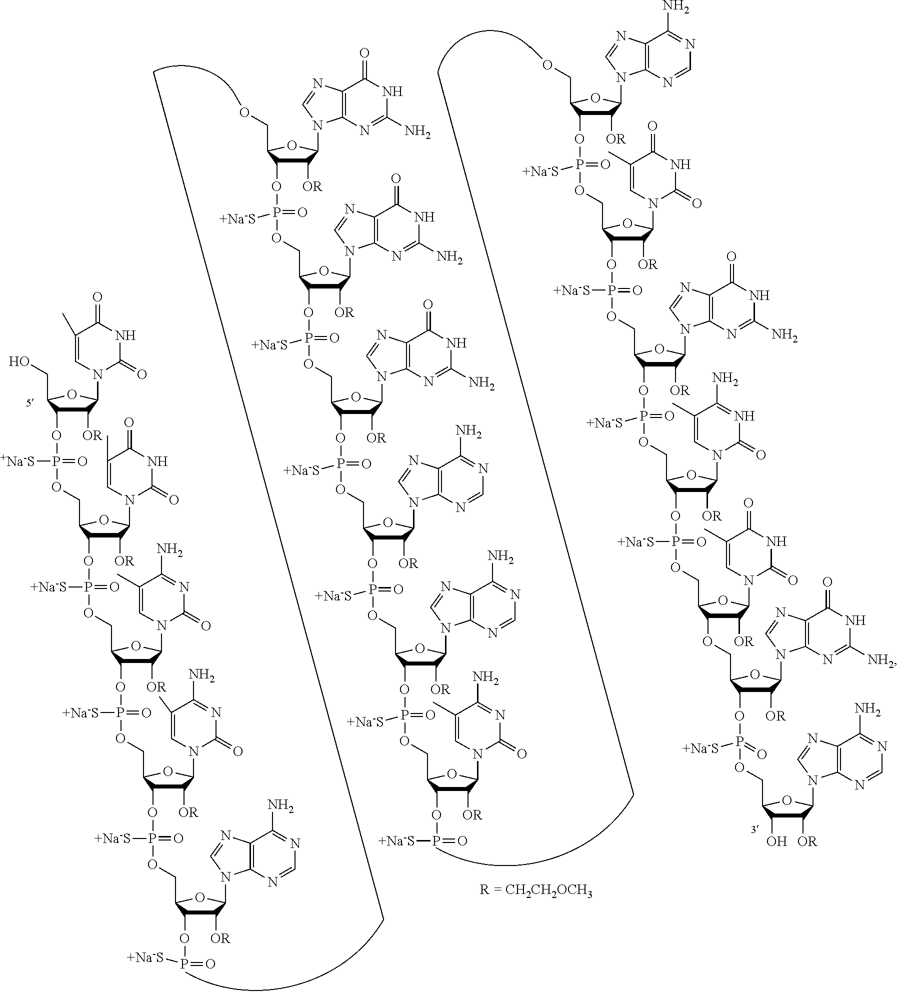

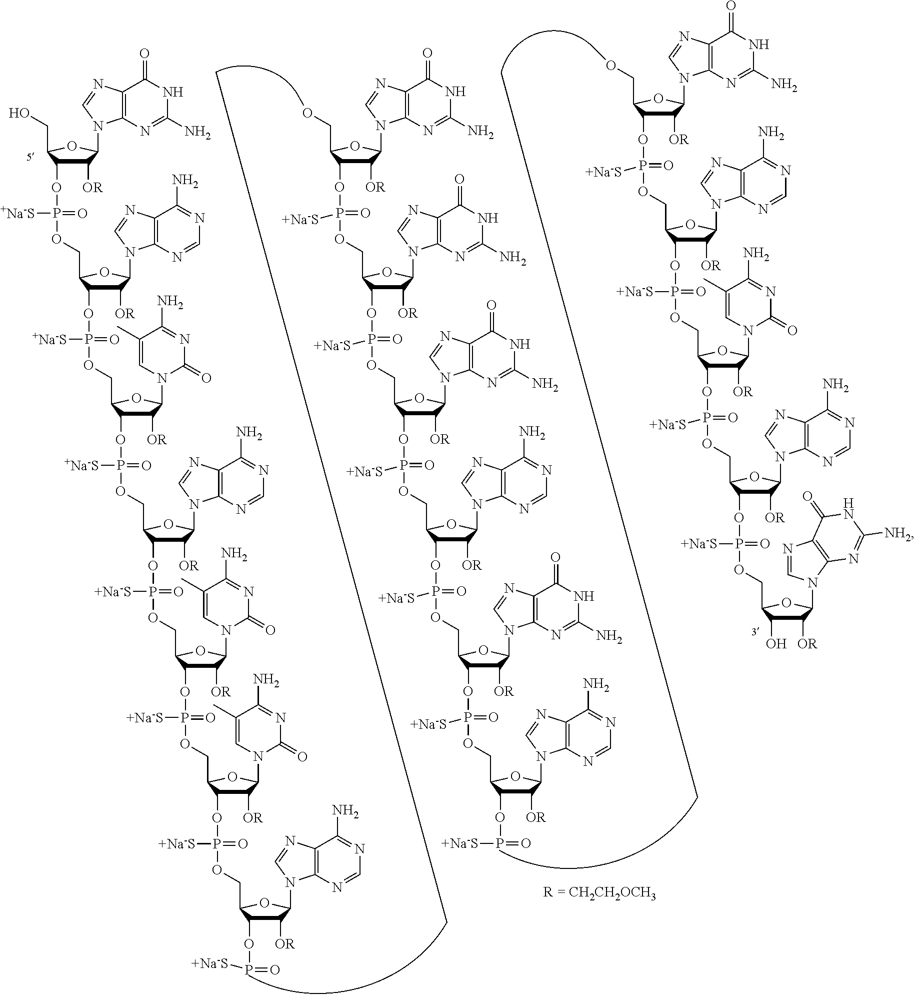

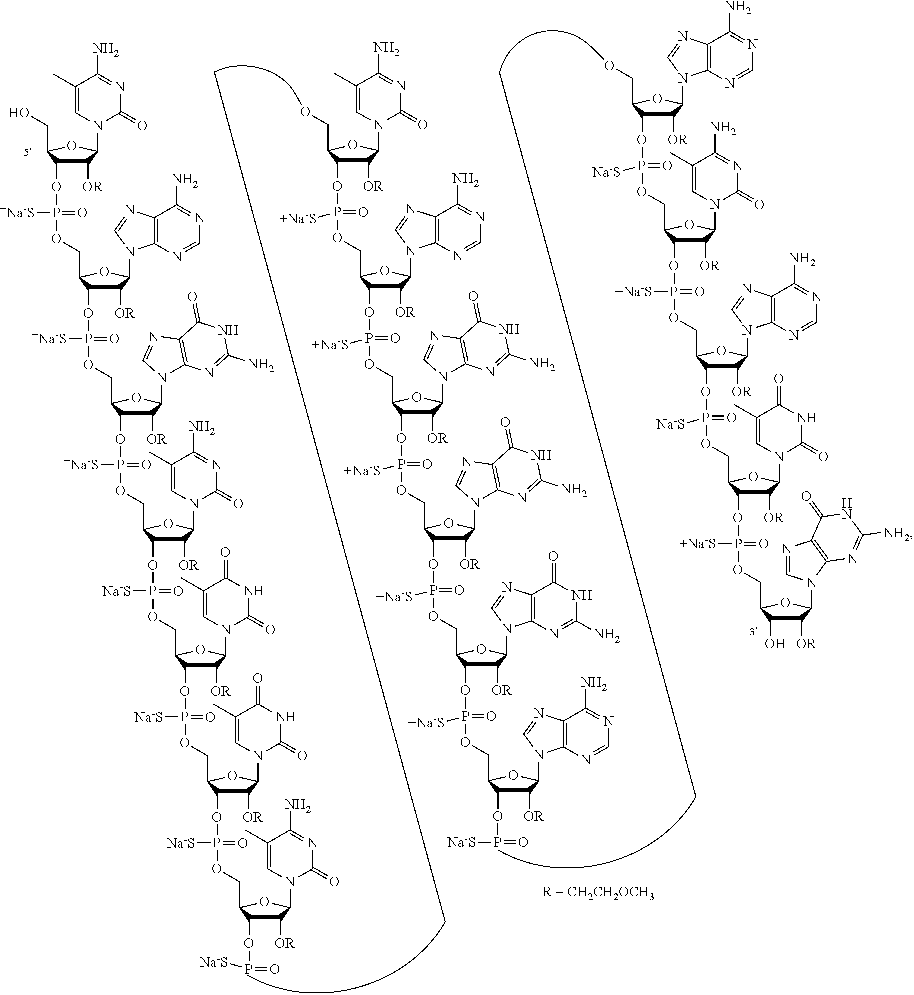

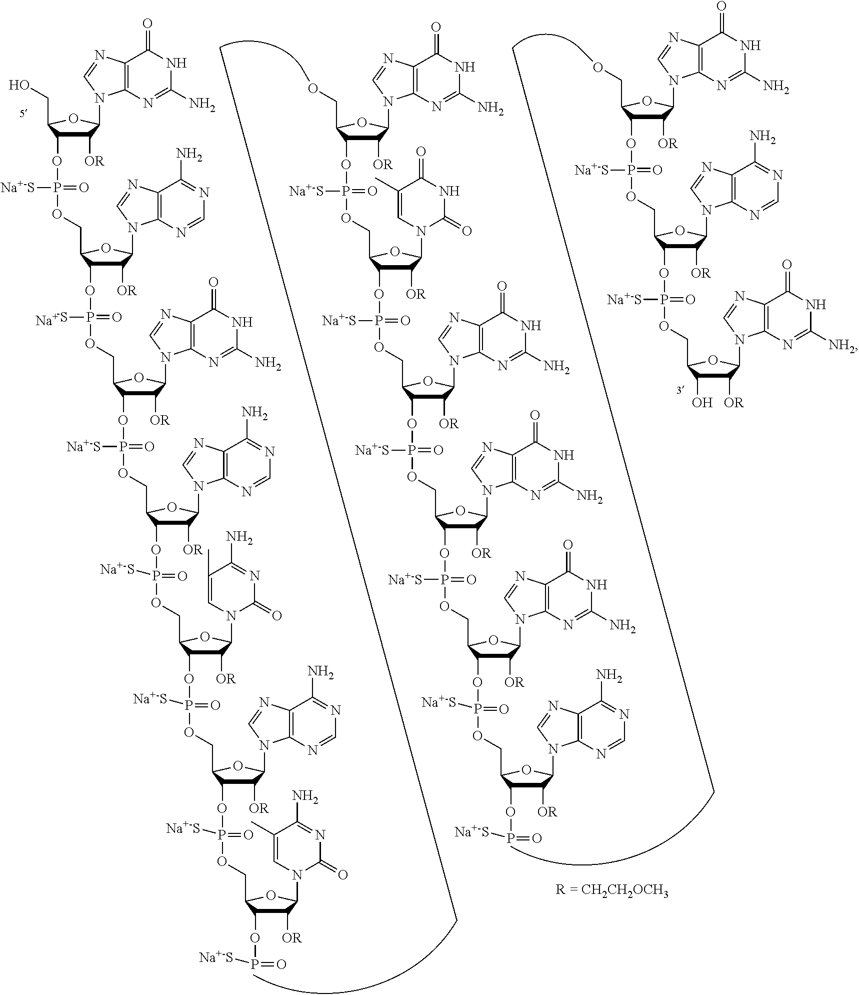

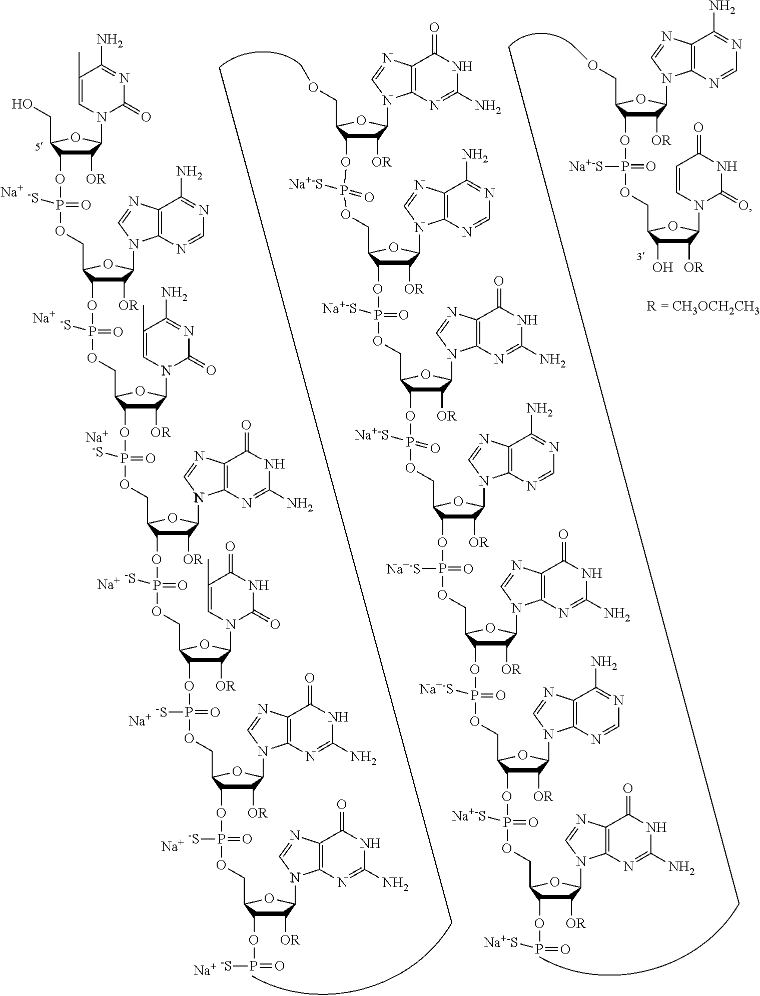

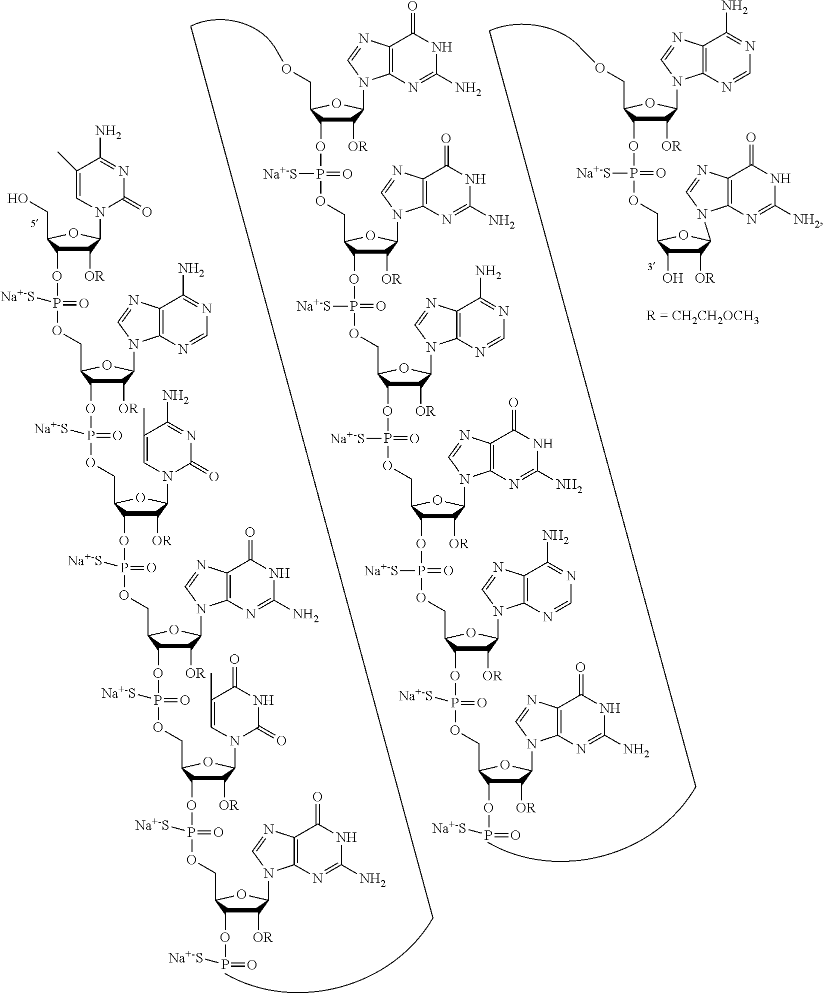

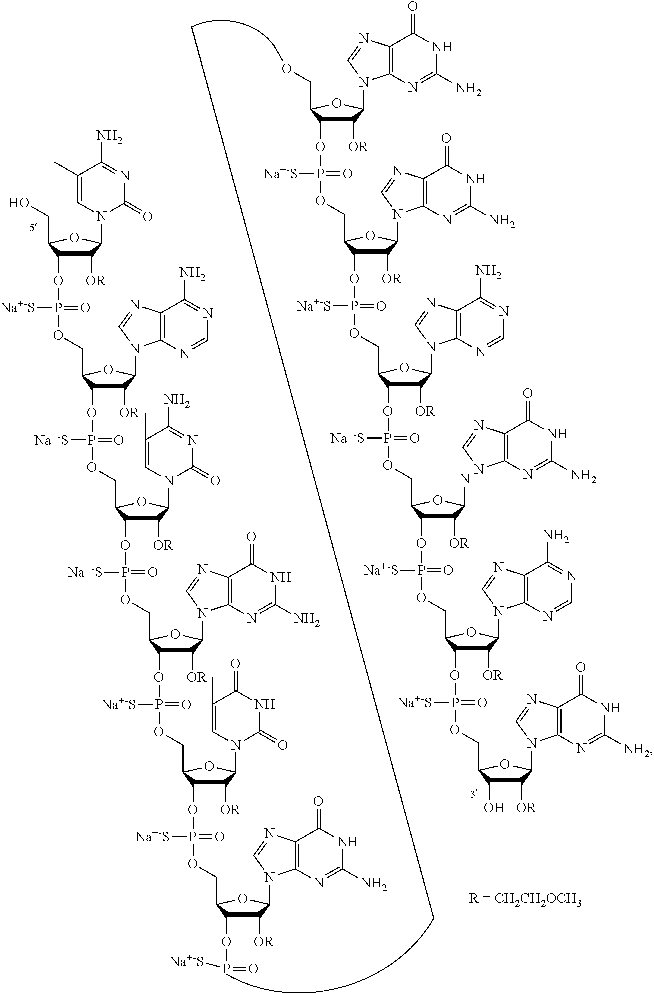

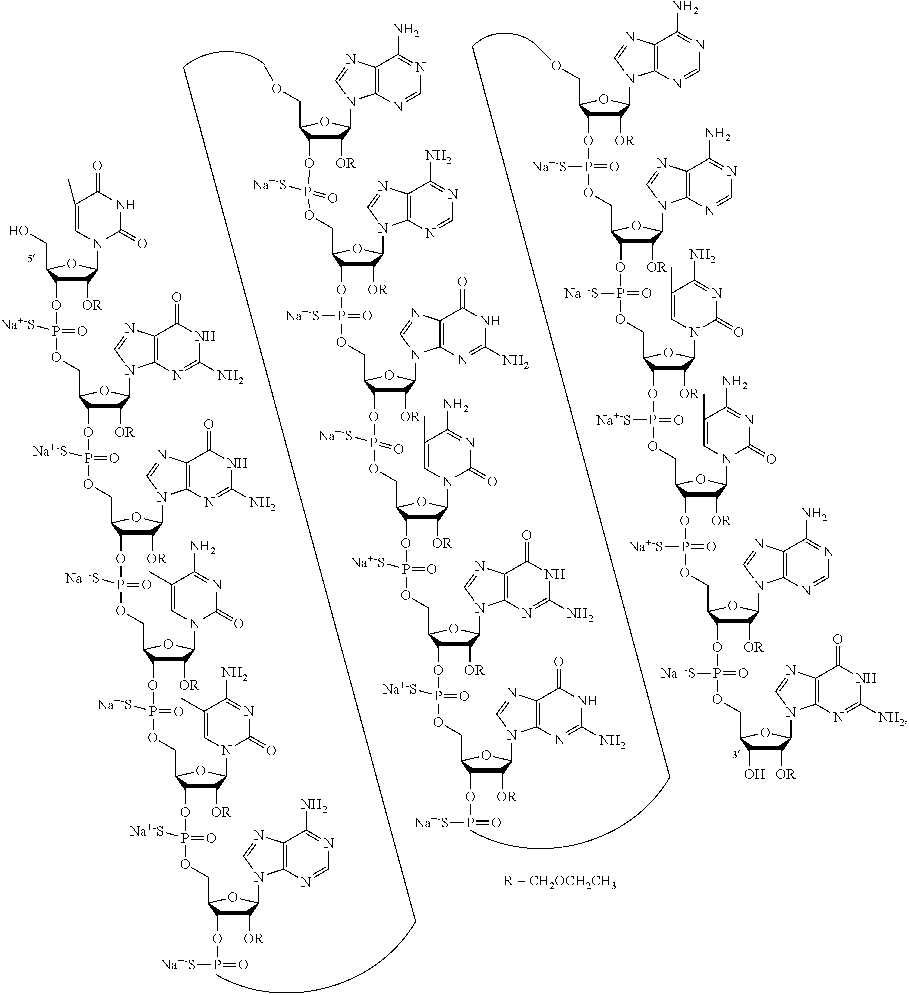

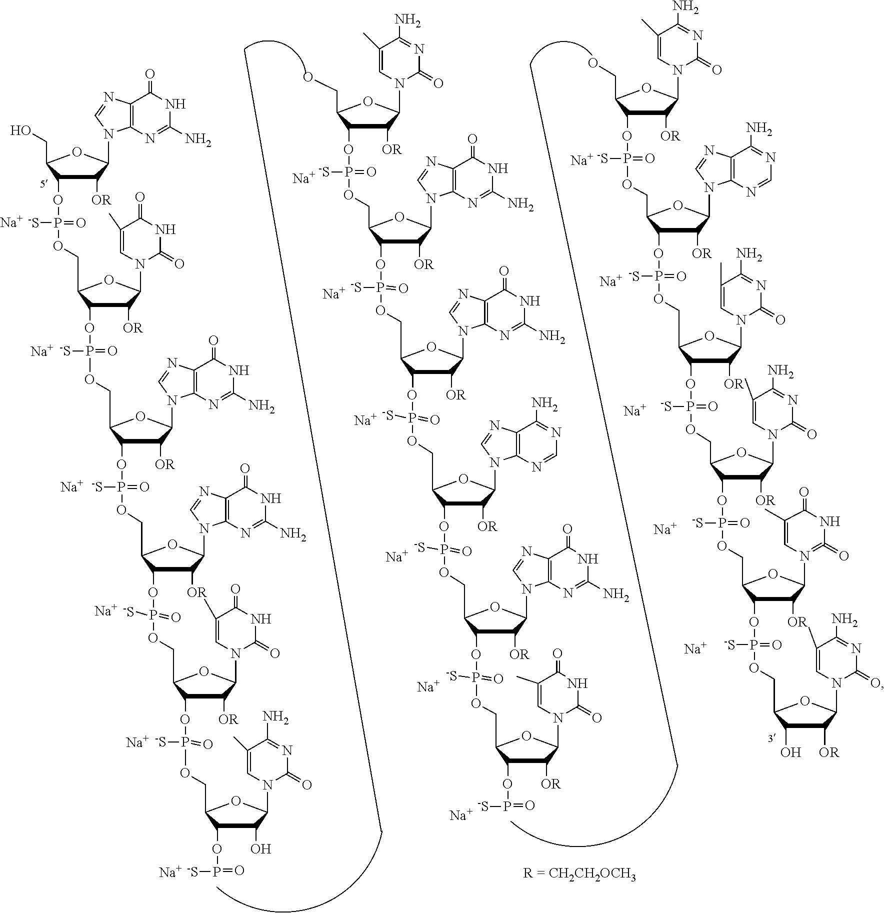

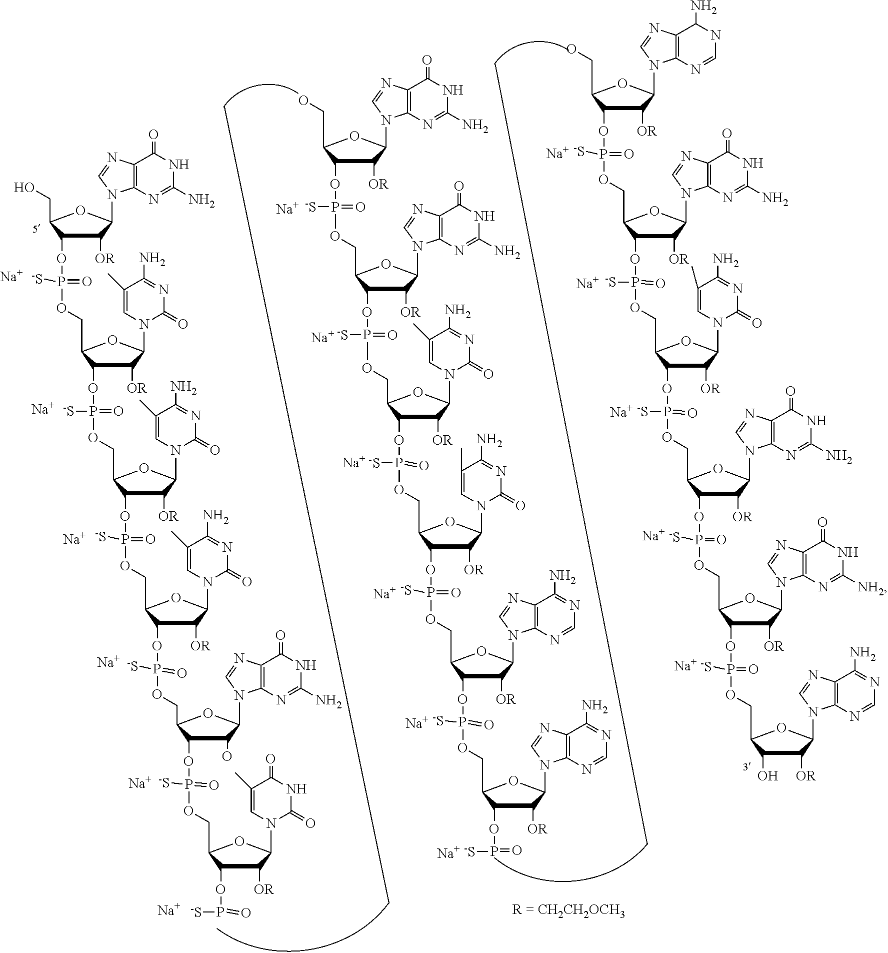

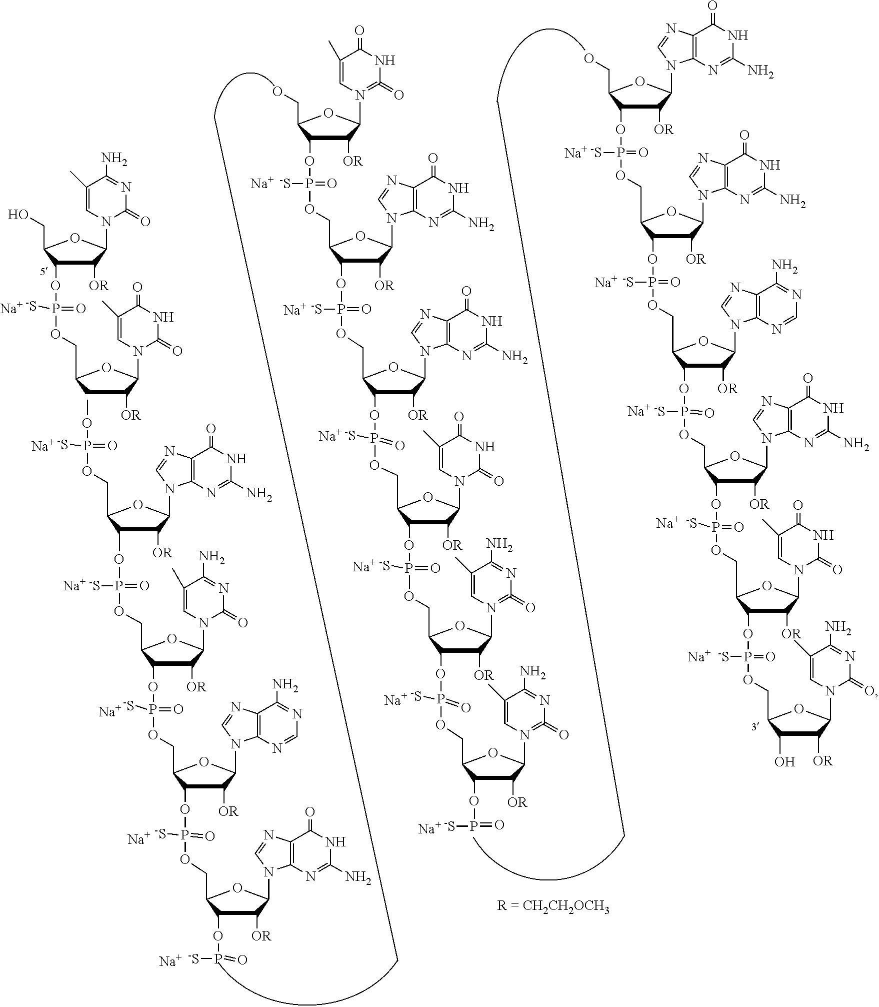

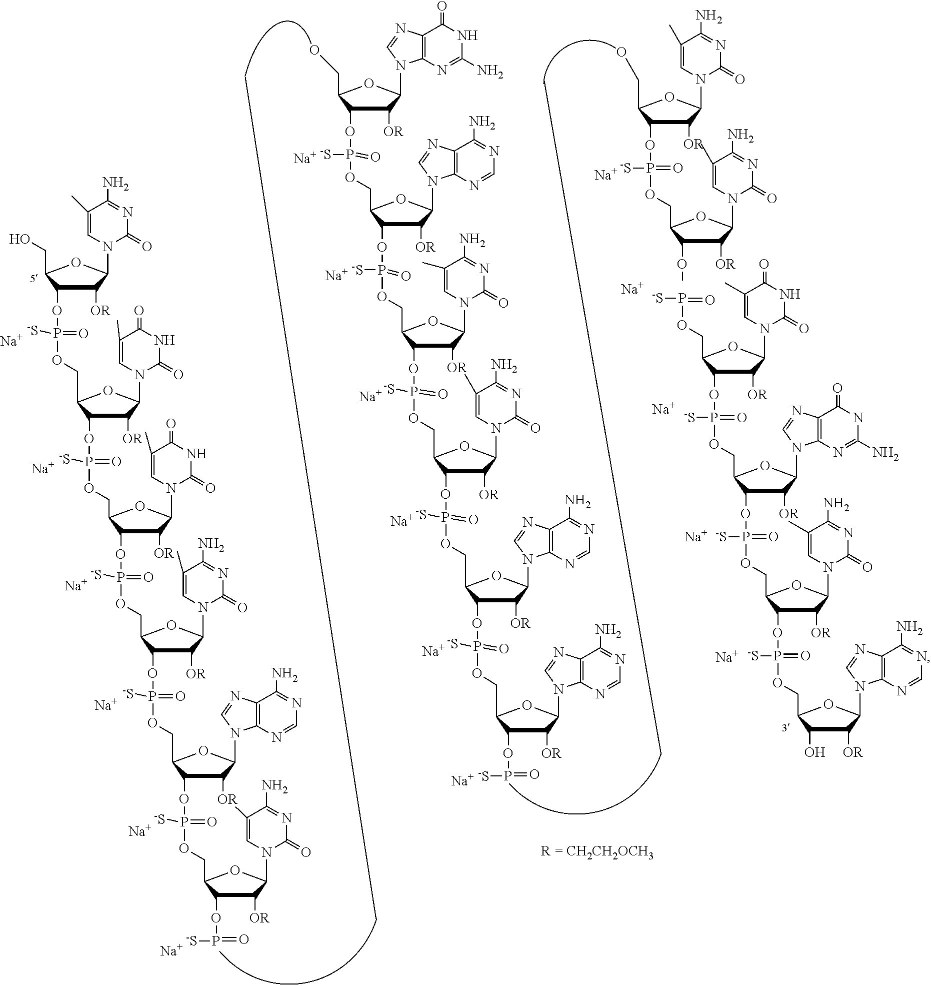

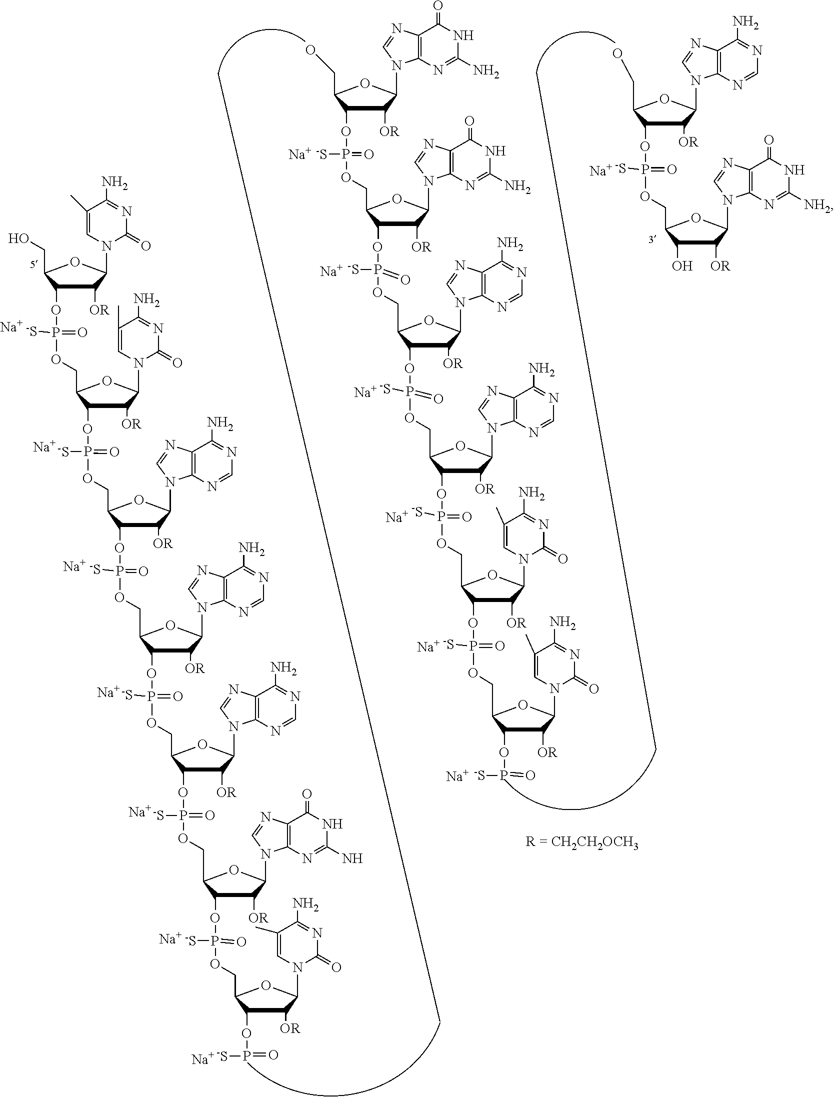

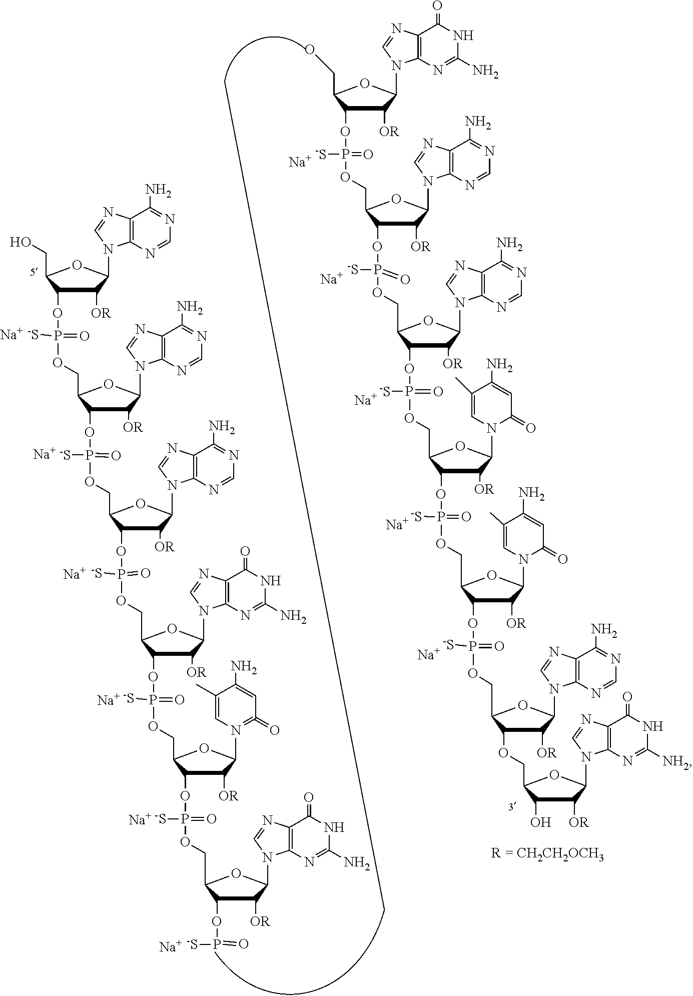

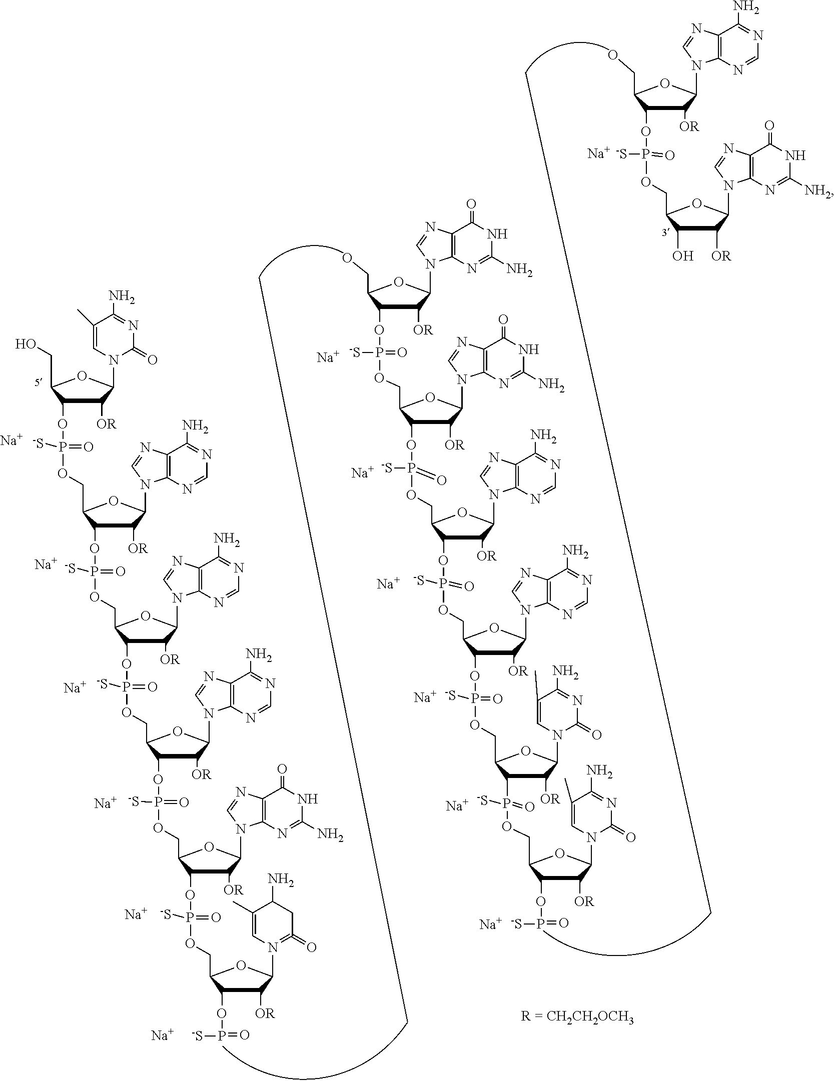

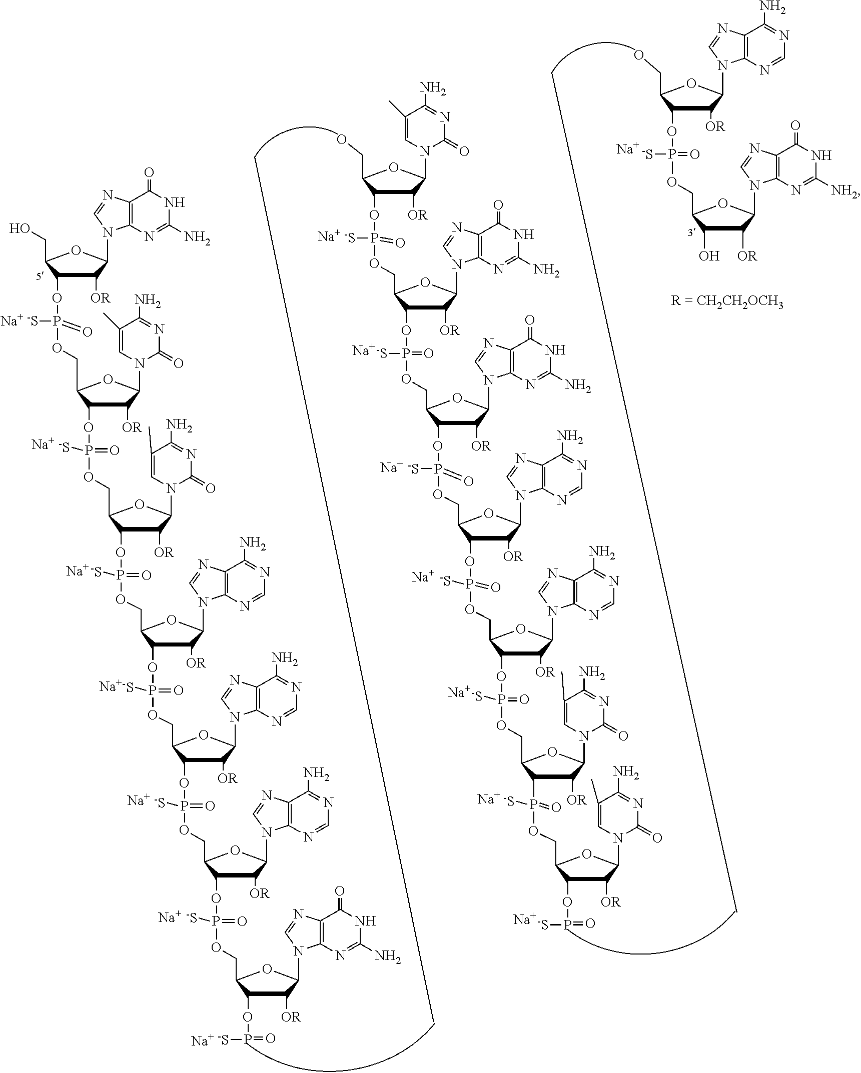

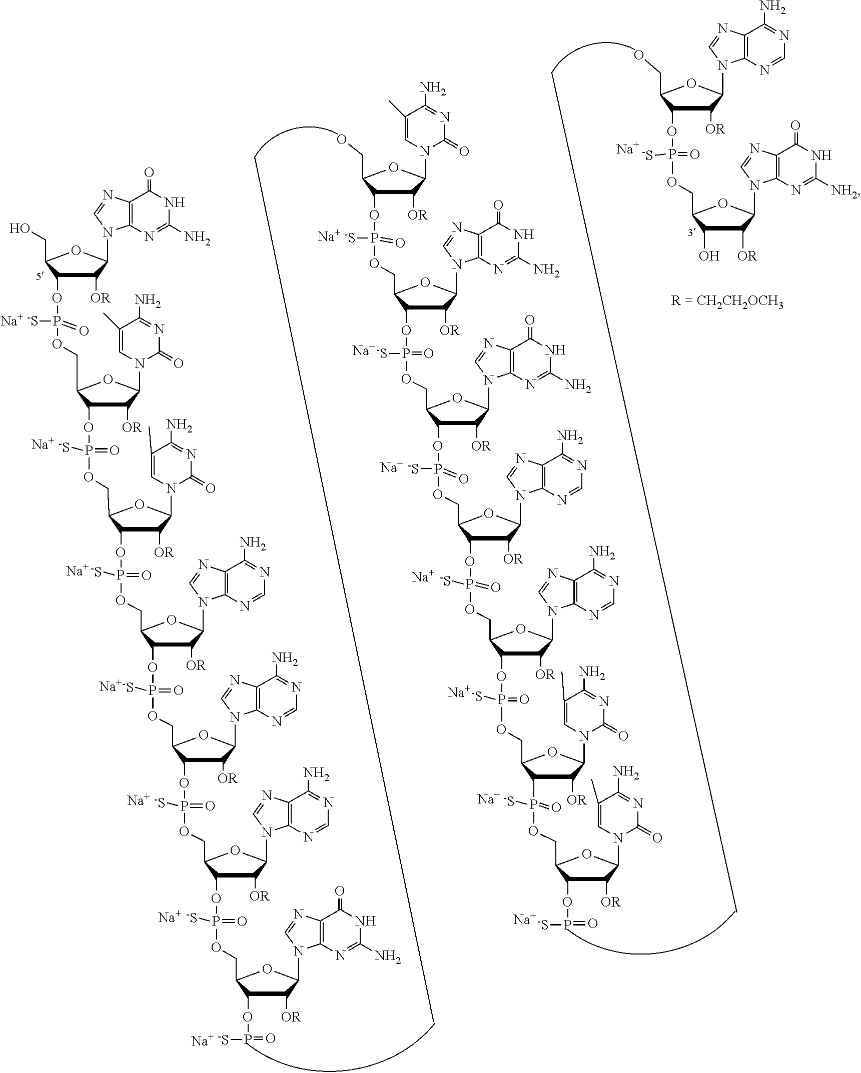

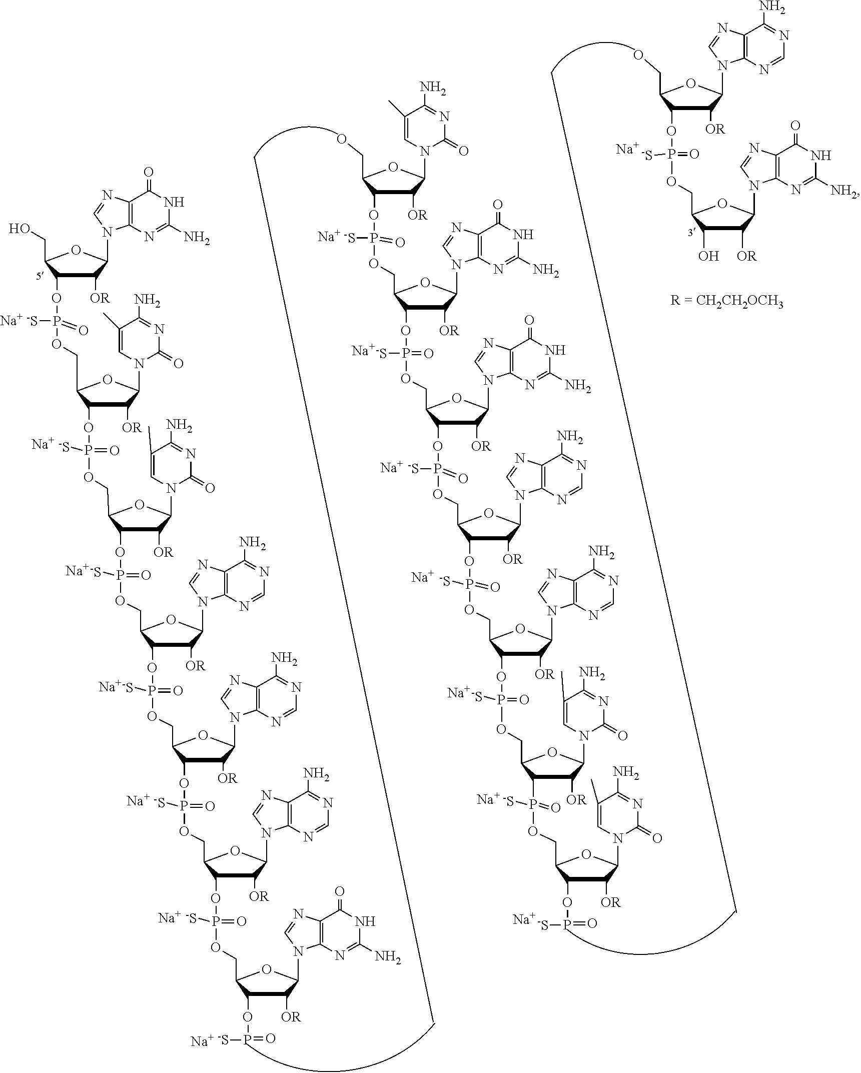

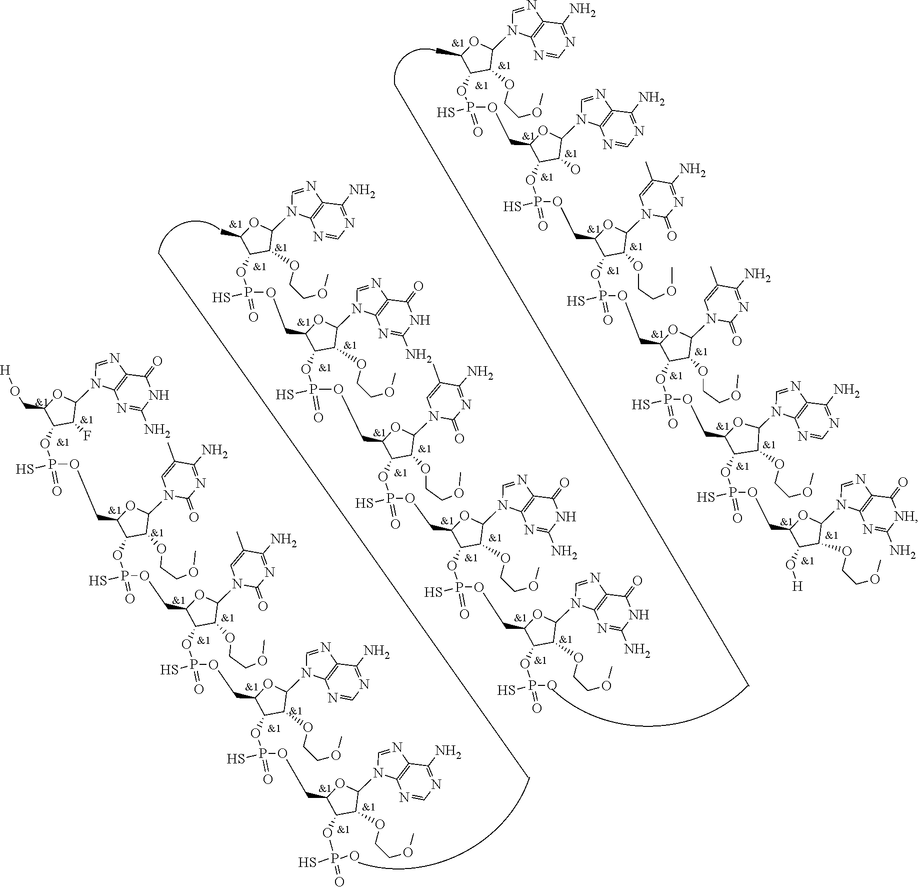

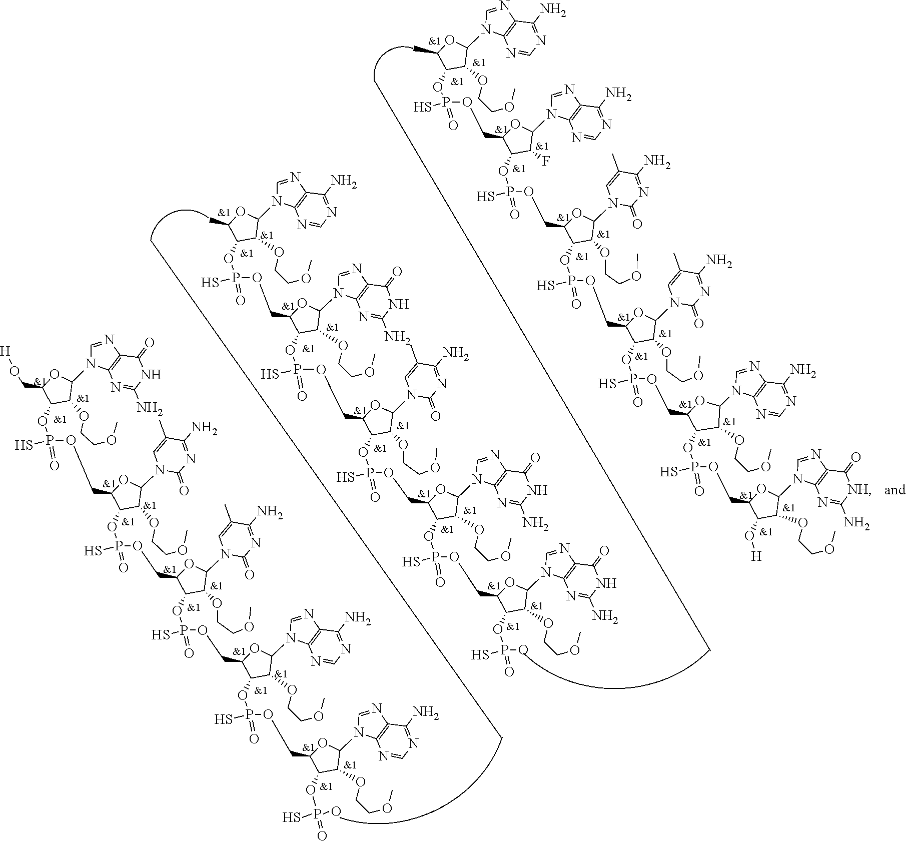

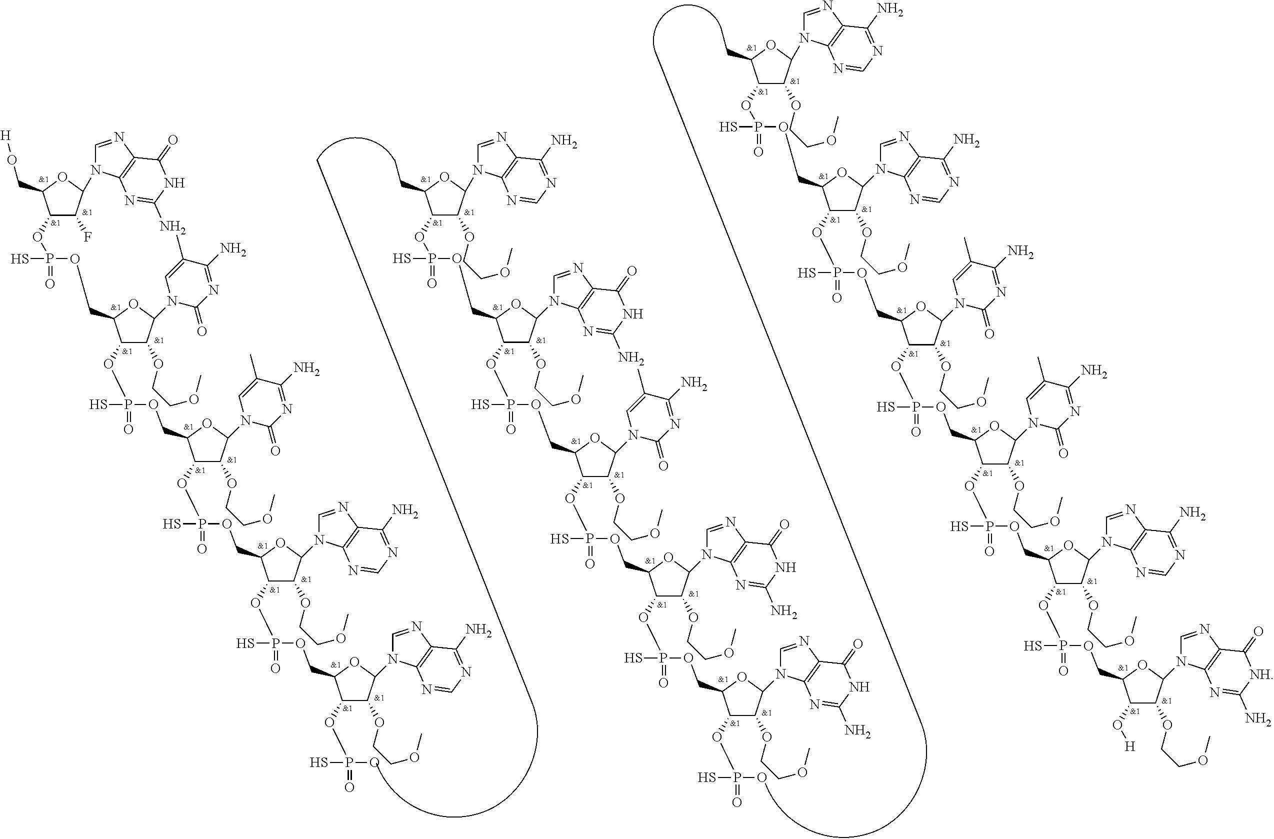

In some aspects, provided herein is a composition comprising a nonsense-mediated RNA decay alternative exon (NSAE)-modulating agent that interacts with a target motif within a pre-processed mRNA transcript to modulate exclusion of an NSAE from a processed mRNA transcript and to modulate inclusion of a canonical exon in the processed mRNA transcript, wherein the target motif is located (i) in an intronic region between two canonical exons, (ii) in one of the two canonical exons, or (iii) in a region spanning both an intron and canonical exon; and wherein the NSAE comprises (i) only a portion of a canonical exon, or (ii) a canonical exon and at least a portion of an intron adjacent to the canonical exon; wherein the NSAE-modulating agent modulates exclusion of an NSAE from the processed mRNA transcript and modulates inclusion of a canonical exon in the processed mRNA transcript.

In some aspects, provided herein is a composition comprising a non-sense mediated RNA decay alternative exon (NSAE)-modulating agent that modulates expression of a target protein in a cell comprising a pre-processed mRNA transcript (pre-mRNA) that encodes the target protein and comprises: an alternative nonsense mediated RNA decay-inducing (NMD) exon comprising an alternative 5′ splice site downstream of the 5′ splice site of a canonical exon and within the canonical exon, or upstream of the 5′ splice site of the canonical exon and within an intron, wherein the NSAE-modulating agent modulates processing of an mRNA transcript from the pre-processed mRNA transcript by modulating splicing of the pre-mRNA at the 5′ alternative splice site, wherein the splicing of the pre-mRNA at the 5′ alternative splice site modulates the expression of the target protein in a cell.

In some aspects, provided herein is a composition comprising a non-sense mediated RNA decay alternative 5′ or 3′ splice site (NSASS)-modulating agent that interacts with a target motif within a pre-processed mRNA transcript to modulate splicing at an alternative 5′ or 3′ splice site of a pre-processed mRNA transcript and to modulate inclusion of a canonical exon in a processed mRNA transcript that is processed from the pre-processed mRNA transcript, wherein the target motif is located in an intronic region between two canonical exons, in one of the two canonical exons, or in a region spanning both an intron and canonical exon; wherein modulating splicing at the alternative 5′ or 3′ splice site of the pre-processed mRNA transcript modulates exclusion of an alternative exon from the processed mRNA transcript, wherein the alternative exon comprises only a portion of a canonical exon, or a canonical exon and at least a portion of an intron adjacent to the canonical exon; and wherein the NSASS-modulating agent modulates exclusion of the alternative exon from the processed mRNA transcript and modulates inclusion of a canonical exon in the processed mRNA transcript.

In some aspects, provided herein is a composition comprising a non-sense mediated RNA decay alternative 5′ or 3′ splice site (NSASS)-modulating agent that modulates expression of a target protein in a cell comprising a pre-processed mRNA transcript (pre-mRNA) that encodes the target protein, wherein the pre-mRNA comprises an alternative exon comprising an alternative 5′ splice site downstream of the 5′ splice site of a canonical exon and within the canonical exon, or upstream of the 5′ splice site of the canonical exon and within an intron; wherein the NSASS-modulating agent modulates processing of an mRNA transcript from the pre-processed mRNA transcript by modulating splicing of the pre-mRNA at the 5′ alternative splice site, wherein the splicing of the pre-mRNA at the 5′ alternative splice site modulates the expression of the target protein in a cell.

In some embodiments, the agent is a small molecule. In some embodiments the agent is a polypeptide. In some embodiments, the polypeptide is a nucleic acid binding protein. In some embodiments, the nucleic acid binding protein contains a TAL-effector or zinc finger binding domain. In some embodiments, the nucleic acid binding protein is a Cas family protein. In some embodiments, the polypeptide is accompanied by or complexed with one or more nucleic acid molecules. In some embodiments, the agent is an antisense oligomer (ASO) complementary to the targeted region of the pre-mRNA. In some embodiments, the agent is at least 75%, at least 80%, at least 85%, at least 90%, at least 95%, at least 98%, at least 99%, or 100%, complementary to the targeted region of the pre-mRNA encoding the target protein. In some embodiments, the agent is an antisense oligomer, and wherein the agent comprises a backbone modification comprising a phosphorothioate linkage or a phosphorodiamidate linkage. In some embodiments, the agent is an antisense oligomer, and wherein the agent comprises a phosphorodiamidate morpholino. In some embodiments, the agent is an antisense oligomer, and wherein the agent comprises a locked nucleic acid. In some embodiments, the agent is an antisense oligomer, and wherein the agent comprises a peptide nucleic acid. In some embodiments, the agent is an antisense oligomer, and wherein the agent comprises a 2′-O-methyl. In some embodiments, the agent is an antisense oligomer, and wherein the agent comprises a 2′-Fluoro, or a 2′-O-methoxyethyl moiety. In some embodiments, the agent is an antisense oligomer, and wherein the agent comprises at least one modified sugar moiety. In some embodiments, each sugar moiety is a modified sugar moiety. In some embodiments, the agent is an antisense oligomer, and wherein the agent consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases. Described herein, in some aspects, is a composition of a nucleic acid molecule that encodes for the NSAE-modulating agent. In some embodiments, the nucleic acid molecule is incorporated into a viral delivery system. In some embodiments, the viral delivery system is an adenovirus-associated vector.

In some aspects, provided herein is a method of modulating protein expression, comprising: (a) contacting a non-sense mediated RNA decay alternative exon (NSAE)-modulating agent to a target motif within a pre-processed mRNA transcript, wherein the NSAE comprises (i) only a portion of a canonical exon, or (ii) a canonical exon and at least a portion of an intron adjacent to the canonical exon; (b) processing a mRNA transcript from the pre-processed mRNA transcript thereby forming a processed mRNA transcript, wherein the NSAE-modulating agent modulates exclusion of an NSAE from the processed mRNA transcript and modulates inclusion of the canonical exon in the processed mRNA transcript; and (c) translating the processed mRNA transcript wherein the exclusion of the NSAE and inclusion of the canonical exon modulates protein expression relative to the protein expression of an equivalent mRNA transcript comprising the NSAE instead of the canonical exon. In some embodiments, the target motif is located in an intronic region between two canonical exons. In some embodiments, the target motif is located in one of the two canonical exons. In some embodiments, the target motif is located in a region spanning both an intron and a canonical exon.

In some aspects, provided herein is a method of modulating expression of a target protein by a cell having a pre-processed mRNA transcript (pre-mRNA) that encodes the target protein and comprises: an alternative nonsense mediated RNA decay-inducing (NMD) exon comprising an alternative 5′ splice site downstream of the 5′ splice site of a canonical exon and within the canonical exon, or upstream of the 5′ splice site of the canonical exon and within an intron, the method comprising contacting a non-sense mediated RNA decay alternative exon (NSAE)-modulating agent to the cell, wherein the non-sense mediated RNA decay alternative exon (NSAE)-modulating agent modulates processing of an mRNA transcript from the pre-processed mRNA transcript by modulating splicing of the pre-mRNA at the 5′ alternative splice site, and wherein the splicing of the pre-mRNA at the 5′ alternative splice site modulates the expression of the target protein.

In some embodiments, the non-sense mediated RNA decay alternative exon (NSAE)-modulating agent binds to a targeted portion of the pre-processed mRNA transcript. In some embodiments, the wherein the non-sense mediated RNA decay alternative exon (NSAE)-modulating agent binds to a factor involved in splicing of the NSAE or NMD exon. In some embodiments, the wherein the non-sense mediated RNA decay alternative exon (NSAE)-modulating agent inhibits activity of a factor involved in splicing of the NMD exon. In some embodiments, the wherein the non-sense mediated RNA decay alternative exon (NSAE)-modulating agent interferes with binding of a factor involved in splicing of the NMD exon to a region of the targeted portion of the pre-processed mRNA transcript. In some embodiments, modulation of splicing of the pre-mRNA increases the expression of the target protein. In some embodiments, the level the target protein in the cell is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell. In some embodiments, modulation of splicing of the pre-mRNA increases production of the processed mRNA encoding the target protein. In some embodiments, the level of processed mRNA encoding the target protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell. In some embodiments, the target protein is the canonical isoform of the protein. In some embodiments, the target protein is SynGAP1. In some embodiments, the non-sense mediated RNA decay alternative exon (NSAE)-modulating agent is a composition described herein.

In some aspects, provided herein is a pharmaceutical composition comprising: a therapeutic agent comprising a composition described herein; and a pharmaceutically acceptable excipient and/or a delivery vehicle.

In some aspects, provided herein is a method of treating or preventing a disease or condition in a subject in need thereof, the method comprising: administering to the subject a pharmaceutical composition described herein.

In some aspects, provided herein is a method of treating or preventing a disease or condition in a subject in need thereof, the method comprising: administering to the subject a pharmaceutical composition comprising: (a) a non-sense mediated RNA decay alternative exon (NSAE)-modulating agent that interacts with a target motif within a pre-processed mRNA transcript to modulate exclusion of an NSAE from a processed mRNA transcript and to modulate inclusion of a canonical exon in the processed mRNA transcript, wherein the NSAE comprises (i) only a portion of a canonical exon, or (ii) a canonical exon and at least a portion of an intron adjacent to the canonical exon; and (b) a pharmaceutically acceptable excipient and/or a delivery vehicle, wherein the disease or condition is treated or prevented in the subject by the administration of the NSAE-modulating agent by a modulation in expression of a protein translated from the processed mRNA transcript.

In some aspects, provided herein is a method of treating a disease or condition in a subject in need thereof by modulating expression of a target protein in a cell of the subject, wherein the cell of the subject has a pre-processed mRNA transcript (pre-mRNA) that encodes the target protein and comprises: (a) an exon preceded by an intron flanking a 5′ splice site of the exon; and (b) an alternative nonsense mediated RNA decay-inducing (NMD) exon comprising an alternative 5′ splice site downstream of the 5′ splice site of the exon and within the exon, or upstream of the 5′ splice site of the exon and within the intron, the method comprising contacting a therapeutic agent to the cell, wherein the therapeutic agent modulates processing of an mRNA transcript from the pre-processed mRNA transcript by modulating splicing of the pre-mRNA at the 5′ alternative splice site, and wherein the splicing of the pre-mRNA at the 5′ alternative splice site modulates the expression of the target protein in the cell of the subject.

In some embodiments the disease is autosomal dominant mental retardation, epileptic encephalopathy, or autism. In some embodiments, the disease or the condition is caused by a deficient amount or activity of the target protein. In some embodiments, the therapeutic agent increases the level of the processed mRNA encoding the target protein in the cell. In some embodiments, the therapeutic agent increases the expression of the target protein in the cell. In some embodiments, the level of processed mRNA encoding the target protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell. In some embodiments, the level the target protein in the cell is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell. In some embodiments, the method further comprises assessing mRNA levels or expression levels of the target protein. In some embodiments, the method further comprises assessing the subject's genome for at least one genetic mutation associated with the disease.

In some embodiments, the disease or the condition is caused by an excess amount or activity of the target protein. In some embodiments, the therapeutic agent decreases the level of the processed mRNA encoding the target protein in the cell. In some embodiments, the therapeutic agent decreases the expression of the target protein in the cell. In some embodiments, the level of processed mRNA encoding the target protein in the cell contacted with the therapeutic agent is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell. In some embodiments, the level the target protein in the cell is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell.

In some embodiments, at least one genetic mutation is within a locus of a gene associated with the disease. In some embodiments, at least one genetic mutation is within a locus associated with expression of a gene associated with the disease. In some embodiments, at least one genetic mutation is within the SYNGAP1 gene locus. In some embodiments, at least one genetic mutation is within a locus associated with SYNGAP1 gene expression. In some embodiments, at least one genetic mutation is within a target gene locus, wherein the target gene locus is a SYNGAP1 gene. In some embodiments, the subject is a human. In some embodiments, the subject is a non-human animal. In some embodiments, the subject is a fetus, an embryo, or a child. In some embodiments, the cell or the cells is ex vivo, or in a tissue, or organ ex vivo. In some embodiments, the therapeutic agent is administered to the subject by intracerebroventricular injection, intraperitoneal injection, intramuscular injection, intrathecal injection, subcutaneous injection, oral administration, synovial injection, intravitreal administration, subretinal injection, topical application, implantation, or intravenous injection.

In some aspects, provided herein is a therapeutic agent for use in a method described herein.

In some aspects, provided herein is a pharmaceutical composition comprising a therapeutic agent described herein and a pharmaceutically acceptable excipient.

In some aspects, provided herein is a method of treating a subject in need thereof, comprising administering a pharmaceutical composition described herein by intracerebroventricular injection, intraperitoneal injection, intramuscular injection, intrathecal injection, subcutaneous injection, oral administration, synovial injection, intravitreal administration, subretinal injection, topical application, implantation, or intravenous injection to the subject.

### BRIEF DESCRIPTION OF THE DRAWINGS

FIG. 1 depicts exemplary TaqMan qPCR results using RNA from HEK293 cells transfected for 24 hrs with 80 nM of the indicated ASOs. The exemplary TaqMan probe spans exons 10 and 11 junction and measures productive mRNA.

FIG. 2 depicts TaqMan qPCR results for productive mRNA and RT-PCR results for nonproductive mRNA using RNA from HEK293 cells transfected for 24 hrs with 80 nM of the indicated ASOs. The exemplary TaqMan probe spans exons 10 and 11 junction and measures productive mRNA.

FIG. 3 depicts TaqMan qPCR results for productive mRNA and RT-PCR results for nonproductive mRNA using RNA from HEK293 cells transfected for 24 hrs with 80 nM of the indicated ASOs. The exemplary TaqMan probe spans exons 10 and 11 junction and measures productive mRNA.

FIG. 4 depicts RT-PCR results for nonproductive mRNA using RNA from HEK293 cells transfected for 24 hrs with 80 nM of the indicated ASOs.

FIG. 5 depicts RT-PCR results for nonproductive mRNA using RNA from HEK293 cells. Cells were transfected for 24 hrs with 80 nM of the indicated ASOs (left) or treated with 20 μM of the indicated ASOs and allowed to freely uptake the ASOs in 2D culture for 72 hours.

FIGS. 6A-6H depict RT-PCR results for dose response decrease of nonproductive mRNA using RNA from HEK293 cells. Cells were treated with increasing concentrations of the indicated ASOs and allowed to freely uptake the ASOs in 3D culture for 72 hours. Data for ASO 59 can be found in FIG. 6A; ASO 59-1 in FIG. 6B; ASO 59-2 in FIG. 6C; ASO 59-3 in 6D; ASO 59-4 in FIG. 6E; ASO 59-5 in FIG. 6F; ASO 59-6 in FIG. 6G; and ASO 59-7 in FIG. 6H. The abundance of the Exon18X containing transcripts was plotted against the logarithmic value (log 10) of the ASO dose using a nonlinear regression fit of a variable slope four parameter dose-response curve as calculated by GraphPad Prism 8. Biological replicates were plotted for each concentration with standard deviation (S.D.) indicated. The values of the control group comprising no ASO were used to plot the upper end of the curve at a concentration representative of 0 μM. Some of the projected curve analysis used constrain between 0 and 0.05. The EC50 and log EC50 values were calculated by GraphPad Prism 8.

FIG. 7 depicts RT-PCR results for nonproductive mRNA using RNA extracted from HEK293 cells 24 hrs after transfection with 80 nM of the indicated ASOs (upper panel), or RNA extracted from HEK293 cells 72 hours after the cells were allowed to freely uptake 20 μM of the indicated ASOs in 3D culture (lower panel).

FIG. 8 depicts RT-PCR results for dose-responsive decrease of nonproductive mRNA using RNA from HEK293 cells. Cells were treated with increasing concentrations of the indicated ASOs and evaluated 72 hours after they were allowed to freely uptake the ASOs in 3D culture. The abundance of the Exon18X containing transcripts was plotted against the logarithmic value (log 10) of the ASO dose using a nonlinear regression fit of a variable slope four parameter dose-response curve as calculated by GraphPad Prism 8. Biological replicates were plotted for each concentration with standard deviation (S.D.) indicated. The values of the control group comprising no ASO were used to plot the upper end of the curve at a concentration representative of 0 μM. Some of the projected curve analyses used constraints between 0 and 0.05. The EC50 and log EC50 values were calculated by GraphPad Prism 8.

FIG. 9 depicts RT-PCR results for nonproductive mRNA and results using a ProteinSimple Jess assay to detect the a1 isoform of SYNGAP1. 20 mg of indicated ASO was intracerebroventricular injected (ICV) into P2 Syngap1 heterozygous mice. 4 weeks later, brains were dissected, and hippocampi were assayed for Exon 18X inclusion by RT-PCR. The levels of the a1-isoform of SYNGAP1 protein were normalized to total protein via a ProteinSimple JESS assay.

FIGS. 10A-10B are histograms illustrating the fold change in ASO compound target engagement (FIG. 10A) and SynGAP alpha 1 protein (FIG. 10B) in the cerebral cortex after intracerebroventricular injection of Syngap1 heterozygous (Syngap1^{+/-}) mouse pups with various amounts (3 μg, 10 μg, 20 μg, and 30 μg) of four different ASO compounds (compounds mm59, mm60, mm61, and mm62). ASO target engagement was evaluated as the fold change in nonproductive mRNA normalized to mitochondrial ribosomal protein L16 (MRPL16) RNA. SynGAP alpha 1 protein levels were normalized to actin.

FIGS. 11A-11B are histograms illustrating the fold change in ASO compound target engagement (FIG. 11A) and SynGAP alpha 1 protein (FIG. 11B) in the hippocampus after intracerebroventricular injection of Syngap1 heterozygous (Syngap1^{+/-}) mouse pups with various amounts (3 μg, 10 μg, 20 μg, and 30 μg) of four different ASO compounds (compounds mm59, mm60, mm61, and mm62). ASO target engagement was evaluated as the fold change in nonproductive mRNA normalized to mitochondrial ribosomal protein L16 (MRPL16) RNA. SynGAP alpha 1 protein levels were normalized to actin.

FIGS. 12A-12B are histograms illustrating the fold change in ASO compound target engagement (FIG. 12A) and SynGAP alpha 1 protein (FIG. 12B) in the midbrain after intracerebroventricular injection of Syngap1 heterozygous (Syngap1^{+/-}) mouse pups with various amounts (3 μg, 10 μg, 20 μg, and 30 μg) of four different ASO compounds (compounds mm59, mm60, mm61, and mm62). ASO target engagement was evaluated as the fold change in nonproductive mRNA normalized to mitochondrial ribosomal protein L16 (MRPL16) RNA. SynGAP alpha 1 protein levels were normalized to actin.

FIGS. 13A-13B are histograms illustrating the fold change in ASO compound target engagement (FIG. 13A) and SynGAP alpha 1 protein (FIG. 13B) in the basal ganglia/thalamus after intracerebroventricular injection of Syngap1 heterozygous (Syngap1^{+/-}) mouse pups with various amounts (3 μg, 10 μg, 20 μg, and 30 μg) of four different ASO compounds (compounds mm59, mm60, mm61, and mm62). ASO target engagement was evaluated as the fold change in nonproductive mRNA normalized to mitochondrial ribosomal protein L16 (MRPL16) RNA. SynGAP alpha 1 protein levels were normalized to actin.

FIGS. 14A-14C depict RT-PCR results for dose-responsive decrease of nonproductive mRNA using RNA from mouse cortical neurons (MCNs). Cells were treated with increasing concentrations of the indicated ASOs and allowed to freely uptake the ASOs in 3D culture for 72 hours in three different biological replicates (replicate 1 can be found in FIG. 14A; replicate 2 can be found in FIG. 14B; and replicate 3 can be found in FIG. 14C). The abundance of the Exon18X containing transcripts was plotted against the logarithmic value (log 10) of the ASO dose using a nonlinear regression fit of a variable slope four parameter dose-response curve as calculated by GraphPad Prism 8. Biological replicates were plotted for each concentration with standard deviation (S.D.) indicated. The EC50 and log EC50 values were calculated by GraphPad Prism 8.

### DETAILED DESCRIPTION

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The coordinate as used herein refers to the coordinate of the genome reference assembly GRCh38 (Genome Research Consortium human build 38), also known as Hg38 (Human genome build 38).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

As used herein, an alternative 3′ splice site of an intron is equivalent to an alternative 5′ splice site of the exon immediately downstream of that intron.

As used herein, an alternative 5′ splice site of an intron is equivalent to an alternative 3′ splice site of the exon immediately upstream of that intron.

Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis.

Unless otherwise indicated, the following terms have the following meanings:

“Administering” can mean providing a pharmaceutical agent to an animal, and includes, but is not limited to administering by a medical professional and self-administering. “Amelioration” refers to a lessening, slowing, stopping, or reversing of at least one indicator of the severity of a syndrome or condition. The severity of indicators may be determined by subjective or objective measures, which are known to those skilled in the art.

“Animal” can refer to a human or non-human animal, including, but not limited to, mice, rats, rabbits, dogs, cats, pigs, and non-human primates, including, but not limited to, monkeys and chimpanzees.

“Antisense oligomer” can mean an oligomeric compound that is capable of undergoing hybridization to a target nucleic acid through hydrogen bonding. Examples of antisense oligomers include single-stranded and double-stranded compounds, such as, antisense oligonucleotides, siRNAs, shRNAs, and ssRNAs.

“Antisense inhibition” or “inhibition” can mean reduction of target nucleic acid levels in the presence of an antisense oligomer complementary to a target nucleic acid compared to target nucleic acid levels or in the absence of the antisense oligomer.

“Antisense mechanisms” can refer to all those mechanisms involving hybridization of a compound with a target nucleic acid, wherein the outcome or effect of the hybridization is either target degradation or target occupancy with concomitant stalling of the cellular machinery involving, for example, transcription or splicing. An antisense oligomer provided herein can be “antisense” to a target nucleic acid, meaning that the antisense oligomer is capable of undergoing hybridization to a target nucleic acid through hydrogen bonding.

“Antisense oligonucleotide” can mean a single-stranded oligonucleotide having a nucleobase sequence that permits hybridization to a corresponding segment of a target nucleic acid.

“Base complementarity” can refer to the capacity for the precise base pairing of nucleobases of an antisense oligonucleotide with corresponding nucleobases in a target nucleic acid (i.e., hybridization), and is mediated by Watson-Crick, Hoogsteen or reversed Hoogsteen hydrogen binding between corresponding nucleobases.

“Bicyclic sugar” can mean a furanose ring modified by the bridging of two atoms. A bicyclic sugar is a modified sugar.

“Bicyclic nucleoside” (also “BNA”) can mean a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

“Cap structure” or “terminal cap moiety” can mean chemical modifications, which have been incorporated at either terminus of an antisense oligomer. “cEt” or “constrained ethyl” can mean a bicyclic nucleoside having a sugar moiety comprising a bridge connecting the 4′-carbon and the 2′-carbon, wherein the bridge has the formula: 4′-CH(CH3)-O-2.

“Constrained ethyl nucleoside” (also cEt nucleoside) can mean a nucleoside comprising a bicyclic sugar moiety comprising a 4′-CH(CH3)-O-2′ bridge.

“Chimeric antisense oligomer” can mean an antisense oligomer that has at least two chemically distinct regions, each position having a plurality of subunits.

“Complementarity” can mean the capacity for pairing between nucleobases of a first nucleic acid and a second nucleic acid.

“Contiguous nucleobases” can mean nucleobases immediately adjacent to each other.

“Diluent” can mean an ingredient in a composition that lacks pharmacological activity but is pharmaceutically necessary or desirable. For example, in drugs that are injected, the diluent may be a liquid, e.g., saline solution.

“Effective amount” in the context of modulating an activity or of treating or preventing a condition can mean the administration of that amount of pharmaceutical agent to an individual in need of such modulation, treatment, or prophylaxis, either in a single dose or as part of a series, that is effective for modulation of that effect, or for treatment or prophylaxis or improvement of that condition. The effective amount may vary among individuals depending on the health and physical condition of the individual to be treated, the taxonomic group of the individuals to be treated, the formulation of the composition, assessment of the individual's medical condition, and other relevant factors.

“Efficacy” or “potency,” which are used herein interchangeably, can mean the ability to produce a desired effect.

“Expression” can include all the processes by which a gene's coded information is converted into structures present and operating in a cell. Such structures include, but are not limited to, the products of transcription and translation.

“Gapmer” can mean a chimeric antisense oligomer in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region can be referred to as a “gap” and the external regions can be referred to as the “wings.”

“Hybridization” can mean the annealing of complementary nucleic acid molecules. In certain embodiments, complementary nucleic acid molecules include, but are not limited to, an antisense oligomer and a target nucleic acid. In certain embodiments, complementary nucleic acid molecules include, but are not limited to, an antisense oligonucleotide and a nucleic acid target.

“Individual” can mean a human or non-human animal selected for treatment or therapy.

“Inhibiting SYNGAP” or “inhibiting SYNGAP” can mean reducing the level or expression of a SYNGAP mRNA and/or SYNGAP protein. In certain embodiments, SYNGAP mRNA and/or SYNGAP protein levels are inhibited in the presence of an antisense oligomer targeting SYNGAP, including an antisense oligonucleotide targeting SYNGAP, as compared to expression of SYNGAP mRNA and/or SYNGAP protein levels in the absence of a SYNGAP antisense oligomer, such as an antisense oligonucleotide.

“Inhibiting the expression or activity” can refer to a reduction or blockade of the expression or activity and does not necessarily indicate a total elimination of expression or activity.

“Internucleoside linkage” can refer to the chemical bond between nucleosides.

“Intra-cisterna magna” or “ICM” injection or delivery can refer to injection of an agent or pharmaceutical composition provided herein in the cerebrospinal fluid (CSF)-filled subarachnoid space between the cerebellum and the dorsal side of the medulla oblongata.

“Linked nucleosides” can refer to adjacent nucleosides linked together by an internucleoside linkage.

“SYNGAP antisense oligomer” can mean an antisense oligomer targeting SYNGAP mRNA.

“Mismatch” or “non-complementary nucleobase” can refer to the case when a nucleobase of a first nucleic acid is not capable of pairing with the corresponding nucleobase of a second or target nucleic acid.

“Modified internucleoside linkage” can refer to a substitution or any change from a naturally occurring internucleoside bond (i.e., a phosphodiester internucleoside bond).

“Modified nucleobase” can refer to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleoside” can refer to a nucleoside having, independently, a modified sugar moiety and/or modified nucleobase.

“Modified nucleotide” can refer to a nucleotide having, independently, a modified sugar moiety, modified internucleoside linkage, and/or modified nucleobase.

“Modified antisense oligonucleotide” can refer to an oligonucleotide comprising at least one modified internucleoside linkage, modified sugar, and/or modified nucleobase.

“Modified sugar” can refer to substitution and/or any change from a natural sugar moiety.

“Monomer” can refer to a single unit of an oligomer. Monomers include, but are not limited to, nucleosides and nucleotides, whether naturally occurring or modified. “Motif means the pattern of unmodified and modified nucleosides in an antisense oligomer.

“Natural sugar moiety” can refer to a sugar moiety found in DNA (2′-H) or RNA (2′-OH).

“Naturally occurring internucleoside linkage” can refer to a 3′ to 5′ phosphodiester linkage.

“Non-complementary nucleobase” can refer to a pair of nucleobases that do not form hydrogen bonds with one another or otherwise support hybridization.

“Nucleic acid” can refer to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” can mean a heterocyclic moiety capable of pairing with a base of another nucleic acid. “Nucleobase complementarity” can refer to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense oligomer that is capable of base pairing with a nucleobase of its target nucleic acid. For example, if a nucleobase at a certain position of an antisense oligomer is capable of hydrogen bonding with a nucleobase at a certain position of a target nucleic acid, then the position of hydrogen bonding between the oligonucleotide and the target nucleic acid is considered to be complementary at that nucleobase pair.

“Nucleobase sequence” can refer to the order of contiguous nucleobases independent of any sugar, linkage, and/or nucleobase modification.

“Nucleoside” can refer to a nucleobase linked to a sugar.

“Nucleoside mimetic” can include those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo, or tricyclo sugar mimetics, e.g., non furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” can refer to groups that are substituted for a sugar, a nucleobase, and/or internucleoside linkage. Generally, a mimetic can be used in place of the sugar or sugar-internucleoside linkage combination, and the nucleobase is maintained for hybridization to a selected target.

“Nucleotide” can refer to a nucleoside having a phosphate group covalently linked to the sugar portion of the nucleoside.

“Oligomeric compound” or “oligomer,” which are used herein interchangeably, can refer to a polymer of linked monomeric subunits which is capable of hybridizing to at least a region of a nucleic acid molecule.

“Oligonucleotide” can refer to a polymer of linked nucleosides each of which can be modified or unmodified, independent one from another.

“Parenteral administration” can refer to administration through injection (e.g., bolus injection) or infusion. Parenteral administration can include subcutaneous administration, intravenous administration, intramuscular administration, intraarterial administration, intraperitoneal administration, or intracranial administration, e.g., intrathecal, intracerebroventricular, or intra cisterna magna administration.

“Peptide” can refer to a molecule formed by linking at least two amino acids by amide bonds. Without limitation, as used herein, peptide refers to polypeptides and proteins.

“Pharmaceutical agent” can refer to a substance that provides a therapeutic benefit when administered to an individual. For example, in certain embodiments, an antisense oligonucleotide targeted to SYNGAP is a pharmaceutical agent.

“Pharmaceutical composition” can refer to a mixture of substances suitable for administering to an individual. For example, a pharmaceutical composition can comprise an antisense oligonucleotide and a sterile aqueous solution.

“Pharmaceutically acceptable salts” can refer to physiologically and pharmaceutically acceptable salts of a pharmaceutically active ingredient (e.g., an antisense oligomer provided herein), such as salts that retain the desired biological activity of the active ingredient and do not impart undesired toxicological effects thereto.

“Phosphorothioate linkage” can mean a linkage between nucleosides where the phosphodiester bond is modified by replacing one of the non-bridging oxygen atoms with a sulfur atom. A phosphorothioate linkage is a modified internucleoside linkage.

“Portion” can mean a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense oligomer.

“Prevent” or “preventing” can mean delaying or forestalling the onset or development of a disorder or syndrome for a period of time from minutes to days, weeks to months, or indefinitely.

“Prophylactically effective amount” can mean an amount of a pharmaceutical agent that provides a prophylactic or preventative benefit to an animal.

“Ribonucleotide” can mean a nucleotide having a hydroxy at the 2′ position of the sugar portion of the nucleotide. Ribonucleotides may be modified with any of a variety of substituents.

“Segments” are defined as smaller or sub-portions of regions within a target nucleic acid.

“Targeting” or “targeted” can mean the process of design and selection of an antisense oligomer that will specifically hybridize to a target nucleic acid and induce a desired effect.

“Target nucleic acid,” “target RNA,” and “target RNA transcript” and “nucleic acid target” all can mean a nucleic acid capable of being targeted by antisense oligomers. In certain embodiments, the target nucleic acid is a UBE2A nucleic acid.

“Target region” can mean a portion of a target nucleic acid to which one or more antisense oligomers is targeted.

“Target segment” can mean the sequence of nucleotides of a target nucleic acid to which an antisense oligomer is targeted. “5′ target site” refers to the 5′-most nucleotide of a target segment. “3′ target site” refers to the 3′-most nucleotide of a target segment.

“Therapeutically effective amount” can mean an amount of a pharmaceutical agent that provides a therapeutic benefit to an individual. “Treat” or “treating” or “treatment” can refer to administering a composition to effect an alteration or improvement of the disorder or syndrome.

“Unmodified nucleobases” can mean the purine bases adenine (A) and guanine (G), and the pyrimidine bases thymine (T), cytosine (C) and uracil (U).

“Unmodified nucleotide” can mean a nucleotide composed of naturally occurring nucleobases, sugar moieties, and internucleoside linkages. In certain embodiments, an unmodified nucleotide is an RNA nucleotide (i.e., β-D-ribonucleosides) or a DNA nucleotide (i.e., β-D-deoxyribonucleoside).

“Wing segment” can mean a plurality of nucleosides modified to impart to an oligonucleotide properties such as enhanced inhibitory activity, increased binding affinity for a target nucleic acid, or resistance to degradation by in vivo nucleases.

In certain embodiments, an antisense oligomer targeted to a target nucleic acid is 12 to 30 subunits in length. In certain embodiments, an antisense oligomer targeted to a target nucleic acid is 12 to 25 subunits in length. In certain embodiments, an antisense oligomer targeted to a target nucleic acid is 12 to 22 subunits in length. In certain embodiments, an antisense oligomer targeted to a target nucleic acid is 14 to 20 subunits in length. In certain embodiments, an antisense oligomer targeted to a target nucleic acid is 15 to 25 subunits in length. In certain embodiments, an antisense oligomer targeted to a target nucleic acid is 18 to 22 subunits in length. In certain embodiments, an antisense oligomer targeted to a target nucleic acid is 19 to 21 subunits in length. In certain embodiments, the antisense oligomer is 8 to 80, 12 to 50, 13 to 30, 13 to 50, 14 to 30, 14 to 50, 15 to 30, 15 to 50, 16 to 30, 16 to 50, 17 to 30, 17 to 50, 18 to 30, 18 to 50, 19 to 30, 19 to 50, or 20 to 30 linked subunits in length.

In certain embodiments, an antisense oligomer targeted to a target nucleic acid is 12 subunits in length. In certain embodiments, an antisense oligomer targeted to a target nucleic acid is 13 subunits in length. In certain embodiments, an antisense oligomer targeted to a target nucleic acid is 14 subunits in length. In certain embodiments, an antisense oligomer targeted to a target nucleic acid is 15 subunits in length. In certain embodiments, an antisense oligomer targeted to a target nucleic acid is 16 subunits in length. In certain embodiments, an antisense oligomer targeted to a target nucleic acid is 17 subunits in length. In certain embodiments, an antisense oligomer targeted to a target nucleic acid is 18 subunits in length. In certain embodiments, an antisense oligomer targeted to a target nucleic acid is 19 subunits in length. In certain embodiments, an antisense oligomer targeted to a target nucleic acid is 20 subunits in length. In certain embodiments, an antisense oligomer targeted to a target nucleic acid is 21 subunits in length. In certain embodiments, an antisense oligomer targeted to a target nucleic acid is 22 subunits in length. In certain embodiments, an antisense oligomer targeted to a target nucleic acid is 23 subunits in length. In certain embodiments, an antisense oligomer targeted to a target nucleic acid is 24 subunits in length. In certain embodiments, an antisense oligomer targeted to a target nucleic acid is 25 subunits in length. In certain embodiments, an antisense oligomer targeted to a target nucleic acid is 26 subunits in length. In certain embodiments, an antisense oligomer targeted to a target nucleic acid is 27 subunits in length. In certain embodiments, an antisense oligomer targeted to a target nucleic acid is 28 subunits in length. In certain embodiments, an antisense oligomer targeted to a target nucleic acid is 29 subunits in length. In certain embodiments, an antisense oligomer targeted to a target nucleic acid is 30 subunits in length. In certain embodiments, the antisense oligomer targeted to a target nucleic acid is 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In certain embodiments the antisense oligomer is an antisense oligonucleotide, and the linked subunits are nucleosides.

Antisense oligomers provided herein can have nucleotides that mismatch the target sequence. For instance, an antisense oligonucleotide of 25 nucleobases in length can have 8 or 11 mismatch bases near the ends of the antisense oligonucleotides, while still being able to direct specific cleavage of the target mRNA, albeit to a lesser extent than the antisense oligonucleotides that contained no mismatches. In some cases, the antisense oligonucleotide provided herein has 12 to 30 subunits in length (e.g., nucleobases), including those with 1 or 3 mismatches.

### Chemically Modified Antisense Oligomer

In certain embodiments, antisense oligomers provided herein have chemically modified subunits arranged in patterns, or motifs, to confer to the antisense oligomers properties such as enhanced inhibitory activity, increased binding affinity for a target nucleic acid, or resistance to degradation by in vivo nucleases.

In some cases, provided herein are chimeric antisense oligomers. For instance, chimeric antisense oligomers can contain at least one region modified so as to confer increased resistance to nuclease degradation, increased cellular uptake, increased binding affinity for the target nucleic acid, and/or increased inhibitory activity. A second region of a chimeric antisense oligomer can optionally serve as a substrate for the cellular endonuclease RNase H, which cleaves the RNA strand of an RNA:DNA duplex.

In some cases, the antisense oligomers provided herein have a gapmer motif. Antisense oligomers having a gapmer motif can be considered chimeric antisense oligomers. In a gapmer an internal region having a plurality of nucleotides that supports RNaseH cleavage can be positioned between external regions having a plurality of nucleotides that are chemically distinct from the nucleosides of the internal region. In the case of an antisense oligonucleotide having a gapmer motif, the gap segment can serve as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer can include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH₂)n-O-2′ bridge, where n=1 or n=2 and 4′-CH₂—O—CH₂-2′). In certain embodiments, wings include several modified sugar moieties, including, for example 2′-MOE. In certain embodiments, wings include several modified and unmodified sugar moieties. In certain embodiments, wings include various combinations of 2′-MOE nucleosides and 2′-deoxynucleosides.

Each distinct region can comprise uniform sugar moieties, variant, or alternating sugar moieties. The wing-gap-wing motif is frequently described as “X—Y—Z”, where “X” represents the length of the 5′ wing, “Y” represents the length of the gap, and “Z” represents the length of the 3′ wing. “X” and “Z” can comprise uniform, variant, or alternating sugar moieties. In certain embodiments, “X” and “Y” include one or more 2′-deoxynucleosides. “Y” can comprise 2′-deoxynucleosides. As used herein, a gapmer described as “X—Y—Z” can have a configuration such that the gap is positioned immediately adjacent to each of the 5′ wing and the 3′ wing. Thus, no intervening nucleotides can exist between the 5′ wing and gap, or the gap and the 3′ wing. Any of the antisense oligomers described herein can have a gapmer motif. In certain embodiments, “X” and “Z” are the same; in other cases, they are different.

In certain cases, gapmers provided herein include, for example 20-mers having a motif of 5-10-5 in the form of “X—Y—Z” as described herein. In certain embodiments, gapmers provided herein include, for example 19-mers having a motif of 5-9-5 in the form of “X—Y—Z” as described herein. In certain embodiments, gapmers provided herein include, for example 18-mers having a motif of 5-8-5 in the form of “X—Y—Z” as described herein. In certain embodiments, gapmers provided herein include, for example 18-mers having a motif of 4-8-6 in the form of “X—Y—Z” as described herein. In certain embodiments, gapmers provided herein include, for example 18-mers having a motif of 6-8-4 in the form of “X—Y—Z” as described herein. In certain embodiments, gapmers provided herein include, for example 18-mers having a motif of 5-7-6 in the form of “X—Y—Z” as described herein.

In some cases, the antisense oligomer comprises: a 5′ region consisting of three, four, five, or six linked nucleosides (e.g., “X” part discussed above); a central region consisting of eight, nine, ten, eleven, or twelve linked nucleosides (e.g., “Y” part discussed above); and a 3′ region consisting of three, four, five, or six linked nucleosides (e.g., “Z” part discussed above). In some cases, each of the three, four, five, or six linked nucleosides in the 5′ region and each of three, four, five, or six linked nucleosides in the 3′ region comprise a modified sugar moiety, and each of the eight, nine, ten, eleven, or twelve linked nucleosides in the central region is a deoxyribonucleoside. In some cases, the modified sugar moiety includes a 2′-O-methyl moiety, a 2′-Fluoro moiety, a 2′-O-methoxyethyl moiety, 2′-NMA moiety, or any combination thereof. In some cases, one or more the nucleosides in the 5′ region and in the 3′ region further comprise other modification as disclosed herein. In some cases, all the nucleosides in the 5′ region and in the 3′ region further comprise other modification as disclosed herein.

### Complementarity

An agent provided herein can have a polynucleotide sequence complementary to a target nucleic acid when a sufficient number of nucleobases in the polynucleotide sequence (for instance antisense oligomer) can hydrogen bond with the corresponding nucleobases of the target nucleic acid, such that a desired effect can occur (e.g., antisense inhibition of a target nucleic acid, such as a SYNGAP nucleic acid).

Non-complementary nucleobases between an agent (e.g., an antisense oligomer) and a target nucleic acid may be tolerated provided that the agent (e.g., antisense oligomer) remains able to specifically hybridize to a target nucleic acid. Moreover, an agent (e.g., antisense oligomer) can hybridize to one or more segments of a target nucleic acid such that intervening or adjacent segments are not involved in the hybridization event (e.g., a loop structure, mismatch, or hairpin structure).

In certain embodiments, the agents (e.g., antisense oligomers) provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a SYNGAP nucleic acid, a target region, target segment, or specified portion thereof. Percent complementarity of an antisense oligomer with a target nucleic acid can be determined using routine methods, such as using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the agents (e.g., antisense oligomers) provided herein, or specified portions thereof, are fully complementary (i.e., 100% complementary) to a target nucleic acid, or specified portion thereof. For example, agents (e.g., antisense oligomers) provided herein can be fully complementary to a SYNGAP nucleic acid, or a target region, or a target segment or target sequence thereof. As used herein, “fully complementary” can mean that each nucleobase of an antisense oligomer is capable of precise base pairing with the corresponding nucleobases of a target nucleic acid.

The location of a non-complementary nucleobase can be at the 5′ end or 3′ end of the antisense oligomer. Alternatively, the non-complementary nucleobase or nucleobases can be at an internal position of the antisense oligomer. When two or more non-complementary nucleobases are present, they can be contiguous (i.e., linked) or non-contiguous. In one embodiment, a non-complementary nucleobase is located in the wing segment of a gapmer antisense oligonucleotide.

In certain embodiments, antisense oligomers provided herein that are, or are up to 1 1, 12, 13, 14, 15, 16, 17, 18, 19, or 20 nucleobases in length comprise no more than 4, no more than 3, no more than 2, or no more than 1 non-complementary nucleobase(s) relative to a target nucleic acid, or specified portion thereof.

In certain embodiments, antisense oligomers provided herein that are, or are up to 1 1, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, or specified portion thereof.

The agents (e.g., antisense oligomers) provided herein can also include those which are complementary to a portion of a target nucleic acid. As used herein, “portion” can refer to a defined number of contiguous (i.e., linked) nucleobases within a region or segment of a target nucleic acid. A “portion” can also refer to a defined number of contiguous nucleobases of an antisense oligomer. In certain embodiments, the agents (e.g., antisense oligomers) are complementary to at least an 8-nucleobase portion of a target segment. In certain embodiments, the agents (e.g., antisense oligomers) are complementary to at least a 9-nucleobase portion of a target segment. In certain embodiments, the agents (e.g., antisense oligomers) are complementary to at least a 10-nucleobase portion of a target segment. In certain embodiments, the agents (e.g., antisense oligomers) are complementary to at least an 11-nucleobase portion of a target segment. In certain embodiments, the agents (e.g., antisense oligomers) are complementary to at least a 12-nucleobase portion of a target segment. In certain embodiments, the agents (e.g., antisense oligomers) are complementary to at least a 13-nucleobase portion of a target segment. In certain embodiments, the agents (e.g., antisense oligomers) are complementary to at least a 14-nucleobase portion of a target segment. In certain embodiments, the agents (e.g., antisense oligomers) are complementary to at least a 15-nucleobase portion of a target segment. Also contemplated are antisense oligomers that are complementary to at least a 9, 10, 11, 12, 13, 14, 15, 16, 17, 18, 19, 20, or more nucleobase portion of a target segment, or a range defined by any two of these values.

### Sequence Identity

The agents (e.g., antisense oligomers) provided herein can also have a defined percent identity to a particular nucleotide sequence, SEQ ID NO, or portion thereof. As used herein, an antisense oligomer is identical to the sequence disclosed herein if it has the same nucleobase pairing ability. For example, an RNA which contains uracil in place of thymidine in a disclosed DNA sequence would be considered identical to the DNA sequence since both uracil and thymidine pair with adenine. Shortened and lengthened versions of the antisense oligomers described herein as well as oligomers having non-identical bases relative to the antisense oligomers provided herein also are contemplated. The non-identical bases can be adjacent to each other or dispersed throughout the antisense oligomer. Percent identity of an antisense oligomer is calculated according to the number of bases that have identical base pairing relative to the sequence to which it is being compared.

In certain embodiments, the agents (e.g., antisense oligomers), or portions thereof, are at least 70%, 75%, 80%, 85%, 90%, 95%, 96%, 97%, 98%, 99% or 100% identical to one or more of the agents (e.g., antisense oligomers) or SEQ ID NOs, or a portion thereof, disclosed herein.

In certain embodiments, a portion of the agent (e.g., antisense oligomer) is compared to an equal length portion of the target nucleic acid. In certain embodiments, an 8, 9, 10, 1 1, 12, 13, 14, 15, 16, 17, 18, 19, 20, 21, 22, 23, 24, or 25 nucleobase portion is compared to an equal length portion of the target nucleic acid.

In certain embodiments, a portion of the antisense oligonucleotide is compared to an equal length portion of the target nucleic acid. In certain embodiments, an 8, 9, 10, 1 1, 12, 13, 14, 15, 16, 17, 18, 19, 20, 21, 22, 23, 24, or 25 nucleobase portion is compared to an equal length portion of the target nucleic acid.

### Modifications

In some embodiments, an antisense oligomer provided herein can have one or more chemical modifications as compared to a naturally occurring nucleotide (or a native form of the antisense oligomer) that has the same or comparable polynucleotide sequence. Modifications to antisense oligomers encompass substitutions or changes to internucleoside linkages, sugar moieties, or nucleobases. Modified antisense oligomers can be preferred over native forms because of desirable properties such as, for example, enhanced cellular uptake, enhanced affinity for nucleic acid target, increased stability in the presence of nucleases, or increased inhibitory activity.

Chemically modified nucleosides can be employed to increase the binding affinity of a shortened or truncated antisense oligonucleotide for its target nucleic acid. Consequently, comparable results can often be obtained with shorter antisense oligomers that have such chemically modified nucleosides.

A nucleoside can be a base-sugar combination. The nucleobase (also known as base) portion of the nucleoside can be a heterocyclic base moiety in native form. Nucleotides are nucleosides that further include a phosphate group covalently linked to the sugar portion of the nucleoside. For those nucleosides that include a pentofuranosyl sugar, the phosphate group can be linked to the 2′, 3′, or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

### Modified Internucleoside Linkages

The naturally occurring internucleoside linkage of RNA and DNA is a 3′ to 5′ phosphodiester linkage. Antisense oligomers provided herein can have one or more modified, i.e., non-naturally occurring, internucleoside linkages. Antisense oligomers having one or more modified internucleotide linkages can have desirable properties such as, for example, enhanced cellular uptake, enhanced affinity for target nucleic acids, and increased stability in the presence of nucleases.

Oligonucleotides having modified internucleoside linkages can include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates.

In certain embodiments, antisense oligomers targeted to a SYNGAP nucleic acid comprise one or more modified internucleoside linkages. In certain embodiments, the modified internucleoside linkages are interspersed throughout the antisense oligomer. In certain embodiments, the modified internucleoside linkages are phosphorothioate linkages. In certain embodiments, each internucleoside linkage of an antisense oligomer is a phosphorothioate internucleoside linkage.

### Modified Sugar Moieties

Antisense oligomers provided herein can contain one or more nucleosides wherein the sugar group has been modified. Such sugar modified nucleosides can impart enhanced nuclease stability, increased binding affinity, or some other beneficial biological property to the antisense oligomers. In certain embodiments, nucleosides comprise chemically modified ribofuranose ring moieties.

Examples of chemically modified ribofuranose rings include without limitation, addition of substitute groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH₃, 2′-OCH₂CH₃, 2′-OCH₂CH₂F, 2′-NMA, and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(Rₗ)—(CH₂)₂—N(Rₘ)(Rₙ), where each Rₗ, Rₘ and Rₙ is, independently, H or substituted or unsubstituted C₁-C₁₀ alkyl.

As used herein, “bicyclic nucleosides” can refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense oligomers provided herein include one or more bicyclic nucleosides comprising a 4′ to 2′ bridge. Examples of such 4′ to 2′ bridged bicyclic nucleosides, include but are not limited to one of those described in U.S. Pat. Nos. 7,399,845, 8,278,283, 7,696,345, 7,427,672, 8,278,426, 6,268,490; 6,525,191; 6,670,461; 6,770,748; 6,794,499; 7,034,133; 7,053,207, 7,399,845, 7,547,684, 7,741,457, and 7,696,345; U.S. Patent Nos.; U.S. Patent Publication No. US2008-0039618; and Chattopadhyaya et al., J. Org. Chem., 2009, 74, 1 18-134); Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc, 2007, 129(26) 8362-8379; Elayadi et al., Curr. Opinion Invest. Drugs, 2001, 2, 558-561; Braasch et al., Chem. Biol, 2001, 8, 1-7; and Orum et al., Curr. Opinion Mol. Ther., 2001, 3, 239-243. Each of the foregoing bicyclic nucleosides can be prepared having one or more stereochemical sugar configurations including for example a-L-ribofuranose and β-D-ribofuranose (see PCT international application PCT/DK98/00393, published on Mar. 25, 1999 as WO 99/14226). In certain embodiments, bicyclic sugar moieties of BNA nucleosides include, but are not limited to, described in U.S. Pat. No. 11,129,844.

The synthesis and preparation of the methyleneoxy (4′-CH₂—O-2′) BNA monomers adenine, cytosine, guanine, 5-methyl-cytosine, thymine, and uracil, along with their oligomerization, and nucleic acid recognition properties have been described (Koshkin et al., Tetrahedron, 1998, 54, 3607-3630).

As used herein, “4′-2′ bicyclic nucleoside” or “4′ to 2′ bicyclic nucleoside” can refer to a bicyclic nucleoside comprising a furanose ring comprising a bridge connecting two carbon atoms of the furanose ring connects the 2′ carbon atom and the 4′ carbon atom of the sugar ring.

As used herein, “monocyclic nucleosides” can refer to nucleosides comprising modified sugar moieties that are not bicyclic sugar moieties. In certain embodiments, the sugar moiety, or sugar moiety analogue, of a nucleoside is modified or substituted at any position.

As used herein, “2′-modified sugar” can mean a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙF, O(CH₂)ₙONH₂, OCH₂C(═O)N(H)CH₃ and O(CH₂)ₙON[(CH₂)ₙCH₃]₂, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, CI, Br, CN, F, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense oligomer, and other substituents having similar properties. In certain embodiments, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, “2′-NMA” can mean a —O—CH₂—C(═O)—NH—CH₃ group in place of the 2′-OH group of a ribosyl sugar moiety. A “2′-NMA sugar moiety” or “2′-NMA moiety” is a sugar moiety with a 2′-O—CH₂—C(═O)—NH—CH₃ group in place of the 2′-OH group of a ribosyl sugar moiety. Unless otherwise indicated, a 2′-NMA sugar moiety is in the β-D configuration. “NMA” can mean O—N-methyl acetamide.

As used herein, “2′-NMA nucleoside” can mean a nucleoside comprising a 2′-NMA sugar moiety.

As used herein, “2′-F” can refer to a nucleoside comprising a sugar comprising a fluoro group at the 2′ position.

As used herein, “2′-OMe” or “2′-OCH₃” or “2′-O-methyl” each can refer to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

In certain embodiments, one or more of the plurality of nucleosides is modified. In certain embodiments, an oligonucleotide comprises one or more ribonucleosides (RNA) and/or deoxyribonucleosides (DNA). In certain embodiments, an oligonucleotide comprises a mix of one or more ribonucleosides (RNA) and deoxyribonucleosides (DNA).

Many other bicyclo and tricyclo sugar surrogate ring systems are also known in the art that can be used to modify nucleosides for incorporation into antisense oligomers (see for example review article: Leumann, Bioorg. Med. Chem., 2002, 10, 841-854). Such ring systems can undergo various additional substitutions to enhance activity.

Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense oligomers comprise one or more nucleosides having modified sugar moieties. In certain embodiments, the modified sugar moiety is 2′-MOE. In certain embodiments, the 2′-MOE modified nucleosides are arranged in a gapmer motif. In certain embodiments, the modified sugar moiety is a bicyclic nucleoside having a (4′-CH(CH₃)—O-2′) bridging group. In certain embodiments, the (4′-CH(CH₃)—O-2′) modified nucleosides are arranged throughout the wings of a gapmer motif.

“5′-methylcytosine” can mean a cytosine modified with a methyl group attached to the 5′ position. A 5′-methylcytosine is a modified nucleobase.

“5′-methyluracil” can mean a uracil modified with a methyl group attached to the 5′ position. A 5′-methyluracil is a modified nucleobase.

“5′-methylthymine” can mean a thymine modified with a methyl group attached to the 5′ position. A 5′-methylthymine is a modified nucleobase.

In some cases, antisense oligomers provided herein comprise 5′-methylcytosine, 5′-methyluracil, 5′-methylthymine, or a combination thereof. In some cases, each cytosine in the antisense oligomer is methylated, i.e., having a methyl group attached to the 5′ position. In some cases, each uracil in the antisense oligomer is methylated, i.e., having a methyl group attached to the 5′ position. In some cases, the antisense oligomer has 1, 2, 3, 4, 5, 6, 7, 8, or more 5′-methylcytosine. In some cases, the antisense oligomer has 1, 2, 3, 4, 5, 6, 7, 8, or more 5′-methyluracil. In some cases, the antisense oligomer has both methylcytosine and methyluracil.

### Pharmaceutical Compositions and Methods of Treatment

In some aspects, provided herein are pharmaceutical compositions comprising an agent of the present disclosure, e.g., an antisense oligomer, or a vector encoding the agent.

Pharmaceutical compositions or formulations comprising the agent, e.g., antisense oligomer, or a vector encoding the agent, of the described compositions and for use in any of the described methods can be prepared according to conventional techniques well known in the pharmaceutical industry and described in the published literature. In some embodiments, a pharmaceutical composition or formulation for treating a subject comprises an effective amount of any antisense oligomer as described herein, or a pharmaceutically acceptable salt, solvate, hydrate or ester thereof. The pharmaceutical formulation comprising an antisense oligomer may further comprise a pharmaceutically acceptable excipient, diluent, or carrier.

Agents (e.g., antisense oligomers) or vectors provided herein can be admixed with pharmaceutically acceptable active or inert substances for the preparation of pharmaceutical compositions or formulations. An agent (e.g., antisense oligomer) targeted to a SYNGAP nucleic acid can be utilized in pharmaceutical compositions by combining the agent with a suitable pharmaceutically acceptable diluent or carrier. A pharmaceutically acceptable diluent can include phosphate-buffered saline (PBS), artificial cerebrospinal fluid (aCSF), physiological saline, or any other suitable solutions.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

In some embodiments, the compositions are formulated into any of many possible dosage forms such as, but not limited to, tablets, capsules, gel capsules, liquid syrups, soft gels, suppositories, and enemas. In embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In embodiments, a pharmaceutical formulation or composition of the present disclosure includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In embodiments, liposomes also include sterically stabilized liposomes, e.g., liposomes comprising one or more specialized lipids. These specialized lipids result in liposomes with enhanced circulation lifetimes. In embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In embodiments, the present disclosure employs a penetration enhancer to effect the efficient delivery of the antisense oligonucleotide, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple agents (e.g., antisense oligomers). In embodiments, the agent (e.g., antisense oligomer) or a vector encoding the agent is administered in combination with another drug or therapeutic agent.

Pharmaceutical compositions comprising antisense oligomers can encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense oligomers, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense oligomer which are cleaved by endogenous nucleases within the body, to form the active antisense oligomer.

Antisense oligomers disclosed herein can be covalently linked to one or more moieties or conjugates which enhance the activity, cellular distribution, or cellular uptake of the resulting antisense oligomers. Conjugate groups can include cholesterol moieties and lipid moieties. Additional conjugate groups can include carbohydrates, phospholipids, biotin, phenazine, folate, phenanthridine, anthraquinone, acridine, fluoresceins, rhodamines, coumarins, and dyes.

Antisense oligomers of the present disclosure can also be modified to have one or more stabilizing groups that are generally attached to one or both termini of antisense oligomers to enhance properties such as, for example, nuclease stability. Included in stabilizing groups are cap structures. These terminal modifications can protect the antisense oligomer having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures can include, for example, inverted deoxy abasic caps. Further 3′ and 5′-stabilizing groups that can be used to cap one or both ends of an antisense oligomer to impart nuclease stability can include those disclosed in WO 03/004602 published on Jan. 16, 2003.

### Autosomal Dominant Mental Retardation and SYNGAP1 Gene

Autosomal dominant mental retardation is an autosomal dominant neurodevelopmental disorder characterized by significantly impaired intellectual functioning and adaptive behaviors, one cause of which is haploinsufficiency of SYNGAP1 gene expression. SYNGAP1 codes for SYNGAP1 protein, which is a ras GTPase-activating protein involved in cognitive development and proper synapse function amongst neurons. SYNGAP1 is known to be involved in synaptic plasticity, axon formation, and AMPA receptor trafficking. Generally, SYNGAP1 activity is essential for development and survival. SYNGAP1 deficiencies may be caused by sporadic mutation or alternative splicing events. Deficient SYNGAP1 activity may result in mental retardation, or intellectual disability, epileptic encephalopathy, or autism.

The present disclosure provides compositions and methods for modulating alternative splicing of SYNGAP1 to modulate the production of functional protein-coding mature mRNA, and thus, translated functional SYNGAP1 protein. These compositions and methods include antisense oligomers (ASOs) that can promote canonical splicing of SYNGAP1 pre-mRNA. In various embodiments, functional SYNGAP1 protein can be increased using the methods of the disclosure to treat a condition caused by SYNGAP1 protein deficiency. In some embodiments, the condition is autosomal dominant mental retardation. In other embodiments, the condition is epileptic encephalopathy or autism.

In some embodiments, the methods of the invention are used to increase functional SYNGAP1 protein production to treat a condition in a subject in need thereof. In some embodiments, the subject has a condition in which SYNGAP1 is not necessarily deficient relative to wild-type, but where an increase in SYNGAP1 mitigates the condition nonetheless. In some embodiments, the condition is caused by sporadic mutation. In some embodiments, the methods of the invention are used to reduce functional SYNGAP1 protein production to treat a condition in a subject in need thereof. In some embodiments, the methods of the invention are used to modulate functional SYNGAP1 protein production to treat a condition in a subject in need thereof.

### Target Genes

The present disclosure provides compositions and methods for modulating alternative splicing of a target to modulate the production of functional protein-coding mature mRNA, and thus, translated functional the target protein, wherein the target is SYNGAP1. These compositions and methods include antisense oligomers (ASOs) that can promote canonical splicing of the target pre-mRNA, wherein the target is SYNGAP1. In various embodiments, functional target protein can be increased using the methods of the disclosure to treat a condition caused by target protein deficiency, wherein the target is SYNGAP1. In some embodiments, the condition is autosomal dominant mental retardation. In other embodiments, the condition is epileptic encephalopathy or autism.

In some embodiments, the methods of the invention are used to increase functional the target protein production to treat a condition in a subject in need thereof, wherein the target is any one selected from the group consisting of SYNGAP1. In some embodiments, the subject has a condition in which the target protein is not necessarily deficient relative to wild-type, but where an increase in the target protein mitigates the condition nonetheless, wherein the target is SYNGAP1. In some embodiments, the condition is caused by sporadic mutation. In some embodiments, the methods of the invention are used to reduce functional target protein production to treat a condition in a subject in need thereof, wherein the target is SYNGAP1. In some embodiments, the methods of the invention are used to modulate functional target protein production to treat a condition in a subject in need thereof, wherein the target is SYNGAP1.

### Splicing

Intervening sequences in RNA sequences or introns are removed by a large and highly dynamic RNA-protein complex termed the spliceosome, which orchestrates complex interactions between primary transcripts, small nuclear RNAs (snRNAs) and a large number of proteins. Spliceosomes assemble ad hoc on each intron in an ordered manner, starting with recognition of the 5′ splice site (5′ss) by U1 snRNA or the 3′splice site (3′ss) by the U2 pathway, which involves binding of the U2 auxiliary factor (U2AF) to the 3′ss region to facilitate U2 binding to the branch point sequence (BPS). U2AF is a stable heterodimer composed of a U2AF2-encoded 65-kD subunit (U2AF65), which binds the polypyrimidine tract (PPT), and a U2AF1-encoded 35-kD subunit (U2AF35), which interacts with highly conserved AG dinucleotides at 3′ss and stabilizes U2AF65 binding. In addition to the BPS/PPT unit and 3′ss/5′ss, accurate splicing requires auxiliary sequences or structures that activate or repress splice site recognition, known as intronic or exonic splicing enhancers or silencers. These elements allow genuine splice sites to be recognized among a vast excess of cryptic or pseudo-sites in the genome of higher eukaryotes, which have the same sequences but outnumber authentic sites by an order of magnitude. Although they often have a regulatory function, the exact mechanisms of their activation or repression are poorly understood.

The decision of whether to splice or not to splice can be typically modeled as a stochastic rather than deterministic process, such that even the most defined splicing signals can sometimes splice incorrectly. However, under normal conditions, pre-mRNA splicing proceeds at surprisingly high fidelity. This is attributed in part to the activity of adjacent cis-acting auxiliary exonic and intronic splicing regulatory elements (ESRs or ISRs). Typically, these functional elements are classified as either exonic or intronic splicing enhancers (ESEs or ISEs) or silencers (ESSs or ISSs) based on their ability to stimulate or inhibit splicing, respectively. Although there is now evidence that some auxiliary cis-acting elements may act by influencing the kinetics of spliceosome assembly, such as the arrangement of the complex between U1 snRNP and the 5′ss, it seems very likely that many elements function in concert with trans-acting RNA-binding proteins (RBPs). For example, the serine- and arginine-rich family of RBPs (SR proteins) is a conserved family of proteins that have a key role in defining exons. SR proteins promote exon recognition by recruiting components of the pre-spliceosome to adjacent splice sites or by antagonizing the effects of ESSs in the vicinity. The repressive effects of ESSs can be mediated by members of the heterogeneous nuclear ribonucleoprotein (hnRNP) family and can alter recruitment of core splicing factors to adjacent splice sites. In addition to their roles in splicing regulation, silencer elements are suggested to have a role in repression of pseudo-exons, sets of decoy intronic splice sites with the typical spacing of an exon but without a functional open reading frame. ESEs and ESSs, in cooperation with their cognate trans-acting RBPs, represent important components in a set of splicing controls that specify how, where and when mRNAs are assembled from their precursors.

Alternative splicing is a regulated process during gene expression that can result in multiple isoforms of mature mRNA transcripts that are processed from a single primary mRNA transcript that is transcribed from a single gene, and the resultant multiple proteins that are translated from at least some of the multiple mature mRNA isoforms. In this process, particular exons of a gene may be included within or excluded from the final, processed mRNA produced from that gene. Consequently, the proteins translated from alternatively spliced mRNAs will contain differences in their amino acid sequence and, in some cases, in their biological functions.

The sequences marking the exon-intron boundaries are degenerate signals of varying strengths that can occur at high frequency within human genes. In multi-exon genes, different pairs of splice sites can be linked together in many different combinations, creating a diverse array of transcripts from a single gene. This is commonly referred to as alternative pre-mRNA splicing. Although most mRNA isoforms produced by alternative splicing can be exported from the nucleus and translated into functional polypeptides, different mRNA isoforms from a single gene can vary greatly in their translation efficiency. Those mRNA isoforms with premature termination codons (PTCs) at least 50 bp upstream of an exon junction complex are likely to be targeted for degradation by the nonsense-mediated mRNA decay (NMD) pathway. Mutations in traditional (BPS/PPT/3′ss/5′ss) and auxiliary splicing motifs can cause aberrant splicing, such as exon skipping or cryptic (or pseudo-) exon inclusion or splice-site activation, and contribute significantly to human morbidity and mortality. Both aberrant and alternative splicing patterns can be influenced by natural DNA variants in exons and introns.

Given that exon-intron boundaries can occur at any of the three positions of a codon, it is clear that only a subset of alternative splicing events can maintain the canonical open reading frame. For example, only exons that are evenly divisible by 3 can be skipped or included in the mRNA without any alteration of reading frame. Splicing events that do not have compatible phases will induce a frame-shift. Unless reversed by downstream events, frame-shifts can certainly lead to one or more PTCs, probably resulting in subsequent degradation by NMD. NMD is a translation-coupled mechanism that eliminates mRNAs containing PTCs. NMD can function as a surveillance pathway that exists in all eukaryotes. NMD can reduce errors in gene expression by eliminating mRNA transcripts that contain premature stop codons. Translation of these aberrant mRNAs could, in some cases, lead to deleterious gain-of-function or dominant-negative activity of the resulting proteins. NMD targets not only transcripts with PTCs but also a broad array of mRNA isoforms expressed from many endogenous genes, suggesting that NMD is a master regulator that drives both fine and coarse adjustments in steady-state RNA levels in the cell.

An NMD-inducing exon (“NIE” or “NMD exon”) is an exon or a pseudo-exon that is a region within an intron and can activate the NMD pathway if included in a mature RNA transcript. In constitutive splicing events, the intron containing an NMD exon is usually spliced out, but the intron or a portion thereof (e.g., NMD exon) may be retained during alternative or aberrant splicing events. Mature mRNA transcripts containing such an NMD exon may be non-productive due to frame shifts which induce the NMD pathway. Inclusion of a NMD exon in mature RNA transcripts may downregulate gene expression. mRNA transcripts containing an NMD exon may be referred to as “NIE-containing mRNA” or “NMD exon mRNA” in the current disclosure.

Cryptic (or pseudo-splice sites) have the same splicing recognition sequences as genuine splice sites but are not used in splicing reactions. They outnumber genuine splice sites in the human genome by an order of magnitude and are normally repressed by thus far poorly understood molecular mechanisms. Cryptic 5′ splice sites have the consensus NNN/GUNNNN or NNN/GCNNNN where N is any nucleotide and/is the exon-intron boundary. Cryptic 3′ splice sites have the consensus NAG/N. Their activation is positively influenced by surrounding nucleotides that make them more similar to the optimal consensus of authentic splice sites, namely MAG/GURAGU and YAG/G, respectively, where M is C or A, R is G or A, and Y is C or U.

Splice sites and their regulatory sequences can be readily identified by a skilled person using suitable algorithms publicly available, listed for example in Kralovicova, J. and Vorechovsky, I. (2007) Global control of aberrant splice site activation by auxiliary splicing sequences: evidence for a gradient in exon and intron definition. Nucleic Acids Res., 35, 6399-6413.

The cryptic splice sites or splicing regulatory sequences may compete for RNA-binding proteins, such as U2AF, with a splice site of the NMD exon. In some embodiments, an agent may bind to a cryptic splice site or splicing regulatory sequence to prevent binding of RNA-binding proteins and thereby favor binding of RNA-binding proteins to the NMD exon splice sites.

In some embodiments, the cryptic splice site may not comprise the 5′ or 3′ splice site of the NMD exon. In some embodiments, the cryptic splice site may be at least 10 nucleotides, at least 20 nucleotides, at least 50 nucleotides, at least 100 nucleotides or at least 200 nucleotides upstream of the NMD exon 5′ splice site. In some embodiments, the cryptic splice site may be at least 10 nucleotides, at least 20 nucleotides, at least 50 nucleotides, at least 100 nucleotides, at least 200 nucleotides downstream of the NMD exon 3′ splice site.

### Target Transcripts

In some embodiments, the methods of the present disclosure exploit the presence of non-sense mediated RNA decay alternative exon-containing pre-mRNA (NSAE pre-mRNA) transcribed from a gene. In some embodiments, the methods of the present disclosure exploit the presence of non-sense mediated RNA decay alternative exon-containing pre-mRNA (NSAE pre-mRNA) transcribed from the SYNGAP1 gene. Canonical splicing of the identified SYNGAP1 NSAE pre-mRNA transcripts to produce functional, mature SYNGAP1 mRNA can be induced using a therapeutic agent, such as an ASO, that promotes constitutive splicing of SYNGAP1 NSAE pre-mRNA at the canonical splice sites. In some embodiments, the resulting functional, mature SYNGAP1 mRNA can be translated normally, thereby increasing the amount of functional SYNGAP1 protein in the patient's cells and preventing symptoms of SYNGAP1 associated disease. In some embodiments, canonical splicing of the identified SYNGAP1 NSAE pre-mRNA transcripts to produce functional, mature SYNGAP1 mRNA may be reduced using a therapeutic agent, such as an ASO, that inhibits constitutive splicing of SYNGAP1 NSAE pre-mRNA at the canonical splice sites. In some embodiments, the resulting functional, mature SYNGAP1 mRNA can be translated abnormally, thereby decreasing the amount of functional SYNGAP1 protein in the patient's cells and preventing symptoms of SYNGAP1 associated disease.

In some embodiments, the methods of the present disclosure exploit the presence of non-sense mediated RNA decay alternative exon-containing pre-mRNA (NSAE pre-mRNA) transcribed from the target gene, wherein the target is SYNGAP1. Canonical splicing of the identified target NSAE pre-mRNA transcripts to produce the functional, mature target mRNA can be induced using a therapeutic agent, such as an ASO, that promotes constitutive splicing of the target NSAE pre-mRNA at the canonical splice sites. In some embodiments, the resulting functional, mature target mRNA can be translated normally, thereby increasing the amount of the functional target protein in the patient's cells, and preventing symptoms of the target associated disease. In some embodiments, canonical splicing of the identified target NSAE pre-mRNA transcripts to produce functional, mature target mRNA may be reduced using a therapeutic agent, such as an ASO, that inhibits constitutive splicing of target NSAE pre-mRNA at the canonical splice sites. In some embodiments, the resulting functional, mature target mRNA can be translated abnormally, thereby decreasing the amount of functional target protein in the patient's cells, and preventing symptoms of the target associated disease.

In various embodiments, the present disclosure provides a therapeutic agent that can target SYNGAP1 pre-mRNA to modulate splicing or protein expression level. The therapeutic agent can be a small molecule, nucleic acid oligomer, or polypeptide. In some embodiments, the therapeutic agent is an ASO. Various regions or sequences on the SYNGAP1 pre-mRNA can be targeted by a therapeutic agent, such as an ASO. In some embodiments, the ASO targets a SYNGAP1 NSAE pre-mRNA transcribed from the SYNGAP1 gene. In some embodiments, the ASO targets a SYNGAP1 NSAE pre-mRNA transcribed from the SYNGAP1 gene comprising non-sense mediated RNA decay alternative exons (NSAEs). In some embodiments, the NSAE is comprises a portion of canonical exon 11 or the entire canonical exon 11 of a SYNGAP1 pre-mRNA transcript. In some embodiments, the NSAE is comprises only a portion of canonical exon 11 of a SYNGAP1 pre-mRNA transcript, or canonical exon 11 of a SYNGAP1 pre-mRNA transcript and at least a portion of the intron upstream of exon 11 of the SYNGAP1 pre-mRNA transcript. In some embodiments, the NSAE is included in a SYNGAP1 pre-mRNA transcript due to aberrant splicing. In some embodiments, the aberrant splicing is caused by a mutation in the SYNGAP1 gene. In some embodiments, the mutation is a G→A mutation at position −1 of the exon 11 splice donor (E8SJM, Exon 11 Splice Junction Mutation, c.894G>A). In some embodiments, the ASO targets a sequence within a NSAE of a SYNGAP1 pre-mRNA transcript. In some embodiments, the NSAE is comprises a portion of canonical exon 11 or the entire canonical exon 11 of a SYNGAP1 pre-mRNA transcript. In some embodiments, the ASO targets a sequence within exon 10 or 11 of a SYNGAP1 pre-mRNA transcript. In some embodiments, the ASO targets an exon sequence upstream (or 5′) from the 5′ splice site of exon 11 of a SYNGAP1 pre-mRNA transcript. In some embodiments, the ASO targets an exon sequence downstream (or 3′) from the 3′ splice site of exon 11 of a SYNGAP1 pre-mRNA transcript. In some embodiments, the ASO targets a sequence within an intron flanking the 3′ splice site of a NSAE of a SYNGAP1 pre-mRNA transcript. In some embodiments, the ASO targets a sequence within intron 10 or 11 of a SYNGAP1 pre-mRNA transcript. In some embodiments, the ASO targets an intron sequence upstream (or 5′) from the 3′ splice site of intron 10 or 11 of a SYNGAP1 pre-mRNA transcript. In some embodiments, the ASO targets an intron sequence downstream (or 3′) from the 5′ splice site of intron 10 or 11 of a SYNGAP1 pre-mRNA transcript. In some embodiments, the ASO targets a sequence within an intron flanking the 5′ splice site of a NSAE of a SYNGAP1 pre-mRNA transcript. In some embodiments, the ASO targets a sequence within intron 10 of a SYNGAP1 pre-mRNA transcript. In some embodiments, the ASO targets a sequence within intron 11 of a SYNGAP1 pre-mRNA transcript. In some embodiments, the ASO targets an intron sequence upstream (or 5′) from the 3′ splice site of intron 10 or 11 of a SYNGAP1 pre-mRNA transcript. In some embodiments, the ASO targets an intron sequence downstream (or 3′) from the 5′ splice site of intron 10 or 11 of a SYNGAP1 pre-mRNA transcript. In some embodiments, the ASO targets a sequence comprising an exon-intron boundary of a SYNGAP1 pre-mRNA transcript. In some embodiments, the exon is a NSAE. An exon-intron boundary can refer to the junction of an exon sequence and an intron sequence. In some embodiments, the intron sequence can flank the 5′ end of the NSAE, or the 3′ end of the exon. In some embodiments, the ASO targets a sequence comprising an exon 10-intron 11 boundary of a SYNGAP1 pre-mRNA transcript. In some embodiments, the ASO targets a sequence comprising an intron 10-exon 11 boundary of a SYNGAP1 NSAE pre-mRNA transcript. In some embodiments, the ASO targets a sequence comprising both a portion of an intron and a portion of an exon.

In various embodiments, the present disclosure provides a therapeutic agent that can target a target pre-mRNA to modulate splicing or protein expression level, wherein the target is SYNGAP1. The therapeutic agent can be a small molecule, nucleic acid oligomer, or polypeptide. In some embodiments, the therapeutic agent is an ASO. Various regions or sequences on the target pre-mRNA can be targeted by a therapeutic agent, such as an ASO. In some embodiments, the ASO targets a target NSAE pre-mRNA transcribed from the target gene. In some embodiments, the ASO targets a target NSAE pre-mRNA transcribed from the target gene comprising non-sense mediated RNA decay alternative exons (NSAEs). In some embodiments, the NSAE is comprises a portion of a canonical exon or the entire canonical exon of a target pre-mRNA transcript. In some embodiments, the NSAE is comprises only a portion of a canonical exon of a target pre-mRNA transcript, or a canonical exon of a target pre-mRNA transcript and at least a portion of the intron upstream of the exon of the target pre-mRNA transcript. In some embodiments, the NSAE is included in a target pre-mRNA transcript due to aberrant splicing. In some embodiments, the aberrant splicing is caused by a mutation in the target gene. In some embodiments, the ASO targets a sequence within a NSAE of a target pre-mRNA transcript. In some embodiments, the NSAE is comprises a portion of a canonical exon or the entire canonical exon of a target pre-mRNA transcript. In some embodiments, the ASO targets a sequence within an exon of a target pre-mRNA transcript. In some embodiments, the ASO targets an exon sequence upstream (or 5′) from the 5′ splice site of an exon of a target pre-mRNA transcript. In some embodiments, the ASO targets an exon sequence downstream (or 3′) from the 3′ splice site of an exon of a target pre-mRNA transcript. In some embodiments, the ASO targets a sequence within an intron flanking the 3′ splice site of a NSAE of a target pre-mRNA transcript. In some embodiments, the ASO targets a sequence within an intron of a target pre-mRNA transcript. In some embodiments, the ASO targets an intron sequence upstream (or 5′) from the 3′ splice site of an intron of a target pre-mRNA transcript. In some embodiments, the ASO targets an intron sequence downstream (or 3′) from the 5′ splice site of an intron of a target pre-mRNA transcript. In some embodiments, the ASO targets a sequence within an intron flanking the 5′ splice site of a NSAE of a target pre-mRNA transcript. In some embodiments, the ASO targets a sequence within an intron of a target pre-mRNA transcript. In some embodiments, the ASO targets an intron sequence upstream (or 5′) from the 3′ splice site of an intron of a target pre-mRNA transcript. In some embodiments, the ASO targets an intron sequence downstream (or 3′) from the 5′ splice site of an intron of a target pre-mRNA transcript. In some embodiments, the ASO targets a sequence comprising an exon-intron boundary of a target pre-mRNA transcript. In some embodiments, the exon is a NSAE. An exon-intron boundary can refer to the junction of an exon sequence and an intron sequence. In some embodiments, the intron sequence can flank the 5′ end of the NSAE, or the 3′ end of the exon. In some embodiments, the ASO targets a sequence comprising an exon-intron boundary of a target pre-mRNA transcript. In some embodiments, the ASO targets a sequence comprising an intron-exon boundary of a target NSAE pre-mRNA transcript. In some embodiments, the ASO targets a sequence comprising both a portion of an intron and a portion of an exon.

In some embodiments, the ASO targets a sequence about 4 to about 300 nucleotides upstream (or 5′) from the 3′ splice site of a NSAE of a SYNGAP1 pre-mRNA transcript. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream (or 5′) from the 3′ splice site of the NSAE of a SYNGAP1 pre-mRNA transcript. In some embodiments, the ASO may target a sequence more than 300 nucleotides upstream from the 3′ splice site of the NSAE of a SYNGAP1 pre-mRNA transcript. In some embodiments, the ASO targets a sequence about 4 to about 300 nucleotides downstream (or 3′) from the 3′ splice site of a NSAE of a SYNGAP1 pre-mRNA transcript. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream (or 3′) from the 3′ splice site of the NSAE of a SYNGAP1 pre-mRNA transcript. In some embodiments, the ASO may target a sequence more than 300 nucleotides downstream from the 3′ splice site of the NSAE of a SYNGAP1 pre-mRNA transcript. In some embodiments, the ASO targets a sequence about 4 to about 300 nucleotides downstream (or 3′) from the 5′ splice site of the NSAE of a SYNGAP1 pre-mRNA transcript. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream from the 5′ splice site of the NSAE of a SYNGAP1 pre-mRNA transcript. In some embodiments, the ASO targets a sequence more than 300 nucleotides downstream from the 5′ splice site of the NSAE of a SYNGAP1 pre-mRNA transcript. In some embodiments, the ASO targets a sequence about 4 to about 300 nucleotides upstream (or 5′) from the 5′ splice site of the NSAE of a SYNGAP1 pre-mRNA transcript. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream from the 5′ splice site of the NSAE exon of a SYNGAP1 pre-mRNA transcript. In some embodiments, the ASO targets a sequence more than 300 nucleotides upstream from the 5′ splice site of the NSAE exon of a SYNGAP1 pre-mRNA transcript.

In some embodiments, the ASO targets a sequence about 4 to about 300 nucleotides upstream (or 5′) from the 3′ splice site of a NSAE of a target pre-mRNA transcript, wherein the target is SYNGAP1. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream (or 5′) from the 3′ splice site of the NSAE of a target pre-mRNA transcript. In some embodiments, the ASO may target a sequence more than 300 nucleotides upstream from the 3′ splice site of the NSAE of a target pre-mRNA transcript. In some embodiments, the ASO targets a sequence about 4 to about 300 nucleotides downstream (or 3′) from the 3′ splice site of a NSAE of a target pre-mRNA transcript. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream (or 3′) from the 3′ splice site of the NSAE of a target pre-mRNA transcript. In some embodiments, the ASO may target a sequence more than 300 nucleotides downstream from the 3′ splice site of the NSAE of a target pre-mRNA transcript. In some embodiments, the ASO targets a sequence about 4 to about 300 nucleotides downstream (or 3′) from the 5′ splice site of the NSAE of a target pre-mRNA transcript. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream from the 5′ splice site of the NSAE of a target pre-mRNA transcript. In some embodiments, the ASO targets a sequence more than 300 nucleotides downstream from the 5′ splice site of the NSAE of a target pre-mRNA transcript. In some embodiments, the ASO targets a sequence about 4 to about 300 nucleotides upstream (or 5′) from the 5′ splice site of the NSAE of a target pre-mRNA transcript. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream from the 5′ splice site of the NSAE exon of a target pre-mRNA transcript. In some embodiments, the ASO targets a sequence more than 300 nucleotides upstream from the 5′ splice site of the NSAE exon of a target pre-mRNA transcript.

In some embodiments, the ASOs disclosed herein target a NSAE pre-mRNA transcribed from a SYNGAP1 genomic sequence. In some embodiments, the ASO targets a NSAE pre-mRNA transcript from a SYNGAP1 genomic sequence comprising a NSAE exon. In some embodiments, the ASO targets a NSAE pre-mRNA transcript from a SYNGAP1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of the pre-mRNA transcripts of Table 6. In some embodiments, the ASO targets a SYNGAP1 pre-mRNA sequence comprising a NSAE exon. In some embodiments, the ASO targets a SYNGAP1 pre-mRNA sequence comprising an intron flanking the 3′ splice site of the NSAE exon. In some embodiments, the ASO targets a SYNGAP1 pre-mRNA sequence comprising an intron flanking the 5′ splice site of the NSAE exon. In some embodiments, the transcript is selected from the group consisting of the transcripts of Table 6.

In some embodiments, the pre-mRNA transcript comprises a sequence with at least about 80%, 85%, 90%, 95%, 96%, 97%, 98%, 99% or 100% sequence identity to a SYNGAP1 pre-mRNA transcript or a complement thereof described herein.

In some embodiments, the targeted portion of the SYNGAP1 pre-mRNA comprises a sequence with at least 80%, 85%, 90%, 95%, 97%, or 100% sequence identity to a region comprising at least 8 contiguous nucleic acids of a sequence of the pre-mRNA transcripts of Table 6 or a sequence of Table 3, Table 4, Table 5A, Table 5B, or complements thereof.

In some embodiments, the targeted portion of the SYNGAP1 pre-mRNA comprises a sequence with at least 80%, 85%, 90%, 95%, 97%, or 100% sequence identity to a region comprising at least 8 contiguous nucleic acids of a sequence selected from the group consisting of the sequences of Table 3, Table 4, Table 5A, Table 5B, and complements thereof. In some embodiments, the targeted portion of the SYNGAP1 pre-mRNA comprises a sequence with at least 80%, 85%, 90%, 95%, 97%, or 100% sequence identity to a region comprising at least 8, 9, 10, 11, 12, 13, 14, 15, 16, 17, 18, 19 or 20 contiguous nucleic acids of a sequence of Table 5A or Table 5B, or a complement thereof.

In some embodiments, the ASOs disclosed herein target a NSAE pre-mRNA transcribed from a SYNGAP1 genomic sequence. In some embodiments, the ASO targets a NSAE pre-mRNA transcript from a SYNGAP1 genomic sequence comprising a NSAE exon. In some embodiments the NSAE exon is exon 11. In some embodiments, the ASO targets a NSAE pre-mRNA transcript from a SYNGAP1 genomic sequence comprising exon 11. In some embodiments, the ASO targets a NSAE pre-mRNA transcript from a SYNGAP1 genomic sequence comprising an intron flanking the 3′ splice site of the NSAE exon and an intron flanking the 5′ splice site of a NSAE exon. In some embodiments, the intron flanking the 3′ splice site of the NSAE exon is intron 10 and the intron flanking the 5′ splice site of a NSAE exon is intron 11. In some embodiments, the ASO targets a NSAE pre-mRNA transcript from a SYNGAP1 genomic sequence comprising intron 10, exon 11 and intron 11. In some embodiments, the ASO targets a NSAE pre-mRNA transcript comprising a sequence selected from the group consisting of premrna_ENST00000293748.9, premrna_ENST00000418600.7, premrna_ENST00000428982.4, premrna_ENST00000449372.7, premrna_ENST00000470232.1, premrna_ENST00000479510.2, premrna_ENST00000628646.2, premrna_ENST00000629380.3, premrna_ENST00000635885.1, premrna_ENST00000636075.1, premrna_ENST00000636116.1, premrna_ENST00000636146.1, premrna_ENST00000636193.1, premrna_ENST00000636436.1, premrna_ENST00000636443.1, premrna_ENST00000636640.1, premrna_ENST00000636731.1, premrna_ENST00000636905.1, premrna_ENST00000637052.1, premrna_ENST00000637194.1, premrna_ENST00000637490.1, premrna_ENST00000637587.1, premrna_ENST00000637671.1, premrna_ENST00000637721.1, premrna_ENST00000637911.1, premrna_ENST00000638127.1, premrna_ENST00000638142.2, premrna_ENST00000644458.1, premrna_ENST00000645250.1, premrna_ENST00000646630.1. In some embodiments, the ASO targets a NSAE pre-mRNA transcript comprising exon 10 and exon 11. In some embodiments, the ASO targets a SYNGAP1 pre-mRNA sequence comprising a NSAE exon. In some embodiments, the ASO targets a SYNGAP1 pre-mRNA sequence comprising exon 11. In some embodiments, the ASO targets a SYNGAP1 pre-mRNA sequence comprising an intron flanking the 3′ splice site of the NSAE exon. In some embodiments, the ASO targets a SYNGAP1 pre-mRNA sequence comprising intron 10. In some embodiments, the ASO targets a SYNGAP1 pre-mRNA sequence comprising an intron flanking the 5′ splice site of the NSAE exon. In some embodiments, the transcript is selected from the group consisting of transcript_ENST00000293748.9, transcript_ENST00000418600.7, transcript_ENST00000428982.4, transcript_ENST00000449372.7, transcript_ENST00000470232.1, transcript_ENST00000479510.2, transcript_ENST00000628646.2, transcript_ENST00000629380.3, transcript_ENST00000635885.1, transcript_ENST00000636075.1, transcript_ENST00000636116.1, transcript_ENST00000636146.1, transcript_ENST00000636193.1, transcript_ENST00000636436.1, transcript_ENST00000636443.1, transcript_ENST00000636640.1, transcript_ENST00000636731.1, transcript_ENST00000636905.1, transcript_ENST00000637052.1, transcript_ENST00000637194.1, transcript_ENST00000637490.1, transcript_ENST00000637587.1, transcript_ENST00000637671.1, transcript_ENST00000637721.1, transcript_ENST00000637911.1, transcript_ENST00000638127.1, transcript_ENST00000638142.2, transcript_ENST00000644458.1, transcript_ENST00000645250.1 and transcript_ENST00000646630.1.

In some embodiments, the SYNGAP1 pre-mRNA transcript is encoded by a genetic sequence with at least about 80%, 85%, 90%, 95%, 96%, 97%, 98%, 99% or 100% sequence identity to the Ensembl reference number ENSG00000227460.8 or ENSG00000197283.17 or a complement thereof. In some embodiments, the SYNGAP1 pre-mRNA transcript comprises a sequence with at least about 80%, 85%, 90%, 95%, 96%, 97%, 98%, 99% or 100% sequence identity to a SYNGAP1 pre-mRNA transcript or a complement thereof described herein.

In some embodiments, the targeted portion of the SYNGAP1 pre-mRNA comprises a sequence with at least 80%, 85%, 90%, 95%, 97%, or 100% sequence identity to a region comprising at least 8 contiguous nucleic acids of a sequence of premrna_ENST00000293748.9, premrna_ENST00000418600.7, premrna_ENST00000428982.4, premrna_ENST00000449372.7, premrna_ENST00000470232.1, premrna_ENST00000479510.2, premrna_ENST00000628646.2, premrna_ENST00000629380.3, premrna_ENST00000635885.1, premrna_ENST00000636075.1, premrna_ENST00000636116.1, premrna_ENST00000636146.1, premrna_ENST00000636193.1, premrna_ENST00000636436.1, premrna_ENST00000636443.1, premrna_ENST00000636640.1, premrna_ENST00000636731.1, premrna_ENST00000636905.1, premrna_ENST00000637052.1, premrna_ENST00000637194.1, premrna_ENST00000637490.1, premrna_ENST00000637587.1, premrna_ENST00000637671.1, premrna_ENST00000637721.1, premrna_ENST00000637911.1, premrna_ENST00000638127.1, premrna_ENST00000638142.2, premrna_ENST00000644458.1, premrna_ENST00000645250.1, premrna_ENST00000646630.1 or a sequence of Table 3 or complements thereof. In some embodiments, the targeted portion of the SYNGAP1 pre-mRNA comprises a sequence with at least 80%, 85%, 90%, 95%, 97%, or 100% sequence identity to a region comprising at least 8 contiguous nucleic acids of a sequence selected from the group consisting of the sequences of Table 3 or Table 6 or complements thereof. In some embodiments, the ASO comprises a sequence that is at least about 80%, 85%, 90%, 95%, 97%, or 100% identical to any one the sequences of Table 1 or complements thereof.

In some embodiments, the ASOs disclosed herein target a NSAE pre-mRNA transcribed from a target genomic sequence. In some embodiments, the ASO targets a NSAE pre-mRNA transcript from a target genomic sequence comprising a NSAE exon. In some embodiments, the ASO targets a NSAE pre-mRNA transcript from a target genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the pre-mRNA transcript sequences of Table 6. In some embodiments, the ASO targets a NSAE pre-mRNA transcript comprising a sequence selected from the pre-mRNA transcript sequences of Table 6 as represented by the Ensembl reference numbers. In some embodiments, the ASO targets a target pre-mRNA sequence comprising a NSAE exon. In some embodiments, the ASO targets a target pre-mRNA sequence comprising an intron flanking the 3′ splice site of the NSAE exon. In some embodiments, the ASO targets a target pre-mRNA sequence comprising an intron flanking the 5′ splice site of the NSAE exon. In some embodiments, the transcript is selected from the group consisting of the transcript sequences of Table 6. In some embodiments, the transcript is selected from the group consisting of the transcript sequences of Table 6 as represented by the Ensembl reference numbers.

In some embodiments, the target pre-mRNA transcript is encoded by a genetic sequence with at least about 80%, 85%, 90%, 95%, 96%, 97%, 98%, 99% or 100% sequence identity to the gene sequence as represented by the Ensembl reference number or a complement thereof. In some embodiments, the target pre-mRNA transcript comprises a sequence with at least about 80%, 85%, 90%, 95%, 96%, 97%, 98%, 99% or 100% sequence identity to target pre-mRNA transcript or a complement thereof described herein.

In some embodiments, the targeted portion of the target pre-mRNA comprises a sequence with at least 80%, 85%, 90%, 95%, 97%, or 100% sequence identity to a region comprising at least 8 contiguous nucleic acids of a sequence of Table 4 or a sequence of Table 3 or complements thereof. In some embodiments, the targeted portion of the target pre-mRNA comprises a sequence with at least 80%, 85%, 90%, 95%, 97%, or 100% sequence identity to a region comprising at least 8 contiguous nucleic acids of a sequence of Table 6 as represented by the Ensembl reference numbers or complements thereof. In some embodiments, the targeted portion of the target pre-mRNA comprises a sequence with at least 80%, 85%, 90%, 95%, 97%, or 100% sequence identity to a region comprising at least 8 contiguous nucleic acids of a sequence selected from the group consisting of the sequences of the transcripts of Table 6 or complements thereof. In some embodiments, the targeted portion of the target pre-mRNA comprises a sequence that is complementary to at least 8, 9, 10, 11, 12, 13, 14, 15, 16, 17, 18, 19 or 20 contiguous nucleic acids of a sequence of the transcripts of Table 6 or a complement thereof.

In some embodiments, the ASO targets exon 11 of a SYNGAP1 pre-mRNA comprising a NSAE exon. In some embodiments, the ASO targets a sequence about 2 nucleotides downstream (or 3′) from the 3′ splice site of exon 11 to about 4 nucleotides upstream (or 5′) from the 5′ splice site of exon 11.

In some embodiments, the ASO targets an exon of a target pre-mRNA comprising a NSAE exon, wherein the target is SYNGAP1. In some embodiments, the ASO targets a sequence about 2 nucleotides downstream (or 3′) from the 3′ splice site of the exon to about 4 nucleotides upstream (or 5′) from the 5′ splice site of the exon.

In some embodiments, the ASO targets intron 10 of a SYNGAP1 pre-mRNA comprising a NSAE exon. In some embodiments, the ASO targets a sequence about 4 to about 300 nucleotides upstream (or 5′) from the 3′ splice site of exon 11. In some embodiments, the ASO targets a sequence about 16 to about 100 nucleotides upstream (or 5′) from the 3′ splice site of exon 11. In some embodiments, the ASO targets a sequence about 4 to about 300 nucleotides downstream (or 3′) from the 5′ splice site of exon 10.

In some embodiments, the ASO has a sequence according to any sequence of Table 1 or complements thereof.

In some embodiments, the ASO targets an intron of a target pre-mRNA comprising a NSAE exon, wherein the target is SYNGAP1. In some embodiments, the ASO targets a sequence about 4 to about 300 nucleotides upstream (or 5′) from the 3′ splice site of the exon. In some embodiments, the ASO targets a sequence about 16 to about 100 nucleotides upstream (or 5′) from the 3′ splice site of the exon. In some embodiments, the ASO targets a sequence about 4 to about 300 nucleotides downstream (or 3′) from the 5′ splice site of the exon. In some embodiments, the ASO targets intron 10 of a SYNGAP1 pre-mRNA comprising a NSAE exon. In some embodiments, the ASO targets a sequence about 4 to about 300 nucleotides upstream (or 5′) from the 3′ splice site of exon 11. In some embodiments, the ASO targets a sequence about 4 to about 300 nucleotides downstream (or 3′) from the 5′ splice site of intron 10. In some embodiments, the ASO targets a sequence about 6 to about 100 nucleotides downstream (or 5′) from the 5′ splice site of intron 10.

In some embodiments, the ASO targets an intron of a pre-mRNA comprising a NSAE exon, wherein the target is SYNGAP1. In some embodiments, the ASO targets a sequence about 4 to about 300 nucleotides upstream (or 5′) from the 3′ splice site of the exon. In some embodiments, the ASO targets a sequence about 4 to about 300 nucleotides downstream (or 3′) from the 5′ splice site of the intron. In some embodiments, the ASO targets a sequence about 6 to about 100 nucleotides downstream (or 5′) from the 5′ splice site of the intron.

In some embodiments, the targeted portion of the SYNGAP1 pre-mRNA is in intron 9, 10, 11 or 12 In some embodiments, the targeted portion of the SYNGAP1 pre-mRNA is in exon 9, 10, 11 or 12. In some embodiments, hybridization of an ASO to the targeted portion of the NSAE pre-mRNA results in inclusion of canonical exon 11, and subsequently increases SYNGAP1 protein production. In some embodiments, hybridization of an ASO to the targeted portion of the NSAE pre-mRNA results in exclusion of a canonical exon, and subsequently decreases SYNGAP1 protein production. In some embodiments, hybridization of an ASO to the targeted portion of the NSAE pre-mRNA results in inclusion or exclusion of a canonical exon, and subsequently modulates SYNGAP1 protein production. In some embodiments, the targeted portion of the SYNGAP1 pre-mRNA is in exon 10 or 11. In some embodiments, the targeted portion of the SYNGAP1 pre-mRNA is in intron 10. In some embodiments, the targeted portion of the SYNGAP1 pre-mRNA is in intron 11.

In some embodiments, the targeted portion of the target pre-mRNA is in an intron, wherein the target is SYNGAP1. In some embodiments, the targeted portion of the target pre-mRNA is in an exon. In some embodiments, hybridization of an ASO to the targeted portion of the NSAE pre-mRNA results in inclusion of a canonical exon, and subsequently increases target protein production. In some embodiments, hybridization of an ASO to the targeted portion of the NSAE pre-mRNA results in exclusion of a canonical exon, and subsequently decreases target protein production. In some embodiments, hybridization of an ASO to the targeted portion of the NSAE pre-mRNA results in inclusion or exclusion of a canonical exon, and subsequently modulates target protein production.

In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from GRCh38/hg38: chr6 33448868 of SYNGAP1.

In some embodiments, the ASO targets a sequence upstream from the 5′ end of an NIE. For example, ASOs targeting a sequence upstream from the 5′ end of an NIE comprises a sequence that is at least about 80%, 85%, 90%, 95%, 97%, or 100% complimentary to at least 8 contiguous nucleic acids of any one of the ASOs in Table 2. For example, ASOs targeting a sequence upstream from the 5′ end of an NIE (e.g., exon (GRCh38/hg38: chr6 33448789 33448868) of SYNGAP1) can comprise a sequence with at least 80%, 85%, 90%, 95%, 97%, or 100% sequence identity to any one of the ASOs in Table 2.

In some embodiments, the ASOs target a sequence containing an exon-intron boundary (or junction). For example, ASOs targeting a sequence containing an exon-intron boundary can comprise a sequence that is at least about 80%, 85%, 90%, 95%, 97%, or 100% complimentary to at least 8 contiguous nucleic acids of any one of the sequences of Table 4 or Table 6. In some embodiments, the ASOs target a sequence downstream from the 3′ end of an NIE. For example, ASOs targeting a sequence downstream from the 3′ end of an NIE can comprise a sequence with at least 80%, 85%, 90%, 95%, 97%, or 100% sequence identity to any one of the ASOs in Table 2. For example, ASOs targeting a sequence downstream from the 3′ end of an NIE (e.g., exon (GRCh38/hg38: chr6 33448789 33448868) of SYNGAP1) can comprise a sequence with at least 80%, 85%, 90%, 95%, 97%, or 100% sequence identity to any one of the ASOs in Table 2. In some embodiments, ASOs target a sequence within an NIE.

In some embodiments, the targeted portion of the SYNGAP1 NIE containing pre-mRNA is intron (GRCh38/hg38: chr6 33447934 33451759) of SYNGAP1.

In some embodiments, the methods and compositions of the present disclosure are used to increase the expression of SYNGAP1 by inducing exon skipping of a pseudo-exon of a SYNGAP1 NIE containing pre-mRNA. In some embodiments, the pseudo-exon can be any SYNGAP1 intron or a portion thereof. In some embodiments, the pseudo-exon is within intron (GRCh38/hg38: chr6 33447934 33451759) of SYNGAP1.

In some embodiments, the methods described herein are used to increase the production of a functional SYNGAP1 protein or RNA. As used herein, the term “functional” refers to the amount of activity or function of a SYNGAP1 protein or RNA that is necessary to eliminate any one or more symptoms of a treated condition or disease, e.g., mental retardation, autosomal dominant 5. In some embodiments, the methods are used to increase the production of a partially functional SYNGAP1 protein or RNA. As used herein, the term “partially functional” refers to any amount of activity or function of the SYNGAP1 protein or RNA that is less than the amount of activity or function that is necessary to eliminate or prevent any one or more symptoms of a disease or condition. In some embodiments, a partially functional protein or RNA will have at least 10%, at least 20%, at least 30%, at least 40%, at least 50%, at least 60%, at least 70%, at least 75%, at least 80%, at least 85%, at least 90%, or at least 95% less activity relative to the fully functional protein or RNA.

In some embodiments, the method is a method of increasing the expression of the SYNGAP1 protein by cells of a subject having a NIE containing pre-mRNA encoding the SYNGAP1 protein, wherein the subject has Mental retardation, autosomal dominant 5 caused by a deficient amount of activity of SYNGAP1 protein, and wherein the deficient amount of the SYNGAP1 protein is caused by haploinsufficiency of the SYNGAP1 protein. In such an embodiment, the subject has a first allele encoding a functional SYNGAP1 protein, and a second allele from which the SYNGAP1 protein is not produced. In another such embodiment, the subject has a first allele encoding a functional SYNGAP1 protein, and a second allele encoding a nonfunctional SYNGAP1 protein. In another such embodiment, the subject has a first allele encoding a functional SYNGAP1 protein, and a second allele encoding a partially functional SYNGAP1 protein. In any of these embodiments, the antisense oligomer binds to a targeted portion of the NIE containing pre-mRNA transcribed from the second allele, thereby inducing exon skipping of the pseudo-exon from the pre-mRNA, and causing an increase in the level of mature mRNA encoding functional SYNGAP1 protein, and an increase in the expression of the SYNGAP1 protein in the cells of the subject.

In some embodiments, the method is a method of increasing the expression of the SYNGAP1 protein by cells of a subject having a NIE containing pre-mRNA encoding the SYNGAP1 protein, wherein the subject has Mental retardation, autosomal dominant 5 caused by a deficient amount of activity of SYNGAP1 protein, and wherein the deficient amount of the SYNGAP1 protein is caused by autosomal recessive inheritance.

In some embodiments, the method is a method of increasing the expression of the SYNGAP1 protein by cells of a subject having a NIE containing pre-mRNA encoding the SYNGAP1 protein, wherein the subject has Mental retardation, autosomal dominant 5 caused by a deficient amount of activity of SYNGAP1 protein, and wherein the deficient amount of the SYNGAP1 protein is caused by autosomal dominant inheritance.

In some embodiments, the method is a method of increasing the expression of the SYNGAP1 protein by cells of a subject having a NIE containing pre-mRNA encoding the SYNGAP1 protein, wherein the subject has Mental retardation, autosomal dominant 5 caused by a deficient amount of activity of SYNGAP1 protein, and wherein the deficient amount of the SYNGAP1 protein is caused by X-linked dominant inheritance.

In related embodiments, the method is a method of using an ASO to increase the expression of a protein or functional RNA. In some embodiments, an ASO may be used to increase the expression of SYNGAP1 protein in cells of a subject having a NIE containing pre-mRNA encoding SYNGAP1 protein, wherein the subject has a deficiency, e.g., Mental retardation, autosomal dominant 5, in the amount or function of a SYNGAP1 protein.

In various embodiments, the present disclosure provides a therapeutic agent which can target SYNGAP1 mRNA transcripts to modulate splicing or protein expression level. The therapeutic agent can be a small molecule, polynucleotide, or polypeptide. In some embodiments, the therapeutic agent is an ASO. Various regions or sequences on the SYNGAP1 pre-mRNA can be targeted by a therapeutic agent, such as an ASO. In some embodiments, the ASO targets a SYNGAP1 pre-mRNA transcript containing an NIE. In some embodiments, the ASO targets a sequence within an NIE of a SYNGAP1 pre-mRNA transcript. In some embodiments, the ASO targets a sequence upstream (or 5′) from the 5′ end of an NIE (3′ss) of a SYNGAP1 pre-mRNA transcript. In some embodiments, the ASO targets a sequence downstream (or 3′) from the 3′ end of an NIE (5′ss) of a SYNGAP1 pre-mRNA transcript. In some embodiments, the ASO targets a sequence that is within an intron flanking on the 5′ end of the NIE of a SYNGAP1 pre-mRNA transcript. In some embodiments, the ASO targets a sequence that is within an intron flanking the 3′ end of the NIE of a SYNGAP1 pre-mRNA transcript. In some embodiments, the ASO targets a sequence comprising an NIE-intron boundary of a SYNGAP1 pre-mRNA transcript. An NIE-intron boundary can refer to the junction of an intron sequence and an NIE region. The intron sequence can flank the 5′ end of the NIE, or the 3′ end of the NIE. In some embodiments, the ASO targets a sequence within an exon of a SYNGAP1 pre-mRNA transcript. In some embodiments, the ASO targets a sequence within an intron of a SYNGAP1 pre-mRNA transcript. In some embodiments, the ASO targets a sequence comprising both a portion of an intron and a portion of an exon of a SYNGAP1 pre-mRNA transcript.

In some embodiments, the ASO targets a sequence about 4 to about 300 nucleotides upstream (or 5′) from the 5′ end of the NIE. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream (or 5′) from the 5′ end of the NIE region. In some embodiments, the ASO may target a sequence more than 300 nucleotides upstream from the 5′ end of the NIE. In some embodiments, the ASO targets a sequence about 4 to about 300 nucleotides downstream (or 3′) from the 3′ end of the NIE. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream from the 3′ end of the NIE. In some embodiments, the ASO targets a sequence more than 300 nucleotides downstream from the 3′ end of the NIE.

In some embodiments, the SYNGAP1 NIE containing pre-mRNA transcript is encoded by a genetic sequence with at least about 80%, 85%, 90%, 95%, 96%, 97%, 98%, 99% or 100% sequence identity to a SYNGAP gene. In some embodiments, the SYNGAP1 NIE pre-mRNA transcript comprises a sequence with at least about 80%, 85%, 90%, 95%, 96%, 97%, 98%, 99% or 100% sequence identity to any one of the sequences of the transcripts of Table 6.

In some embodiments, SYNGAP1 NIE containing pre-mRNA transcript (or NMD exon mRNA) is encoded by a sequence with at least about 80%, 85%, 90%, 95%, 97%, or 100% sequence identity to any one of the sequences of the transcripts of Table 6. In some embodiments, the targeted portion of the NMD exon mRNA comprises a sequence with at least 80%, 85%, 90%, 95%, 97%, or 100% sequence identity to a region comprising at least 8 contiguous nucleic acids of any one of the sequences of the transcripts of Table 4. In some embodiments, the targeted portion of the NMD exon mRNA comprises a sequence with at least 80%, 85%, 90%, 95%, 97%, or 100% sequence identity to a region comprising at least 8 contiguous nucleic acids of any one of the sequences of the transcripts of Table 4.

### SYNGAP1 Protein

In some embodiments, a mutation occurs in both alleles. In some embodiments, a mutation occurs in one of the two alleles. In some embodiments, additional mutation occurs in one of the two alleles. In some embodiments, the additional mutation occurs in the same allele as the first mutation. In other embodiments, the additional mutation occurs is a trans mutation.

In some embodiments, the methods described herein are used to increase the production of a functional SYNGAP1 protein. In some embodiments, the methods described herein are used to decrease the production of a functional SYNGAP1 protein. In some embodiments, the methods described herein are used to modulate the production of a functional SYNGAP1 protein. As used herein, the term “functional” refers to the amount of activity or function of a SYNGAP1 protein that is necessary to prevent or eliminate any one or more symptoms of a treated disease or condition, e.g., autosomal dominant mental retardation. Alternatively, overexpression of a functional SYNGAP1 protein may induce or increase any one or more symptoms of a treated disease or condition. In some embodiments, the methods are used to increase the production of a partially functional SYNGAP1 protein. In some embodiments, the methods are used to decrease the production of a partially functional SYNGAP1 protein. In some embodiments, the methods are used to modulate the production of a partially functional SYNGAP1 protein. As used herein, the term “partially functional” refers to any amount of activity or function of the SYNGAP1 protein that is less than the amount of activity or function that is necessary to eliminate or prevent any one or more symptoms of a disease or condition, e.g., autosomal dominant mental retardation. Alternatively, expression or overexpression of a functional SYNGAP1 protein may induce or increase any one or more symptoms of a treated disease or condition. In some embodiments, a partially functional protein or RNA will have at least 10%, at least 20%, at least 30%, at least 40%, at least 50%, at least 60%, at least 70%, at least 75%, at least 80%, at least 85%, at least 90%, or at least 95% less activity relative to the fully functional protein or RNA.

In some embodiments, the method is a method of increasing the expression of the SYNGAP1 protein by cells of a subject having a NSAE pre-mRNA encoding the SYNGAP1 protein, wherein the subject has autosomal dominant mental retardation caused by a deficient amount of activity of SYNGAP1 protein, and wherein the deficient amount of the SYNGAP1 protein is caused by sporadic mutation. In such an embodiment, the subject has a first allele carrying the mutation and a second allele from which the SYNGAP1 protein is not produced. In another such embodiment, the subject has a first allele carrying a mutation and a second allele encoding a nonfunctional SYNGAP1 protein. In another such embodiment, the subject has a first allele carrying a mutation and a second allele encoding a partially functional SYNGAP1 protein. In another such an embodiment, the subject has a first allele carrying a mutation and a second allele carrying the mutation. In any of these embodiments, the antisense oligomer binds to a targeted portion of the NSAE pre-mRNA transcribed from the allele carrying a mutation, thereby prevent alternate splicing of NSAEs into the pre-mRNA, and causing an increase in the level of mature mRNA encoding functional SYNGAP1 protein, and an increase in the expression of the SYNGAP1 protein in the cells of the subject.

In related embodiments, the method is a method of using an ASO to increase the expression of a functional protein or functional RNA. In some embodiments, an ASO is used to increase the expression of SYNGAP1 protein in cells of a subject having a NSAE pre-mRNA encoding SYNGAP1 protein, wherein the subject has a deficiency, e.g., autosomal dominant mental retardation, in the amount or function of SYNGAP1 protein.

In some embodiments, the method is a method of decreasing the expression of the SYNGAP1 protein by cells of a subject having a NSAE pre-mRNA encoding the SYNGAP1 protein, wherein the subject has a disease caused by an excess amount of activity of SYNGAP1 protein, and wherein the excess amount of SYNGAP1 protein is caused by a mutation. In some embodiments, the antisense oligomer binds to a targeted portion of the NSAE pre-mRNA transcribed from the allele carrying a mutation, thereby increasing alternate splicing of NSAEs into the pre-mRNA, and causing a decrease in the level of mature mRNA encoding functional SYNGAP1 protein, and an decrease in the expression of the SYNGAP1 protein in the cells of the subject. In related embodiments, the method is a method of using an ASO to decrease the expression of a functional protein or functional RNA. In some embodiments, an ASO is used to decrease the expression of SYNGAP1 protein in cells of a subject having a NSAE pre-mRNA encoding the SYNGAP1 protein, wherein the subject has an excess in the amount or function of SYNGAP1 protein.

In some embodiments, the method is a method of modulating the expression of the SYNGAP1 protein by cells of a subject having a NSAE pre-mRNA encoding the SYNGAP1 protein, wherein the subject has a disease caused by a deficient or excess amount of activity of SYNGAP1 protein, and wherein the deficient or excess amount of the SYNGAP1 protein is caused by a mutation. In some embodiments, the antisense oligomer binds to a targeted portion of the NSAE pre-mRNA transcribed from the allele carrying a mutation, thereby modulating alternate splicing of NSAEs into the pre-mRNA, and causing a modulation in the level of mature mRNA encoding functional SYNGAP1 protein, and a modulation in the expression of SYNGAP1 protein in the cells of the subject. In related embodiments, the method is a method of using an ASO to modulate the expression of a functional protein or functional RNA. In some embodiments, an ASO is used to modulate the expression of SYNGAP1 protein in cells of a subject having a NSAE pre-mRNA encoding the SYNGAP1 protein, wherein the subject has an abnormality in the amount or function of the SYNGAP1 protein.

In some embodiments, the NSAE pre-mRNA transcript that encodes the protein that is causative of the disease or condition is targeted by the ASOs described herein. In some embodiments, a NSAE pre-mRNA transcript that encodes a protein that is not causative of the disease is targeted by the ASOs. For example, a disease that is the result of a mutation or deficiency of a first protein in a particular pathway may be ameliorated by targeting a NSAE containing pre-mRNA that encodes a second protein, thereby increasing production of the second protein. In some embodiments, the function of the second protein is able to compensate for the mutation or deficiency of the first protein (which is causative of the disease or condition).

In some embodiments, the subject has:
(a) a first mutant allele carrying a mutation from which the SYNGAP1 protein is produced at a reduced level compared to production from a wild-type allele, and
(b) a second mutant allele from which
   (i) the SYNGAP1 protein is produced at a reduced level compared to production from a wild-type allele due to the mutation,
   (ii) the SYNGAP1 protein is produced at a reduced level compared to production from a wild-type allele,
   (iii) the SYNGAP1 protein is produced in a form having reduced function compared to an equivalent wild-type protein, or
   (iv) the SYNGAP1 protein is not produced, and
wherein the NSAE pre-mRNA is transcribed from the first allele and/or the second allele carrying the mutation. In these embodiments, the ASO binds to a targeted portion of the NSAE pre-mRNA transcribed from the first allele or the second allele, thereby promoting exon inclusion from the NSAE pre-mRNA, and causing an increase in the level of full-length mRNA encoding SYNGAP1 protein and an increase in the expression of the target protein or functional RNA in the cells of the subject. In these embodiments, the target protein or functional RNA having an increase in expression level resulting from exon inclusion from the NSAE pre-mRNA has full function compared to the equivalent wild-type protein (fully-functional). In these embodiments, the ASO binds to a targeted portion of the NSAE pre-mRNA transcribed from the first allele or the second allele, thereby reducing exon inclusion from the NSAE pre-mRNA, and causing a decrease in the level of full-length mRNA encoding SYNGAP1 protein and a decrease in the expression of the target protein or functional RNA in the cells of the subject. In these embodiments, the target protein or functional RNA having a decrease in expression level resulting from exon exclusion from the NSAE pre-mRNA has full function compared to the equivalent wild-type protein (fully-functional).

In some embodiments, contacting cells with an ASO that is complementary to a targeted portion of a SYNGAP1 pre-mRNA transcript results in an increase in the amount of SYNGAP1 protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of SYNGAP1 protein produced by the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of SYNGAP1 protein produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, the level of mRNA encoding SYNGAP1 protein is increased 1.1 to 10-fold, when compared to the amount of mRNA encoding SYNGAP1 protein that is produced in a control cell, e.g., one that is not treated with the antisense oligomer or one that is treated with an antisense oligomer that does not bind to the targeted portion of the SYNGAP1 containing pre-mRNA.

In some embodiments, the level of mRNA encoding SYNGAP1 protein is increased 1.1 to 10-fold, when compared to the amount of mRNA encoding SYNGAP1 protein that is produced in a control cell, e.g., one that is not treated with the antisense oligomer or one that is treated with an antisense oligomer that does not bind to the targeted portion of the SYNGAP1 pre-mRNA.

In some embodiments of the present invention, a subject can have a mutation in SYNGAP1. A variety of pathogenic variants have been reported to cause SYNGAP1 deficiency, including missense variants, nonsense variants, single- and double-nucleotide insertions and deletions, complex insertion/deletions, and splice site variants. In the presence of this pathogenic variant approximately 2%-5% of transcripts are correctly spliced, allowing for residual enzyme activity. In some embodiments, disease results from loss of function of SYNGAP1 caused by SYNGAP1 pathogenic variants that generate truncated proteins or proteins with altered conformations or reduced activity.

In some embodiments, a subject having any SYNGAP1 mutation known in the art and described as above can be treated using the methods and compositions described herein. In some embodiments, the mutation is within any SYNGAP1 intron or exon. In some embodiments, the mutation is within SYNGAP1 exon 10 or 11.

In some embodiments, contacting cells with an ASO that is complementary to a targeted portion of a SYNGAP1 pre-mRNA transcript results in a decrease in the amount of SYNGAP1 protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of SYNGAP1 protein produced by the cell to which the antisense oligomer is contacted is decreased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of SYNGAP1 protein produced by the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, the level of mRNA encoding SYNGAP1 protein is decreased 1.1 to 10-fold, when compared to the amount of mRNA encoding SYNGAP1 protein that is produced in a control cell, e.g., one that is not treated with the antisense oligomer or one that is treated with an antisense oligomer that does not bind to the targeted portion of the SYNGAP1 containing pre-mRNA.

In some embodiments, the level of mRNA encoding SYNGAP1 protein is decreased 1.1 to 10-fold, when compared to the amount of mRNA encoding SYNGAP1 protein that is produced in a control cell, e.g., one that is not treated with the antisense oligomer or one that is treated with an antisense oligomer that does not bind to the targeted portion of the SYNGAP1 pre-mRNA.

In some embodiments of the present invention, a subject can have a mutation in SYNGAP1. A variety of pathogenic variants may cause SYNGAP1 overexpression, including missense variants, nonsense variants, single- and double-nucleotide insertions and deletions, complex insertion/deletions, and splice site variants. In some embodiments, disease results from an overexpression of function of SYNGAP1 caused by SYNGAP1 pathogenic variants that generate truncated proteins or proteins with altered conformations or increased activity.

In some embodiments, a subject having any SYNGAP1 mutation known in the art and described as above can be treated using the methods and compositions described herein. In some embodiments, the mutation is within any SYNGAP1 intron or exon. In some embodiments, the mutation is within SYNGAP1 exon 10 or 11.

### Target Protein

In some embodiments, a mutation occurs in both alleles. In some embodiments, a mutation occurs in one of the two alleles, wherein the target is SYNGAP1. In some embodiments, additional mutation occurs in one of the two alleles. In some embodiments, the additional mutation occurs in the same allele as the first mutation. In other embodiments, the additional mutation occurs is a trans mutation.

In some embodiments, the methods described herein are used to increase the production of a functional target protein, wherein the target is SYNGAP1. In some embodiments, the methods described herein are used to decrease the production of a functional target protein. In some embodiments, the methods described herein are used to modulate the production of a functional target protein. As used herein, the term “functional” refers to the amount of activity or function of a target protein that is necessary to prevent or eliminate any one or more symptoms of a treated disease or condition, e.g., autosomal dominant mental retardation. Alternatively, overexpression of a functional target protein may induce or increase any one or more symptoms of a treated disease or condition. In some embodiments, the methods are used to increase the production of a partially functional target protein. In some embodiments, the methods are used to decrease the production of a partially functional target protein. In some embodiments, the methods are used to modulate the production of a partially functional target protein. As used herein, the term “partially functional” refers to any amount of activity or function of the target protein that is less than the amount of activity or function that is necessary to eliminate or prevent any one or more symptoms of a disease or condition, e.g., autosomal dominant mental retardation. Alternatively, expression or overexpression of a functional target protein may induce or increase any one or more symptoms of a treated disease or condition. In some embodiments, a partially functional protein or RNA will have at least 10%, at least 20%, at least 30%, at least 40%, at least 50%, at least 60%, at least 70%, at least 75%, at least 80%, at least 85%, at least 90%, or at least 95% less activity relative to the fully functional protein or RNA.

In some embodiments, the method is a method of increasing the expression of the target protein by cells of a subject having a NSAE pre-mRNA encoding the target protein, wherein the subject has autosomal dominant mental retardation caused by a deficient amount of activity of target protein, wherein the deficient amount of the target protein is caused by sporadic mutation, and wherein the target is SYNGAP1. In such an embodiment, the subject has a first allele carrying a mutation and a second allele from which the target protein is not produced. In another such embodiment, the subject has a first allele carrying a mutation and a second allele encoding a nonfunctional target protein. In another such embodiment, the subject has a first allele carrying a mutation and a second allele encoding a partially functional target protein. In another such an embodiment, the subject has a first allele carrying a mutation and a second allele carrying the mutation. In any of these embodiments, the antisense oligomer binds to a targeted portion of the NSAE pre-mRNA transcribed from the allele carrying a mutation, thereby prevent alternate splicing of NSAEs into the pre-mRNA, and causing an increase in the level of mature mRNA encoding functional target protein, and an increase in the expression of the target protein in the cells of the subject. In related embodiments, the method is a method of using an ASO to increase the expression of a functional protein or functional RNA. In some embodiments, an ASO is used to increase the expression of target protein in cells of a subject having a NSAE pre-mRNA encoding the target protein, wherein the subject has a deficiency, e.g., autosomal dominant mental retardation, in the amount or function of the target protein.

In some embodiments, the method is a method of decreasing the expression of the target protein by cells of a subject having a NSAE pre-mRNA encoding the target protein, wherein the subject has a disease caused by an excess amount of activity of the target protein, wherein the excess amount of the target protein is caused by a mutation, and wherein the target is SYNGAP1. In some embodiments, the antisense oligomer binds to a targeted portion of the NSAE pre-mRNA transcribed from the allele carrying a mutation, thereby increasing alternate splicing of NSAEs into the pre-mRNA, and causing a decrease in the level of mature mRNA encoding the functional target protein, and a decrease in the expression of the target protein in the cells of the subject. In related embodiments, the method is a method of using an ASO to decrease the expression of a functional protein or functional RNA. In some embodiments, an ASO is used to decrease the expression of the target protein in cells of a subject having a NSAE pre-mRNA encoding the target protein, wherein the subject has an excess in the amount or function of the target protein.

In some embodiments, the method is a method of modulating the expression of the target protein by cells of a subject having a NSAE pre-mRNA encoding the target protein, wherein the subject has a disease caused by a deficient or excess amount of activity of the target protein, wherein the deficient or excess amount of the target protein is caused by a mutation, and wherein the target is SYNGAP1. In some embodiments, the antisense oligomer binds to a targeted portion of the NSAE pre-mRNA transcribed from the allele carrying a mutation, thereby modulating alternate splicing of NSAEs into the pre-mRNA, and causing a modulation in the level of mature mRNA encoding the functional target protein, and a modulation in the expression of the target protein in the cells of the subject. In related embodiments, the method is a method of using an ASO to modulate the expression of a functional protein or functional RNA. In some embodiments, an ASO is used to modulate the expression of the target protein in cells of a subject having a NSAE pre-mRNA encoding the target protein, wherein the subject has an abnormality in the amount or function of the target protein.

In some embodiments, the NSAE pre-mRNA transcript that encodes the protein that is causative of the disease or condition is targeted by the ASOs described herein. In some embodiments, a NSAE pre-mRNA transcript that encodes a protein that is not causative of the disease is targeted by the ASOs. For example, a disease that is the result of a mutation or deficiency of a first protein in a particular pathway may be ameliorated by targeting a NSAE containing pre-mRNA that encodes a second protein, thereby increasing production of the second protein. In some embodiments, the function of the second protein is able to compensate for the mutation or deficiency of the first protein (which is causative of the disease or condition).

In some embodiments, the subject has:
(a) a first mutant allele carrying a mutation from which the target protein is produced at a reduced level compared to production from a wild-type allele, and
(b) a second mutant allele from which
   (i) the target protein is produced at a reduced level compared to production from a wild-type allele due to the mutation,
   (ii) the target protein is produced at a reduced level compared to production from a wild-type allele,
   (iii) the target protein is produced in a form having reduced function compared to an equivalent wild-type protein, or
   (iv) the target protein is not produced, and
wherein the NSAE pre-mRNA is transcribed from the first allele and/or the second allele carrying the mutation and wherein the target is SYNGAP1. In these embodiments, the ASO binds to a targeted portion of the NSAE pre-mRNA transcribed from the first allele or the second allele, thereby promoting exon inclusion from the NSAE pre-mRNA, and causing an increase in the level of full-length mRNA encoding the target protein and an increase in the expression of the target protein or functional RNA in the cells of the subject. In these embodiments, the target protein or functional RNA having an increase in expression level resulting from exon inclusion from the NSAE pre-mRNA has full function compared to the equivalent wild-type protein (fully-functional). In these embodiments, the ASO binds to a targeted portion of the NSAE pre-mRNA transcribed from the first allele or the second allele, thereby reducing exon inclusion from the NSAE pre-mRNA, and causing a decrease in the level of full-length mRNA encoding the target protein and a decrease in the expression of the target protein or functional RNA in the cells of the subject. In these embodiments, the target protein or functional RNA having a decrease in expression level resulting from exon exclusion from the NSAE pre-mRNA has full function compared to the equivalent wild-type protein (fully-functional).

In some embodiments, contacting cells with an ASO that is complementary to a targeted portion of a target pre-mRNA transcript results in an increase in the amount of the target protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment, wherein the target is SYNGAP1. In some embodiments, the total amount of the target protein produced by the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of the target protein produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, the level of mRNA encoding the target protein is increased 1.1 to 10-fold, when compared to the amount of mRNA encoding the target protein that is produced in a control cell, e.g., one that is not treated with the antisense oligomer or one that is treated with an antisense oligomer that does not bind to the targeted portion of the target containing pre-mRNA, wherein the target is SYNGAP1. In some embodiments, the level of mRNA encoding the target protein is increased 1.1 to 10-fold, when compared to the amount of mRNA encoding the target protein that is produced in a control cell, e.g., one that is not treated with the antisense oligomer or one that is treated with an antisense oligomer that does not bind to the targeted portion of the target pre-mRNA.

In some embodiments of the present invention, a subject can have a mutation in the target gene, wherein the target is SYNGAP1. A variety of pathogenic variants have been reported to cause the target deficiency, including missense variants, nonsense variants, single- and double-nucleotide insertions and deletions, complex insertion/deletions, and splice site variants. In some embodiments, disease results from loss of function of the target caused by the target pathogenic variants that generate truncated proteins or proteins with altered conformations or reduced activity.

In some embodiments, a subject having any mutation in the target gene known in the art and described as above can be treated using the methods and compositions described herein, wherein the target is SYNGAP1. In some embodiments, the mutation is within any intron or exon in the target gene.

In some embodiments, contacting cells with an ASO that is complementary to a targeted portion of a target pre-mRNA transcript results in a decrease in the amount of the target protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment, wherein the target is SYNGAP1. In some embodiments, the total amount of the target protein produced by the cell to which the antisense oligomer is contacted is decreased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of the target protein produced by the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, the level of mRNA encoding the target protein is decreased 1.1 to 10-fold, when compared to the amount of mRNA encoding the target protein that is produced in a control cell, e.g., one that is not treated with the antisense oligomer or one that is treated with an antisense oligomer that does not bind to the targeted portion of the target containing pre-mRNA, wherein the target is SYNGAP1.

In some embodiments, the level of mRNA encoding the target protein is decreased 1.1 to 10-fold, when compared to the amount of mRNA encoding the target protein that is produced in a control cell, e.g., one that is not treated with the antisense oligomer or one that is treated with an antisense oligomer that does not bind to the targeted portion of the target pre-mRNA, wherein the target is SYNGAP1.

In some embodiments of the present invention, a subject can have a mutation in the target gene, wherein the target is SYNGAP1. A variety of pathogenic variants may cause the target protein overexpression, including missense variants, nonsense variants, single- and double-nucleotide insertions and deletions, complex insertion/deletions, and splice site variants. In some embodiments, disease results from an overexpression of function of the target protein caused by the pathogenic variants in the target that generate truncated proteins or proteins with altered conformations or increased activity.

In some embodiments, a subject having any mutation in the target gene known in the art and described as above can be treated using the methods and compositions described herein, wherein the target is SYNGAP1. In some embodiments, the mutation is within any intron or exon in the target gene.

### Alternative Splicing

As used herein, a “non-sense mediated RNA decay alternative exon” (or “NSAE”) is an exon created from alternative splicing events that contains a premature stop codon or other sequences that facilitate degradation of the mRNA containing the of the instant exon. “NSAE pre-mRNA” is a pre-mRNA transcript that contains at least one non-sense mediated RNA decay alternative exon. Alternative or aberrant splicing of the pre-mRNA can result in the inclusion of at least one NSAE in the mature mRNA transcripts. The terms “mature mRNA,” “fully processed,” and “fully-spliced mRNA,” are used interchangeably herein to describe a fully processed mRNA. Inclusion of at least one NSAE can result in non-productive mRNA. Mature NSAE-containing mRNA may sometimes lead to aberrant protein expression. The term “canonical exon” refers to the exon that does not lead to non-sense mediated decay, or default exon, or exon that is not the product of alternative splicing. The term “canonical splicing” refers to a splicing process that results in the preservation of the canonical exon in the mature RNA.

A NSAE can be created as a result of splicing out additional base pairs.

The degree of alternative splicing can be expressed as percent alternative splicing, e.g., the percentage of transcripts in which a given NSAE is included. In brief, percent alternative splicing can be calculated as the percentage of the amount of RNA transcripts with the NSAE, over the sum of the average of the amount of RNA transcripts with a NSAE plus the average of the amount of RNA transcripts with only the canonical exons.

In some embodiments, a NSAE is an exon that is identified as a NSAE based on a determination of at least about 5%, at least about 10%, at least about 15%, at least about 20%, at least about 25%, at least about 30%, at least about 35%, at least about 40%, at least about 45%, or at least about 50%, exclusion from the RNA transcript. In some embodiments, a NSAE is an exon that is identified as a NSAE based on a determination of about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 10% to about 100%, about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 15% to about 100%, about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 20% to about 100%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 25% to about 100%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, or about 25% to about 35%, exclusion from the RNA transcript. ENCODE data (described by, e.g., Tilgner, et al., 2012, “Deep sequencing of subcellular RNA fractions shows splicing to be predominantly co-transcriptional in the human genome but inefficient for lncRNAs,” Genome Research 22(9):1616-25) can be used to aid in identifying NSAE's created from alternative splicing events.

In some embodiments, contacting cells with an ASO that is complementary to a targeted portion of a SYNGAP1 pre-mRNA transcript results in an increase in the amount of SYNGAP1 protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of SYNGAP1 protein produced by the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of SYNGAP1 protein produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a SYNGAP1 pre-mRNA transcript results in a decrease in the amount of SYNGAP1 protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the mutation is within any intron or exon in the target gene. In some embodiments, the total amount of SYNGAP1 protein produced by the cell to which the antisense oligomer is contacted is decreased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of SYNGAP1 protein produced by the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a SYNGAP1 pre-mRNA transcript results in an increase in the amount of mRNA encoding SYNGAP1, including the mature mRNA encoding the target protein. In some embodiments, the amount of mRNA encoding SYNGAP1 protein, or the mature mRNA encoding the SYNGAP1 protein, is increased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding SYNGAP1 protein, or the mature mRNA encoding SYNGAP1 protein produced in the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of mature RNA produced in an untreated cell, e.g., an untreated cell or a cell treated with a control compound. In some embodiments, the total amount of the mRNA encoding SYNGAP1 protein, or the mature mRNA encoding SYNGAP1 protein produced in the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the SYNGAP1 pre-mRNA.

In some embodiments, contacting cells with an ASO that is complementary to a targeted portion of a SYNGAP1 pre-mRNA transcript results in a decrease in the amount of mRNA encoding SYNGAP1, including the mature mRNA encoding the target protein. In some embodiments, the mutation is within any intron or exon in the target gene. In some embodiments, the amount of mRNA encoding SYNGAP1 protein, or the mature mRNA encoding the SYNGAP1 protein, is decreased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding SYNGAP1 protein, or the mature mRNA encoding SYNGAP1 protein produced in the cell to which the antisense oligomer is contacted is decreased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of mature RNA produced in an untreated cell, e.g., an untreated cell or a cell treated with a control compound. In some embodiments, the total amount of the mRNA encoding SYNGAP1 protein, or the mature mRNA encoding SYNGAP1 protein produced in the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the SYNGAP1 pre-mRNA.

In some embodiments, contacting cells with an ASO that is complementary to a targeted portion of a target pre-mRNA transcript results in an increase in the amount of the target protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment, wherein the target is SYNGAP1. In some embodiments, the mutation is within any intron or exon in the target gene. In some embodiments, the total amount of the target protein produced by the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of the target protein produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a target pre-mRNA transcript results in a decrease in the amount of the target protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment, wherein the target is SYNGAP1. In some embodiments, the mutation is within any intron or exon in the target gene. In some embodiments, the total amount of the target protein produced by the cell to which the antisense oligomer is contacted is decreased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of the target protein produced by the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a target pre-mRNA transcript results in an increase in the amount of mRNA encoding the target protein, including the mature mRNA encoding the target protein, wherein the target is SYNGAP1. In some embodiments, the mutation is within any intron or exon in the target gene. In some embodiments, the amount of mRNA encoding the target protein, or the mature mRNA encoding the target protein, is increased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding the target protein, or the mature mRNA encoding the target protein produced in the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of mature RNA produced in an untreated cell, e.g., an untreated cell or a cell treated with a control compound. In some embodiments, the total amount of the mRNA encoding the target protein, or the mature mRNA encoding the target protein produced in the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the target pre-mRNA.

In some embodiments, contacting cells with an ASO that is complementary to a targeted portion of a target pre-mRNA transcript results in a decrease in the amount of mRNA encoding the target protein, including the mature mRNA encoding the target protein, wherein the target is SYNGAP1. In some embodiments, the mutation is within any intron or exon in the target gene. In some embodiments, the amount of mRNA encoding the target protein, or the mature mRNA encoding the target protein, is decreased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding the target protein, or the mature mRNA encoding the target protein produced in the cell to which the antisense oligomer is contacted is decreased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of mature RNA produced in an untreated cell, e.g., an untreated cell or a cell treated with a control compound. In some embodiments, the total amount of the mRNA encoding the target protein, or the mature mRNA encoding the target protein produced in the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the target pre-mRNA.

### Therapeutic Agents

In various embodiments of the present disclosure, compositions and methods comprising a therapeutic agent are provided to modulate protein expression level of SYNGAP1. In some embodiments, provided herein are compositions and methods to modulate alternative splicing of SYNGAP1 pre-mRNA. In some embodiments, provided herein are compositions and methods to modulate alternative splicing in the splicing of SYNGAP1 pre-mRNA, e.g., to prevent inclusion of a NSAE during splicing of SYNGAP1 pre-mRNA and preserve canonical exons.

In various embodiments of the present disclosure, compositions and methods comprising a therapeutic agent are provided to modulate protein expression level of the target. In some embodiments, provided herein are compositions and methods to modulate alternative splicing of the target pre-mRNA. In some embodiments, provided herein are compositions and methods to modulate alternative splicing in the splicing of the target pre-mRNA, e.g., to prevent inclusion of a NSAE during splicing of the target pre-mRNA and preserve canonical exons, wherein the target is SYNGAP1.

A therapeutic agent disclosed herein can be an alternative splicing repressor agent. In some embodiments, a therapeutic agent may comprise a polynucleic acid polymer. In other embodiments, a therapeutic agent may comprise a small molecule. In other embodiments, a therapeutic agent may comprise a polypeptide. In some embodiments, the therapeutic agent is a nucleic acid binding protein, with or without being complexed with a nucleic acid molecule. In other embodiments, the therapeutic agent is a nucleic acid molecule that encodes for another therapeutic agent. In further embodiments, the therapeutic agent is incorporated into a viral delivery system, such as an adenovirus-associated vector.

According to one aspect of the present disclosure, provided herein is a method of treatment or prevention of a condition associated with a functional-SYNGAP1 protein deficiency, comprising administering an alternative splicing repressor agent to a subject to increase levels of functional SYNGAP1 protein, wherein the agent binds to a region of the pre-mRNA transcript to decrease inclusion of the NSAE in the mature transcript. For example, provided herein is a method of treatment or prevention of a condition associated with a functional-SYNGAP1 protein deficiency, comprising administering an alternative splicing repressor agent to a subject to increase levels of functional SYNGAP1 protein, wherein the agent binds to a region of an exon or an intron (e.g., exon 10 or 11, intron 10 or intron 11 in human SYNGAP1 gene) of the pre-mRNA transcript.

According to one aspect of the present disclosure, provided herein is a method of treatment or prevention of a condition associated with a functional target protein deficiency, comprising administering an alternative splicing repressor agent to a subject to increase levels of functional target protein, wherein the agent binds to a region of the pre-mRNA transcript to decrease inclusion of the NSAE in the mature transcript. For example, provided herein is a method of treatment or prevention of a condition associated with a functional target protein deficiency, comprising administering an alternative splicing repressor agent to a subject to increase levels of functional target protein, wherein the agent binds to a region of an exon or an intron of the pre-mRNA transcript, wherein the target is SYNGAP1.

In some embodiments, provided herein is a method of treatment or prevention of a condition associated with a functional-SYNGAP1 protein overexpression, comprising administering an alternative splicing modulator agent to a subject to decrease levels of functional SYNGAP1 protein, wherein the agent binds to a region of the pre-mRNA transcript to increase inclusion of the NSAE in the mature transcript. For example, provided herein is a method of treatment or prevention of a condition associated with a functional-SYNGAP1 protein overexpression, comprising administering an alternative splicing modulator agent to a subject to decrease levels of functional SYNGAP1 protein, wherein the agent binds to a region of an exon or an intron (e.g., exon 10 or 11, intron 10 or intron 11 in human SYNGAP1 gene) of the pre-mRNA transcript.

According to one aspect of the present disclosure, provided herein is a method of treatment or prevention of a condition associated with a functional target protein overexpression, comprising administering an alternative splicing modulator agent to a subject to decrease levels of functional target protein, wherein the agent binds to a region of the pre-mRNA transcript to increase inclusion of the NSAE in the mature transcript. For example, provided herein is a method of treatment or prevention of a condition associated with a functional target protein overexpression, comprising administering an alternative splicing repressor agent to a subject to decrease levels of functional target protein, wherein the agent binds to a region of an exon or an intron of the pre-mRNA transcript, wherein the target is SYNGAP1.

Where reference is made to reducing alternative splicing in the mature mRNA, the reduction may be complete, e.g., 100%, or may be partial. The reduction may be clinically significant. The reduction/correction may be relative to the level of alternative splicing in the subject without treatment, or relative to the amount of alternative splicing in a population of similar subjects. The reduction/correction may be at least 10% less alternative splicing relative to the average subject, or the subject prior to treatment. The reduction may be at least 20% less alternative splicing relative to an average subject, or the subject prior to treatment. The reduction may be at least 40% less alternative splicing relative to an average subject, or the subject prior to treatment. The reduction may be at least 50% less alternative splicing relative to an average subject, or the subject prior to treatment. The reduction may be at least 60% less alternative splicing relative to an average subject, or the subject prior to treatment. The reduction may be at least 80% less alternative splicing relative to an average subject, or the subject prior to treatment. The reduction may be at least 90% less alternative splicing relative to an average subject, or the subject prior to treatment.

Where reference is made to increasing alternative splicing in the mature mRNA, the increase may be complete, e.g., 100%, or may be partial. The increase may be clinically significant. The increase/correction may be relative to the level of alternative splicing in the subject without treatment, or relative to the amount of alternative splicing in a population of similar subjects. The increase/correction may be at least 10% more alternative splicing relative to the average subject, or the subject prior to treatment. The increase may be at least 20% more alternative splicing relative to an average subject, or the subject prior to treatment. The increase may be at least 40% more alternative splicing relative to an average subject, or the subject prior to treatment. The increase may be at least 50% more alternative splicing relative to an average subject, or the subject prior to treatment. The increase may be at least 60% more alternative splicing relative to an average subject, or the subject prior to treatment. The increase may be at least 80% more alternative splicing relative to an average subject, or the subject prior to treatment. The increase may be at least 90% more alternative splicing relative to an average subject, or the subject prior to treatment.

Where reference is made to increasing functional-SYNGAP1 protein levels, the increase may be clinically significant. The increase may be relative to the level of functional-SYNGAP1 protein in the subject without treatment, or relative to the amount of functional-SYNGAP1 protein in a population of similar subjects. The increase may be at least 10% more functional-SYNGAP1 protein relative to the average subject, or the subject prior to treatment. The increase may be at least 20% more functional-SYNGAP1 protein relative to the average subject, or the subject prior to treatment. The increase may be at least 40% more functional-SYNGAP1 protein relative to the average subject, or the subject prior to treatment. The increase may be at least 50% more functional-SYNGAP1 protein relative to the average subject, or the subject prior to treatment. The increase may be at least 80% more functional-SYNGAP1 protein relative to the average subject, or the subject prior to treatment. The increase may be at least 100% more functional-SYNGAP1 protein relative to the average subject, or the subject prior to treatment. The increase may be at least 200% more functional-SYNGAP1 protein relative to the average subject, or the subject prior to treatment. The increase may be at least 500% more functional-SYNGAP1 protein relative to the average subject, or the subject prior to treatment.

Where reference is made to decreasing functional-SYNGAP1 protein levels, the decrease may be clinically significant. The decrease may be relative to the level of functional-SYNGAP1 protein in the subject without treatment, or relative to the amount of functional-SYNGAP1 protein in a population of similar subjects. The decrease may be at least 10% less functional-SYNGAP1 protein relative to the average subject, or the subject prior to treatment. The decrease may be at least 20% less functional-SYNGAP1 protein relative to the average subject, or the subject prior to treatment. The decrease may be at least 40% less functional-SYNGAP1 protein relative to the average subject, or the subject prior to treatment. The decrease may be at least 50% less functional-SYNGAP1 protein relative to the average subject, or the subject prior to treatment. The decrease may be at least 80% less functional-SYNGAP1 protein relative to the average subject, or the subject prior to treatment. The decrease may be at least 100% less functional-SYNGAP1 protein relative to the average subject, or the subject prior to treatment.

Where reference is made to increasing functional target protein levels, the increase may be clinically significant, wherein the target is SYNGAP1. The increase may be relative to the level of functional target protein in the subject without treatment, or relative to the amount of functional target protein in a population of similar subjects. The increase may be at least 10% more functional target protein relative to the average subject, or the subject prior to treatment. The increase may be at least 20% more functional target protein relative to the average subject, or the subject prior to treatment. The increase may be at least 40% more functional target protein relative to the average subject, or the subject prior to treatment. The increase may be at least 50% more functional target protein relative to the average subject, or the subject prior to treatment. The increase may be at least 80% more functional target protein relative to the average subject, or the subject prior to treatment. The increase may be at least 100% more functional target protein relative to the average subject, or the subject prior to treatment. The increase may be at least 200% more functional target protein relative to the average subject, or the subject prior to treatment. The increase may be at least 500% more functional target protein relative to the average subject, or the subject prior to treatment.

Where reference is made to decreasing functional target protein levels, the decrease may be clinically significant, wherein the target is SYNGAP1. The decrease may be relative to the level of functional target protein in the subject without treatment, or relative to the amount of functional target protein in a population of similar subjects. The decrease may be at least 10% less functional target protein relative to the average subject, or the subject prior to treatment. The decrease may be at least 20% less functional target protein relative to the average subject, or the subject prior to treatment. The decrease may be at least 40% less functional target protein relative to the average subject, or the subject prior to treatment. The decrease may be at least 50% less functional target protein relative to the average subject, or the subject prior to treatment. The decrease may be at least 80% less functional target protein relative to the average subject, or the subject prior to treatment. The decrease may be at least 100% less functional target protein relative to the average subject, or the subject prior to treatment.

In embodiments wherein the alternative splicing repressor agent comprises a polynucleic acid polymer, the polynucleic acid polymer may be about 50 nucleotides in length. In embodiments wherein the alternative splicing modulator agent comprises a polynucleic acid polymer, the polynucleic acid polymer may be about 50 nucleotides in length. The polynucleic acid polymer may be about 45 nucleotides in length. The polynucleic acid polymer may be about 40 nucleotides in length. The polynucleic acid polymer may be about 35 nucleotides in length. The polynucleic acid polymer may be about 30 nucleotides in length. The polynucleic acid polymer may be about 24 nucleotides in length. The polynucleic acid polymer may be about 25 nucleotides in length. The polynucleic acid polymer may be about 20 nucleotides in length. The polynucleic acid polymer may be about 19 nucleotides in length. The polynucleic acid polymer may be about 18 nucleotides in length. The polynucleic acid polymer may be about 17 nucleotides in length. The polynucleic acid polymer may be about 16 nucleotides in length. The polynucleic acid polymer may be about 15 nucleotides in length. The polynucleic acid polymer may be about 14 nucleotides in length. The polynucleic acid polymer may be about 13 nucleotides in length. The polynucleic acid polymer may be about 12 nucleotides in length. The polynucleic acid polymer may be about 11 nucleotides in length. The polynucleic acid polymer may be about 10 nucleotides in length. The polynucleic acid polymer may be between about 10 and about 50 nucleotides in length. The polynucleic acid polymer may be between about 10 and about 45 nucleotides in length. The polynucleic acid polymer may be between about 10 and about 40 nucleotides in length. The polynucleic acid polymer may be between about 10 and about 35 nucleotides in length. The polynucleic acid polymer may be between about 10 and about 30 nucleotides in length. The polynucleic acid polymer may be between about 10 and about 25 nucleotides in length. The polynucleic acid polymer may be between about 10 and about 20 nucleotides in length. The polynucleic acid polymer may be between about 15 and about 25 nucleotides in length. The polynucleic acid polymer may be between about 15 and about 30 nucleotides in length. The polynucleic acid polymer may be between about 12 and about 30 nucleotides in length.

The sequence of the polynucleic acid polymer may be at least 50%, 55%, 60%, 65%, 70%, 75%, 80%, 85%, 90%, 91%, 92%, 93%, 94%, 95%, 96%, 97%, 98%, 99%, or 99.5% complementary to a target sequence of an mRNA transcript, e.g., a partially processed mRNA transcript. The sequence of the polynucleic acid polymer may be 100% complementary to a target sequence of a pre-mRNA transcript.

The sequence of the polynucleic acid polymer may have 4 or fewer mismatches to a target sequence of the pre-mRNA transcript. The sequence of the polynucleic acid polymer may have 3 or fewer mismatches to a target sequence of the pre-mRNA transcript. The sequence of the polynucleic acid polymer may have 2 or fewer mismatches to a target sequence of the pre-mRNA transcript. The sequence of the polynucleic acid polymer may have 1 or fewer mismatches to a target sequence of the pre-mRNA transcript. The sequence of the polynucleic acid polymer may have no mismatches to a target sequence of the pre-mRNA transcript.

In some embodiments, the polynucleic acid polymer may specifically hybridize to a target sequence of the pre-mRNA transcript. For example, the polynucleic acid polymer may have 91%, 92%, 93%, 94%, 95%, 96%, 97%, 98%, 99%, 99.5%, or 100% sequence complementarity to a target sequence of the pre-mRNA transcript. The hybridization may be under high stringent hybridization conditions.

The polynucleic acid polymer may have a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence of Table 1 or Table 2. The polynucleic acid polymer may have a sequence with 100% sequence identity to a sequence selected from the group consisting of the sequences of Table 1 and Table 2. In some embodiments, the polynucleic acid polymer comprises a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to any one selected from the group consisting of the sequences of Table 1 and Table 2. In some embodiments, the polynucleic acid polymer comprises a sequence with 100% sequence identity to any one selected from the group consisting of the sequences of Table 1 and Table 2. In some embodiments the polynucleic acid polymer is a polynucleic acid polymer of at least 50%, 55%, 60%, 65%, 70%, 75%, 80%, 85%, 90%, 91%, 92%, 93%, 94%, 95%, 96%, 97%, 98%, 99%, or 99.5% sequence identity to any one selected from the group consisting of the sequences of Table 1 and Table 2. In some embodiments, the polynucleic acid polymer is a polynucleic acid polymer of any one selected from the group consisting of the sequences of Table 1 and Table 2.

TABLE 1
Exemplary ASOs targeting NSAE event

| ASO | | | | | | | |
|---|---|---|---|---|---|---|---|
| (Bold = 2'MOE, | | | | | | | |
| Italicized = LNA, | | ASO | | | | | |
| underlined C = | SEQ | sequence with | SEQ | | | | |
| 5methylC) | ID | no mismatches | ID | Coordinate | | | Name |
| Sequence 5'-3' | NO: | Sequence 5'-3' | NO: | Chr | Start | End | Position ID |
| ATGGGAGAGAGATG | 30 | | | chr6 | 33440369 | 33440387 | SYNGAP1-IVS10- |
| GAGG | | | | | | | 166 |
| CAGAGATGGGAGAG | 31 | | | chr6 | 33440374 | 33440392 | SYNGAP1-IVS10- |
| AGAT | | | | | | | 161 |
| AGAGACAGAGATGG | 32 | | | chr6 | 33440379 | 33440397 | SYNGAP1-IVS10- |
| GAGA | | | | | | | 156 |
| CAGGGAGAGACAGA | 33 | | | chr6 | 33440384 | 33440402 | SYNGAP1-IVS10- |
| GATG | | | | | | | 151 |
| GACACCAGGGAGAG | 34 | | | chr6 | 33440389 | 33440407 | SYNGAP1-IVS10- |
| ACAG | | | | | | | 146 |
| AAACAGACACCAGG | 35 | | | chr6 | 33440394 | 33440412 | SYNGAP1-IVS10- |
| GAGA | | | | | | | 141 |
| AAGAAAAACAGACA | 36 | | | chr6 | 33440399 | 33440417 | SYNGAP1-IVS10- |
| CCAG | | | | | | | 136 |
| AGGAGAAGAAAAAC | 37 | | | chr6 | 33440404 | 33440422 | SYNGAP1-IVS10- |
| AGAC | | | | | | | 131 |
| AGAGGAGGAGAAGA | 38 | | | chr6 | 33440409 | 33440427 | SYNGAP1-IVS10- |
| AAAA | | | | | | | 126 |
| CAAGGAGAGGAGGA | 39 | | | chr6 | 33440414 | 33440432 | SYNGAP1-IVS10- |
| GAAG | | | | | | | 121 |
| AGAGACAAGGAGAG | 40 | | | chr6 | 33440419 | 33440437 | SYNGAP1-IVS10- |
| GAGG | | | | | | | 116 |
| GGGAGAGAGACAAG | 41 | | | chr6 | 33440424 | 33440442 | SYNGAP1-IVS10- |
| GAGA | | | | | | | 111 |
| GGTGTGGGAGAGAG | 42 | | | chr6 | 33440429 | 33440447 | SYNGAP1-IVS10- |
| ACAA | | | | | | | 106 |
| GGAGGGTTGTGGGA | 43 | GGAGGGGTGTG | 173 | chr6 | 33440434 | 33440452 | SYNGAP1-IVS10- |
| GAGA | | GGAGAGA | | | | | 101 |
| GAGATGGATGGGTG | 44 | GAGATGGAGGG | 174 | chr6 | 33440439 | 33440457 | SYNGAP1-IVS10- |
| TGGG | | GTGTGGG | | | | | 96 |
| GGAGAGAGATGGAT | 45 | GGAGAGAGATG | 175 | chr6 | 33440444 | 33440462 | SYNGAP1-IVS10- |
| GGGT | | GAGGGGT | | | | | 91 |
| ACGTGGGAGAGAGA | 46 | | | chr6 | 33440449 | 33440467 | SYNGAP1-IVS10- |
| TGGA | | | | | | | 86 |
| GAGACACGTGGGAG | 47 | | | chr6 | 33440454 | 33440472 | SYNGAP1-IVS10- |
| AGAG | | | | | | | 81 |
| AGGGAGAGACACGT | 48 | GGGGAGAGACA | 176 | chr6 | 33440459 | 33440477 | SYNGAP1-IVS10- |
| GGGA | | CGTGGGA | | | | | 76 |
| GGTGAGGGAAGAGA | 49 | GGTGAGGGGAG | 177 | chr6 | 33440464 | 33440482 | SYNGAP1-IVS10- |
| CACG | | AGACACG | | | | | 71 |
| GAGAAGGTGAAGGG | 50 | GAGAAGGTGAG | 178 | chr6 | 33440469 | 33440487 | SYNGAP1-IVS10- |
| AGAG | | GGGAGAG | | | | | 66 |
| AGGGAGAGAAGGTG | 51 | GGGGAGAGAAG | 179 | chr6 | 33440474 | 33440492 | SYNGAP1-IVS10- |
| AGGG | | GTGAGGG | | | | | 61 |
| TGGAGGGAGAGAGA | 52 | TGGAGGGGGAG | 180 | chr6 | 33440479 | 33440497 | SYNGAP1-IVS10- |
| AGGT | | AGAAGGT | | | | | 56 |
| AGAAATGGAGAGGG | 53 | AGAAATGGAGG | 181 | chr6 | 33440484 | 33440502 | SYNGAP1-IVS10- |
| AGAG | | GGGAGAG | | | | | 51 |
| GAGAGAGAAATGGA | 54 | GAGAGAGAAAT | 182 | chr6 | 33440489 | 33440507 | SYNGAP1-IVS10- |
| GGGA | | GGAGGGG | | | | | 46 |
| TTAGGGAGAGAGAA | 55 | | | chr6 | 33440494 | 33440512 | SYNGAP1-IVS10- |
| ATGG | | | | | | | 41 |
| ACAGATTAGGGAGA | 56 | | | chr6 | 33440499 | 33440517 | SYNGAP1-IVS10- |
| GAGA | | | | | | | 36 |
| AACAGACAGATTAG | 57 | | | chr6 | 33440504 | 33440522 | SYNGAP1-IVS10- |
| GGAG | | | | | | | 31 |
| GAGGGAACAGACAG | 58 | | | chr6 | 33440509 | 33440527 | SYNGAP1-IVS10- |
| ATTA | | | | | | | 26 |
| TGGCAGAGGGAACA | 59 | | | chr6 | 33440514 | 33440532 | SYNGAP1-IVS10- |
| GACA | | | | | | | 21 |
| GGCCATGGCAGAGG | 60 | | | chr6 | 33440519 | 33440537 | SYNGAP1-IVS10- |
| GAAC | | | | | | | 16 |
| AAGGGAGCCATGGC | 61 | AAGGGGGCCAT | 183 | chr6 | 33440524 | 33440542 | SYNGAP1-IVS10- |
| AGAG | | GGCAGAG | | | | | 11 |
| TGAAGAAGGAGGCC | 62 | TGAAGAAGGGG | 184 | chr6 | 33440529 | 33440547 | SYNGAP1-IVS10- |
| ATGG | | GCCATGG | | | | | 6 |
| CTGCTTGAAGAAGA | 63 | CTGCTTGAAGA | 185 | chr6 | 33440534 | 33440552 | SYNGAP1-IVS10- |
| GGGC | | AGGGGGC | | | | | 1 |
| GGAGGCTGCTTGAA | 64 | | | chr6 | 33440539 | 33440557 | SYNGAP1- |
| GAAG | | | | | | | I10AltE11 + 5 |
| AGATGGGAGGCTGC | 65 | | | chr6 | 33440544 | 33440562 | SYNGAP1- |
| TTGA | | | | | | | I10AltE11 + 10 |
| GAGCAAGATGGGAG | 66 | | | chr6 | 33440549 | 33440567 | SYNGAP1- |
| GCTG | | | | | | | I10AltE11 + 15 |
| CAGGAGCAAGATGG | 67 | | | chr6 | 33440552 | 33440570 | SYNGAP1- |
| GAGG | | | | | | | AltEx11 + 1 |
| GACCGCAGGAGCAA | 68 | | | chr6 | 33440557 | 33440575 | SYNGAP1- |
| GATG | | | | | | | AltEx11 + 6 |
| GGAGGGACCGCAGG | 69 | | | chr6 | 33440562 | 33440580 | SYNGAP1- |
| AGCA | | | | | | | AltEx11 + 11 |
| GGGAAGGAGGGACC | 70 | | | chr6 | 33440567 | 33440585 | SYNGAP1- |
| GCAG | | | | | | | AltEx11 + 16 |
| AGACAGGGAAGGAG | 71 | | | chr6 | 33440572 | 33440590 | SYNGAP1- |
| GGAC | | | | | | | AltEx11 + 21 |
| GAGAGAGACAGGGA | 72 | | | chr6 | 33440577 | 33440595 | SYNGAP1- |
| AGGA | | | | | | | AltEx11 + 26 |
| AGGGTGAGAGAGAC | 73 | GGGGTGAGAGA | 186 | chr6 | 33440582 | 33440600 | SYNGAP1- |
| AGGG | | GACAGGG | | | | | AltEx11 + 31 |
| AAACAGGGATGAGA | 74 | AAACAGGGGTG | 187 | chr6 | 33440587 | 33440605 | SYNGAP1- |
| GAGA | | AGAGAGA | | | | | AltEx11 + 36 |
| TGTGGAAACAAGGG | 75 | TGTGGAAACAG | 188 | chr6 | 33440592 | 33440610 | SYNGAP1- |
| TGAG | | GGGTGAG | | | | | AltEx11 + 41 |
| GAGGGTGTGGAAAC | 76 | | | chr6 | 33440597 | 33440615 | SYNGAP1- |
| AGGG | | | | | | | AltEx11 + 46 |
| GAGGTGAGGGTGTG | 77 | | | chr6 | 33440602 | 33440620 | SYNGAP1- |
| GAAA | | | | | | | AltEx11 + 51 |
| GGTAGGAGGTGAGG | 78 | | | chr6 | 33440607 | 33440625 | SYNGAP1- |
| GTGT | | | | | | | AltEx11 + 56 |
| AGGGTGGTAGGAGG | 79 | GGGGTGGTAGG | 189 | chr6 | 33440612 | 33440630 | SYNGAP1- |
| TGAG | | AGGTGAG | | | | | AltEx11 + 61 |
| TGAGGGAGGTGGTA | 80 | TGAGGGGGGTG | 190 | chr6 | 33440617 | 33440635 | SYNGAP1- |
| GGAG | | GTAGGAG | | | | | AltEx11 + 66 |
| CATGCTGAGGAGGG | 81 | CATGCTGAGGG | 191 | chr6 | 33440622 | 33440640 | SYNGAP1- |
| TGGT | | GGGTGGT | | | | | AltEx11 + 71 |
| GGGAACATGCTGAG | 82 | GGGAACATGCT | 192 | chr6 | 33440627 | 33440645 | SYNGAP1- |
| AGGG | | GAGGGGG | | | | | AltEx11 + 76 |
| TTCCAGGGAACATG | 83 | | | chr6 | 33440632 | 33440650 | SYNGAP1- |
| CTGA | | | | | | | AltEx11 + 81 |
| TCAGCTTCCAGGGA | 84 | | | chr6 | 33440637 | 33440655 | SYNGAP1- |
| ACAT | | | | | | | AltEx11 + 86 |
| GGGAGAGAGATGGA | 85 | GGGAGAGAGAT | 193 | chr6 | 33440445 | 33440463 | SYNGAP1-IVS10- |
| TGGG | | GGAGGGG | | | | | 90 |
| TGGGAGAGAGATGG | 86 | | | chr6 | 33440446 | 33440464 | SYNGAP1-IVS10- |
| AGGG | | | | | | | 89 |
| GTGGGAGAGAGATG | 87 | | | chr6 | 33440447 | 33440465 | SYNGAP1-IVS10- |
| GAGG | | | | | | | 88 |
| CGTGGGAGAGAGAT | 88 | | | chr6 | 33440448 | 33440466 | SYNGAP1-IVS10- |
| GGAG | | | | | | | 87 |
| CACGTGGGAGAGAG | 89 | | | chr6 | 33440450 | 33440468 | SYNGAP1-IVS10- |
| ATGG | | | | | | | 85 |
| ACACGTGGGAGAGA | 90 | | | chr6 | 33440451 | 33440469 | SYNGAP1-IVS10- |
| GATG | | | | | | | 84 |
| GACACGTGGGAGAG | 91 | | | chr6 | 33440452 | 33440470 | SYNGAP1-IVS10- |
| AGAT | | | | | | | 83 |
| AGACACGTGGGAGA | 92 | | | chr6 | 33440453 | 33440471 | SYNGAP1-IVS10- |
| GAGA | | | | | | | 82 |
| AGAGACACGTGGGA | 93 | | | chr6 | 33440455 | 33440473 | SYNGAP1-IVS10- |
| GAGA | | | | | | | 80 |
| GAGAGACACGTGGG | 94 | | | chr6 | 33440456 | 33440474 | SYNGAP1-IVS10- |
| AGAG | | | | | | | 79 |
| GGAGAGACACGTGG | 95 | | | chr6 | 33440457 | 33440475 | SYNGAP1-IVS10- |
| GAGA | | | | | | | 78 |
| GGGAGAGACACGTG | 96 | | | chr6 | 33440458 | 33440476 | SYNGAP1-IVS10- |
| GGAG | | | | | | | 77 |
| AGGGAACATGCTGA | 97 | AGGGAACATGC | 194 | chr6 | 33440628 | 33440646 | SYNGAP1- |
| TGGG | | TGAGGGG | | | | | AltEX11 + 77 |
| CAGGGAACATGCTG | 98 | | | chr6 | 33440629 | 33440647 | SYNGAP1- |
| AGGG | | | | | | | AltEX11 + 78 |
| CCAGGGAACATGCT | 99 | | | chr6 | 33440630 | 33440648 | SYNGAP1- |
| GAGG | | | | | | | AltEX11 + 79 |
| TCCAGGGAACATGC | 100 | | | chr6 | 33440631 | 33440649 | SYNGAP1- |
| TGAG | | | | | | | AltEX11 + 80 |
| CTTCCAGGGAACAT | 101 | | | chr6 | 33440633 | 33440651 | SYNGAP1- |
| GCTG | | | | | | | AltEX11 + 82 |
| GCTTCCAGGGAACA | 102 | | | chr6 | 33440634 | 33440652 | SYNGAP1- |
| TGCT | | | | | | | AltEX11 + 83 |
| AGCTTCCAGGGAAC | 103 | | | chr6 | 33440635 | 33440653 | SYNGAP1- |
| ATGC | | | | | | | AltEX11 + 84 |
| CAGCTTCCAGGGAA | 104 | | | chr6 | 33440636 | 33440654 | SYNGAP1- |
| CATG | | | | | | | AltEX11 + 85 |
| GAGACACGTGGGAG | 105 | | | chr6 | 33440456 | 33440472 | SYNGAP1-IVS10- |
| AG | | | | | | | 81(16) |
| GACACGTGGGAGAG | 106 | | | chr6 | 33440454 | 33440470 | SYNGAP1-IVS10- |
| AG | | | | | | | 83(16) |
| CACGTGGGAGAGAG | 107 | | | chr6 | 33440452 | 33440468 | SYNGAP1-IVS10- |
| AT | | | | | | | 85(16) |
| CGTGGGAGAGAGAT | 108 | | | chr6 | 33440450 | 33440466 | SYNGAP1-IVS10- |
| GG | | | | | | | 87(16) |
| GTGGGAGAGAGATG | 109 | | | chr6 | 33440449 | 33440465 | SYNGAP1-IVS10- |
| GA | | | | | | | 88(16) |
| GAGACACGTGGGAG | 110 | | | chr6 | 33440458 | 33440472 | SYNGAP1-IVS10- |
| | | | | | | | 81(14) |
| GACACGTGGGAGAG | 111 | | | chr6 | 33440456 | 33440470 | SYNGAP1-IVS10- |
| | | | | | | | 83(14) |
| CACGTGGGAGAGAG | 112 | | | chr6 | 33440454 | 33440468 | SYNGAP1-IVS10- |
| | | | | | | | 85(14) |
| CGTGGGAGAGAGAT | 113 | | | chr6 | 33440452 | 33440466 | SYNGAP1-IVS10- |
| | | | | | | | 87(14) |
| TGGGAGAGAGATGG | 114 | | | chr6 | 33440450 | 33440464 | SYNGAP1-IVS10- |
| | | | | | | | 89(14) |
| GGGAGAGAGATGGA | 115 | | | chr6 | 33440449 | 33440463 | SYNGAP1-IVS10- |
| | | | | | | | 90(14) |
| GAGACACGTGGG | 116 | | | chr6 | 33440460 | 33440472 | SYNGAP1-IVS10- |
| | | | | | | | 81(12) |
| GACACGTGGGAG | 117 | | | chr6 | 33440458 | 33440470 | SYNGAP1-IVS10- |
| | | | | | | | 83(12) |
| CACGTGGGAGAG | 118 | | | chr6 | 33440456 | 33440468 | SYNGAP1-IVS10- |
| | | | | | | | 85(12) |
| CGTGGGAGAGAG | 119 | | | chr6 | 33440454 | 33440466 | SYNGAP1-IVS10- |
| | | | | | | | 87(12) |
| TGGGAGAGAGAT | 120 | | | chr6 | 33440452 | 33440464 | SYNGAP1-IVS10- |
| | | | | | | | 89(12) |
| GGAGAGAGATGG | 121 | | | chr6 | 33440450 | 33440462 | SYNGAP1-IVS10- |
| | | | | | | | 91(12) |
| GAGAGAGATGGA | 122 | | | chr6 | 33440449 | 33440461 | SYNGAP1-IVS10- |
| | | | | | | | 92(12) |
| CCAGGGAGAGACAG | 123 | | | chr6 | 33440385 | 33440403 | SYNGAP1-IVS10- |
| AGAT | | | | | | | 150 |
| ACCAGGGAGAGACA | 124 | | | chr6 | 33440386 | 33440404 | SYNGAP1-IVS10- |
| GAGA | | | | | | | 149 |
| CACCAGGGAGAGAC | 125 | | | chr6 | 33440387 | 33440405 | SYNGAP1-IVS10- |
| AGAG | | | | | | | 148 |
| ACACCAGGGAGAGA | 126 | | | chr6 | 33440388 | 33440406 | SYNGAP1-IVS10- |
| CAGA | | | | | | | 147 |
| AGACACCAGGGAGA | 127 | | | chr6 | 33440390 | 33440408 | SYNGAP1-IVS10- |
| GACA | | | | | | | 145 |
| CAGACACCAGGGAG | 128 | | | chr6 | 33440391 | 33440409 | SYNGAP1-IVS10- |
| AGAC | | | | | | | 144 |
| ACAGACACCAGGGA | 129 | | | chr6 | 33440392 | 33440410 | SYNGAP1-IVS10- |
| GAGA | | | | | | | 143 |
| AACAGACACCAGGG | 130 | | | chr6 | 33440393 | 33440411 | SYNGAP1-IVS10- |
| AGAG | | | | | | | 142 |
| AAAACAGACACCAG | 131 | | | chr6 | 33440395 | 33440413 | SYNGAP1-IVS10- |
| GGAG | | | | | | | 140 |
| AAAAACAGACACCA | 132 | | | chr6 | 33440396 | 33440414 | SYNGAP1-IVS10- |
| GGGA | | | | | | | 139 |
| GAAAAACAGACACC | 133 | | | chr6 | 33440397 | 33440415 | SYNGAP1-IVS10- |
| AGGG | | | | | | | 138 |
| AGAAAAACAGACAC | 134 | | | chr6 | 33440398 | 33440416 | SYNGAP1-IVS10- |
| CAGG | | | | | | | 137 |
| GAGACAGGGAAGGA | 135 | | | chr6 | 33440573 | 33440591 | SYNGAP1- |
| GGGA | | | | | | | AltEx11 + 22 |
| AGAGACAGGGAAGG | 136 | | | chr6 | 33440574 | 33440592 | SYNGAP1- |
| AGGG | | | | | | | AltEx11 + 23 |
| GAGAGACAGGGAAG | 137 | | | chr6 | 33440575 | 33440593 | SYNGAP1- |
| GAGG | | | | | | | AltEx11 + 24 |
| AGAGAGACAGGGAA | 138 | | | chr6 | 33440576 | 33440594 | SYNGAP1- |
| GGAG | | | | | | | AltEx11 + 25 |
| TGAGAGAGACAGGG | 139 | | | chr6 | 33440578 | 33440596 | SYNGAP1- |
| AAGG | | | | | | | AltEx11 + 27 |
| GTGAGAGAGACAGG | 140 | | | chr6 | 33440579 | 33440597 | SYNGAP1- |
| GAAG | | | | | | | AltEx11 + 28 |
| GGTGAGAGAGACAG | 141 | | | chr6 | 33440580 | 33440598 | SYNGAP1- |
| GGAA | | | | | | | AltEx11 + 29 |
| GGGTGAGAGAGACA | 142 | | | chr6 | 33440581 | 33440599 | SYNGAP1- |
| GGGA | | | | | | | AltEx11 + 30 |
| TTCCAGGGAACATG | 143 | | | chr6 | 33440630 | 33440650 | SYNGAP1- |
| CTGAGG | | | | | | | AltEX11 + 79 |
| AGGGAACATGCTGA | 144 | | | chr6 | 33440630 | 33440646 | SYNGAP1- |
| GG | | | | | | | AltEX11 + 79 |
| CAGGGAACATGCTG | 145 | | | chr6 | 33440631 | 33440647 | SYNGAP1- |
| AG | | | | | | | AltEX11 + 80 |
| CCAGGGAACATGCT | 146 | | | chr6 | 33440632 | 33440648 | SYNGAP1- |
| GA | | | | | | | AltEX11 + 81 |
| TCCAGGGAACATGC | 147 | | | chr6 | 33440633 | 33440649 | SYNGAP1- |
| TG | | | | | | | AltEX11 + 82 |
| TTCCAGGGAACATG | 148 | | | chr6 | 33440634 | 33440650 | SYNGAP1- |
| CT | | | | | | | AltEx11 + 83 |
| CCAGGGAACATGCT | 149 | | | chr6 | 33440630 | 33440648 | SYNGAP1- |
| GAGG | | | | | | | AltEX11 + 79 |
| TCCAGGGAACATGC | 150 | | | chr6 | 33440631 | 33440649 | SYNGAP1- |
| TGAG | | | | | | | AltEX11 + 80 |
| TTCCAGGGAACATG | 151 | | | chr6 | 33440632 | 33440650 | SYNGAP1- |
| CTGA | | | | | | | AltEx11 + 81 |
| AGGGAACATGCTGA | 152 | | | chr6 | 33440630 | 33440646 | SYNGAP1- |
| GG | | | | | | | AltEX11 + 79 |
| CAGGGAACATGCTG | 153 | | | chr6 | 33440631 | 33440647 | SYNGAP1- |
| AG | | | | | | | AltEX11 + 80 |
| CCAGGGAACATGCT | 154 | | | chr6 | 33440632 | 33440648 | SYNGAP1- |
| GA | | | | | | | AltEX11 + 81 |
| TCCAGGGAACATGC | 155 | | | chr6 | 33440633 | 33440649 | SYNGAP1- |
| TG | | | | | | | AltEX11 + 82 |
| TTCCAGGGAACATG | 156 | | | chr6 | 33440634 | 33440650 | SYNGAP1- |
| CT | | | | | | | AltEx11 + 83 |
| CCAGGGAACATGCT | 157 | | | chr6 | 33440630 | 33440648 | SYNGAP1- |
| GAGG | | | | | | | AltEX11 + 79a |
| TCCAGGGAACATGC | 158 | | | chr6 | 33440631 | 33440649 | SYNGAP1- |
| TGAG | | | | | | | AltEX11 + 80a |
| TTCCAGGGAACATG | 159 | | | chr6 | 33440632 | 33440650 | SYNGAP1- |
| CTGA | | | | | | | AltEx11 + 81a |
| AGGGAACATGCTGA | 160 | | | chr6 | 33440630 | 33440646 | SYNGAP1- |
| GG | | | | | | | AltEX11 + 79a |
| CAGGGAACATGCTG | 161 | | | chr6 | 33440631 | 33440647 | SYNGAP1- |
| AG | | | | | | | AltEX11 + 80a |
| CCAGGGAACATGCT | 162 | | | chr6 | 33440632 | 33440648 | SYNGAP1- |
| GA | | | | | | | AltEX11 + 81a |
| TCCAGGGAACATGC | 163 | | | chr6 | 33440633 | 33440649 | SYNGAP1- |
| TG | | | | | | | AltEX11 + 82a |
| TTCCAGGGAACATG | 164 | | | chr6 | 33440634 | 33440650 | SYNGAP1- |
| CT | | | | | | | AltEx11 + 83a |
| CAGACACCAGGGAG | 165 | | | chr6 | 33440389 | 33440409 | SYNGAP1-IVS10- |
| AGACAG | | | | | | | 146(20) |
| ACAGACACCAGGGA | 166 | | | chr6 | 33440389 | 33440410 | SYNGAP1-IVS10- |
| GAGACAG | | | | | | | 146(21) |
| AACAGACACCAGGG | 167 | | | chr6 | 33440389 | 33440411 | SYNGAP1-IVS10- |
| AGAGACAG | | | | | | | 146(22) |
| AAACAGACACCAGG | 168 | | | chr6 | 33440389 | 33440412 | SYNGAP1-IVS10- |
| GAGAGACAG | | | | | | | 146(23) |
| CAGACACCAGGGAG | 169 | | | chr6 | 33440389 | 33440409 | SYNGAP1-IVS10- |
| AGACAG | | | | | | | 146(20)-P |
| ACAGACACCAGGGA | 170 | | | chr6 | 33440389 | 33440410 | SYNGAP1-IVS10- |
| GAGACAG | | | | | | | 146(21)-P |
| AACAGACACCAGGG | 171 | | | chr6 | 33440389 | 33440411 | SYNGAP1-IVS10- |
| AGAGACAG | | | | | | | 146(22)-P |
| AAACAGACACCAGG | 172 | | | chr6 | 33440389 | 33440412 | SYNGAP1-IVS10- |
| GAGAGACAG | | | | | | | 146(23)-P |
| ATGGGAGAGAGATG | 30 | | | chr6 | 33440369 | 33440387 | SYNGAP1-IVS10- |
| GAGG | | | | | | | 166 |
| CAGAGATGGGAGAG | 31 | | | chr6 | 33440374 | 33440392 | SYNGAP1-IVS10- |
| AGAT | | | | | | | 161 |
| AGAGACAGAGATGG | 32 | | | chr6 | 33440379 | 33440397 | SYNGAP1-IVS10- |
| GAGA | | | | | | | 156 |
| CAGGGAGAGACAGA | 33 | | | chr6 | 33440384 | 33440402 | SYNGAP1-IVS10- |
| GATG | | | | | | | 151 |
| GACACCAGGGAGAG | 34 | | | chr6 | 33440389 | 33440407 | SYNGAP1-IVS10- |
| ACAG | | | | | | | 146 |
| AAACAGACACCAGG | 35 | | | chr6 | 33440394 | 33440412 | SYNGAP1-IVS10- |
| GAGA | | | | | | | 141 |
| AAGAAAAACAGACA | 36 | | | chr6 | 33440399 | 33440417 | SYNGAP1-IVS10- |
| CCAG | | | | | | | 136 |
| AGGAGAAGAAAAAC | 37 | | | chr6 | 33440404 | 33440422 | SYNGAP1-IVS10- |
| AGAC | | | | | | | 131 |
| AGAGGAGGAGAAGA | 38 | | | chr6 | 33440409 | 33440427 | SYNGAP1-IVS10- |
| AAAA | | | | | | | 126 |
| CAAGGAGAGGAGGA | 39 | | | chr6 | 33440414 | 33440432 | SYNGAP1-IVS10- |
| GAAG | | | | | | | 121 |
| AGAGACAAGGAGAG | 40 | | | chr6 | 33440419 | 33440437 | SYNGAP1-IVS10- |
| GAGG | | | | | | | 116 |
| GGGAGAGAGACAAG | 41 | | | chr6 | 33440424 | 33440442 | SYNGAP1-IVS10- |
| GAGA | | | | | | | 111 |
| GGTGTGGGAGAGAG | 42 | | | chr6 | 33440429 | 33440447 | SYNGAP1-IVS10- |
| ACAA | | | | | | | 106 |
| GGAGGGTTGTGGGA | 43 | | | chr6 | 33440434 | 33440452 | SYNGAP1-IVS10- |
| GAGA | | | | | | | 101 |
| GAGATGGATGGGTG | 44 | | | chr6 | 33440439 | 33440457 | SYNGAP1-IVS10- |
| TGGG | | | | | | | 96 |
| GGAGAGAGATGGAT | 45 | | | chr6 | 33440444 | 33440462 | SYNGAP1-IVS10- |
| GGGT | | | | | | | 91 |
| ACGTGGGAGAGAGA | 46 | | | chr6 | 33440449 | 33440467 | SYNGAP1-IVS10- |
| TGGA | | | | | | | 86 |
| GAGACACGTGGGAG | 47 | | | chr6 | 33440454 | 33440472 | SYNGAP1-IVS10- |
| AGAG | | | | | | | 81 |
| AGGGAGAGACACGT | 48 | | | chr6 | 33440459 | 33440477 | SYNGAP1-IVS10- |
| GGGA | | | | | | | 76 |
| GGTGAGGGAAGAGA | 49 | | | chr6 | 33440464 | 33440482 | SYNGAP1-IVS10- |
| CACG | | | | | | | 71 |
| GAGAAGGTGAAGGG | 50 | | | chr6 | 33440469 | 33440487 | SYNGAP1-IVS10- |
| AGAG | | | | | | | 66 |
| AGGGAGAGAAGGTG | 51 | | | chr6 | 33440474 | 33440492 | SYNGAP1-IVS10- |
| AGGG | | | | | | | 61 |
| TGGAGGGAGAGAGA | 52 | | | chr6 | 33440479 | 33440497 | SYNGAP1-IVS10- |
| AGGT | | | | | | | 56 |
| AGAAATGGAGAGGG | 53 | | | chr6 | 33440484 | 33440502 | SYNGAP1-IVS10- |
| AGAG | | | | | | | 51 |
| GAGAGAGAAATGGA | 54 | | | chr6 | 33440489 | 33440507 | SYNGAP1-IVS10- |
| GGGA | | | | | | | 46 |
| TTAGGGAGAGAGAA | 55 | | | chr6 | 33440494 | 33440512 | SYNGAP1-IVS10- |
| ATGG | | | | | | | 41 |
| ACAGATTAGGGAGA | 56 | | | chr6 | 33440499 | 33440517 | SYNGAP1-IVS10- |
| GAGA | | | | | | | 36 |
| AACAGACAGATTAG | 57 | | | chr6 | 33440504 | 33440522 | SYNGAP1-IVS10- |
| GGAG | | | | | | | 31 |
| GAGGGAACAGACAG | 58 | | | chr6 | 33440509 | 33440527 | SYNGAP1-IVS10- |
| ATTA | | | | | | | 26 |
| TGGCAGAGGGAACA | 59 | | | chr6 | 33440514 | 33440532 | SYNGAP1-IVS10- |
| GACA | | | | | | | 21 |
| GGCCATGGCAGAGG | 60 | | | chr6 | 33440519 | 33440537 | SYNGAP1-IVS10- |
| GAAC | | | | | | | 16 |
| AAGGGAGCCATGGC | 61 | | | chr6 | 33440524 | 33440542 | SYNGAP1-IVS10- |
| AGAG | | | | | | | 11 |
| TGAAGAAGGAGGCC | 62 | | | chr6 | 33440529 | 33440547 | SYNGAP1-IVS10- |
| ATGG | | | | | | | 6 |
| CTGCTTGAAGAAGA | 63 | | | chr6 | 33440534 | 33440552 | SYNGAP1-IVS10- |
| GGGC | | | | | | | 1 |
| GGAGGCTGCTTGAA | 64 | | | chr6 | 33440539 | 33440557 | SYNGAP1- |
| GAAG | | | | | | | I10AltE11 + 5 |
| AGATGGGAGGCTGC | 65 | | | chr6 | 33440544 | 33440562 | SYNGAP1- |
| TTGA | | | | | | | I10AltE11 + 10 |
| GAGCAAGATGGGAG | 66 | | | chr6 | 33440549 | 33440567 | SYNGAP1- |
| GCTG | | | | | | | I10AltE11 + 15 |
| CAGGAGCAAGATGG | 67 | | | chr6 | 33440554 | 33440572 | SYNGAP1- |
| GAGG | | | | | | | AltEx11 + 1 |
| GACCGCAGGAGCAA | 68 | | | chr6 | 33440559 | 33440577 | SYNGAP1- |
| GATG | | | | | | | AltEx11 + 6 |
| GGAGGGACCGCAGG | 69 | | | chr6 | 33440564 | 33440582 | SYNGAP1- |
| AGCA | | | | | | | AltEx11 + 11 |
| GGGAAGGAGGGACC | 70 | | | chr6 | 33440569 | 33440587 | SYNGAP1- |
| GCAG | | | | | | | AltEx11 + 16 |
| AGACAGGGAAGGAG | 71 | | | chr6 | 33440574 | 33440592 | SYNGAP1- |
| GGAC | | | | | | | AltEx11 + 21 |
| GAGAGAGACAGGGA | 72 | | | chr6 | 33440579 | 33440597 | SYNGAP1- |
| AGGA | | | | | | | AltEx11 + 26 |
| AGGGTGAGAGAGAC | 73 | | | chr6 | 33440584 | 33440602 | SYNGAP1- |
| AGGG | | | | | | | AltEx11 + 31 |
| AAACAGGGATGAGA | 74 | | | chr6 | 33440589 | 33440607 | SYNGAP1- |
| GAGA | | | | | | | AltEx11 + 36 |
| TGTGGAAACAAGGG | 75 | | | chr6 | 33440594 | 33440612 | SYNGAP1- |
| TGAG | | | | | | | AltEx11 + 41 |
| GAGGGTGTGGAAAC | 76 | | | chr6 | 33440599 | 33440617 | SYNGAP1- |
| AGGG | | | | | | | AltEx11 + 46 |
| GAGGTGAGGGTGTG | 77 | | | chr6 | 33440604 | 33440622 | SYNGAP1- |
| GAAA | | | | | | | AltEx11 + 51 |
| GGTAGGAGGTGAGG | 78 | | | chr6 | 33440609 | 33440627 | SYNGAP1- |
| GTGT | | | | | | | AltEx11 + 56 |
| AGGGTGGTAGGAGG | 79 | | | chr6 | 33440614 | 33440632 | SYNGAP1- |
| TGAG | | | | | | | AltEx11 + 61 |
| TGAGGGAGGTGGTA | 80 | | | chr6 | 33440619 | 33440637 | SYNGAP1- |
| GGAG | | | | | | | AltEx11 + 66 |
| CATGCTGAGGAGGG | 81 | | | chr6 | 33440624 | 33440642 | SYNGAP1- |
| TGGT | | | | | | | AltEx11 + 71 |
| GGGAACATGCTGAG | 82 | | | chr6 | 33440629 | 33440647 | SYNGAP1- |
| AGGG | | | | | | | AltEx11 + 76 |
| TTCCAGGGAACATG | 83 | | | chr6 | 33440634 | 33440652 | SYNGAP1- |
| CTGA | | | | | | | AltEx11 + 81 |
| TCAGCTTCCAGGGA | 84 | | | chr6 | 33440639 | 33440657 | SYNGAP1- |
| ACAT | | | | | | | AltEx11 + 86 |
| GGGAGAGAGATGGA | 85 | | | chr6 | 33408222 | 33408239 | SYNGAP1-IVS10- |
| TGGG | | | | | | | 90 |
| TGGGAGAGAGATGG | 86 | | | chr6 | 33408223 | 33408240 | SYNGAP1-IVS10- |
| AGGG | | | | | | | 89 |
| GTGGGAGAGAGATG | 87 | | | chr6 | 33408224 | 33408241 | SYNGAP1-IVS10- |
| GAGG | | | | | | | 88 |
| CGTGGGAGAGAGAT | 88 | | | chr6 | 33408225 | 33408242 | SYNGAP1-IVS10- |
| GGAG | | | | | | | 87 |
| CACGTGGGAGAGAG | 89 | | | chr6 | 33408227 | 33408244 | SYNGAP1-IVS10- |
| ATGG | | | | | | | 85 |
| ACACGTGGGAGAGA | 90 | | | chr6 | 33408228 | 33408245 | SYNGAP1-IVS10- |
| GATG | | | | | | | 84 |
| GACACGTGGGAGAG | 91 | | | chr6 | 33408229 | 33408246 | SYNGAP1-IVS10- |
| AGAT | | | | | | | 83 |
| AGACACGTGGGAGA | 92 | | | chr6 | 33408230 | 33408247 | SYNGAP1-IVS10- |
| GAGA | | | | | | | 82 |
| AGAGACACGTGGGA | 93 | | | chr6 | 33408232 | 33408249 | SYNGAP1-IVS10- |
| GAGA | | | | | | | 80 |
| GAGAGACACGTGGG | 94 | | | chr6 | 33408233 | 33408250 | SYNGAP1-IVS10- |
| AGAG | | | | | | | 79 |
| GGAGAGACACGTGG | 95 | | | chr6 | 33408234 | 33408251 | SYNGAP1-IVS10- |
| GAGA | | | | | | | 78 |
| GGGAGAGACACGTG | 96 | | | chr6 | 33408235 | 33408252 | SYNGAP1-IVS10- |
| GGAG | | | | | | | 77 |
| AGGGAACATGCTGA | 97 | | | chr6 | 33408405 | 33408422 | SYNGAP1- |
| TGGG | | | | | | | AltEX11 + 77 |
| CAGGGAACATGCTG | 98 | | | chr6 | 33408406 | 33408423 | SYNGAP1- |
| AGGG | | | | | | | AltEX11 + 78 |
| CCAGGGAACATGCT | 99 | | | chr6 | 33408407 | 33408424 | SYNGAP1- |
| GAGG | | | | | | | AltEX11 + 79 |
| TCCAGGGAACATGC | 100 | | | chr6 | 33408408 | 33408425 | SYNGAP1- |
| TGAG | | | | | | | AltEX11 + 80 |
| CTTCCAGGGAACAT | 101 | | | chr6 | 33408410 | 33408427 | SYNGAP1- |
| GCTG | | | | | | | AltEX11 + 82 |
| GCTTCCAGGGAACA | 102 | | | chr6 | 33408411 | 33408428 | SYNGAP1- |
| TGCT | | | | | | | AltEX11 + 83 |
| AGCTTCCAGGGAAC | 103 | | | chr6 | 33408412 | 33408429 | SYNGAP1- |
| ATGC | | | | | | | AltEX11 + 84 |
| CAGCTTCCAGGGAA | 104 | | | chr6 | 33408413 | 33408430 | SYNGAP1- |
| CATG | | | | | | | AltEX11 + 85 |
| GAGACACGTGGGAG | 105 | | | chr6 | 33408233 | 33408248 | SYNGAP1-IVS10- |
| AG | | | | | | | 81(16) |
| GACACGTGGGAGAG | 106 | | | chr6 | 33408231 | 33408246 | SYNGAP1-IVS10- |
| AG | | | | | | | 83(16) |
| CACGTGGGAGAGAG | 107 | | | chr6 | 33408229 | 33408244 | SYNGAP1-IVS10- |
| AT | | | | | | | 85(16) |
| CGTGGGAGAGAGAT | 108 | | | chr6 | 33408227 | 33408242 | SYNGAP1-IVS10- |
| GG | | | | | | | 87(16) |
| GTGGGAGAGAGATG | 109 | | | chr6 | 33408226 | 33408241 | SYNGAP1-IVS10- |
| GA | | | | | | | 88(16) |
| GAGACACGTGGGAG | 110 | | | chr6 | 33408235 | 33408248 | SYNGAP1-IVS10- |
| | | | | | | | 81(14) |
| GACACGTGGGAGAG | 111 | | | chr6 | 33408233 | 33408246 | SYNGAP1-IVS10- |
| | | | | | | | 83(14) |
| CACGTGGGAGAGAG | 112 | | | chr6 | 33408231 | 33408244 | SYNGAP1-IVS10- |
| | | | | | | | 85(14) |
| CGTGGGAGAGAGAT | 113 | | | chr6 | 33408229 | 33408242 | SYNGAP1-IVS10- |
| | | | | | | | 87(14) |
| TGGGAGAGAGATGG | 114 | | | chr6 | 33408227 | 33408240 | SYNGAP1-IVS10- |
| | | | | | | | 89(14) |
| GGGAGAGAGATGGA | 115 | | | chr6 | 33408226 | 33408239 | SYNGAP1-IVS10- |
| | | | | | | | 90(14) |
| GAGACACGTGGG | 116 | | | chr6 | 33408237 | 33408248 | SYNGAP1-IVS10- |
| | | | | | | | 81(12) |
| GACACGTGGGAG | 117 | | | chr6 | 33408235 | 33408246 | SYNGAP1-IVS10- |
| | | | | | | | 83(12) |
| CACGTGGGAGAG | 118 | | | chr6 | 33408233 | 33408244 | SYNGAP1-IVS10- |
| | | | | | | | 85(12) |
| CGTGGGAGAGAG | 119 | | | chr6 | 33408231 | 33408242 | SYNGAP1-IVS10- |
| | | | | | | | 87(12) |
| TGGGAGAGAGAT | 120 | | | chr6 | 33408229 | 33408240 | SYNGAP1-IVS10- |
| | | | | | | | 89(12) |
| GGAGAGAGATGG | 121 | | | chr6 | 33408227 | 33408238 | SYNGAP1-IVS10- |
| | | | | | | | 91(12) |
| GAGAGAGATGGA | 122 | | | chr6 | 33408226 | 33408237 | SYNGAP1-IVS10- |
| | | | | | | | 92(12) |

TABLE 2
Exemplary ASOs targeting NMD Exon 18x event

| ASO | | | | | |
|---|---|---|---|---|---|
| (Bold = 2' MOE, | | | | | |
| Italicized = | | | | | |
| 2' O-Me, | | | | | |
| Underlined C = | | | | | |
| Bold and | | | | | |
| italicized = | | | | | |
| 5methylC | | | | | |
| Bold and | | | | | |
| italicized = | | Genomic Coordinates | | | |
| 2'-Fluoro) | SEQ ID | (GRCh38/hg38) | | | Name |
| Sequence 5'-3' | NO: | Chr | Start | End | Position ID |
| AGGAGTATGGGT | 195 | chr6 | 33448685 | 33448703 | SYNGAP1:NM 006772:IVS17: |
| AGGAAA | | | | | -86 |
| CAAAGAGGAGTAT | 196 | chr6 | 33448690 | 33448708 | SYNGAP1:NM 006772:IVS17: |
| GGGTA | | | | | -81 |
| CAGAGCAAAGAG | 197 | chr6 | 33448695 | 33448713 | SYNGAP1:NM 006772:IVS17: |
| GAGTAT | | | | | -76 |
| GCAGTCAGAGCA | 198 | chr6 | 33448700 | 33448718 | SYNGAP1:NM 006772:IVS17: |
| AAGAGG | | | | | -71 |
| GCCTAGCAGTCAG | 199 | chr6 | 33448705 | 33448723 | SYNGAP1:NM 006772:IVS17: |
| AGCAA | | | | | -66 |
| GGGGAGCCTAGC | 200 | chr6 | 33448710 | 33448728 | SYNGAP1:NM_006772:IVS17: |
| AGTCAG | | | | | -61 |
| GGCGGGGGGAGC | 201 | chr6 | 33448715 | 33448733 | SYNGAP1:NM_006772:IVS17: |
| CTAGCA | | | | | -56 |
| TGGCAGGCGGGG | 202 | chr6 | 33448720 | 33448738 | SYNGAP1:NM 006772:IVS17: |
| GGAGCC | | | | | -51 |
| GGGGATGGCAGG | 203 | chr6 | 33448725 | 33448743 | SYNGAP1:NM_006772:IVS17: |
| CGGGGG | | | | | -46 |
| GGTGGGGGGATG | 204 | chr6 | 33448730 | 33448748 | SYNGAP1:NM 006772:IVS17: |
| GCAGGC | | | | | -41 |
| GCCTTGGTGGGG | 205 | chr6 | 33448735 | 33448753 | SYNGAP1:NM_006772:IVS17: |
| GGATGG | | | | | -36 |
| CAGGAGCCTTGGT | 206 | chr6 | 33448740 | 33448758 | SYNGAP1:NM_006772:IVS17: |
| GGGGG | | | | | -31 |
| GGGGTCAGGAGC | 207 | chr6 | 33448745 | 33448763 | SYNGAP1:NM_006772:IVS17: |
| CTTGGT | | | | | -26 |
| GTCATGGGGTCA | 208 | chr6 | 33448750 | 33448768 | SYNGAP1:NM_006772:IVS17: |
| GGAGCC | | | | | -21 |
| GTGGGGTCATGG | 209 | chr6 | 33448755 | 33448773 | SYNGAP1:NM 006772:IVS17: |
| GGTCAG | | | | | -16 |
| AGAGAGTGGGGT | 210 | chr6 | 33448760 | 33448778 | SYNGAP1:NM 006772:IVS17: |
| CATGGG | | | | | -11 |
| GTGGGAGAGAGT | 211 | chr6 | 33448765 | 33448783 | SYNGAP1:NM_006772:IVS17: |
| GGGGTC | | | | | -6 |
| CTGCAGTGGGAG | 212 | chr6 | 33448770 | 33448788 | SYNGAP1:NM_006772:IVS17: |
| AGAGTG | | | | | -1 |
| AGGAGCTGCAGT | 213 | chr6 | 33448775 | 33448793 | SYNGAP1:NM_006772:IVS17- |
| GGGAGA | | | | | EX18x:+5 |
| TGATGAGGAGCT | 214 | chr6 | 33448780 | 33448798 | SYNGAP1:NM_006772:IVS17- |
| GCAGTG | | | | | EX18x:+10 |
| TTACCTGATGAGG | 215 | chr6 | 33448785 | 33448803 | SYNGAP1:NM 006772:IVS17- |
| AGCTG | | | | | EX18x:+15 |
| GAATTACCTGATG | 216 | chr6 | 33448788 | 33448806 | SYNGAP1:NM_006772:EX18x: |
| AGGAG | | | | | +1 |
| CAGGAGAATTACC | 217 | chr6 | 33448793 | 33448811 | SYNGAP1:NM 006772:EX18x: |
| TGATG | | | | | +6 |
| GGAACCAGGAGA | 218 | chr6 | 33448798 | 33448816 | SYNGAP1:NM 006772:EX18x: |
| ATTACC | | | | | +11 |
| AAAGCGGAACCA | 219 | chr6 | 33448803 | 33448821 | SYNGAP1:NM 006772:EX18x: |
| GGAGAA | | | | | +16 |
| TGGCCAAAGCGG | 220 | chr6 | 33448808 | 33448826 | SYNGAP1:NM 006772:EX18x: |
| AACCAG | | | | | +21 |
| GCCCGTGGCCAA | 221 | chr6 | 33448813 | 33448831 | SYNGAP1:NM 006772:EX18x: |
| AGCGGA | | | | | +26 |
| CCTCCGCCCGTGG | 222 | chr6 | 33448818 | 33448836 | SYNGAP1:NM_006772:EX18x: |
| CCAAA | | | | | +31 |
| CCTGTGTCCTCCG | 223 | chr6 | 33448825 | 33448843 | SYNGAP1:NM 006772:EX18x: |
| CCCGT | | | | | -26 |
| CTCCCCCTGTGTC | 224 | chr6 | 33448830 | 33448848 | SYNGAP1:NM_006772:EX18x: |
| CTCCG | | | | | -21 |
| GTCACCTCCCCCT | 225 | chr6 | 33448835 | 33448853 | SYNGAP1:NM_006772:EX18x: |
| GTGTC | | | | | -16 |
| CCGGAGTCACCTC | 226 | chr6 | 33448840 | 33448858 | SYNGAP1:NM_006772:EX18x: |
| CCCCT | | | | | -11 |
| GTGGTCCGGAGT | 227 | chr6 | 33448845 | 33448863 | SYNGAP1:NM_006772:EX18x: |
| CACCTC | | | | | -6 |
| CTGCAGTGGTCCG | 228 | chr6 | 33448850 | 33448868 | SYNGAP1:NM_006772:EX18x: |
| GAGTC | | | | | -1 |
| AACCTGCAGTGGT | 229 | chr6 | 33448853 | 33448871 | SYNGAP1:NM_006772:EX18x- |
| CCGGA | | | | | IVS18:-15 |
| CGACCAACCTGCA | 230 | chr6 | 33448858 | 33448876 | SYNGAP1:NM_006772:EX18x- |
| GTGGT | | | | | IVS18:-10 |
| CTTCACGACCAAC | 231 | chr6 | 33448863 | 33448881 | SYNGAP1:NM_006772:EX18x- |
| CTGCA | | | | | IVS18:-5 |
| GTGGGCTTCACGA | 232 | chr6 | 33448868 | 33448886 | SYNGAP1:NM_006772:IVS18: |
| CCAAC | | | | | +1 |
| AGGGAGTGGGCT | 233 | chr6 | 33448873 | 33448891 | SYNGAP1:NM_006772:IVS18: |
| TCACGA | | | | | +6 |
| GTTGGAGGGAGT | 234 | chr6 | 33448878 | 33448896 | SYNGAP1:NM_006772:IVS18: |
| GGGCTT | | | | | +11 |
| GAGGTGTTGGAG | 235 | chr6 | 33448883 | 33448901 | SYNGAP1:NM 006772:IVS18: |
| GGAGTG | | | | | +16 |
| CTCCGGAGGTGTT | 236 | chr6 | 33448888 | 33448906 | SYNGAP1:NM 006772:IVS18: |
| GGAGG | | | | | +21 |
| AGAGGCTCCGGA | 237 | chr6 | 33448893 | 33448911 | SYNGAP1:NM_006772:IVS18: |
| GGTGTT | | | | | +26 |
| AGGGGAGAGGCT | 238 | chr6 | 33448898 | 33448916 | SYNGAP1:NM_006772:IVS18: |
| CCGGAG | | | | | +31 |
| GTGAGAGGGGAG | 239 | chr6 | 33448903 | 33448921 | SYNGAP1:NM_006772:IVS18: |
| AGGCTC | | | | | +36 |
| CAGCAGTGAGAG | 240 | chr6 | 33448908 | 33448926 | SYNGAP1:NM_006772:IVS18: |
| GGGAGA | | | | | +41 |
| GAGGGCAGCAGT | 241 | chr6 | 33448913 | 33448931 | SYNGAP1:NM_006772:IVS18: |
| GAGAGG | | | | | +46 |
| GTGTGGAGGGCA | 242 | chr6 | 33448918 | 33448936 | SYNGAP1:NM_006772:IVS18: |
| GCAGTG | | | | | +51 |
| TCTGGGTGTGGA | 243 | chr6 | 33448923 | 33448941 | SYNGAP1:NM_006772:IVS18: |
| GGGCAG | | | | | +56 |
| GGTTCTCTGGGTG | 244 | chr6 | 33448928 | 33448946 | SYNGAP1:NM 006772:IVS18: |
| TGGAG | | | | | +61 |
| GTGGAGGTTCTCT | 245 | chr6 | 33448933 | 33448951 | SYNGAP1:NM_006772:IVS18: |
| GGGTG | | | | | +66 |
| AGTCTGTGGAGGT | 246 | chr6 | 33448938 | 33448956 | SYNGAP1:NM_006772:IVS18: |
| TCTCT | | | | | +71 |
| GCTGGAGTCTGTG | 247 | chr6 | 33448943 | 33448961 | SYNGAP1:NM_006772:IVS18: |
| GAGGT | | | | | +76 |
| GGAGGGCTGGAG | 248 | chr6 | 33448948 | 33448966 | SYNGAP1:NM_006772:IVS18: |
| TCTGTG | | | | | +81 |
| GTGTCGGAGGGC | 249 | chr6 | 33448953 | 33448971 | SYNGAP1:NM 006772:IVS18: |
| TGGAGT | | | | | +86 |
| AAAGCGGAACCA | 250 | chr6 | 33448808 | 33448821 | |
| G | | | | | |
| CAAAGCGGAACC | 251 | chr6 | 33448808 | 33448822 | |
| AG | | | | | |
| CCAAAGCGGAAC | 252 | chr6 | 33448808 | 33448823 | |
| CAG | | | | | |
| GCCAAAGCGGAA | 253 | chr6 | 33448808 | 33448824 | |
| CCAG | | | | | |
| GCCAAAGCG7dGA | 254 | chr6 | 33448808 | 33448824 | |
| ACCAG | | | | | |
| GCCAAAGCGGAA | 255 | chr6 | 33448808 | 33448824 | |
| CCAG | | | | | |
| GCCAAAGCGGAA | 256 | chr6 | 33448808 | 33448824 | |
| CCAG | | | | | |
| GCCAAAGCGGAA | 257 | chr6 | 33448808 | 33448824 | |
| CCAG | | | | | |
| GCCAAAGCGGAA | 258 | chr6 | 33448808 | 33448824 | |
| CCAG | | | | | |
| GCCAAAGCGGAA | 259 | chr6 | 33448808 | 33448824 | |
| CCAG | | | | | |
| GCCAAAGCGGAA | 260 | chr6 | 33448808 | 33448824 | |
| CCAG | | | | | |
| CTTCACGACCAAC | 261 | chr6 | 33448865 | 33448881 | |
| CTG | | | | | |
| CTTCACGACCAAC | 262 | chr6 | 33448866 | 33448881 | |
| CT | | | | | |
| CTTCACGACCAAC | 263 | chr6 | 33448867 | 33448881 | |
| C | | | | | |
| CTTCACGACCAAC | 264 | chr6 | 33448868 | 33448881 | |
| TCACGACCAACCT | 265 | chr6 | 33448863 | 33448879 | |
| GCA | | | | | |
| GGCTTCACGACCA | 266 | chr6 | 33448868 | 33448883 | |
| AC | | | | | |
| GCTTCACGACCAA | 267 | chr6 | 33448868 | 33448882 | |
| C | | | | | |
| CTTCACGACCAAC | 268 | chr6 | 33448863 | 33448881 | SYNGAP1:NM_006772:EX18x- |
| CTGCA | | | | | IVS18:-5 |
| CTTCACGACCAAC | 269 | chr6 | 33448863 | 33448881 | SYNGAP1:NM_006772:EX18x- |
| CTGCA | | | | | IVS18:-5 |
| CTTCACGACCAAC | 270 | chr6 | 33448863 | 33448881 | SYNGAP1:NM_006772:EX18x- |
| CT7dGCA | | | | | IVS18:-5 |
| CTTCACGACCAAC | 271 | chr6 | 33448863 | 33448881 | SYNGAP1:NM_006772:EX18x- |
| CTGCA | | | | | IVS18:-5 |
| CUTCACGACCAAC | 272 | chr6 | 33448863 | 33448881 | SYNGAP1:NM 006772:EX18x- |
| CTGCA | | | | | IVS18:-5 |
| CTTCACGACCAAC | 273 | chr6 | 33448863 | 33448881 | SYNGAP1:NM_006772:EX18x- |
| CTGCA | | | | | IVS18:-5 |
| CTTCACCACCAAC | 274 | chr17 | 26967755 | 26967773 | |
| CTGCA | | | | | |
| GTCAAAGCGGAA | 275 | chr17 | 26967700 | 26967716 | |
| CCAG | | | | | |

TABLE 3
SYNGAP1 sequences related to NSAE event
SYNGAP1

| | |
|---|---|
| Canonical intron | gtgagactgggaacgctgggctggggggccagggtcgggggaattatgtgttcatctgttcatctatctgtcc |
| chr6: 33438919- | atcctcaaagaggactgagcaccatttatgggcaaagcattgttctaggcgctatagagcaaacaggtgaaa |
| 33440728 | gaggcctggtccctgccctcagagggcctccaccagaatggggacaaattagaagaaaaaaaaaaaaagc |
| | cacagagccataatggtgtgtaagtgctgagtaagggtccccccaacctctgtgtgacataaggtcagagag |
| | aaggcagagctttgagataagtggggaagaggtgcccccttgggtaggctttgaagactggtttaggttctga |
| | tatatggacatagttggcaagaaagacatttcagaagaaggctgtgagaaaggcacatgtgtgatggtgaaa |
| | aggcccaggagttttcaggggacattaaagtaggttagtagcaattacatcaggtttagtggagcatgtgcctc |
| | ataatggggagtggcgggagagatgtctgggcaggaagattagctttagaaactggaaggcctcaaggagt |
| | ctgaggtcattggtaggccttgggatgccattaaaggtgtcagaaatattgtgatttagaagattaatccatagg |
| | ctgggcacggtggctcacacctgtaataccagcactttgggaggctgaggcgggcagaccacctgaggtca |
| | ggagtttgagaccaggctgaccaacatggagaaaccctgtctctactaaaaatacaaaattagccagatgtgg |
| | tggcacatgcctgtaatcccagctattcgggaggctaaggcaggagaatcacttgaatctgggaggtggag |
| | gttgtagtgagccgagatcacacgattgcactctagcctgggcaacaagagtgaaactccatctcaaaaaaa |
| | aaaaattaatccataagtagagtgtatgtagtagagtagagggatgtatgttgggggatgactggaatagagc |
| | cagctgggggctcggaggcatgaaagtcagatcctgaatcagaacagtgacagaagttaggaaggagcta |
| | ctggaaggaccctctcgggaaaggatcagtaggaattgtcagcttattggaaaggagggagcatgtctgtgg |
| | ggagtcacggatgactcaagaggccatgaggctggtggttgggagaccggtggcctcattgacaaccagg |
| | aaagtcaggaggaggagccagttggaggtgtgggggcgatggtgagctctgctttacaccagccgagttta |
| | aggtgtcagtgggacattgaagtggaactgggaggtgcggggagtgagctctgagccattccagggactg |
| | gggatcatgcctggggcacctccatccccatttccctggaatccagaagagttggggggtccgagctccctg |
| | tacctcaagtgaccctccatctctctcccatctctgtctctccctggtgtctgtttttcttctcctcctctccttgtctct |
| | ctcccacacccctccatctctctcccacgtgtctctcccctcaccttctctccccctccatttctctctccctaatct |
| | gtctgttccctctgccatggcccccttcttcaagcagcctcccatcttgctcctgcggtccctccttccctgtctct |
| | ctcacccctgtttccacaccctcacctcctaccacccccctcagcatgttccctggaagctgagggtctctggg |
| | gctcagtcccggtctctctctttctctctctctctctctgtctccccgacccttccccccag (SEQ ID NO: |
| | 276) |
| Alternative intron | gtgagactgggaacgctgggctggggggccagggtcgggggaattatgtgttcatctgttcatctatctgtcc |
| chr6: 33438919- | atcctcaaagaggactgagcaccatttatgggcaaagcattgttctaggcgctatagagcaaacaggtgaaa |
| 33440552 | gaggcctggtccctgccctcagagggcctccaccagaatggggacaaattagaagaaaaaaaaaaaaagc |
| | cacagagccataatggtgtgtaagtgctgagtaagggtccccccaacctctgtgtgacataaggtcagagag |
| | aaggcagagctttgagataagtggggaagaggtgcccccttgggtaggctttgaagactggtttaggttctga |
| | tatatggacatagttggcaagaaagacatttcagaagaaggctgtgagaaaggcacatgtgtgatggtgaaa |
| | aggcccaggagttttcaggggacattaaagtaggttagtagcaattacatcaggtttagtggagcatgtgcctc |
| | ataatggggagtggcgggagagatgtctgggcaggaagattagctttagaaactggaaggcctcaaggagt |
| | ctgaggtcattggtaggccttgggatgccattaaaggtgtcagaaatattgtgatttagaagattaatccatagg |
| | ctgggcacggtggctcacacctgtaataccagcactttgggaggctgaggcgggcagaccacctgaggtca |
| | ggagtttgagaccaggctgaccaacatggagaaaccctgtctctactaaaaatacaaaattagccagatgtgg |
| | tggcacatgcctgtaatcccagctattcgggaggctaaggcaggagaatcacttgaatctgggaggtggag |
| | gttgtagtgagccgagatcacacgattgcactctagcctgggcaacaagagtgaaactccatctcaaaaaaa |
| | aaaaattaatccataagtagagtgtatgtagtagagtagagggatgtatgttgggggatgactggaatagagc |
| | cagctgggggctcggaggcatgaaagtcagatcctgaatcagaacagtgacagaagttaggaaggagcta |
| | ctggaaggaccctctcgggaaaggatcagtaggaattgtcagcttattggaaaggagggagcatgtctgtgg |
| | ggagtcacggatgactcaagaggccatgaggctggtggttgggagaccggtggcctcattgacaaccagg |
| | aaagtcaggaggaggagccagttggaggtgtgggggcgatggtgagctctgctttacaccagccgagttta |
| | aggtgtcagtgggacattgaagtggaactgggaggtgcggggagtgagctctgagccattccagggactg |
| | gggatcatgcctggggcacctccatccccatttccctggaatccagaagagttggggggtccgagctccctg |
| | tacctcaagtgaccctccatctctctcccatctctgtctctccctggtgtctgtttttcttctcctcctctccttgtctct |
| | ctcccacacccctccatctctctcccacgtgtctctcccctcaccttctctccccctccatttctctctccctaatct |
| | gtctgttccctctgccatggcccccttcttcaagcag (SEQ ID NO: 277) |
| Canonical exon | CGTGTTCCCGAGGGAGCTGAAGGAGGTGTTTGCTTCGTGGCGGCT |
| chr6: 33440729- | GCGCTGCGCAGAGCGAGGCCGGGAGGACATCGCAGACAGGCTTA |
| 33440965 | TCAGCGCCTCACTCTTCCTGCGCTTCCTCTGCCCAGCGATTATGTC |
| | GCCCAGTCTCTTTGGGCTTATGCAGGAGTACCCAGATGAGCAGAC |
| | CTCACGAACCCTCACCCTCATTGCCAAGGTCATCCAGAACCTGGC |
| | CAACTTTTCCAA (SEQ ID NO: 278) |
| Alternative exon | CCTCCCATCTTGCTCCTGCGGTCCCTCCTTCCCTGTCTCTCTCACC |
| chr6: 33440553- | CCTGTTTCCACACCCTCACCTCCTACCACCCCCCTCAGCATGTTCC |
| 33440965 | CTGGAAGCTGAGGGTCTCTGGGGCTCAGTCCCGGTCTCTCTCTTT |
| | CTCTCTCTCTCTCTCTGTCTCCCCGACCCTTCCCCCCAGCGTGTTC |
| | CCGAGGGAGCTGAAGGAGGTGTTTGCTTCGTGGCGGCTGCGCTGC |
| | GCAGAGCGAGGCCGGGAGGACATCGCAGACAGGCTTATCAGCGC |
| | CTCACTCTTCCTGCGCTTCCTCTGCCCAGCGATTATGTCGCCCAGT |
| | CTCTTTGGGCTTATGCAGGAGTACCCAGATGAGCAGACCTCACGA |
| | ACCCTCACCCTCATTGCCAAGGTCATCCAGAACCTGGCCAACTTT |
| | TCCAA (SEQ ID NO: 279) |

TABLE 4
SYNGAP1 sequences related to NMD exon 18x event

Gene: SYNGAP1
Intron: GRCh38/hg38: chr6 33447934 33451759
Intron Sequence:
gtggaaattacaatgtcatttatcttctccgtgtcccatccccatccatcccactgtctttcgtgcactcactacaccagccacctagccccatcaccatctgtctctcatagt
ctgctgtttgtccactggctgctcctggcagccccctagtgaccccatcttcatcccatcgtctgtgcctttgtcactcctggcagcgtcagcccaactcctgtgccttccc
atccagtcttcccactcctctctgcatctcaggaccttctctaccagaaccttggtctttctgcccctagaccccacctagttccaagaacccctgccccttctttgctcact
cctattcaagccacgttgttcagcttcctctgcgctcttgggccagagggctagaagctgccgttttctggaatagagcacagggcagtatgatctgtagtttctccagg
ccctggccggtaccctgaaaacttggggacccatcacctctgttctcttggctccctaattttcctgtctccttggcagctcctgcatagcttcctcttcctgactcttcagat
cttgaaggccttccatcctgtaacctccctttgccctcagtatttaagtccagcctccctctggcctccctcccactctggccctcagaccttcccagctgcctgctgccca
gcctctcttctcacaagccagcttctaggacctcccttctgcacccttaccccttgctttcccaaaattctgctcattttcctacccatactcctctttgctctgactgctaggct
ccccccgcctgccatccccccaccaaggctcctgaccccatgaccccactctctcccactgcagctcctcatcaggtaattctcctggttccgctttggccacggg
cggaggacacagggggaggtgactccggaccactgcaggttggtcgtgaagcccactccctccaacacctccggagcctctcccctctcactgctgccctccac
acccagagaacctccacagactccagccctccgacacctgcacagatccatctcccaagacaccacccaaagagagcatttgctgctgcttcccagaactgtccaac
aataccttagcaacaccaagagttgggccctagatgggcccagcacattcacaggtcacacccacttccctgcaaaacccaccccctcccagcctcctcctgactcta
agccctcctcttcctctacctctccagtgtatgtctgtcaccccccatttcaccagagcgtccttaggggctggggggggtttgttaatggggtggaggcaatgatggg
ttggaggatcttggctataggggctgtgctgactgcagcaggtaggttgggtttccctcttccttccctaatcttggttctctaccctcctttccactcctcacctgattctctc
tcttcctcctccttatatctgtgaggcagaaggcatctgaagctcatattagcccccattgggtgggaattaggagtgggtagttaactcagggagacttgagataccct
ggaaaaaatgctattgagatgtcctgacattaggcagggtggatggaacaagaaggagcaagaaaggaacctcaggcagatgttaggacatggacttgatcatgtg
gcctgggagtttagaaatggggagagacatcctcctagatcagatcgtgggctcagtaggcatgttgattcccagggagaggtgccaggaacagcatggtaaagaa
tgtactcttcacagctcacatccccaggttgctgatgccactcactccccctctcctgccatcgagtggccttgccggacacatcaccctacctaaaaagccagtaaatg
agaacctgtcagctatagccatcatttctgagatgcgattttctttgggattgagctgcagtgggcagtggctccttacactgtaattttaattctctgcctgcccagcctctc
tgtcaaagtagctggtgatctataaagatgctaaaaggcaccaggggactttgccatttaaaggactcctgcagtgaattcttttgtaaaatgaataatggcaccctaattt
atccactttctaaatttgggtccatgggggtgtccagggcatgcttatgtgctgtcaccagcagacaaacagagggaatggaatctgggggttccttccctgctctcccg
ccatactcaggataccctaccataagtgatttcctctcactgacttgcagaaaatgtgtgagatacccagcaagctaagaaggcagttttgctgggtatctcatacccaa
ggctggggtttgggtgatctgagaggttagctccttgatcctaggatggaagggagagcttatatagaagcttttacttggaaggttttgtatcctaaggtcagacatagc
tatattaccaagcctaaatgccatgtggcccaggaaataatttggacatttgttctaaaccacttgtggtaggtattggtctctctgcaactcagccattaattagaaattagt
tttgagcctgaattttaaaaagccaagtgttgcccccagcccacacacacacacacggacatgtacagtacaaaccccagataattacaacagccaaagagagaagg
aagtgaatttcccaaccagaagcgtagggaaattcagatggctttcttttctcccagcagaggaacagaaggggagctaagggcaggaaccaggagttggtcaag
gagctataggaggtgatgagagtagaaccaggggtaggagctggtctggtacccctcaccctctaattgggagcccagggagaaggactgaaaagaagatggga
gtggaaaagaataaagccagtttctgcttcccagggatgcagagattggggcatgctgtgtctgcagaagctcctagtcatttccgccataattgtgagagagagggg
cagccctcccacaagatttttccttcccatatcacttccctgaatccccttcctttccccccagtacagttaaacctctctctaatttggaatgttatatttgagaagatggcc
actgtgaataagtgacaaacggaatgacagtgtctatttaatgaaatgcatgtcttcaaaatatatacagaactcgatgaacaaggctttttccactcctcagggagcatg
cattaatgaatagataggattcaaaagtctgttttctggtatgggttaaatatcccctcctacagacatatttccaccactaatttgcttagtacaccttttcttcacagataaag
gaaaatgcaagctcagtttttcttcagattatgaagaaattccaaatccacaggggtttggattaatgaggttttgctgtactgcctccccttattcctcaacatgaagttccc
acctcggattggggatgggtgggagggggtttcaagaggaggagggtgggatgggcaaggaatatacacaggtgaagccagagaagggttaggttgggggtgc
ggtgggaacttgctgttttgatctggtttcctggtgtgacactctgggttaaaggcttgaaggcccctgttaggagtctaggggtgagattctcttctctctgatcccagag
gacgttaacttctactgcaggtgagaaacaaaataggaggatggtggggactgtcctgggaggagggggtggtccatggcttgtggtgtgggctggctatagggga
ggccactgctaggggactggcatccaggcccccttgaagcgtctcaataagtccgcgctctcctttttggtgtcttgcag (SEQ ID NO: 280)
Gene: SYNGAP1
SYNGAP1 exon 18x: GRCh38/hg38: chr6 33448789 33448868
SYNGAP1 exon 18x sequence:
Ctcctcatcaggtaattctcctggttccgctttggccacgggcggaggacacagggggaggtgactccggaccactgcag (SEQ ID NO: 281)

For example, in some embodiments, SYNGAP1 (Gene ID: 8831) is an exemplary target of the NMD-inducing Alternative 5′ splice site event or the NMD-inducing Alternative 3′ splice site event. In some embodiments, the compositions or the methods as described herein may be used to treat AD mental retardation 5 (OMIM: 612621) related to the haploinsufficiency of SYNGAP1 in the CNS.

In some embodiments, the compositions or the methods as described herein are used to treat a condition or disease selected from the group consisting autosomal dominant mental retardation-5 (MRD5; AD Mental Retardation 5). In some embodiments, the compositions or the methods as described herein are used to treat a condition or disease of Table 5B. In some embodiments, the compositions or the methods as described herein are used to prevent, delay the progression and/or development of a sign or a symptom, or alleviate a sig or a symptom associated with a condition or disease of Table 5B.

TABLE 5A

| | Region coordinates | | | Event coordinates | Event |
|---|---|---|---|---|---|
| Gene | chr | start | end | exon | type* |
| SYNGAP1 | chr6 | 33438920 | 33440552 | chr6 33440553 | alt_3ss |
| | | | | 33440728 + | |

*alt_3ss refers to an alternative 3′ splice site of an intron, which is equivalent to the alternative 5′ splice site of the exon immediately downstream of the intron. coordinate of alternative intron. Region coordinates: coordinates of alternative intron. Event coordinates: coordinates of intronic region that is included due to alternative 3′ss or 5′ss selection.
+ or − refers to the strand in which the gene is located.

TABLE 5B

| | | | Region | Event | Event | Event |
|---|---|---|---|---|---|---|
| Gene | Disease | Genetics | coordinates | coordinates | type* | size (bp) |
| SYNGAP1 | AD MENTAL | Haploinsufficiency | chr6 | chr6 | Alt 5′ss | −37 |
| | RETARDATION 5 | | 33438883 | 33438883 | | |
| | | | 33440728 | 33438919 | | |
| | | | chr6 | chr6 | Alt 3′ss | 179 |
| | | | 33438920 | 33440550 | | |
| | | | 33440549 | 33440728 | | |
| | | | chr6 | chr6 | Alt 3′ss | 176 |
| | | | 33438920 | 33440553 | | |
| | | | 33440552 | 33440728 | | |

*Alt 5ss refers to an alternative 5′ splice site of an intron, which is equivalent to the alternative 3′ splice site of the exon immediately upstream of the intron. Alt 3ss refers to an alternative 3′ splice site of an intron, which is equivalent to the alternative 5′ splice site of the exon immediately downstream of the intron. Region coordinates: coordinates of alternative intron. Event coordinates: coordinates of intronic region that is included due to alternative 3′ss or 5′ss selection.

TABLE 6
SYNGAP1 transcripts
Target: SYNGAP1

| | |
|---|---|
| pre-mRNA | premrna_ENST00000293748.9; premrna_ENST00000418600.7; |
| transcript | premrna_ENST00000428982.4; premrna_ENST00000449372.7; |
| | premrna_ENST00000470232.1; premrna_ENST00000479510.2; |
| | premrna_ENST00000628646.2; premrna_ENST00000629380.3; |
| | premrna_ENST00000635885.1; premrna_ENST00000636075.1; |
| | premrna_ENST00000636116.1; premrna_ENST00000636146.1; |
| | premrna_ENST00000636193.1; premrna_ENST00000636436.1; |
| | premrna_ENST00000636443.1; premrna_ENST00000636640.1; |
| | premrna_ENST00000636731.1; premrna_ENST00000636905.1; |
| | premrna_ENST00000637052.1; premrna_ENST00000637194.1; |
| | premrna_ENST00000637490.1; premrna_ENST00000637587.1; |
| | premrna_ENST00000637671.1; premrna_ENST00000637721.1; |
| | premrna_ENST00000637911.1; premrna_ENST00000638127.1; |
| | premrna_ENST00000638142.2; premrna_ENST00000644458.1; |
| | premrna_ENST00000645250.1; premrna_ENST00000646630.1 |

TABLE 7
SYNGAP1 Gene and Disease Information related
to NSAE event and NMD exon 18x event

| Gene | Gene | | | |
|---|---|---|---|---|
| Symbol | ID No. | Disease | OMIM | Genetics |
| SYNGAP1 | 11497 | Mental retardation, | 612621 | Haploinsufficient |
| | | autosomal | | |
| | | dominant 5 | | |

### Antisense Oligomers

Provided herein is a composition comprising an antisense oligomer that prevents alternative splicing by binding to a targeted portion of a SYNGAP1 pre-mRNA. Provided herein also is a composition comprising an antisense oligomer that prevents alternative splicing by binding to a targeted portion of a target pre-mRNA, wherein the target is SYNGAP1. As used herein, the terms “ASO” and “antisense oligomer” are used interchangeably and refer to an oligomer such as a polynucleotide or polynucleic acid polymer, comprising nucleobases that hybridizes to a target nucleic acid (e.g., a SYNGAP1 pre-mRNA) sequence by Watson-Crick base pairing or wobble base pairing (G-U). The ASO may have exact sequence complementary to the target sequence or near complementarity (e.g., sufficient complementarity to bind the target sequence and enhancing splicing at a splice site). ASOs are designed so that they bind (hybridize) to a target nucleic acid (e.g., a targeted portion of a pre-mRNA transcript) and remain hybridized under physiological conditions. Typically, if they hybridize to a site other than the intended (targeted) nucleic acid sequence, they hybridize to a limited number of sequences that are not a target nucleic acid (to a few sites other than a target nucleic acid). Design of an ASO can take into consideration the occurrence of the nucleic acid sequence of the targeted portion of the pre-mRNA transcript or a sufficiently similar nucleic acid sequence in other locations in the genome or cellular pre-mRNA or transcriptome, such that the likelihood the ASO will bind other sites and cause “off-target” effects is limited. Any antisense oligomers known in the art, for example in PCT Application No. PCT/US2014/054151, published as WO 2015/035091, titled “Reducing Nonsense-Mediated mRNA Decay,” incorporated by reference herein, can be used to practice the methods described herein.

In some embodiments, ASOs “specifically hybridize” to or are “specific” to a target nucleic acid or a targeted portion of a RIC pre-mRNA. Typically, such hybridization occurs with a Tₘ substantially greater than 37° C., preferably at least 50° C., and typically between 60° C. to approximately 90° C. Such hybridization preferably corresponds to stringent hybridization conditions. At a given ionic strength and pH, the Tₘ is the temperature at which 50% of a target sequence hybridizes to a complementary oligonucleotide.

Oligomers, such as oligonucleotides, are “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an antisense oligomer (ASO) need not be 100% complementary to that of its target nucleic acid to hybridize. In certain embodiments, ASOs can comprise at least 70%, at least 75%, at least 80%, at least 85%, at least 90%, at least 95%, at least 96%, at least 97%, at least 98%, or at least 99% sequence complementarity to a target region within the target nucleic acid sequence to which they are targeted. For example, an ASO in which 18 of 20 nucleobases of the oligomeric compound are complementary to a target region, and would therefore specifically hybridize, would represent 90 percent complementarity. In this example, the remaining non-complementary nucleobases may be clustered together or interspersed with complementary nucleobases and need not be contiguous to each other or to complementary nucleobases. Percent complementarity of an ASO with a region of a target nucleic acid can be determined routinely using BLAST programs (basic local alignment search tools) and PowerBLAST programs known in the art (Altschul, et al., J. Mol. Biol., 1990, 215, 403-410; Zhang and Madden, Genome Res., 1997, 7, 649-656).

An ASO need not hybridize to all nucleobases in a target sequence and the nucleobases to which it does hybridize may be contiguous or noncontiguous. ASOs may hybridize over one or more segments of a pre-mRNA transcript, such that intervening or adjacent segments are not involved in the hybridization event (e.g., a loop structure or hairpin structure may be formed). In certain embodiments, an ASO hybridizes to noncontiguous nucleobases in a target pre-mRNA transcript. For example, an ASO can hybridize to nucleobases in a pre-mRNA transcript that are separated by one or more nucleobase(s) to which the ASO does not hybridize.

The ASOs described herein comprise nucleobases that are complementary to nucleobases present in a target portion of a NSAE pre-mRNA. The term ASO embodies oligonucleotides and any other oligomeric molecule that comprises nucleobases capable of hybridizing to a complementary nucleobase on a target mRNA but does not comprise a sugar moiety, such as a peptide nucleic acid (PNA). The ASOs may comprise naturally-occurring nucleotides, nucleotide analogs, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. In some embodiments, all of the nucleotides of the ASO are modified nucleotides. Chemical modifications of ASOs or components of ASOs that are compatible with the methods and compositions described herein will be evident to one of skill in the art and can be found, for example, in U.S. Pat. No. 8,258,109 B2, U.S. Pat. No. 5,656,612, U.S. Patent Publication No. 2012/0190728, and Dias and Stein, Mol. Cancer Ther. 2002, 347-355, herein incorporated by reference in their entirety.

One or more nucleobases of an ASO may be any naturally occurring, unmodified nucleobase such as adenine, guanine, cytosine, thymine and uracil, or any synthetic or modified nucleobase that is sufficiently similar to an unmodified nucleobase such that it is capable of hydrogen bonding with a nucleobase present on a target pre-mRNA. Examples of modified nucleobases include, without limitation, hypoxanthine, xanthine, 7-methylguanine, 5, 6-dihydrouracil, 5-methylcytosine, and 5-hydroxymethoylcytosine.

The ASOs described herein also comprise a backbone structure that connects the components of an oligomer. The term “backbone structure” and “oligomer linkages” may be used interchangeably and refer to the connection between monomers of the ASO. In naturally occurring oligonucleotides, the backbone comprises a 3′-5′ phosphodiester linkage connecting sugar moieties of the oligomer. The backbone structure or oligomer linkages of the ASOs described herein may include (but are not limited to) phosphorothioate, phosphorodithioate, phosphoroselenoate, phosphorodiselenoate, phosphoroanilothioate, phosphoraniladate, phosphoramidate, and the like. See, e.g., LaPlanche, et al., Nucleic Acids Res. 14:9081 (1986); Stec, et al., J. Am. Chem. Soc. 106:6077 (1984), Stein, et al., Nucleic Acids Res. 16:3209 (1988), Zon, et al., Anti-Cancer Drug Design 6:539 (1991); Zon, et al., Oligonucleotides and Analogues: A Practical Approach, pp. 87-108 (F. Eckstein, Ed., Oxford University Press, Oxford England (1991)); Stec, et al., U.S. Pat. No. 5,151,510; Uhlmann and Peyman, Chemical Reviews 90:543 (1990). In some embodiments, the backbone structure of the ASO does not contain phosphorous but rather contains peptide bonds, for example in a peptide nucleic acid (PNA), or linking groups including carbamate, amides, and linear and cyclic hydrocarbon groups. In some embodiments, the backbone modification is a phosphothioate linkage. In some embodiments, the backbone modification is a phosphoramidate linkage.

In some embodiments, the stereochemistry at each of the phosphorus internucleotide linkages of the ASO backbone is random. In some embodiments, the stereochemistry at each of the phosphorus internucleotide linkages of the ASO backbone is controlled and is not random. For example, U.S. Pat. App. Pub. No. 2014/0194610, “Methods for the Synthesis of Functionalized Nucleic Acids,” incorporated herein by reference, describes methods for independently selecting the handedness of chirality at each phosphorous atom in a nucleic acid oligomer. In some embodiments, an ASO used in the methods of the invention, including, but not limited to, any of the ASOs set forth herein in Table 1 or Table 2, comprises an ASO having phosphorus internucleotide linkages that are not random. In some embodiments, a composition used in the methods of the invention comprises a pure diastereomeric ASO. In some embodiments, a composition used in the methods of the invention comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some embodiments, the ASO has a nonrandom mixture of Rp and Sp configurations at its phosphorus internucleotide linkages. For example, it has been suggested that a mix of Rp and Sp is required in antisense oligonucleotides to achieve a balance between good activity and nuclease stability (Wan, et al., 2014, “Synthesis, biophysical properties and biological activity of second-generation antisense oligonucleotides containing chiral phosphorothioate linkages,” Nucleic Acids Research 42(22): 13456-13468, incorporated herein by reference). In some embodiments, an ASO used in the methods of the invention, including, but not limited to, any of the ASOs set forth herein in Table 1 or Table 2, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp. In some embodiments, an ASO used in the methods of the invention, including, but not limited to, any of the ASOs set forth herein in Table 1 or Table 2, comprises about 10% to about 100% Rp, about 15% to about 100% Rp, about 20% to about 100% Rp, about 25% to about 100% Rp, about 30% to about 100% Rp, about 35% to about 100% Rp, about 40% to about 100% Rp, about 45% to about 100% Rp, about 50% to about 100% Rp, about 55% to about 100% Rp, about 60% to about 100% Rp, about 65% to about 100% Rp, about 70% to about 100% Rp, about 75% to about 100% Rp, about 80% to about 100% Rp, about 85% to about 100% Rp, about 90% to about 100% Rp, or about 95% to about 100% Rp, about 20% to about 80% Rp, about 25% to about 75% Rp, about 30% to about 70% Rp, about 40% to about 60% Rp, or about 45% to about 55% Rp, with the remainder Sp.

In embodiments, an ASO used in the methods of the invention, including, but not limited to, any of the ASOs set forth herein in Table 1 or Table 2, comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In some embodiments, an ASO used in the methods of the invention, including, but not limited to, any of the ASOs set forth herein in Table 1 or Table 2, comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

Any of the ASOs described herein may contain a sugar moiety that comprises ribose or deoxyribose, as present in naturally occurring nucleotides, or a modified sugar moiety or sugar analog, including a morpholine ring. Non-limiting examples of modified sugar moieties include 2′ substitutions such as 2′-O-methyl (2′-O-Me), 2′-O-methoxyethyl (2′MOE), 2′-O-aminoethyl, 2′F; N3′->P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. In some embodiments, the sugar moiety modification is selected from 2′-O-Me, 2′F, and 2′MOE. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, the sugar moiety comprises a ribofuranosyl or 2′deoxyribofuransyl modification. In some embodiments, the sugar moiety comprises 2′4′-constrained 2′O-methyloxyethyl (cMOE) modifications. In some embodiments, the sugar moiety comprises cEt 2′, 4′ constrained 2′-0 ethyl BNA modifications. In some embodiments, the sugar moiety comprises tricycloDNA (tcDNA) modifications. In some embodiments, the sugar moiety comprises ethylene nucleic acid (ENA) modifications. In some embodiments, the sugar moiety comprises MCE modifications. Modifications are known in the art and described in the literature, e.g., by Jarver, et al., 2014, “A Chemical View of Oligonucleotides for Exon Skipping and Related Drug Applications,” Nucleic Acid Therapeutics 24(1): 37-47, incorporated by reference for this purpose herein.

In some embodiments, each monomer of the ASO is modified in the same way, for example each linkage of the backbone of the ASO comprises a phosphorothioate linkage or each ribose sugar moiety comprises a 2′O-methyl modification. Such modifications that are present on each of the monomer components of an ASO are referred to as “uniform modifications.” In some embodiments, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries.”

In some embodiments, the ASO comprises one or more backbone modification. In some embodiments, the ASO comprises one or more sugar moiety modification. In some embodiments, the ASO comprises one or more backbone modification and one or more sugar moiety modification. In some embodiments, the ASO comprises 2′MOE modifications and a phosphorothioate backbone. In some embodiments, the ASO comprises a phosphorodiamidate morpholino (PMO). In some embodiments, the ASO comprises a peptide nucleic acid (PNA). Any of the ASOs or any component of an ASO (e.g., a nucleobase, sugar moiety, backbone) described herein may be modified in order to achieve desired properties or activities of the ASO or reduce undesired properties or activities of the ASO. In some embodiments, an ASO or one or more component of any ASO may be modified to enhance binding affinity to a target sequence on a pre-mRNA transcript; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and modulate the half-life of the ASO.

In some embodiments, the ASOs are comprised of 2′-O-(2-methoxyethyl) (MOE) phosphorothioate-modified nucleotides. ASOs comprised of such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein. See e.g., Geary, et al., J Pharmacol Exp Ther. 2001; 296(3):890-7; Geary, et al., J Pharmacol Exp Ther. 2001; 296(3):898-904.

Methods of synthesizing ASOs will be known to one of skill in the art. Alternatively or in addition, ASOs may be obtained from a commercial source.

Unless specified otherwise, the left-hand end of single-stranded nucleic acid (e.g., pre-mRNA transcript, oligonucleotide, ASO, etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, the 5′ direction or end of an mRNA is where the initiation or start codon is located, while the 3′ end or direction is where the termination codon is located. In some embodiments, nucleotides that are upstream of a reference point in a nucleic acid may be designated by a negative number, while nucleotides that are downstream of a reference point may be designated by a positive number. For example, a reference point (e.g., an exon-exon junction in mRNA) may be designated as the “zero” site, and a nucleotide that is directly adjacent and upstream of the reference point is designated “minus one,” e.g., “−1,” while a nucleotide that is directly adjacent and downstream of the reference point is designated “plus one,” e.g., “+1.”

In some embodiments, the ASOs are complementary to (and bind to) a targeted portion of a SYNGAP1 pre-mRNA that is downstream (in the 3′ direction) of the 5′ splice site of the NSAE in a SYNGAP1 pre-mRNA (e.g., the direction designated by positive numbers relative to the 5′ splice site). In some embodiments, the ASOs are complementary to a targeted portion of the SYNGAP1 pre-mRNA that is within the region about +6 to about +500 relative to the 5′ splice site of the NSAE. In some embodiments, the ASO is not complementary to nucleotides +1 to +5 relative to the 5′ splice site (the first five nucleotides located downstream of the 5′ splice site). In some embodiments, the ASOs may be complementary to a targeted portion of a SYNGAP1 pre-mRNA that is within the region between nucleotides +6 and +100 relative to the 5′ splice site of the NSAE. In some aspects, the ASOs are complementary to a targeted portion that is within the region about +6 to about +500, about +6 to about +490, about +6 to about +480, about +6 to about +470, about +6 to about +460, about +6 to about +450, about +6 to about +440, about +6 to about +430, about +6 to about +420, about +6 to about +410, about +6 to about +400, about +6 to about +390, about +6 to about +380, about +6 to about +370, about +6 to about +360, about +6 to about +350, about +6 to about +340, about +6 to about +330, about +6 to about +320, about +6 to about +310, about +6 to about +300, about +6 to about +290, about +6 to about +280, about +6 to about +270, about +6 to about +260, about +6 to about +250, about +6 to about +240, about +6 to about +230, about +6 to about +220, about +6 to about +210, about +6 to about +200, about +6 to about +190, about +6 to about +180, about +6 to about +170, about +6 to about +160, about +6 to about +150, about +6 to about +140, about +6 to about +130, about +6 to about +120, about +6 to about +110, about +6 to about +100, about +6 to about +90, about +6 to about +80, about +6 to about +70, about +6 to about +60, about +6 to about +50, about +6 to about +40, about +6 to about +30, or about +6 to about +20 relative to 5′ splice site of the NSAE.

In some embodiments, the ASOs are complementary to (and bind to) a targeted portion of a target pre-mRNA that is downstream (in the 3′ direction) of the 5′ splice site of the NSAE in a target pre-mRNA (e.g., the direction designated by positive numbers relative to the 5′ splice site), wherein the target is SYNGAP1. In some embodiments, the ASOs are complementary to a targeted portion of the target pre-mRNA that is within the region about +6 to about +500 relative to the 5′ splice site of the NSAE. In some embodiments, the ASO is not complementary to nucleotides +1 to +5 relative to the 5′ splice site (the first five nucleotides located downstream of the 5′ splice site). In some embodiments, the ASOs may be complementary to a targeted portion of a target pre-mRNA that is within the region between nucleotides +6 and +100 relative to the 5′ splice site of the NSAE. In some aspects, the ASOs are complementary to a targeted portion that is within the region about +6 to about +500, about +6 to about +490, about +6 to about +480, about +6 to about +470, about +6 to about +460, about +6 to about +450, about +6 to about +440, about +6 to about +430, about +6 to about +420, about +6 to about +410, about +6 to about +400, about +6 to about +390, about +6 to about +380, about +6 to about +370, about +6 to about +360, about +6 to about +350, about +6 to about +340, about +6 to about +330, about +6 to about +320, about +6 to about +310, about +6 to about +300, about +6 to about +290, about +6 to about +280, about +6 to about +270, about +6 to about +260, about +6 to about +250, about +6 to about +240, about +6 to about +230, about +6 to about +220, about +6 to about +210, about +6 to about +200, about +6 to about +190, about +6 to about +180, about +6 to about +170, about +6 to about +160, about +6 to about +150, about +6 to about +140, about +6 to about +130, about +6 to about +120, about +6 to about +110, about +6 to about +100, about +6 to about +90, about +6 to about +80, about +6 to about +70, about +6 to about +60, about +6 to about +50, about +6 to about +40, about +6 to about +30, or about +6 to about +20 relative to 5′ splice site of the NSAE.

In some embodiments, the ASOs are complementary to (and bind to) a targeted portion of a SYNGAP1 pre-mRNA that is upstream (in the 5′ direction) of the 3′ splice site of the NSAE in a SYNGAP1 pre-mRNA (e.g., the direction designated by negative numbers relative to the 3′ splice site). In some embodiments, the ASOs are complementary to a targeted portion of the SYNGAP1 pre-mRNA that is within the region about −16 to about −500 relative to the 3′ splice site of the NSAE. In some embodiments, the ASO is not complementary to nucleotides −1 to −15 relative to the 5′ splice site (the first five nucleotides located downstream of the 5′ splice site). In some embodiments, the ASOs may be complementary to a targeted portion of a SYNGAP1 pre-mRNA that is within the region between nucleotides −16 and −100 relative to the 3′ splice site of the NSAE. In some aspects, the ASOs are complementary to a targeted portion that is within the region about +6 to about +500, about +6 to about +490, about +6 to about +480, about +6 to about +470, about +6 to about +460, about +6 to about +450, about +6 to about +440, about +6 to about +430, about +6 to about +420, about +6 to about +410, about +6 to about +400, about +6 to about +390, about +6 to about +380, about +6 to about +370, about +6 to about +360, about +6 to about +350, about +6 to about +340, about +6 to about +330, about +6 to about +320, about +6 to about +310, about +6 to about +300, about +6 to about +290, about +6 to about +280, about +6 to about +270, about +6 to about +260, about +6 to about +250, about +6 to about +240, about +6 to about +230, about +6 to about +220, about +6 to about +210, about +6 to about +200, about +6 to about +190, about +6 to about +180, about +6 to about +170, about +6 to about +160, about +6 to about +150, about +6 to about +140, about +6 to about +130, about +6 to about +120, about +6 to about +110, about +6 to about +100, about +6 to about +90, about +6 to about +80, about +6 to about +70, about +6 to about +60, about +6 to about +50, about +6 to about +40, about +6 to about +30, or about +6 to about +20 relative to 3′ splice site of the NSAE.

In some embodiments, the ASOs are complementary to (and bind to) a targeted portion of a target pre-mRNA that is upstream (in the 5′ direction) of the 3′ splice site of the NSAE in a target pre-mRNA (e.g., the direction designated by negative numbers relative to the 3′ splice site), wherein the target is SYNGAP1. In some embodiments, the ASOs are complementary to a targeted portion of the target pre-mRNA that is within the region about −16 to about −500 relative to the 3′ splice site of the NSAE. In some embodiments, the ASO is not complementary to nucleotides −1 to −15 relative to the 5′ splice site (the first five nucleotides located downstream of the 5′ splice site). In some embodiments, the ASOs may be complementary to a targeted portion of a target pre-mRNA that is within the region between nucleotides −16 and −100 relative to the 3′ splice site of the NSAE. In some aspects, the ASOs are complementary to a targeted portion that is within the region about +6 to about +500, about +6 to about +490, about +6 to about +480, about +6 to about +470, about +6 to about +460, about +6 to about +450, about +6 to about +440, about +6 to about +430, about +6 to about +420, about +6 to about +410, about +6 to about +400, about +6 to about +390, about +6 to about +380, about +6 to about +370, about +6 to about +360, about +6 to about +350, about +6 to about +340, about +6 to about +330, about +6 to about +320, about +6 to about +310, about +6 to about +300, about +6 to about +290, about +6 to about +280, about +6 to about +270, about +6 to about +260, about +6 to about +250, about +6 to about +240, about +6 to about +230, about +6 to about +220, about +6 to about +210, about +6 to about +200, about +6 to about +190, about +6 to about +180, about +6 to about +170, about +6 to about +160, about +6 to about +150, about +6 to about +140, about +6 to about +130, about +6 to about +120, about +6 to about +110, about +6 to about +100, about +6 to about +90, about +6 to about +80, about +6 to about +70, about +6 to about +60, about +6 to about +50, about +6 to about +40, about +6 to about +30, or about +6 to about +20 relative to 3′ splice site of the NSAE.

In some embodiments, the targeted portion of the SYNGAP1 pre-mRNA is within the region −4e relative to the 3′ splice site (5′ end) of the NSAE to +2e relative to the 5′ splice site (3′ end) of the NSAE.

In some embodiments, the targeted portion of the target pre-mRNA is within the region −4e relative to the 3′ splice site (5′ end) of the NSAE to +2e relative to the 5′ splice site (3′ end) of the NSAE, wherein the target is SYNGAP1. The ASOs may be of any length suitable for specific binding and effective enhancement of splicing. In some embodiments, the ASOs consist of 8 to 50 nucleobases. For example, the ASO may be 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleobases in length. In some embodiments, the ASOs consist of more than 50 nucleobases. In some embodiments, the ASO is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 13 to 50 nucleobases, 13 to 40 nucleobases, 13 to 35 nucleobases, 13 to 30 nucleobases, 13 to 25 nucleobases, 13 to 20 nucleobases, 14 to 50 nucleobases, 14 to 40 nucleobases, 14 to 35 nucleobases, 14 to 30 nucleobases, 14 to 25 nucleobases, 14 to 20 nucleobases, 15 to 50 nucleobases, 15 to 40 nucleobases, 15 to 35 nucleobases, 15 to 30 nucleobases, 15 to 25 nucleobases, 15 to 20 nucleobases, 20 to 50 nucleobases, 20 to 40 nucleobases, 20 to 35 nucleobases, 20 to 30 nucleobases, 20 to 25 nucleobases, 25 to 50 nucleobases, 25 to 40 nucleobases, 25 to 35 nucleobases, or 25 to 30 nucleobases in length. In some embodiments, the ASOs are 15 nucleotides in length. In some embodiments, the ASOs are 16 nucleotides in length. In some embodiments, the ASOs are 17 nucleotides in length. In some embodiments, the ASOs are 18 nucleotides in length. In some embodiments, the ASOs are 25 nucleotides in length.

In some embodiments, two or more ASOs with different chemistries but complementary to the same targeted portion of the NSAE pre-mRNA are used. In some embodiments, two or more ASOs that are complementary to different targeted portions of the NSAE pre-mRNA are used.

In some embodiments, the antisense oligonucleotides of the invention are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine, or a polyethylene glycol chain, or adamantane acetic acid. Oligonucleotides comprising lipophilic moieties and preparation methods have been described in the published literature. In embodiments, the antisense oligonucleotide is conjugated with a moiety including, but not limited to, an abasic nucleotide, a polyether, a polyamine, a polyamide, a peptide, a carbohydrate, e.g., N-acetylgalactosamine (GalNAc), N-Ac-Glucosamine (GluNAc), or mannose (e.g., mannose-6-phosphate), a lipid, or a polyhydrocarbon compound. Conjugates can be linked to one or more of any nucleotides comprising the antisense oligonucleotide at any of several positions on the sugar, base, or phosphate group, as understood in the art and described in the literature, e.g., using a linker. Linkers can include a bivalent or trivalent branched linker. In embodiments, the conjugate is attached to the 3′ end of the antisense oligonucleotide. Methods of preparing oligonucleotide conjugates are described, e.g., in U.S. Pat. No. 8,450,467, “Carbohydrate conjugates as delivery agents for oligonucleotides,” incorporated by reference herein.

In some embodiments, the antisense oligonucleotides of the disclosure are chemically linked to a lipophilic group. Representative conjugate moieties can include lipophilic molecules (aromatic and non-aromatic) including sterol and steroid molecules. Lipophilic conjugate moieties can be used, for example, to counter the hydrophilic nature of an oligomeric compound and enhance cellular penetration. Lipophilic moieties include, for example, steroids and related compounds such as cholesterol (U.S. Pat. No. 4,958,013 and Letsinger et al., Proc. Natl. Acad. Sci. USA, 1989, 86, 6553), thiocholesterol (Oberhauser et al., Nucl Acids Res., 1992, 20, 533), lanosterol, coprostanol, stigmasterol, ergosterol, calciferol, cholic acid, deoxycholic acid, estrone, estradiol, estratriol, progesterone, stilbestrol, testosterone, androsterone, deoxycorticosterone, cortisone, 17-hydroxycorticosterone, their derivatives, and the like. Other lipophilic conjugate moieties include aliphatic groups, such as, for example, straight chain, branched, and cyclic alkyls, alkenyls, and alkynyls. The aliphatic groups can have, for example, 5 to about 50, 6 to about 50, 8 to about 50, or 10 to about 50 carbon atoms. Example aliphatic groups include undecyl, dodecyl, hexadecyl, heptadecyl, octadecyl, nonadecyl, terpenes, bornyl, adamantyl, derivatives thereof and the like. In some embodiments, one or more carbon atoms in the aliphatic group can be replaced by a heteroatom such as O, S, or N (e.g., geranyloxyhexyl). Further suitable lipophilic conjugate moieties include aliphatic derivatives of glycerols such as alkylglycerols, bis(alkyl)glycerols, tris(alkyl)glycerols, monoglycerides, diglycerides, and triglycerides. In some embodiments, the lipophilic conjugate is di-hexyldecyl-rac-glycerol or 1,2-di-O-hexyldecyl-rac-glycerol (Manoharan et al., Tetrahedron Lett., 1995, 36, 3651; Shea, et al., Nuc. Acids Res., 1990, 18, 3777) or phosphonates thereof. Saturated and unsaturated fatty functionalities, such as, for example, fatty acids, fatty alcohols, fatty esters, and fatty amines, can also serve as lipophilic conjugate moieties. In some embodiments, the fatty functionalities can contain from about 6 carbons to about 30 or about 8 to about 22 carbons. Example fatty acids include, capric, caprylic, lauric, palmitic, myristic, stearic, oleic, linoleic, linolenic, arachidonic, eicosenoic acids and the like. In further embodiments, lipophilic conjugate groups can be polycyclic aromatic groups having from 6 to about 50, 10 to about 50, or 14 to about 40 carbon atoms. Example polycyclic aromatic groups include pyrenes, purines, acridines, xanthenes, fluorenes, phenanthrenes, anthracenes, quinolines, isoquinolines, naphthalenes, derivatives thereof and the like. [0037] Other suitable lipophilic conjugate moieties include menthols, trityls (e.g., dimethoxytrityl (DMT)), phenoxazines, lipoic acid, phospholipids, ethers, thioethers (e.g., hexyl-S-tritylthiol), derivatives thereof and the like. Preparation of lipophilic conjugates of oligomeric compounds are well-described in the art, such as in, for example, Saison-Behmoaras et al., EMBO J., 1991; Kabanov et al., FEBSLett., 1990, 259, 327; Svinarchuk et al., Biochimie, 1993, 75, 49; (Mishra et al., Biochim. Biophys. Acta, 1995, 1264, 229, and Manoharan et al., Tetrahedron Lett., 1995, 36, 3651.

Oligomeric compounds containing conjugate moieties with affinity for low density lipoprotein (LDL) can help provide an effective targeted delivery system. High expression levels of receptors for LDL on tumor cells makes LDL an attractive carrier for selective delivery of drugs to these cells (Rump, et al., Bioconjugate Chem., 1998, 9, 341; Firestone, Bioconjugate Chem., 1994, 5, 105; Mishra, et al., Biochim. Biophys. Acta, 1995, 1264, 229). Moieties having affinity for LDL include many lipophilic groups such as steroids (e.g., cholesterol), fatty acids, derivatives thereof and combinations thereof. In some embodiments, conjugate moieties having LDL affinity can be dioleyl esters of cholic acids such as chenodeoxycholic acid and lithocholic acid.

In some embodiments, the conjugate group is or may comprise a lipophilic moiety, such as a sterol (for example, cholesterol, cholesteryl, cholestanol, stigmasterol, cholanic acid and ergosterol). In some embodiments, the conjugate is or may comprise cholesterol. See for example, Soutschek et al., Nature (2004) 432, 173; KrQtzfeldt Nature 2005, NAR 2007.

In some embodiments, the conjugate is, or may comprise a lipid, a phospholipid or a lipophilic alcohol, such as a cationic lipid, a neutral lipid, a sphingolipid, and fatty acids such as stearic, oleic, elaidic, linoleic, linoleaidic, linolenic, and myristic acids. In some embodiments the fatty acid comprises a C4-C30 saturated or unsaturated alkyl chain. The alkyl chain may be linear or branched.

In some embodiments, the nucleic acid to be targeted by an ASO is a SYNGAP1 pre-mRNA expressed in a cell, such as a eukaryotic cell. In some embodiments, the nucleic acid to be targeted by an ASO is a pre-mRNA expressed in a cell, such as a eukaryotic cell, wherein the target is SYNGAP1. In some embodiments, the term “cell” may refer to a population of cells. In some embodiments, the cell is in a subject. In some embodiments, the cell is isolated from a subject. In some embodiments, the cell is ex vivo. In some embodiments, the cell is a condition or disease-relevant cell or a cell line. In some embodiments, the cell is in vitro (e.g., in cell culture).

Where reference is made to a polynucleic acid polymer sequence, the skilled person will understand that one or more substitutions may be tolerated, optionally two substitutions may be tolerated in the sequence, such that it maintains the ability to hybridize to the target sequence; or where the substitution is in a target sequence, the ability to be recognized as the target sequence. References to sequence identity may be determined by BLAST sequence alignment using standard/default parameters. For example, the sequence may have 99% identity and still function according to the present disclosure. In other embodiments, the sequence may have 98% identity and still function according to the present disclosure. In another embodiment, the sequence may have 95% identity and still function according to the present disclosure. In another embodiment, the sequence may have 90% identity and still function according to the present disclosure.

### Pharmaceutical Compositions

Pharmaceutical compositions or formulations comprising the agent, e.g., antisense oligonucleotide, of the described compositions and for use in any of the described methods can be prepared according to conventional techniques well known in the pharmaceutical industry and described in the published literature. In embodiments, a pharmaceutical composition or formulation for treating a subject comprises an effective amount of any antisense oligomer as described herein, or a pharmaceutically acceptable salt, solvate, hydrate or ester thereof. The pharmaceutical formulation comprising an antisense oligomer may further comprise a pharmaceutically acceptable excipient, diluent, or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reaction between the free base and a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In some embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some embodiments, a pharmaceutical formulation or composition of the present invention includes, but is not limited to, a solution, emulsion, microemulsion, foam or liposome-containing formulation (e.g., cationic or noncationic liposomes).

The pharmaceutical composition or formulation of the present invention may comprise one or more penetration enhancer, carrier, excipients or other active or inactive ingredients as appropriate and well known to those of skill in the art or described in the published literature. In some embodiments, liposomes also include sterically stabilized liposomes, e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In some embodiments, the present invention employs a penetration enhancer to effect the efficient delivery of the antisense oligonucleotide, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple antisense oligonucleotides. In some embodiments, the antisense oligonucleotide is administered in combination with another drug or therapeutic agent.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having the disease, such as any of the diseases described herein. In some embodiments, the individual is at increased risk of having a disease or disorder caused by insufficient amount of a protein or insufficient activity of a protein. In some embodiments, if an individual is “at an increased risk” of having a disease or disorder caused by insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In some embodiments, a fetus is treated in utero, e.g., by administering the ASO composition to the fetus directly or indirectly (e.g., via the mother).

Suitable routes for administration of ASOs of the present invention may vary depending on cell type to which delivery of the ASOs is desired. Multiple tissues and organs can be affected by autosomal dominant mental retardation. In some embodiments, the liver can be the most significantly affected tissue. The ASOs of the present invention may be administered to patients parenterally, for example, by intrathecal injection, intracerebroventricular injection, intraperitoneal injection, intramuscular injection, subcutaneous injection, or intravenous injection.

Methods of Identifying Additional ASOs that Prevent Alternative Splicing.

Also within the scope of the present disclosure are methods for identifying or determining ASOs that prevent alternative splicing of a SYNGAP1 pre-mRNA. For example, a method can comprise identifying or determining ASOs that prevent alternative splicing of a SYNGAP1 pre-mRNA. Also within the scope of the present disclosure are methods for identifying or determining ASOs that prevent alternative splicing of a target pre-mRNA, wherein the target is SYNGAP1. For example, a method can comprise identifying or determining ASOs that prevent alternative splicing of a target pre-mRNA. ASOs that specifically hybridize to different nucleotides within the target region of the pre-mRNA may be screened to identify or determine ASOs that improve the extent of canonical splicing. In some embodiments, the ASO may block or interfere with the binding site(s) of a splicing silencer. Any method known in the art may be used to identify (determine) an ASO that when hybridized to the target region of the NSAE results in the desired effect (e.g., exon inclusion, protein or functional RNA production). These methods also can be used for identifying ASOs that prevent alternative splicing of the NSAE by binding to a targeted region in an intron flanking the NSAE. An example of a method that may be used is provided below.

A round of screening, referred to as an ASO “walk” may be performed using ASOs that have been designed to hybridize to a target region of a pre-mRNA. For example, the ASOs used in the ASO walk can be tiled every 5 nucleotides from approximately 100 nucleotides upstream of the 3′ splice site of the NSAE (e.g., a portion of sequence of the intron located upstream of the target/NSAE) to approximately 100 nucleotides downstream of the 3′ splice site of the target/NSAE and/or from approximately 100 nucleotides upstream of the 5′ splice site of the NSAE to approximately 100 nucleotides downstream of the 5′ splice site of the target/NSAE (e.g., a portion of sequence of the intron located downstream of the target/NSAE). For example, a first ASO of 18 nucleotides in length may be designed to specifically hybridize to nucleotides +6 to +23 relative to the 5′ splice site of the target/NSAE. A second ASO is designed to specifically hybridize to nucleotides +11 to +28 relative to the 5′ splice site of the target/NSAE. ASOs are designed as such spanning the target region of the pre-mRNA. In some embodiments, the ASOs can be tiled more closely, e.g., every 1, 2, 3, or 4 nucleotides. Further, the ASOs can be tiled from 100 nucleotides downstream of the 5′ splice site, to 100 nucleotides upstream of the 3′ splice site. In some embodiments, the ASOs can be tiled from about 572 nucleotides upstream of the 3′ splice site, to about 500 nucleotides downstream of the 5′ splice site. In some embodiments, the ASOs can be tiled from about 500 nucleotides upstream of the 3′ splice site, to about 572 nucleotides downstream of the 3′ splice site.

One or more ASOs, or a control ASO (an ASO with a scrambled sequence, sequence that is not expected to hybridize to the target region) are delivered, for example by transfection, into a disease-relevant cell line that expresses the target pre-mRNA (e.g., a NSAE pre-mRNA described herein). The canonical splicing promoting effects of each of the ASOs may be assessed by any method known in the art, for example by reverse transcriptase (RT)-PCR using primers, such as primers spanning the region containing the NSAE or canonical exon. A decrease or absence of a longer or shorter RT-PCR product than a canonical exon produced using primers spanning the region containing the NSAE or canonical exon in ASO-treated cells as compared to in control ASO-treated cells indicates that exclusion of the NSAE has been increased. In some embodiments, the canonical splicing efficiency or the ratio of canonically spliced mRNA to NSAE containing mRNA may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

A second round of screening, referred to as an ASO “micro-walk” may be performed using ASOs that have been designed to hybridize to a target region of a pre-mRNA. The ASOs used in the ASO micro-walk are tiled every 1 nucleotide to further refine the nucleotide acid sequence of the pre-mRNA that when hybridized with an ASO results in modulation of NSAE exclusion.

Regions defined by ASOs that promote inclusion target NSAE are explored in greater detail by means of an ASO “micro-walk”, involving ASOs spaced in 1-nt steps, as well as longer ASOs, typically 15-25 nt.

As described for the ASO walk above, the ASO micro-walk is performed by delivering one or more ASOs, or a control ASO (an ASO with a scrambled sequence, sequence that is not expected to hybridize to the target region), for example by transfection, into a disease-relevant cell line that expresses the target pre-mRNA. The splicing-inducing effects of each of the ASOs may be assessed by any method known in the art, for example by reverse transcriptase (RT)-PCR using primers that span the NSAE, as described herein. A decrease or absence of a longer or shorter RT-PCR product than a canonical exon produced using primers spanning the region containing the NSAE or canonical exon in ASO-treated cells as compared to in control ASO-treated cells indicates that exclusion of the NSAE has been increased. In some embodiments, the canonical splicing efficiency or the ratio of canonically spliced mRNA to retained NSAE containing mRNA may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

ASOs that when hybridized to a region of a pre-mRNA result in canonical splicing and increased protein production may be tested in vivo using animal models, for example transgenic mouse models in which the full-length human gene has been knocked-in or in humanized mouse models of disease. Suitable routes for administration of ASOs may vary depending on the disease and/or the cell types to which delivery of the ASOs is desired. ASOs may be administered, for example, by intrathecal injection, intracerebroventricular injection, intraperitoneal injection, intramuscular injection, subcutaneous injection, or intravenous injection. Following administration, the cells, tissues, and/or organs of the model animals may be assessed to determine the effect of the ASO treatment by for example evaluating splicing (efficiency, rate, extent) and protein production by methods known in the art and described herein. The animal models may also be any phenotypic or behavioral indication of the disease or disease severity.

### Combinational Therapy

In some embodiments, provided herein is a composition comprising one or more NSAE-modulating agents. In some embodiments, provided herein is a composition comprising two or more NSAE-modulating agents. In some embodiments, provided herein is a composition comprising one or more ASO complementary to a targeted region of SynGAP1 pre-mRNA. In some embodiments, provided herein is a composition comprising two or more ASO complementary to a targeted region of SynGAP1 pre-mRNA. In some embodiments, provided herein is a composition comprising one or more ASO complementary to a same targeted region of SynGAP1 pre-mRNA. In some embodiments, provided herein is a composition comprising two or more ASO complementary to a same targeted region of SynGAP1 pre-mRNA. In some embodiments, provided herein is a composition comprising one or more ASO complementary to different targeted regions of SynGAP1 pre-mRNA. In some embodiments, provided herein is a composition comprising two or more ASO complementary to different targeted regions of SynGAP1 pre-mRNA. In some embodiments, provided herein is a composition comprising one or more ASOs of Table 1. In some embodiments, provided herein is a composition comprising two and more ASOs of in Table 1. In some embodiments, provided herein is a composition comprising one or more ASOs selected from the sequences of Table 1 and Table 2. In some embodiments, provided herein is a composition comprising two and more ASOs selected from the sequences of Table 1 and Table 2.

In some embodiments, provided herein is a composition comprising one or more ASO complementary to a targeted region of a target pre-mRNA, wherein the target is SYNGAP1. In some embodiments, provided herein is a composition comprising two or more ASO complementary to a targeted region of a target pre-mRNA. In some embodiments, provided herein is a composition comprising one or more ASO complementary to a same targeted region of a target pre-mRNA. In some embodiments, provided herein is a composition comprising two or more ASO complementary to a same targeted region of a target pre-mRNA. In some embodiments, provided herein is a composition comprising one or more ASO complementary to different targeted regions of a target pre-mRNA. In some embodiments, provided herein is a composition comprising two or more ASO complementary to different targeted regions of a target pre-mRNA. In some embodiments, provided herein is a composition comprising two or more ASO complementary to a targeted region of two or more target pre-mRNAs. In some embodiments, provided herein is a composition comprising two or more ASO complementary to a targeted region of two or more different target pre-mRNAs.

In some aspects, provided herein is a method of modulating expression of a target protein by administering the compositions as described above. In some aspects, provided herein is a pharmaceutical composition comprising a therapeutic agent comprising the composition as described above; and a pharmaceutically acceptable excipient and/or a delivery vehicle. In some aspects, provided herein is a method of treating or preventing a disease or condition in a subject in need thereof, the method comprising: administering to the subject a pharmaceutical composition comprising a therapeutic agent comprising the composition as described above; and a pharmaceutically acceptable excipient and/or a delivery vehicle. In some aspects, provided herein is a method of treating or preventing a disease or condition in a subject in need thereof, the method comprising: administering to the subject the composition as described above. In some aspects, provided herein is a method of treating a disease or condition in a subject in need thereof by modulating expression of a target protein in a cell of the subject through administering to the subject a pharmaceutical composition comprising a therapeutic agent comprising the composition as described above; and a pharmaceutically acceptable excipient and/or a delivery vehicle. In some aspects, provided herein is a method of treating a disease or condition in a subject to the subject in need thereof by modulating expression of a target protein in a cell of the subject through administering to the subject the composition as described above. In some aspects, provided herein is a therapeutic agent for use in a method described herein. In some aspects, provided herein is a pharmaceutical composition comprising a therapeutic agent described herein and a pharmaceutically acceptable excipient.

The present invention will be more specifically illustrated by the following Examples. However, it should be understood that the present invention is not limited by these examples in any manner.

### EXAMPLES

### Example 1: Identification of NMD-Inducing Alternative Splicing Events in Transcripts by RNAseq Using Next-Generation Sequencing

Non-productive AS events in organs known to be accessible by ASOs were identified by analyzing 83 publicly available RNA-sequencing (RNA-seq) datasets from human liver, kidney, central nervous system (CNS), and eye tissues. Computational analysis discovered 7,819 unique genes containing a total of 13,121 non-productive AS events of various types. By cross-referencing these genes with genetic disease databases such as Orphanet (www.orpha.net/), 1,265 disease-associated genes with non-productive AS events were identified. As many NMD-sensitive transcripts are efficiently degraded in the analyzed tissues and are not detectable by RNAseq, there are many more genes with non-productive AS events than have been identified to date. To prove the TANGO concept, four genes from the bioinformatic analysis representing three types of targets (haploinsufficiency, autosomal recessive and pathway associated with disease), three organs (liver, CNS, and eye), and three types of alternative splicing events (cassette exon, alternative splice site, and alternative intron) were selected.

### Example 2: Confirmation of Alternative Splicing Via Cycloheximide Treatment

To validate the in-silico predictions and to quantify the abundance of potentially targetable non-productive AS events, cells were treated with cycloheximide (CHX), a translation inhibitor known to inhibit NMD. Expectedly, reverse transcriptase (RT)-PCR analysis showed a consistent increase in the predicted non-productive SYNGAP1 splicing events in various cell lines upon CHX treatment compared to DMSO-treated cells. The increase indicates that these non-productive AS events lead to transcript degradation by NMD.

### Example 3: SYNGAP1 Exon Region ASO Walk

To identify ASOs that can prevent the non-productive AS events, an initial systematic ASO walk was performed in 5-nt steps along the AS event of interest. These ASOs have a uniform phosphorothioate backbone and methoxyethyl at the 2′ ribose position (2′MOE-PS). These modifications were previously shown to allow binding to RNA with high affinity and to confer resistance to both nucleases and RNase H cleavage of the target RNA-ASO complex. RT-PCR analysis from transfected HEK293 cells identified several ASOs that reduce AS in the SYNGAP1 mRNA and increase productive mRNA. The observed increase in SYNGAP1 productive mRNA was confirmed by TaqMan qPCR. The fold change of AS may be plotted vs the increase in productive mRNA (qPCR) to demonstrate that the ASOs are functioning on mechanism. These results strongly suggest that gene expression upregulation can be achieved by preventing non-productive AS with ASOs.

### Example 4: Use of ASO's to Increase Cellular Protein Expression in a Dose-Dependent Manner

As the desirable upregulation level varies among target genes and diseases, selected positive ASO hits from the initial walks were used to determine whether the increase in productive mRNA can be titrated across non-productive AS events. SYNGAP1 ASO-55 was transfected in cells at increasing concentrations to demonstrate dose-dependent upregulation. The concentration was selected based on the potency of the ASO. RT-PCR results showed a dose-dependent decrease of the non-productive alternative 3′ss selection in SYNGAP1 compared to a non-targeting ASO control transfected at the same respective doses. Conversely, a dose-dependent increase in productive mRNA was observed as measured by TaqMan qPCR compared to a non-targeting ASO control. To determine whether the observed upregulation in productive mRNAs translates to a dose-dependent increase in protein levels, SynGAP proteins were measured in extracts from transfected cells with increasing concentrations of targeting ASOs. First, antibodies against SynGAP were validated by short interfering (si)RNA-mediated knockdown of protein expression and western blot analysis. Immunoblotting results of extracts from cells transfected with the selected ASOs showed a dose-dependent increase in SynGAP proteins. The level of protein upregulation resulting from the highest ASO concentrations ranged between 1.6- to 2-fold. A non-targeting ASO control had no significant effect on protein levels. Altogether, the data indicate that ASOs targeting various types of non-productive AS events lead to a titratable increase in productive mRNA resulting in an increase in protein expression. The titratable nature of TANGO ASO-mediated protein upregulation suggests that one could tightly control protein levels and reduce the risk of overexpression. This aspect of the TANGO technology makes it especially suited to address autosomal dominant haploinsufficient diseases.

### Example 5: Effects of ASOs on Productive and Nonproductive mRNA Levels

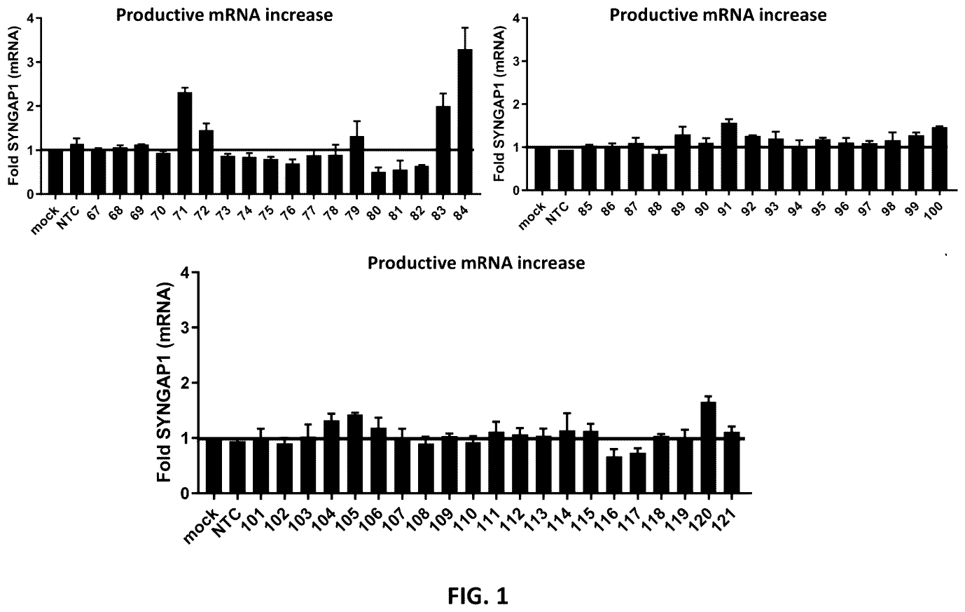

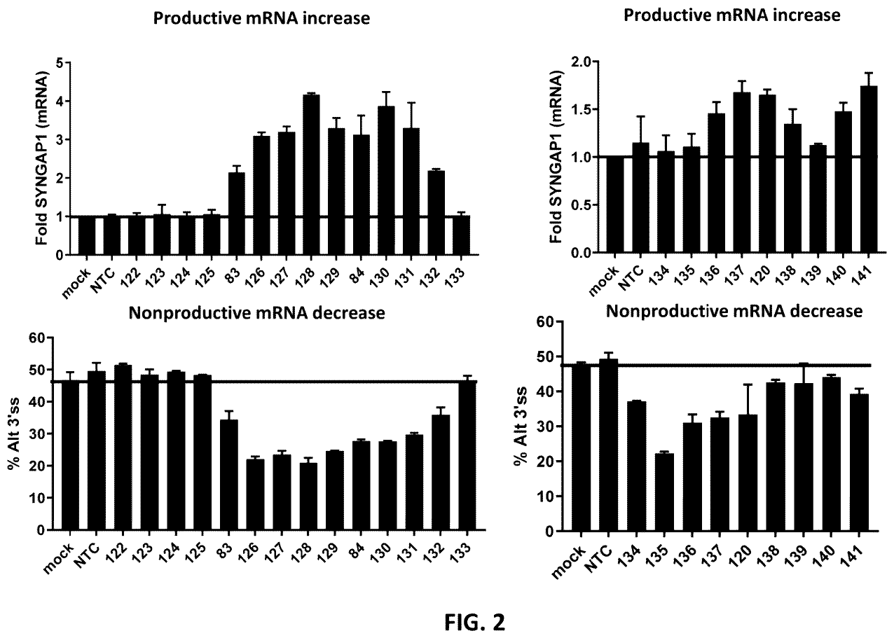

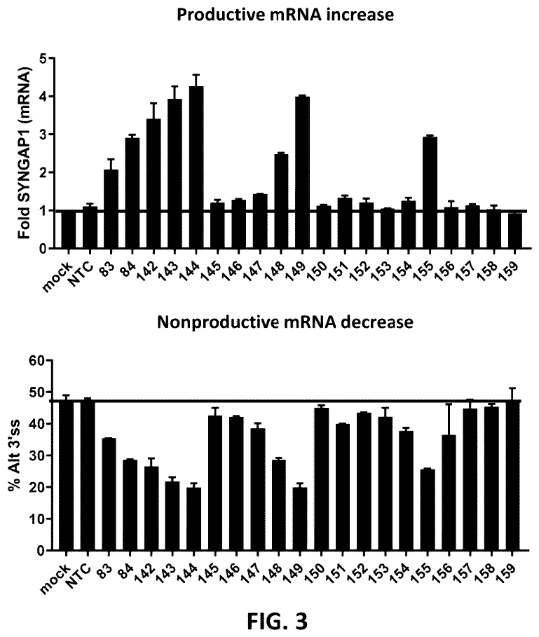

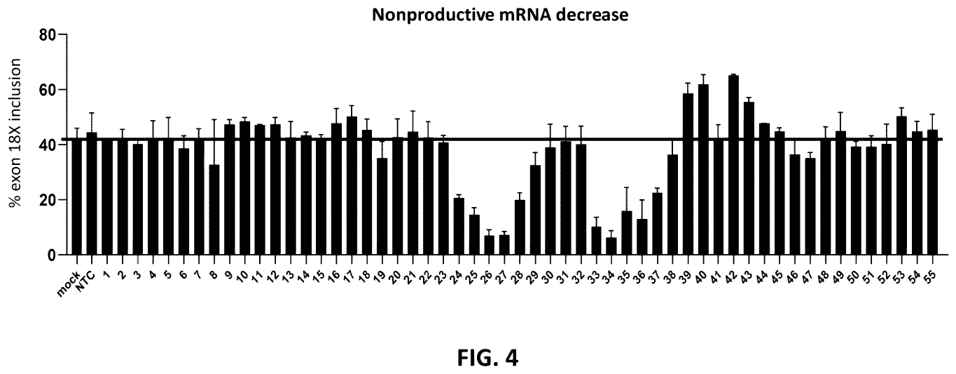

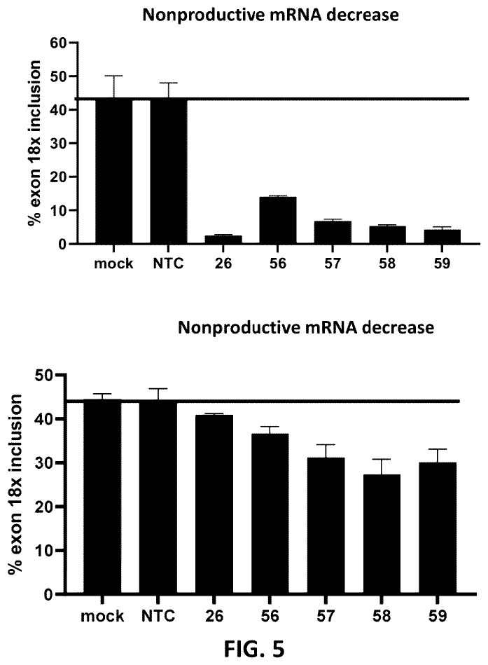

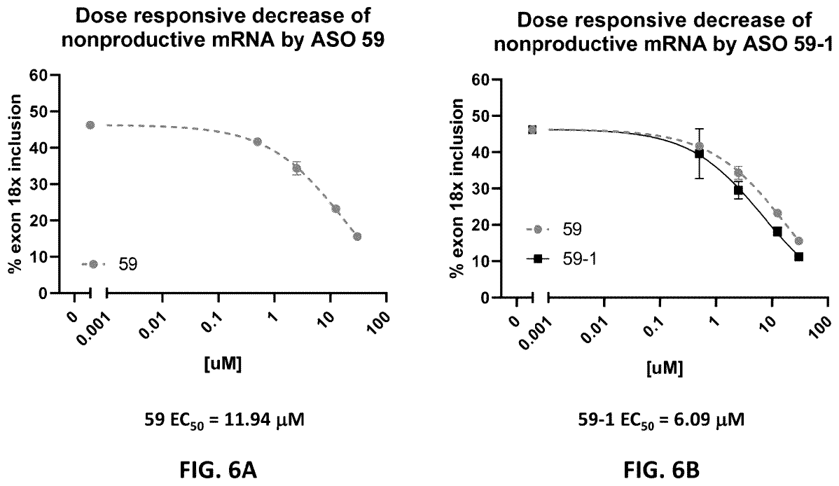

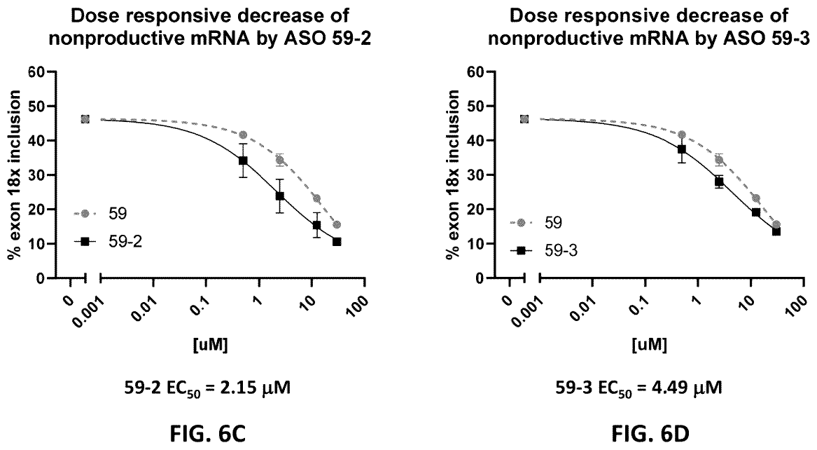

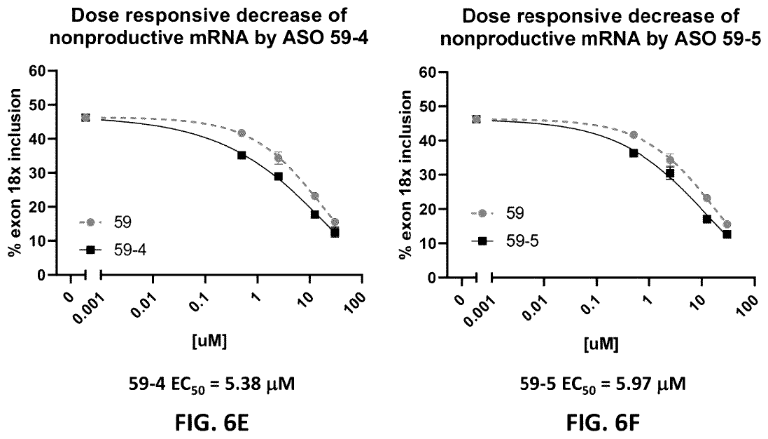

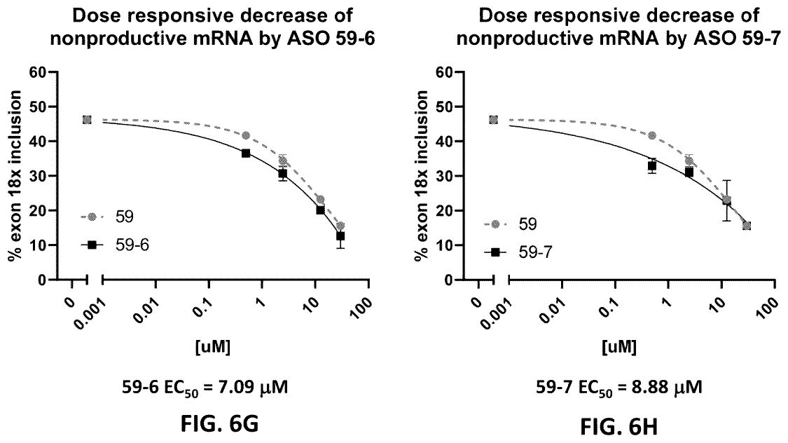

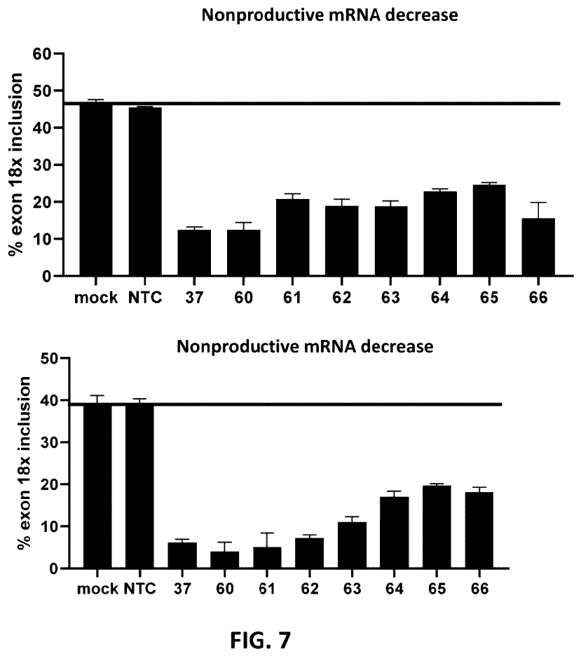

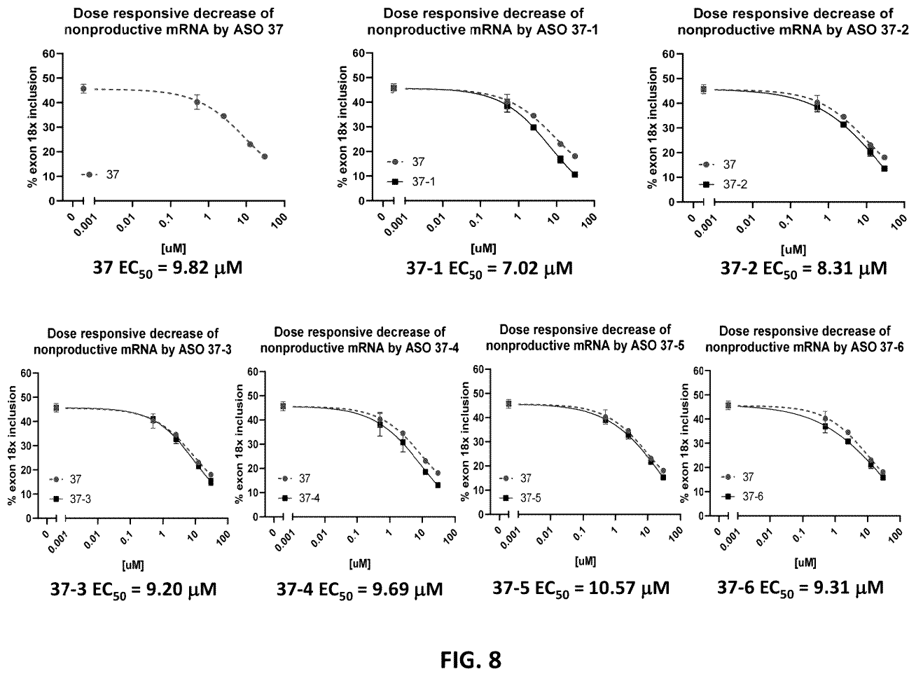

ASOs of different lengths and nucleobase chemistries were tested via RT-PCR analysis from transfected HEK293 cells to identify the effects of the ASO length and nucleobase chemistries on the decrease in nonproductive SYNGAP1 mRNA and increase in productive SYNGAP1 mRNA. FIG. 1 depicts TaqMan qPCR results for productive mRNA and RT-PCR results for nonproductive mRNA using RNA extracted from HEK293 cells 24 hrs after transfection with 80 nM of the indicated ASOs from an alternative 3′ splice site macrowalk. The exemplary TaqMan probe spans across exon 10 and the junction of exon 11 and measures productive mRNA. FIG. 2 depicts TaqMan qPCR results for productive mRNA and RT-PCR results for nonproductive mRNA using RNA extracted from HEK293 cells 24 hrs after transfection with 80 nM of the indicated ASOs from an alternative 3′ splice site microwalk. The exemplary TaqMan probe spans across exon 10 and the junction of exon 11 and measures productive mRNA. FIG. 3 depicts TaqMan qPCR results for productive mRNA and RT-PCR results for nonproductive mRNA using RNA extracted from HEK293 cells 24 hrs after transfection with 80 nM of the indicated ASOs. The exemplary TaqMan probe spans across exon 10 and the junction of exon 11 and measures productive mRNA. FIG. 4 depicts RT-PCR results for nonproductive mRNA using RNA extracted from HEK293 cells 24 hrs after they were transfected with 80 nM of the indicated ASOs. FIG. 5 depicts RT-PCR results for nonproductive mRNA using RNA extracted from HEK293 cells 24 hrs after transfection with 80 nM of the indicated ASOs (upper panel), or RNA extracted from HEK293 cells 72 hours after the cells were allowed to freely uptake 20 μM of the indicated ASOs in 3D culture (lower panel). FIG. 6 depicts RT-PCR results for dose-responsive decrease of nonproductive mRNA using RNA from HEK293 cells. Cells were treated with increasing concentrations of the indicated ASOs and evaluated 72 hours after they were allowed to freely uptake the ASOs in 3D culture. The abundance of the Exon 18X-containing transcripts was plotted against the logarithmic value (log 10) of the ASO dose using a nonlinear regression fit of a variable slope, four-parameter dose-response curve as calculated by GraphPad Prism 8. Biological replicates were plotted for each concentration with standard deviation (S.D.) indicated. The values of the control group comprising no ASO were used to plot the upper end of the curve at a concentration representative of 0 μM. Some of the projected curve analysis used constrain between 0 and 0.05. The EC50 and log EC50 values were calculated by GraphPad Prism 8. FIG. 7 depicts RT-PCR results for nonproductive mRNA using RNA extracted from HEK293 cells 24 hrs after transfection with 80 nM of the indicated ASOs (upper panel), or RNA extracted from HEK293 cells 72 hours after the cells were allowed to freely uptake 20 μM of the indicated ASOs in 3D culture (lower panel). FIG. 8 depicts RT-PCR results for dose-responsive decrease of nonproductive mRNA using RNA from HEK293 cells. Cells were treated with increasing concentrations of the indicated ASOs and evaluated 72 hours after they were allowed to freely uptake the ASOs in 3D culture. The abundance of the Exon 18X-containing transcripts was plotted against the logarithmic value (log 10) of the ASO dose using a nonlinear regression fit of a variable slope four parameter dose-response curve as calculated by GraphPad Prism 8. Biological replicates were plotted for each concentration with standard deviation (S.D.) indicated. The values of the control group comprising no ASO were used to plot the upper end of the curve at a concentration representative of 0 μM. Some of the projected curve analysis used constraints between 0 and 0.05. The EC50 and log EC50 values were calculated by GraphPad Prism 8.

### Example 6: In Vivo Data for ASOs Targeting E18X-Hippocampus

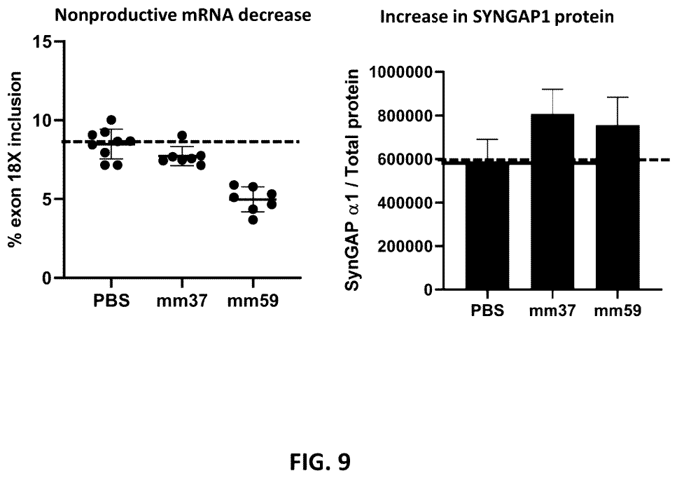

FIG. 9 depicts RT-PCR results for nonproductive mRNA and results using a ProteinSimple Jess assay to detect the alpha 1 (a1) isoform of SYNGAP1. 20 mg of indicated ASO was intracerebroventricularly injected (ICV) into P2 Syngap1 heterozygous (Syngap1^{+/-}) mice. Four weeks later, brains were dissected, and their hippocampi were assayed for Exon 18X inclusion by RT-PCR. The levels of the a1-isoform of SYNGAP1 protein were normalized to total protein via a ProteinSimple JESS assay.

### Example 7: Evaluation of In Vivo ASO Target Engagement and Corresponding Changes in SYNGAP1 Protein Expression Levels in Various Brain Regions of a Syngap1^{+/-} Mouse Model

Male and female Syngap1 heterozygous (Syngap1^{+/-}) mouse pups received intracerebroventricular injections of various amounts (3 μg, 10 μg, 20 μg, and 30 μg) of four different ASO compounds (compounds mm59, mm60, mm61, and mm62) (Table 8) on postnatal day 2 (PND2). Mouse brains were collected four weeks after ASO treatment and dissected such that ASO target engagement was analyzed in regions from the brain's left hemisphere and SYNGAP1 protein expression levels were analyzed in regions from the brain's right hemisphere.

After treatment with various ASO compounds, Exon 18X levels were measured by quantitative RT-PCR of exons 18-19. ASO target engagement was evaluated as the fold change in nonproductive mRNA normalized to mitochondrial ribosomal protein L16 (MRPL16) RNA. Protein levels were measured by capillary immuno-electrophoresis via ProteinSimple JESS assays. Effective ASO-mediated target engagement was measured as a decrease in Exon 18X inclusion and increase in protein levels of the alpha 1 (a1) isoform of SYNGAP1. The levels of the a1-isoform of SYNGAP1 protein were normalized to Actin. PBS was used as a negative control.

TABLE 8
Exemplary ASOs targeting the NMD Exon 18x event in mice and humans

| | Mouse Sequence | | Human Sequence |
|---|---|---|---|
| | (Bold = 2′ MOE, | | (Bold = 2′ MOE, |
| | Underlined C = 5methylC | | Underlined C = 5methylC |
| Mouse ASO | Bold and italicized = 2′-Fluoro) | Human ASO | Bold and italicized = 2′-Fluoro) |
| ID | (Sequence 5′→3′) | ID | (Sequence 5′→3′) |
| mm59 | GTCAAAGCGGAACCAG (SEQ | 59 | GCCAAAGCGGAACCAG (SEQ |
| | ID NO: 275) | | ID NO: 253) |
| mm60 | GTCAAAGCGGAACCAG (SEQ | 59-3 | GCCAAAGCGGAACCAG (SEQ |
| | ID NO: 282) | | ID NO: 256) |
| mm61 | GTCAAAGCGGAACCAG (SEQ | 59-8 | GCCAAAGCGGAACCAG (SEQ |
| | ID NO: 283) | | ID NO: 285) |
| mm62 | GTCAAAGCGGAACCAG (SEQ | 59-9 | GCCAAAGCGGAACCAG (SEQ |
| | ID NO: 284) | | ID NO: 17) |

### Cerebral Cortex

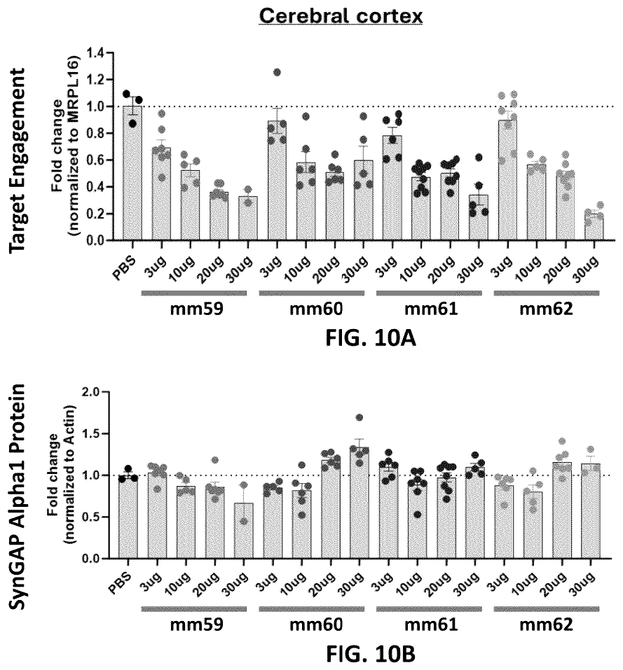

All four tested compounds resulted in an observable, dose-responsive decrease in nonproductive mRNA levels such that higher doses of each ASO resulted in greater decreases in nonproductive mRNA (FIG. 10A) in the cerebral cortex. ASO compound mm59 delivered intracerebroventricularly at 30 μg resulted in a decrease of nonproductive mRNA to about 0.3-fold of PBS-treated Syngap1^{+/-} mice. ASO compound mm60 delivered intracerebroventricularly at 20 μg resulted in a decrease of nonproductive mRNA to about 0.5-fold of PBS-treated Syngap1^{+/-} mice. ASO compound mm61 delivered intracerebroventricularly at 30 μg resulted in a decrease of nonproductive mRNA to about 0.3-fold of PBS-treated Syngap1^{+/-} mice. ASO compound mm62 delivered intracerebroventricularly at 30 μg resulted in a decrease of nonproductive mRNA to about 0.2-fold of PBS-treated Syngap1^{+/-} mice.

Levels of SynGAP a1 protein in the cerebral cortex after treatment of the Syngap1^{+/-} mice with the four ASO compounds are illustrated in FIG. 10B. ASO compound mm60 delivered intracerebroventricularly at 20 μg and 30 μg resulted in respective increases of the a1 SYNGAP1 protein isoform to about 1.15-fold and about 1.3-fold relative to PBS-treated Syngap1^{+/-} mice. ASO compound mm61 delivered intracerebroventricularly at 3 μg and 30 μg resulted in respective increases of the a1 SYNGAP1 protein isoform to about 1.1-fold and about 1.05-fold relative to PBS-treated Syngap1^{+/-} mice. ASO compound mm62 delivered intracerebroventricularly at 20 μg and 30 μg both resulted in increases of the a1 SYNGAP1 protein isoform to about 1.1-fold relative to PBS-treated Syngap1^{+/-} mice.

### Hippocampus

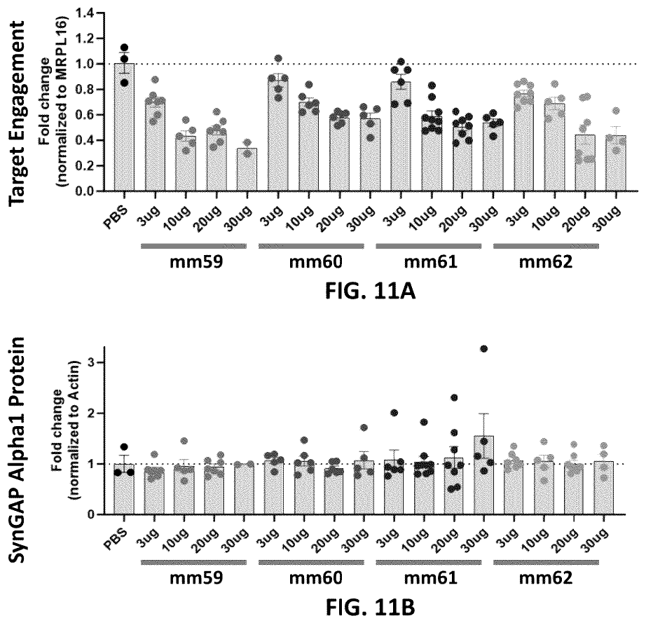

All four tested compounds resulted in an observable, dose-responsive decrease in nonproductive mRNA levels such that higher doses of each ASO resulted in greater decreases in nonproductive mRNA (FIG. 11A) in the hippocampus. ASO compound mm59 delivered intracerebroventricularly at both 10 μg and 20 μg resulted in a decrease of nonproductive mRNA to about 0.4-fold of PBS-treated Syngap1^{+/-} mice. ASO compound mm59 delivered intracerebroventricularly at 30 μg resulted in a decrease of nonproductive mRNA to about 0.3-fold of PBS-treated Syngap1^{+/-} mice. ASO compound mm60 delivered intracerebroventricularly at both 20 μg and 30 μg resulted in a decrease of nonproductive mRNA to about 0.5-fold of PBS-treated Syngap1^{+/-} mice. ASO compound mm61 delivered intracerebroventricularly at both 20 μg and 30 μg resulted in a decrease of nonproductive mRNA to about 0.5-fold of PBS-treated Syngap1^{+/-} mice. ASO compound mm62 delivered intracerebroventricularly at both 20 μg and 30 μg resulted in a decrease of nonproductive mRNA to about 0.4-fold of PBS-treated Syngap1^{+/-} mice.

Levels of SynGAP a1 protein in the hippocampus after treatment of the Syngap1^{+/-} mice with the four ASO compounds are illustrated in FIG. 11B. ASO compound mm60 delivered intracerebroventricularly at 3 μg, 10 μg, and 30 μg all resulted in increases of the a1 SYNGAP1 protein isoform to about 1.05-fold relative to PBS-treated Syngap1^{+/-} mice. ASO compound mm61 delivered intracerebroventricularly at 20 μg and 30 μg resulted in respective increases of the a1 SYNGAP1 protein isoform to about 1.1-fold and about 1.5-fold relative to PBS-treated Syngap1^{+/-} mice.

### Midbrain

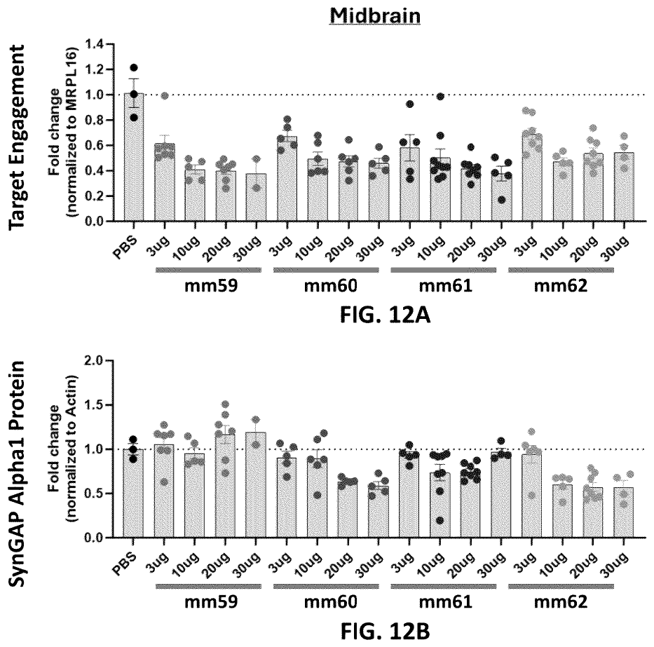

All four tested compounds resulted in an observable, dose-responsive decrease in nonproductive mRNA levels such that higher doses of each ASO resulted in greater decreases in nonproductive mRNA (FIG. 12A) in the midbrain. ASO compound mm59 delivered intracerebroventricularly at 10 μg, 20 μg, and 30 μg resulted in a decrease of nonproductive mRNA to about 0.4-fold of PBS-treated Syngap1^{+/-} mice. ASO compound mm60 delivered intracerebroventricularly at 10 μg, 20 μg, and 30 μg resulted in a decrease of nonproductive mRNA to about 0.5-fold of PBS-treated Syngap1^{+/-} mice. ASO compound mm61 delivered intracerebroventricularly at 10 μg, 20 μg, and 30 μg resulted in a decrease of nonproductive mRNA to about 0.45-fold of PBS-treated Syngap1^{+/-} mice. ASO compound mm62 delivered intracerebroventricularly at 10 μg, 20 μg, and 30 μg resulted in a decrease of nonproductive mRNA to about 0.55-fold of PBS-treated Syngap1^{+/-} mice.

Levels of SynGAP a1 protein in the midbrain after treatment of the Syngap1^{+/-} mice with the four ASO compounds are illustrated in FIG. 12B. ASO compound mm59 delivered intracerebroventricularly at 20 μg and 30 μg resulted in increases of the a1 SYNGAP1 protein isoform to about 1.2-fold and 1.25-fold relative to PBS-treated Syngap1^{+/-} mice.

### Basal Ganglia/Thalamus

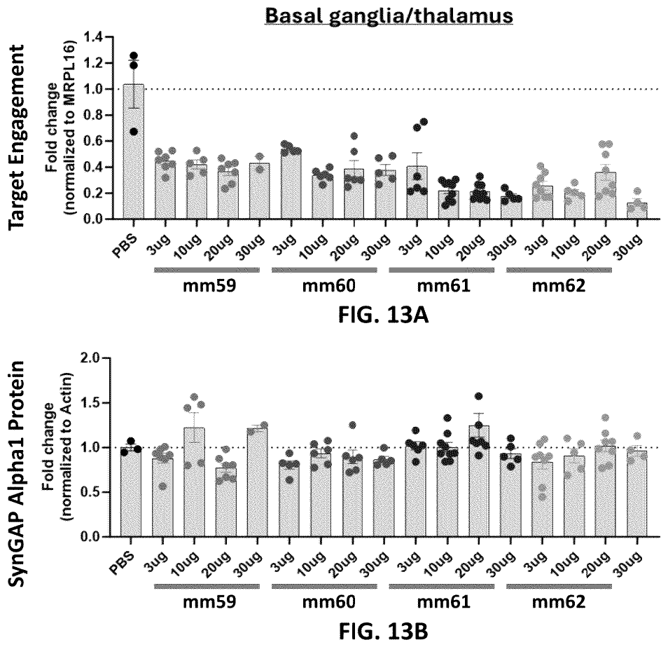

All four tested compounds resulted in an observable, dose-responsive decrease in nonproductive mRNA levels such that higher doses of each ASO resulted in greater decreases in nonproductive mRNA (FIG. 13A) in the basal ganglia/thalamus. ASO compound mm59 delivered intracerebroventricularly at 3 μg, 10 μg, 20 μg, and 30 μg resulted in a decrease of nonproductive mRNA to about 0.4-fold of PBS-treated Syngap1^{+/-} mice. ASO compound mm60 delivered intracerebroventricularly at 3 μg resulted in a decrease of nonproductive mRNA to about 0.5-fold of PBS-treated Syngap1^{+/-} mice. ASO compound mm60 delivered intracerebroventricularly at 10 μg, 20 μg, and 30 μg resulted in a decrease of nonproductive mRNA to about 0.4-fold of PBS-treated Syngap1^{+/-} mice. ASO compound mm61 delivered intracerebroventricularly at 3 μg resulted in a decrease of nonproductive mRNA to about 0.4-fold of PBS-treated Syngap1^{+/-} mice. ASO compound mm61 delivered intracerebroventricularly at 10 μg, 20 μg, and 30 μg resulted in a decrease of nonproductive mRNA to about 0.2-fold of PBS-treated Syngap1^{+/-} mice. ASO compound mm62 delivered intracerebroventricularly at 3 μg, 10 μg, 20 μg, and 30 μg resulted in respective decreases of nonproductive mRNA to about 0.3-fold, about 0.2-fold, about 0.35-fold, and about 0.1-fold of PBS-treated Syngap1^{+/-} mice.

Levels of SynGAP a1 protein in the basal ganglia/thalamus after treatment of the Syngap1^{+/-} mice with the four ASO compounds are illustrated in FIG. 13B. ASO compound mm59 delivered intracerebroventricularly at 10 μg and 30 μg both resulted in increases of the a1 SYNGAP1 protein isoform to about 1.2-fold relative to PBS-treated Syngap1^{+/-} mice. ASO compound mm61 delivered intracerebroventricularly at 20 μg resulted in an increase of the a1 SYNGAP1 protein isoform to about 1.2-fold relative to PBS-treated Syngap1^{+/-} mice.

The signal-to-noise ratio for protein was higher in the midbrain and basal ganglia/thalamus, which may contribute to more variability in the observed responses to ASO treatment.

### Syngap1 ASO Activity Comparisons by EC50s in Mouse Cortical Neurons (MCNs)

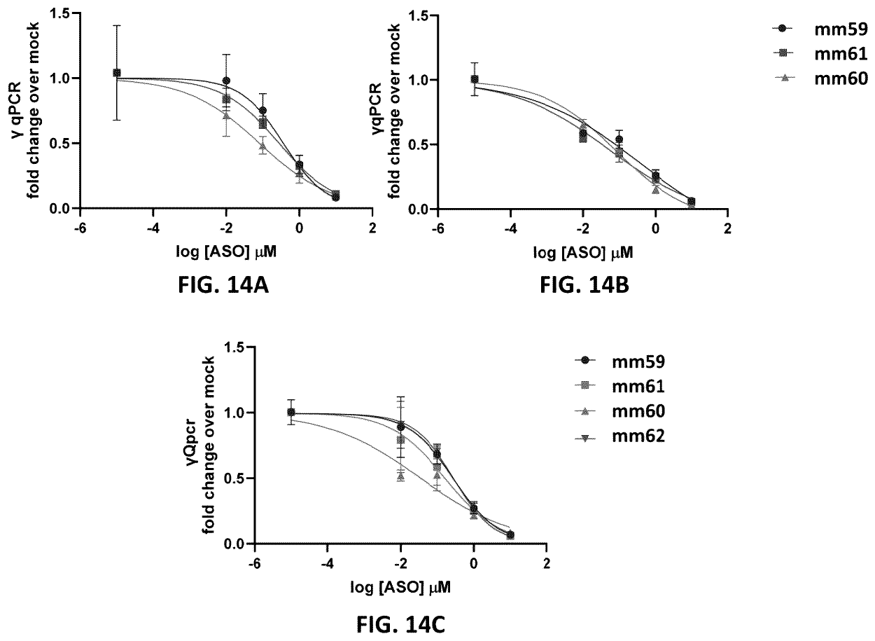

Quantitative RT-PCRs were run for mRNA extracted from wild-type Mouse Cortical Neurons (MCNs) after treatment with various ASOs (FIGS. 14A-14C) at five different concentrations. Dose-responsive decreases of nonproductive mRNA were observed. Cells were treated with increasing concentrations of the indicated ASOs and allowed to freely uptake the ASOs in 3D culture for 72 hours. The fold change of the Exon18X-containing transcripts/gamma for individual ASO concentrations was plotted against the logarithmic value (log 10) of the ASO dose using a nonlinear regression fit of a variable slope four parameter dose-response curve as calculated by GraphPad Prism 8. Biological replicates were plotted for each of the five different ASO concentrations (4-6 replicates/concentration) with standard deviation (S.D.) indicated. The EC50 and log EC50 values were calculated by GraphPad Prism 8.

TABLE 9
Syngap1 ASO activity comparisons by EC50s in MCNs

| | | Confidence | Hill | |
|---|---|---|---|---|
| ASO | EC50 (μM)¹ | interval ² | coefficient³ | N⁴ |
| mm59 | 0.34 | (0.21-0.55) | 0.53 | 3 |
| mm60 | 0.06 | (0.02-0.20) | 0.43 | 3 |
| mm61 | 0.13 | (0.02-1.1) | 0.44 | 3 |
| mm62 | 0.28 | nd | 0.80 | 1 |

¹Geometric mean of EC50;
² 95% confidence interval of EC50 mean;
³Means of Hill coefficients;
⁴Number of independent experiments on separate isolations and days.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A compound of Formula (I): 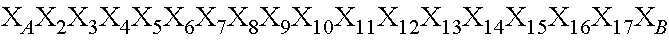
wherein
X_{A} is 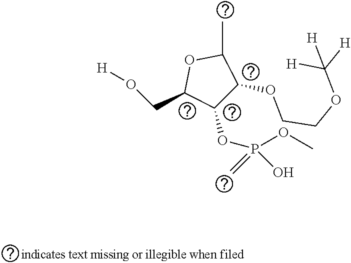 [SMILES: C=P(O)(OC)O[C@@H]1[C@@H](CO)OC(C)[C@@H]1OCCOC]
and B_{A} is 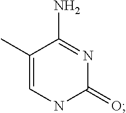
X₂ is 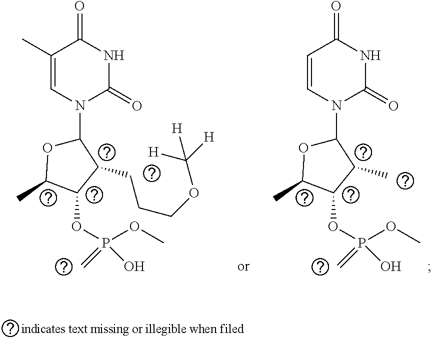
X₃ is 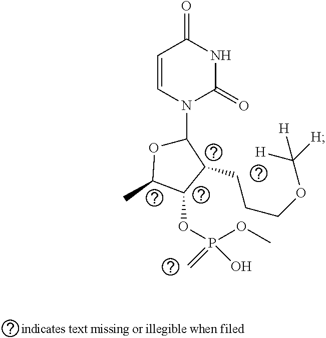
X₄ is 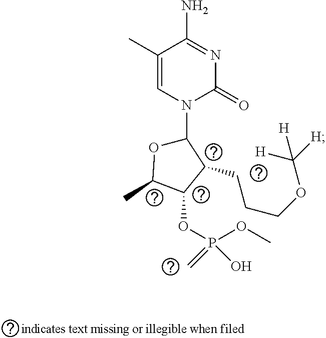
X₅ is 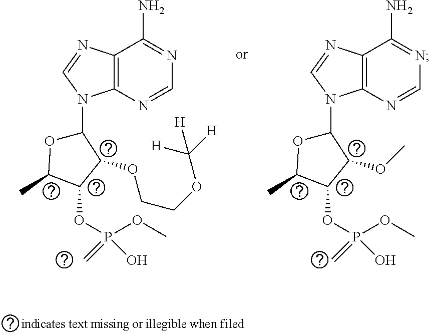 [SMILES: C=P(O)(OC)O[C@@H]1[C@@H](C)OC(n2cnc3c(N)ncnc32)[C@@H]1OCCOC ; "or" ; C=P(O)(OC)O[C@@H]1[C@@H](C)OC(n2cnc3c(N)ncnc32)[C@@H]1OC]
X₆ is 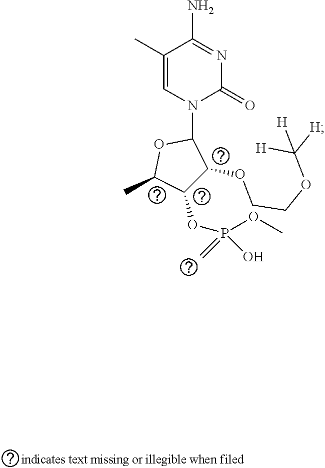
X₇ is 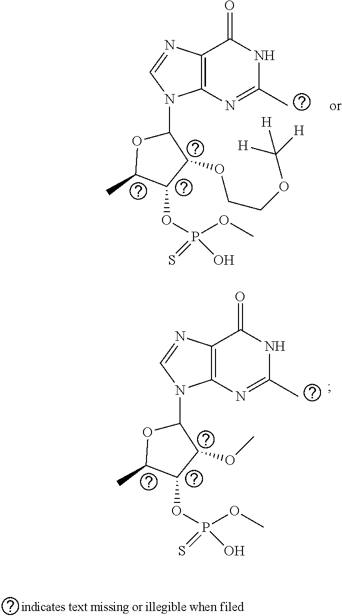
X₈ is 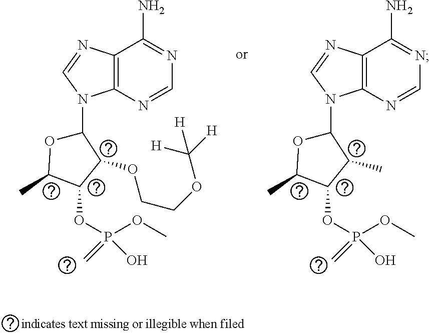 [SMILES: C=P(O)(OC)O[C@@H]1[C@@H](C)OC(n2cnc3c(N)ncnc32)[C@@H]1OCCOC ; "or" ; C=P(O)(OC)O[C@@H]1[C@@H](C)OC(n2cnc3c(N)ncnc32)[C@@H]1C]
X₉ is 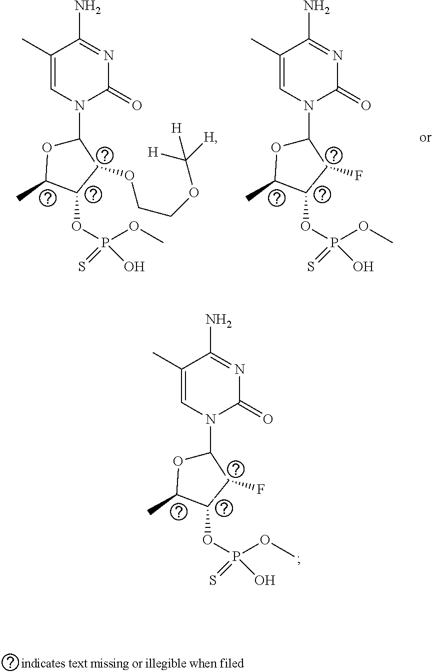
X₁₀ is 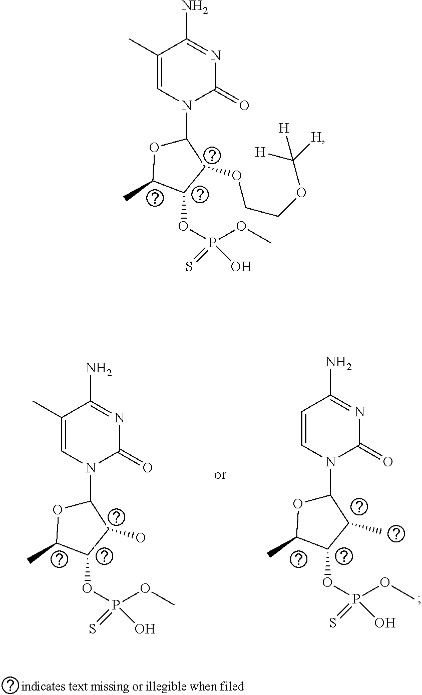
X₁₁ is 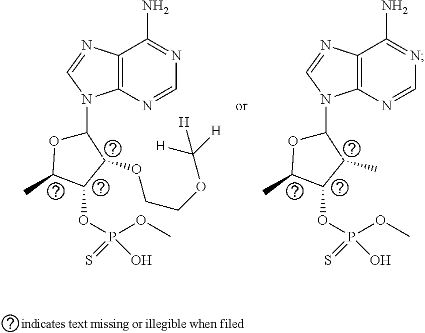 [SMILES: COCCO[C@H]1C(n2cnc3c(N)ncnc32)O[C@H](C)[C@H]1OP(O)(=S)OC ; COP(O)(=S)O[C@@H]1[C@@H](C)OC(n2cnc3c(N)ncnc32)[C@@H]1C]
X₁₂ is 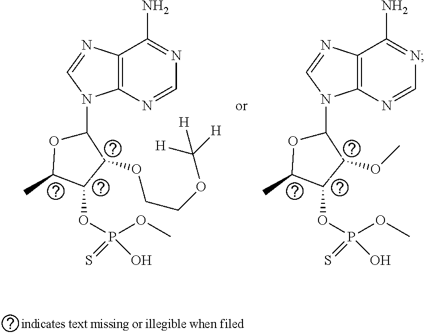 [SMILES: COCCO[C@H]1C(n2cnc3c(N)ncnc32)O[C@H](C)[C@H]1OP(O)(=S)OC ; CO[C@H]1C(n2cnc3c(N)ncnc32)O[C@H](C)[C@H]1OP(O)(=S)OC]
X₁₃ is 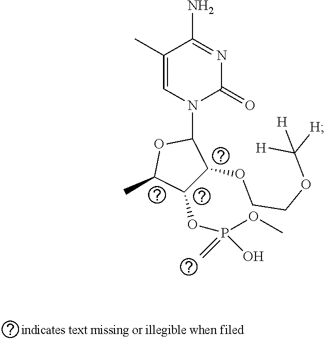
X₁₄ is 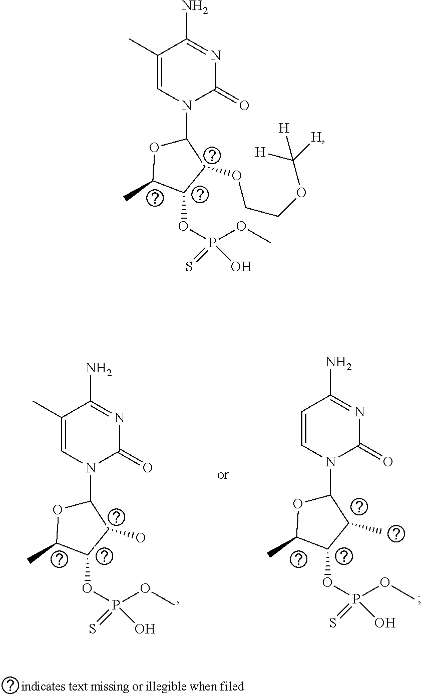
X₁₅ is 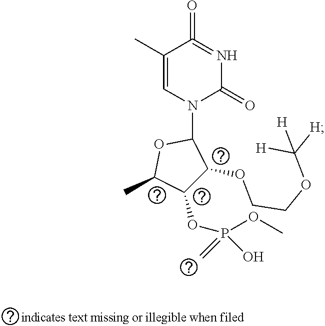
X₁₆ is 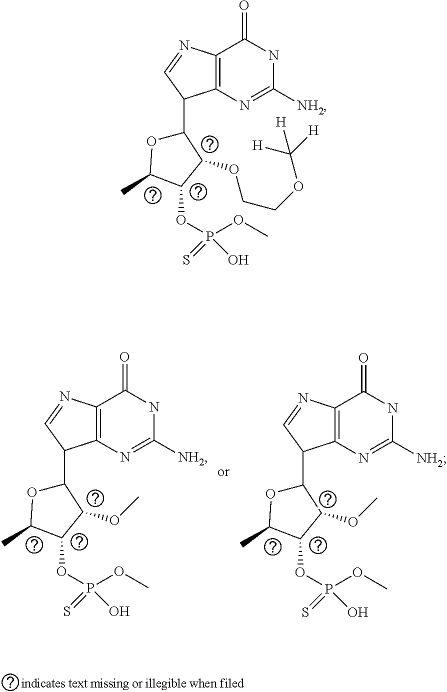 [SMILES: COCCO[C@H]1C(C2C=Nc3c2nc(N)[nH]c3=O)O[C@H](C)[C@H]1OP(O)(=S)OC ; CO[C@H]1C(C2C=Nc3c2nc(N)[nH]c3=O)O[C@H](C)[C@H]1OP(O)(=S)OC ; CO[C@H]1C(C2C=Nc3c2nc(N)[nH]c3=O)O[C@H](C)[C@H]1OP(O)(=S)OC]
X₁₇ is 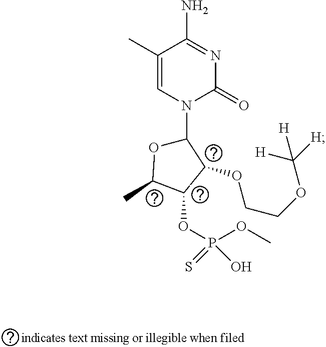
and X_{B} is 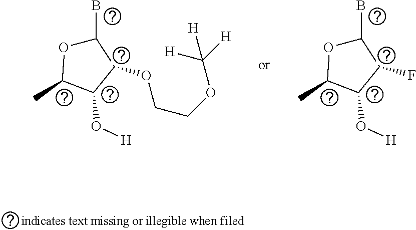
and B_{B} is

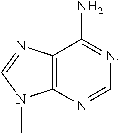

2. The compound of claim 1, wherein
X₂ is 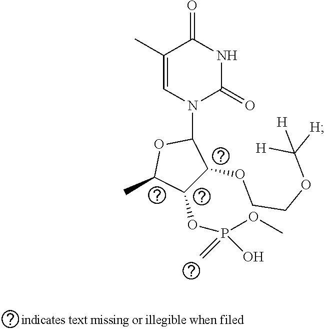
X₅ is 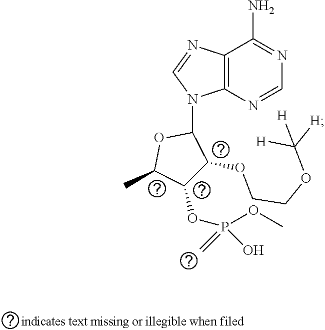
X₇ is 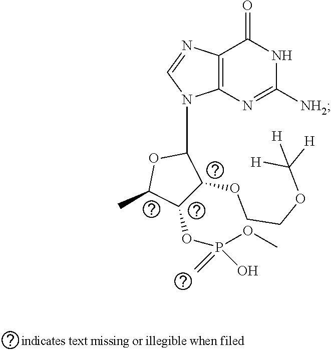 [SMILES: C=P(O)(OC)O[C@@H]1[C@@H](C)OC(n2cnc3c(=O)[nH]c(N)nc32)[C@@H]1OCCOC]
X₈ is 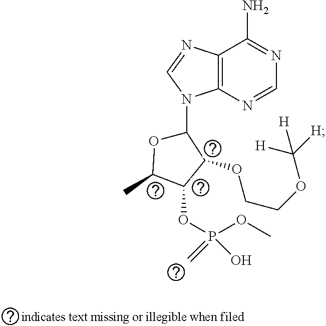
X₉ is 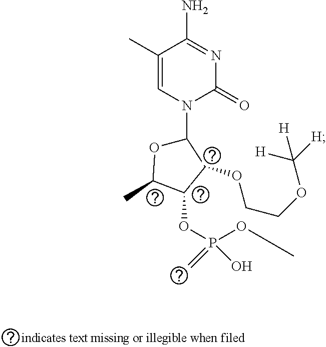
X₁₀ is 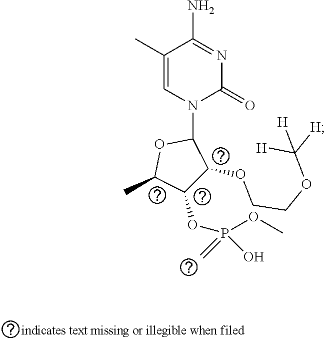
X₁₁ is 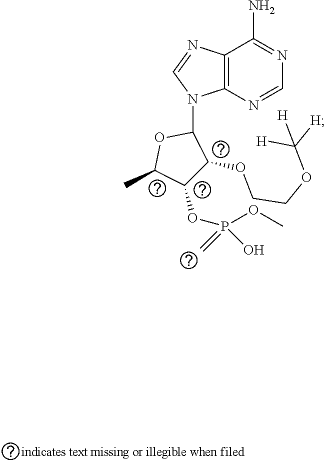
X₁₂ is 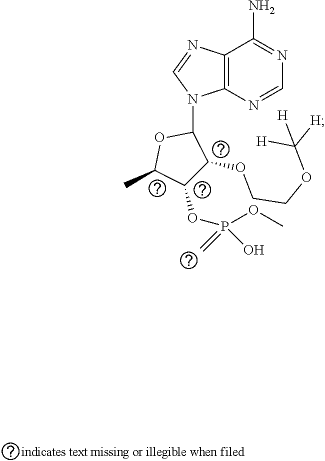
X₁₄ is 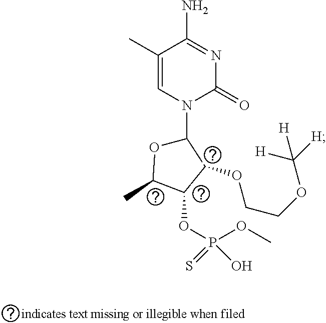
X₁₆ is 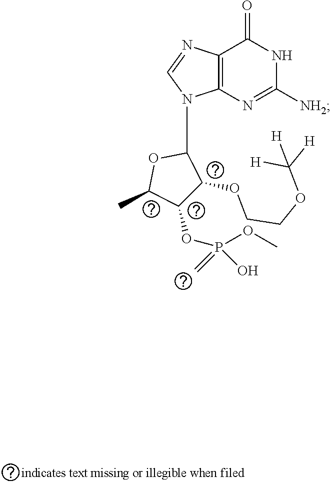 [SMILES: C=P(O)(OC)O[C@@H]1[C@@H](C)OC(n2cnc3c(=O)[nH]c(N)nc32)[C@@H]1OCCOC]
and X_{B} is 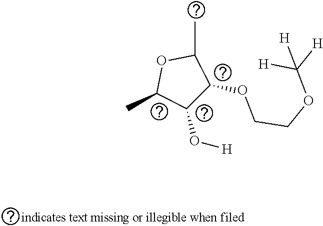
and B_{B} is

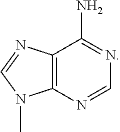

3. The compound of claim 1, wherein
X₂ is 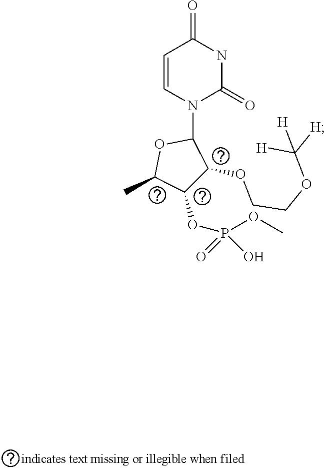
X₅ is 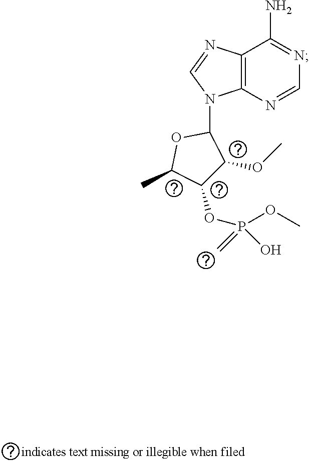
X₇ is 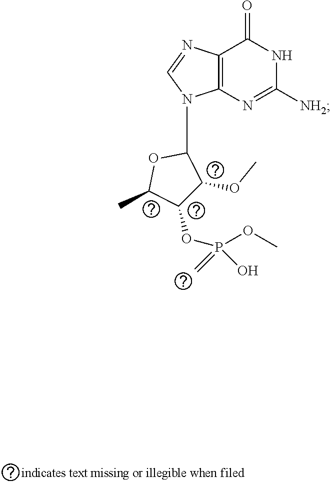 [SMILES: C=P(O)(OC)O[C@@H]1[C@@H](C)OC(n2cnc3c(=O)[nH]c(N)nc32)[C@@H]1OC]
X₈ is 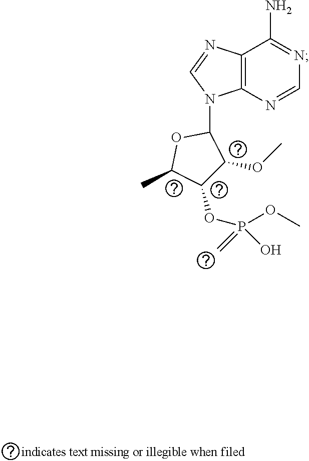
X₉ is 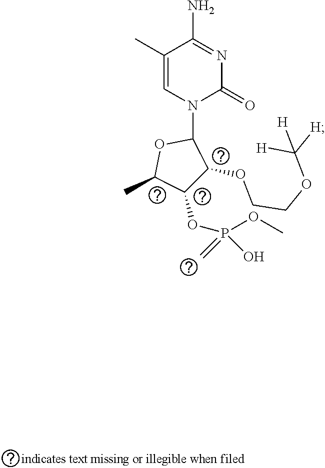
X₁₀ is 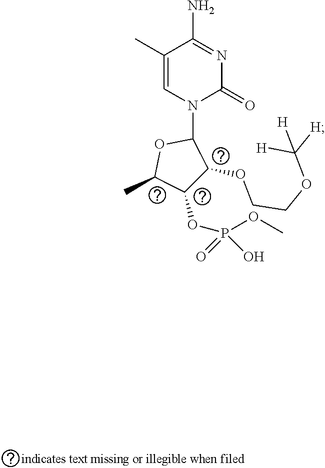
X₁₁ is 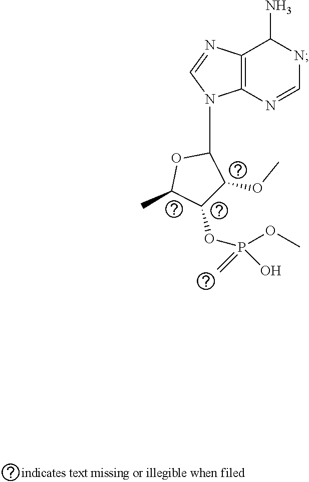
X₁₂ is 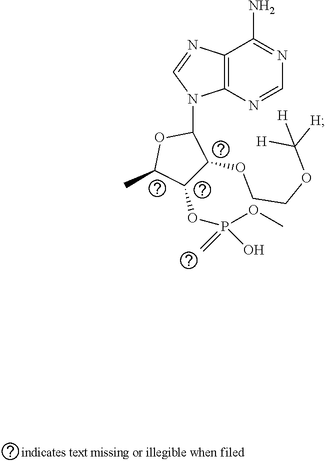
X₁₄ is 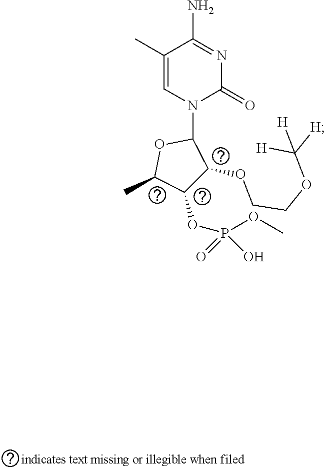
X₁₆ is 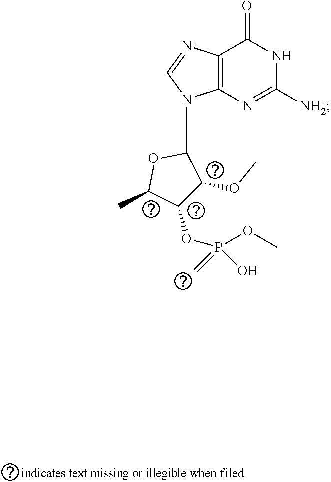 [SMILES: C=P(O)(OC)O[C@@H]1[C@@H](C)OC(n2cnc3c(=O)[nH]c(N)nc32)[C@@H]1OC]
and X_{B} is

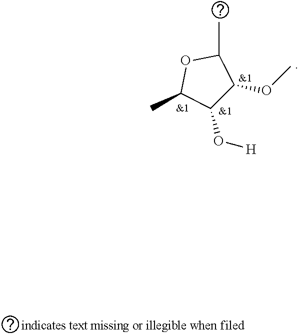

4. The compound of claim 1, wherein
X₂ is 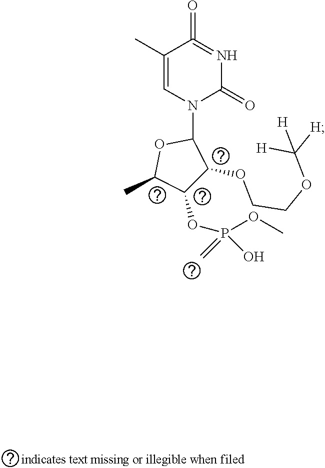
X₅ is 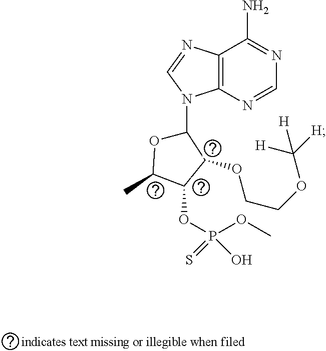
X₇ is 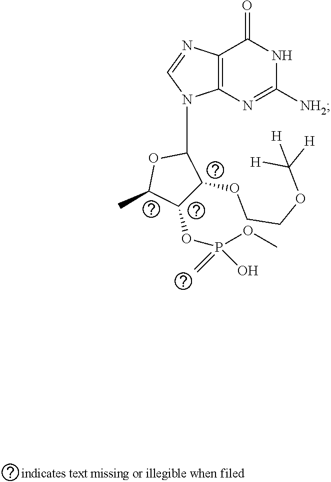 [SMILES: C=P(O)(OC)O[C@@H]1[C@@H](C)OC(n2cnc3c(=O)[nH]c(N)nc32)[C@@H]1OCCOC]
X₈ is 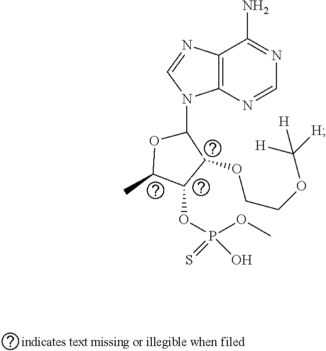
X₉ is 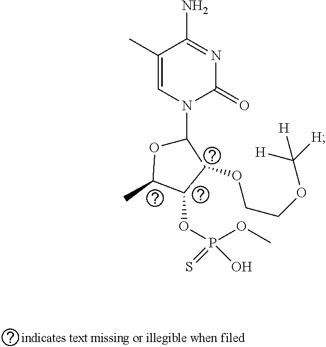
X₁₀ is 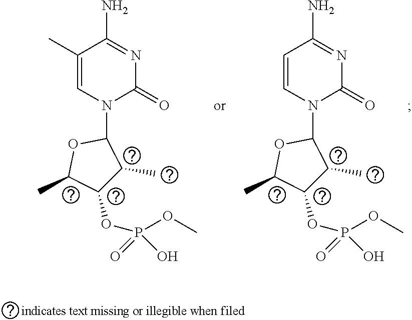
X₁₁ is 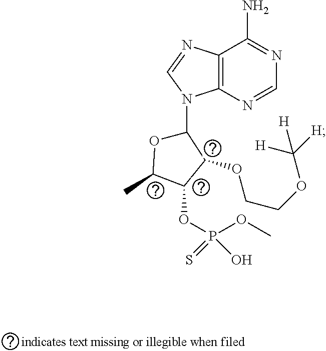
X₁₂ is 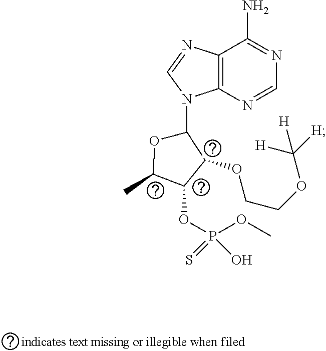
X₁₄ is 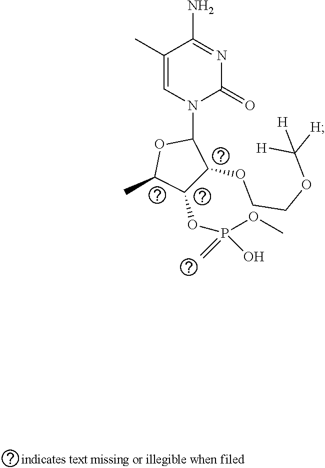
X₁₆ is 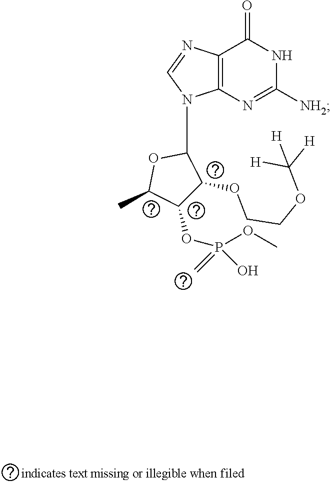 [SMILES: C=P(O)(OC)O[C@@H]1[C@@H](C)OC(n2cnc3c(=O)[nH]c(N)nc32)[C@@H]1OCCOC]
and X_{B} is

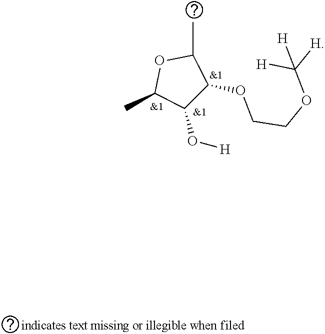

5. The compound of claim 1, wherein
X₂ is 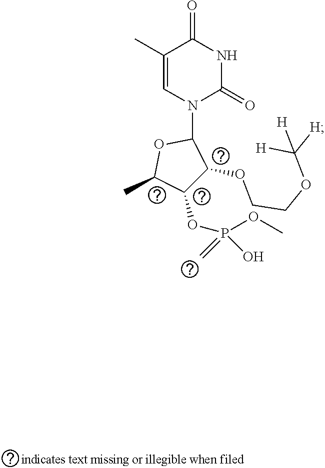
X₅ is 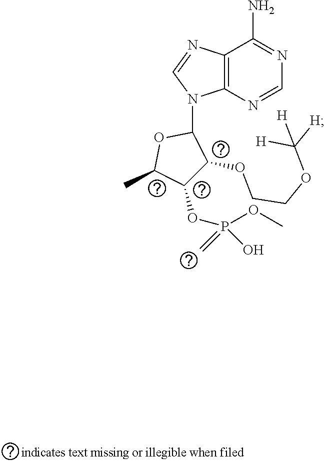
X₇ is 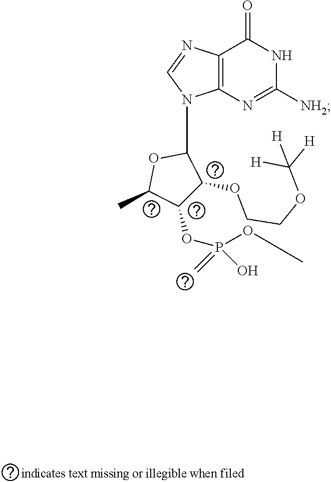 [SMILES: C=P(O)(OC)O[C@@H]1[C@@H](C)OC(n2cnc3c(=O)[nH]c(N)nc32)[C@@H]1OCCOC]
X₈ is 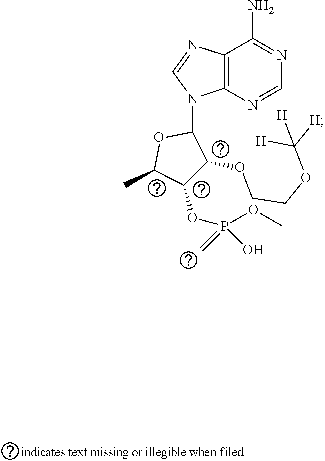
X₉ is 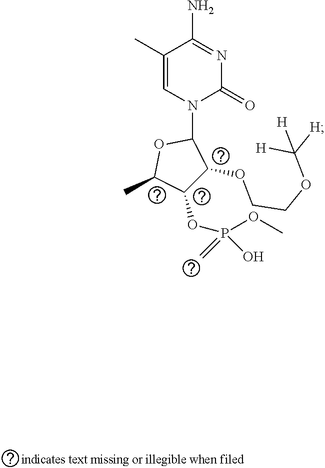
X₁₀ is 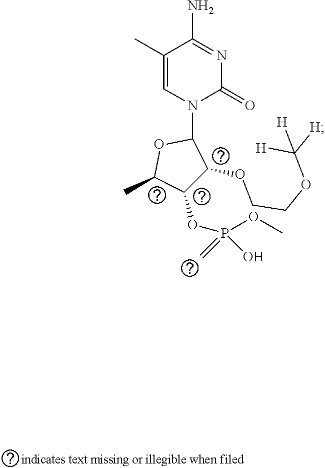
X₁₁ is 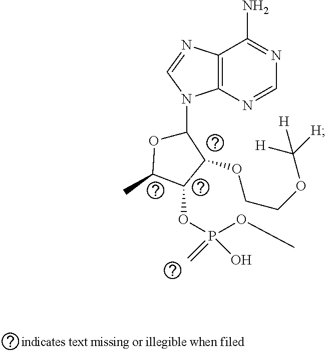
X₁₂ is 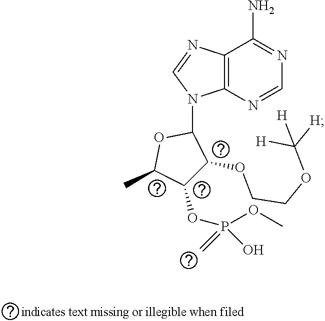
X₁₄ is 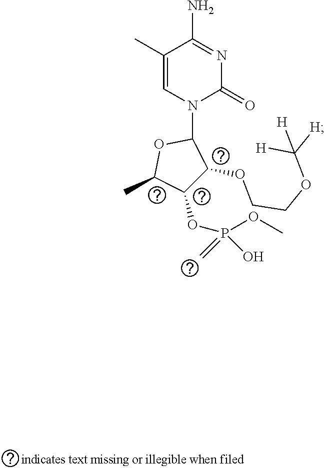
X₁₆ is 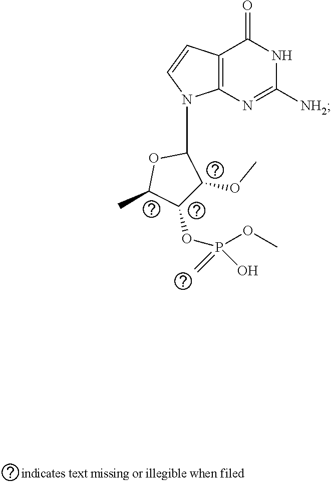 [SMILES: C=P(O)(OC)O[C@@H]1[C@@H](C)OC(n2ccc3c(=O)[nH]c(N)nc32)[C@@H]1OC]
and X_{B} is

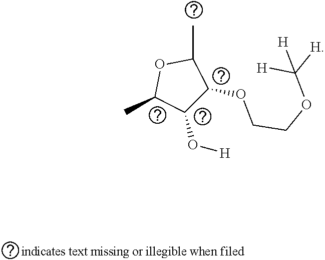

6. The compound o claim 1, wherein
X₂ is 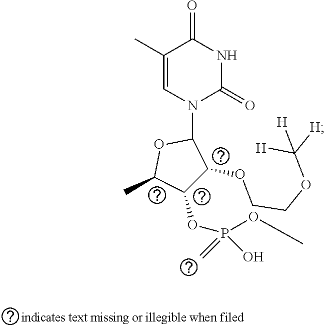
X₅ is 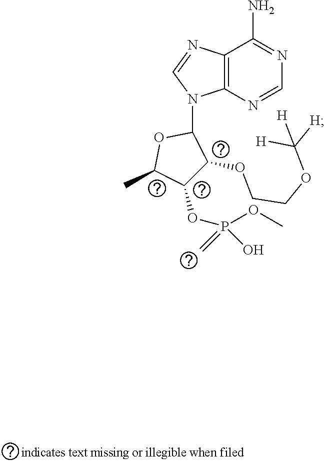
X₇ is 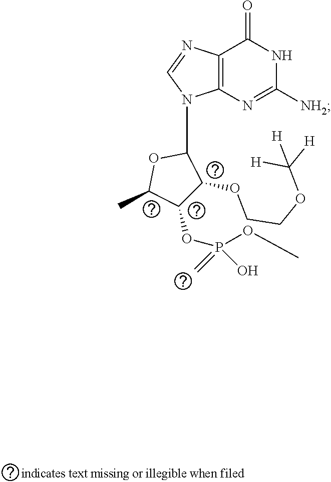 [SMILES: C=P(O)(OC)O[C@@H]1[C@@H](C)OC(n2cnc3c(=O)[nH]c(N)nc32)[C@@H]1OCCOC]
X₈ is 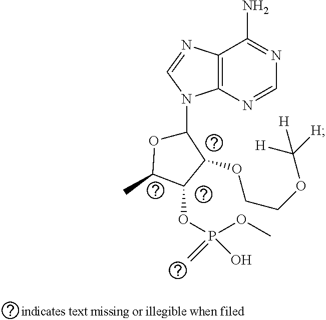
X₉ is 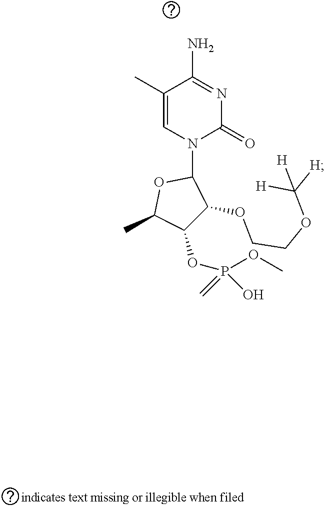
X₁₀ is 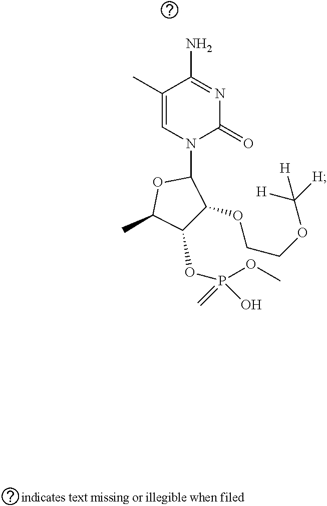
X₁₁ is 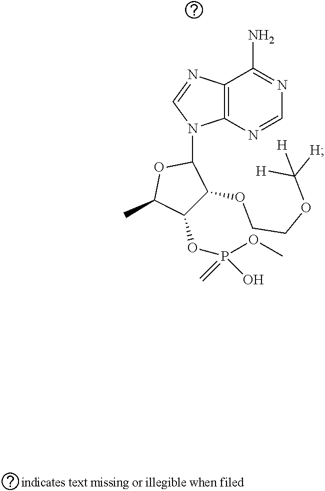
X₁₂ is 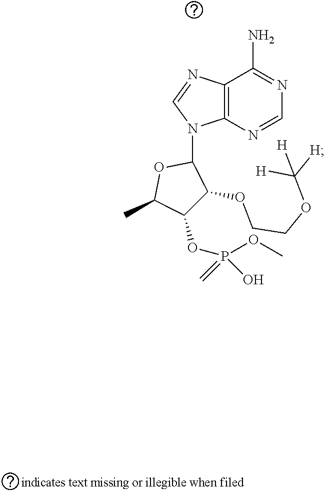
X₁₄ is 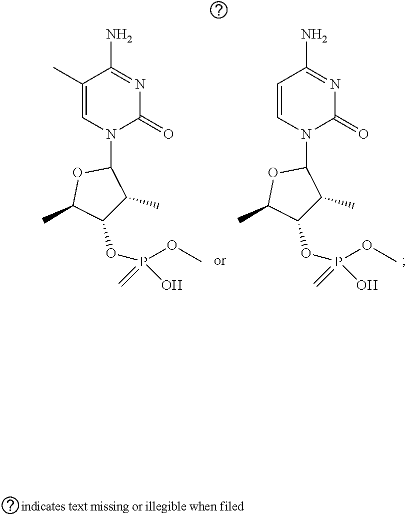
X₁₆ is 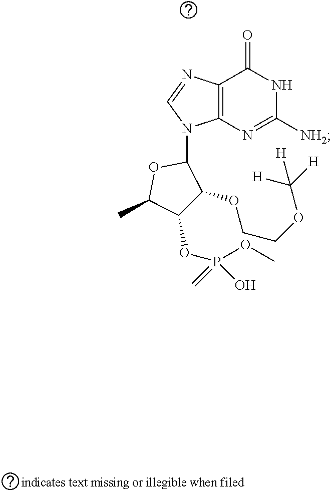 [SMILES: C=P(O)(OC)O[C@@H]1[C@@H](C)OC(n2cnc3c(=O)[nH]c(N)nc32)[C@@H]1OCCOC]
and X_{B} is

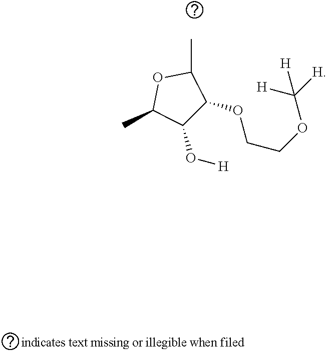

7. The compound of claim 1, wherein
X₂ is 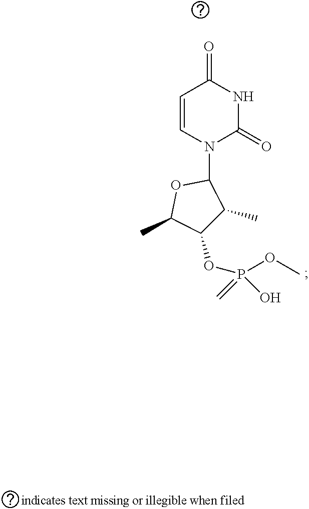
X₅ is 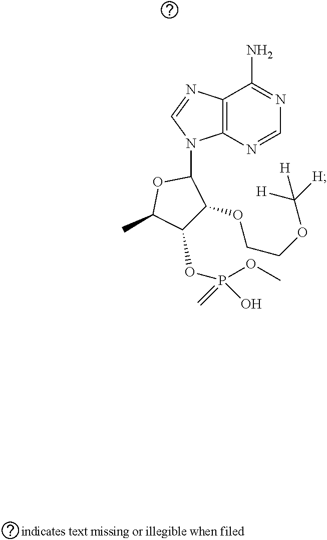
X₇ is 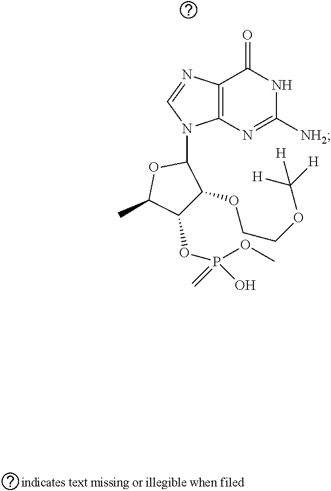 [SMILES: C=P(O)(OC)O[C@@H]1[C@@H](C)OC(n2cnc3c(=O)[nH]c(N)nc32)[C@@H]1OCCOC]
X₈ is 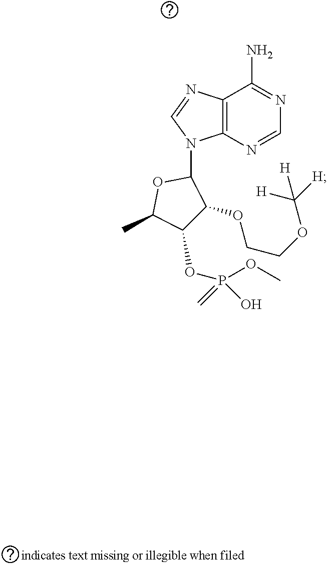
X₉ is 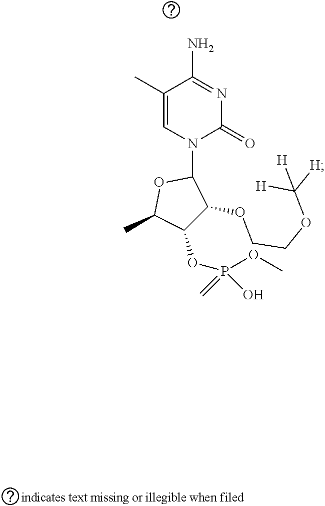
X₁₀ is 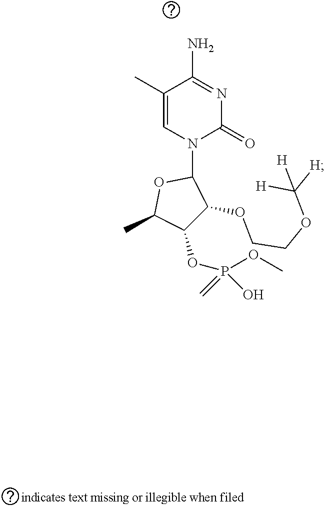
X₁₁ is 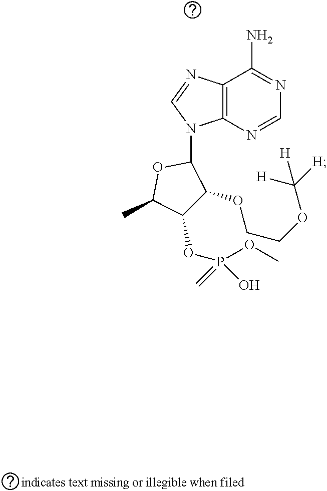
X₁₂ is 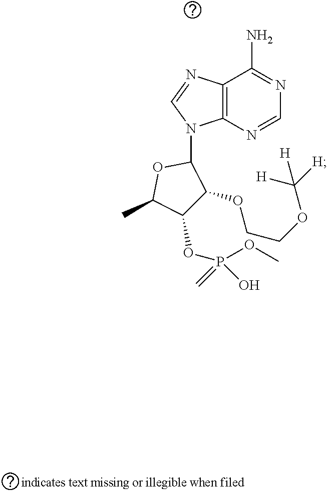
X₁₄ is 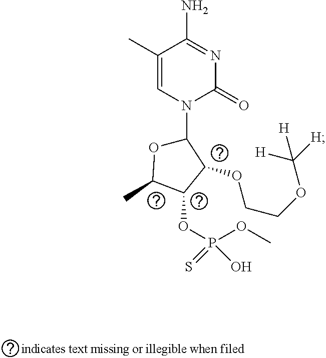
X₁₆ is 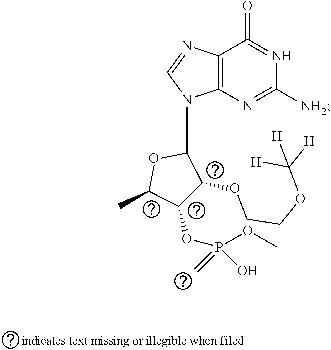
and X_{B} is

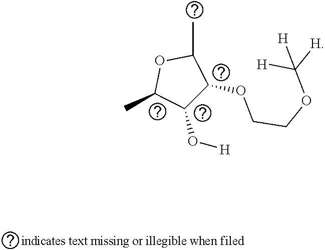

8. The compound of claim 1, wherein
X₂ is 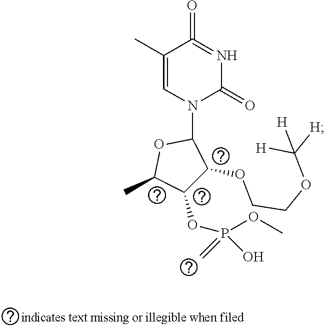
X₅ is 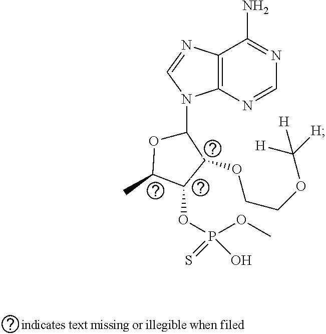
X₇ is 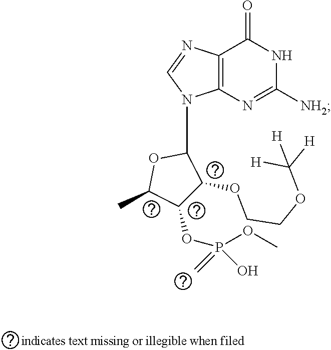 [SMILES: C=P(O)(OC)O[C@@H]1[C@@H](C)OC(n2cnc3c(=O)[nH]c(N)nc32)[C@@H]1OCCOC]
X₈ is 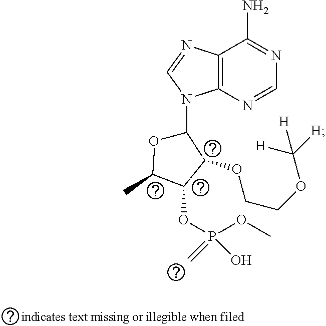
X₉ is 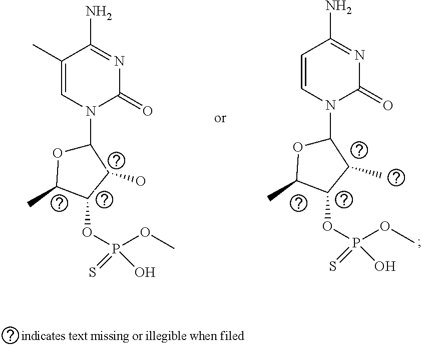
X₁₀ is 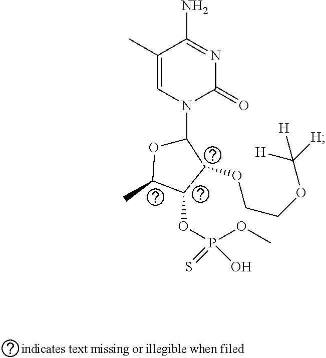
X₁₁ is 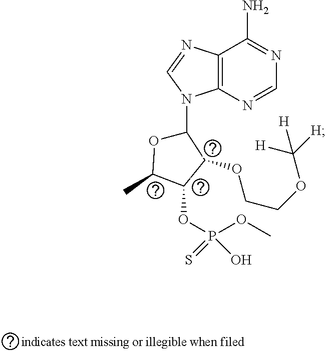
X₁₂ is 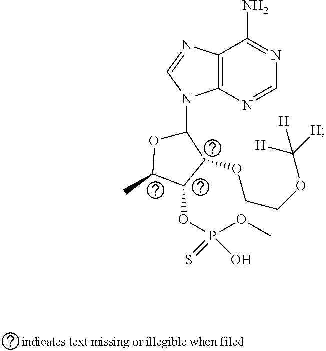
X₁₄ is 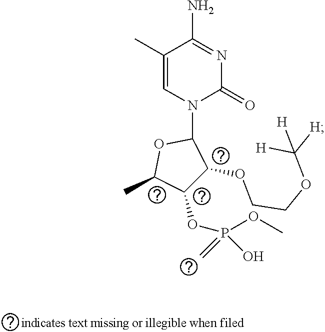
X₁₆ is 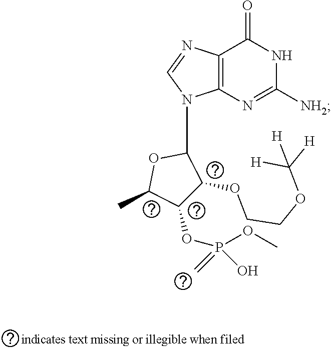 [SMILES: C=P(O)(OC)O[C@@H]1[C@@H](C)OC(n2cnc3c(=O)[nH]c(N)nc32)[C@@H]1OCCOC]
and X_{B} is

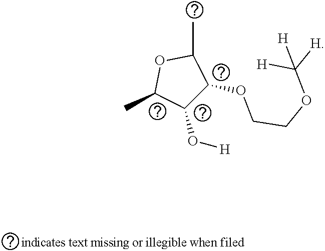

9. A compound of Formula (I):
(SEQ ID NO: 1)
X_{A}X₂X₃X₄X₅X₆X₇X₈X₉X₁₀X₁₁X₁₂X₁₃X₁₄X₁₅X₁₆X₁₇X_{B}
wherein
X_{A} is 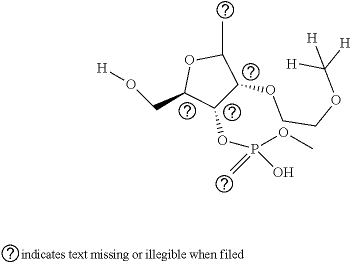 [SMILES: C=P(O)(OC)O[C@@H]1[C@@H](CO)OC(C)[C@@H]1OCCOC]
and B_{A} is 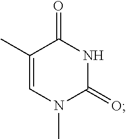
X₂ is 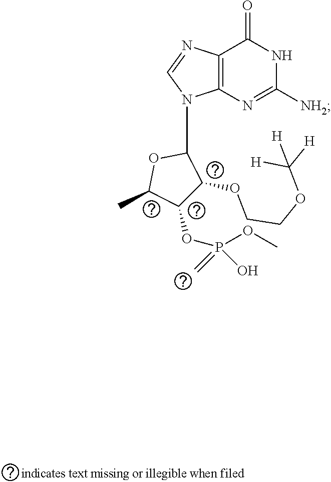 [SMILES: C=P(O)(OC)O[C@@H]1[C@@H](C)OC(n2cnc3c(=O)[nH]c(N)nc32)[C@@H]1OCCOC]
X₃ is 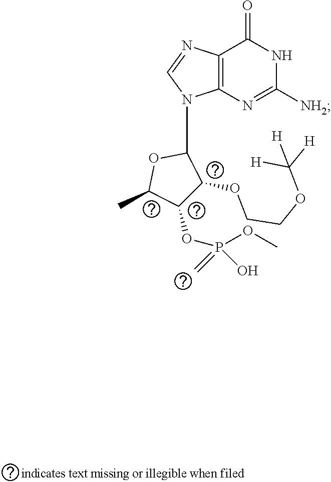 [SMILES: C=P(O)(OC)O[C@@H]1[C@@H](C)OC(n2cnc3c(=O)[nH]c(N)nc32)[C@@H]1OCCOC]
X₄ is 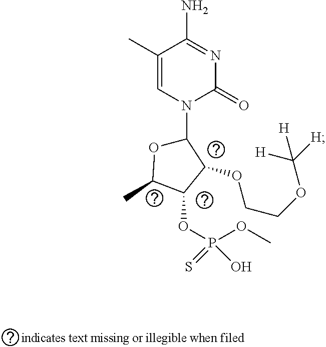
X₅ is 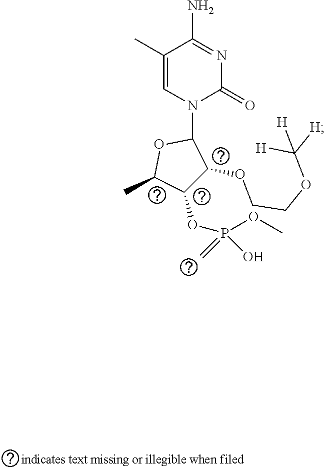
X₆ is 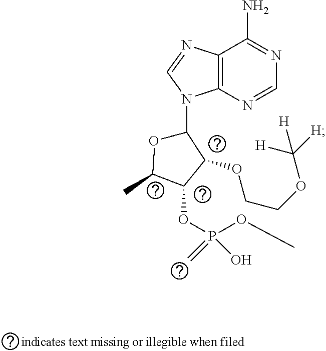
X₇ is 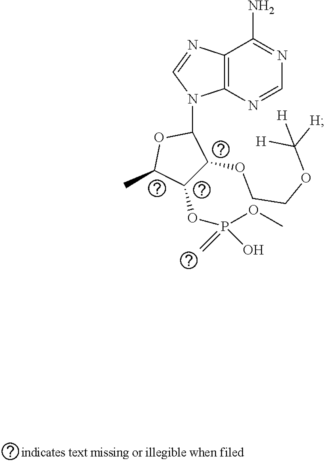
X₈ is 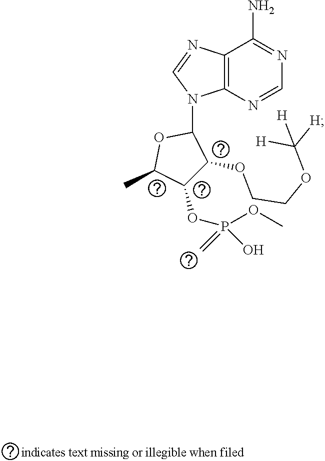
X₉ is 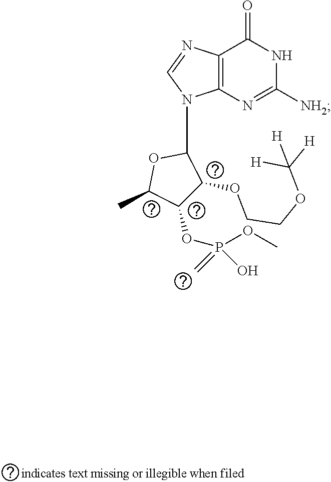 [SMILES: C=P(O)(OC)O[C@@H]1[C@@H](C)OC(n2cnc3c(=O)[nH]c(N)nc32)[C@@H]1OCCOC]
X₁₀ is 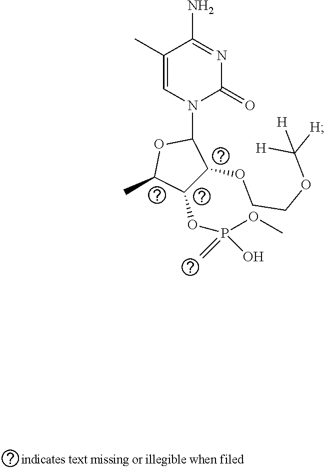
X₁₁ is 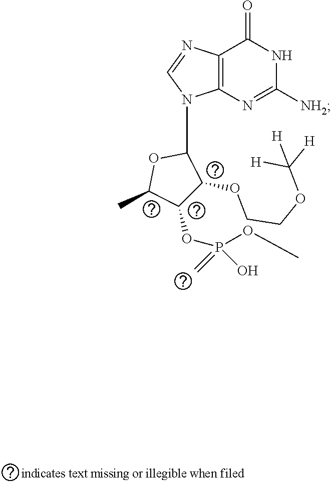 [SMILES: C=P(O)(OC)O[C@@H]1[C@@H](C)OC(n2cnc3c(=O)[nH]c(N)nc32)[C@@H]1OCCOC]
X₁₂ is 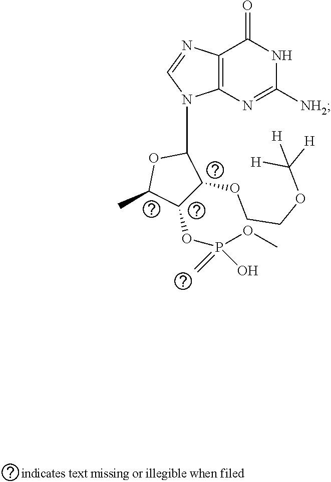 [SMILES: C=P(O)(OC)O[C@@H]1[C@@H](C)OC(n2cnc3c(=O)[nH]c(N)nc32)[C@@H]1OCCOC]
X₁₃ is 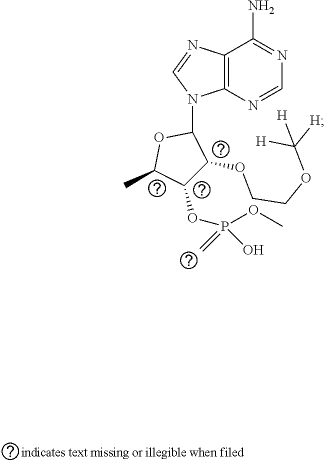
X₁₄ is 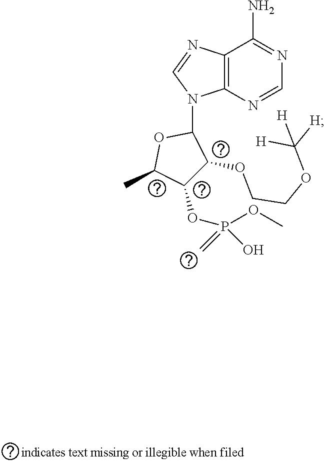
X₁₅ is 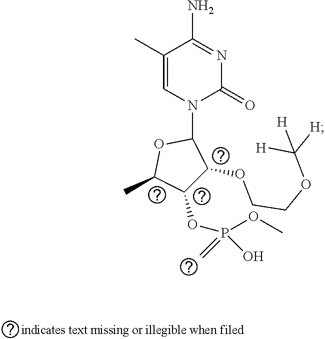
X₁₆ is 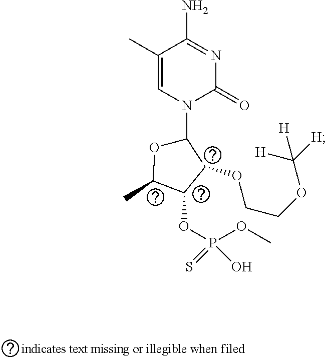
X₁₇ is 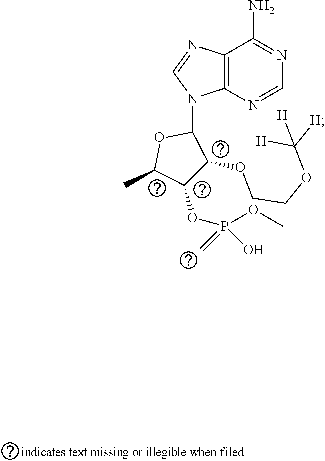
and X_{B} is 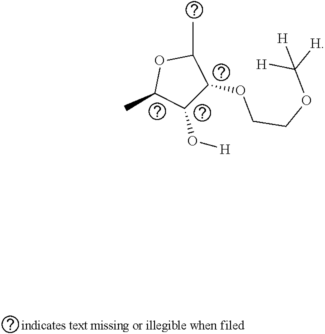
and
B_{B} is

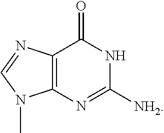

10. A compound of Formula (I):
(SEQ ID NO: 2)
X_{A}X₂X₃X₄X₅X₆X₇X₈X₉X₁₀X₁₁X₁₂X₁₃X₁₄X₁₅X_{B}
wherein
X_{A} is 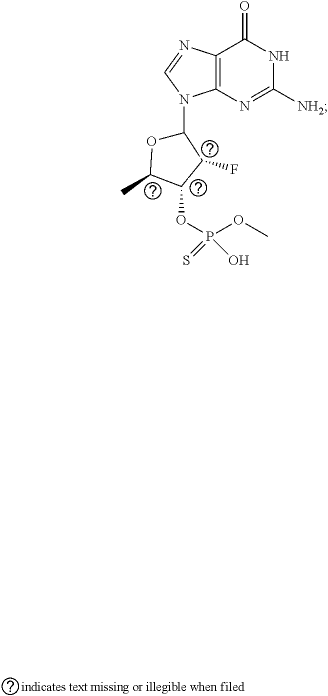 [SMILES: COP(O)(=S)O[C@@H]1[C@@H](C)OC(n2cnc3c(=O)[nH]c(N)nc32)[C@@H]1F]
and B_{A} is 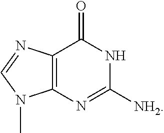
X₂ is 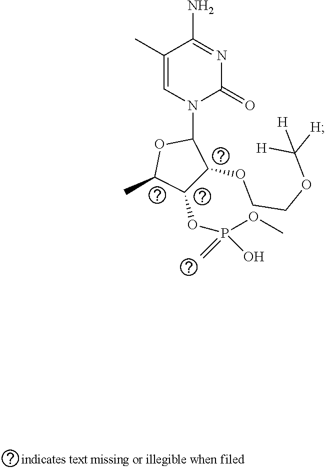
X₃ is 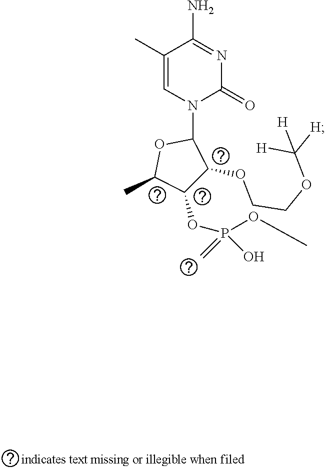
X₄ is 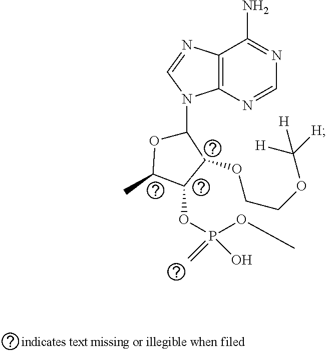
X₅ is 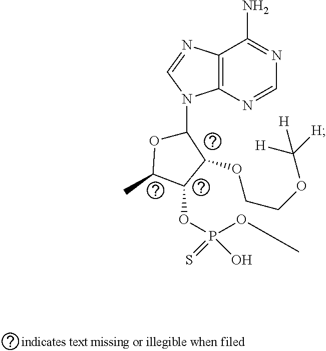
X₆ is 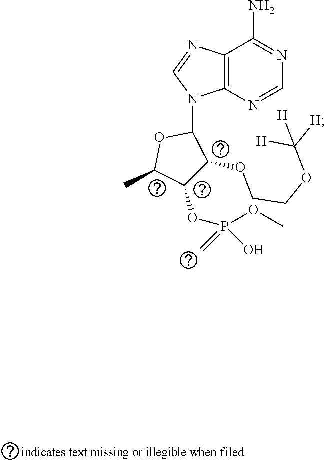
X₇ is 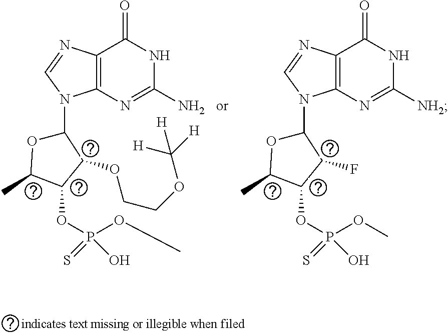 [SMILES: COCCO[C@H]1C(n2cnc3c(=O)[nH]c(N)nc32)O[C@H](C)[C@H]1OP(O)(=S)OC ; COP(O)(=S)O[C@@H]1[C@@H](C)OC(n2cnc3c(=O)[nH]c(N)nc32)[C@@H]1F]
X₈ is 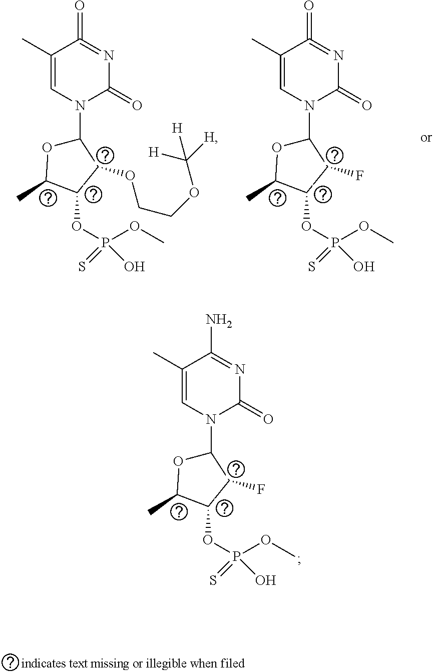
X₉ is 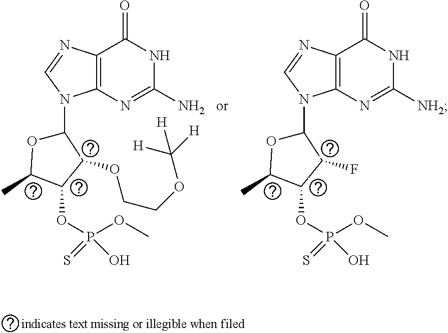 [SMILES: COCCO[C@H]1C(n2cnc3c(=O)[nH]c(N)nc32)O[C@H](C)[C@H]1OP(O)(=S)OC ; COP(O)(=S)O[C@@H]1[C@@H](C)OC(n2cnc3c(=O)[nH]c(N)nc32)[C@@H]1F]
X₁₀ is 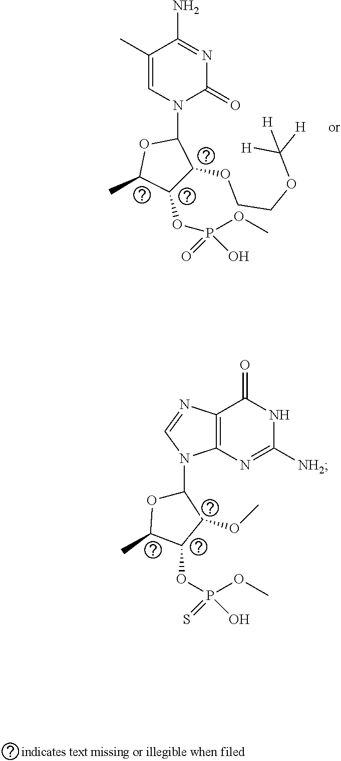 [SMILES: COCCO[C@H]1C(n2cc(C)c(N)nc2=O)O[C@H](C)[C@H]1OP(=O)(O)OC ; CO[C@H]1C(n2cnc3c(=O)[nH]c(N)nc32)O[C@H](C)[C@H]1OP(O)(=S)OC]
X₁₁ is 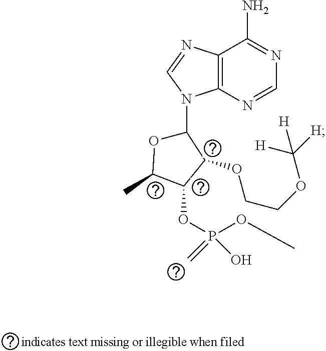
X₁₂ is 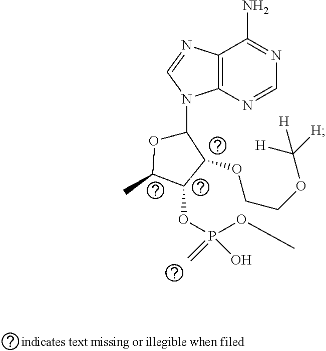
X₁₃ is 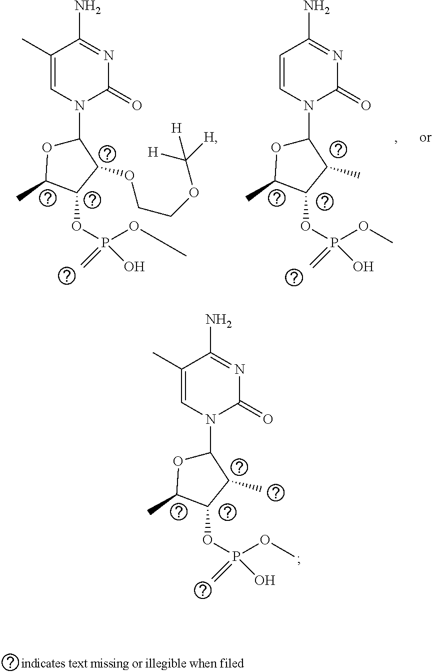
X₁₄ is 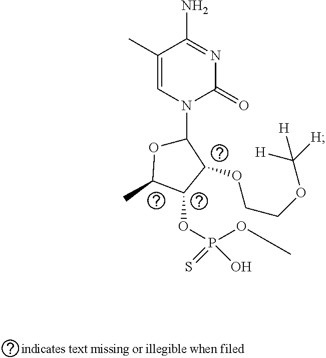
X₁₅ is 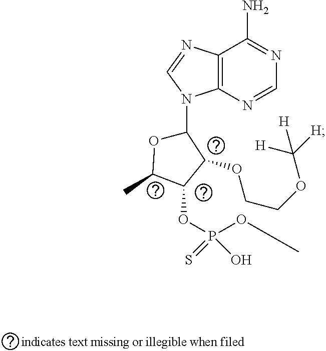
and X_{B} is 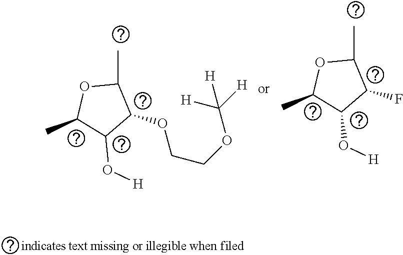
and B_{B} is

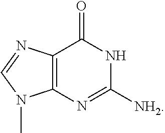

11. The compound of claim 10, wherein
X_{A} is 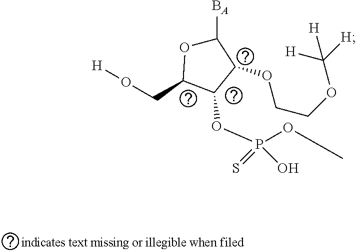
X₇ is 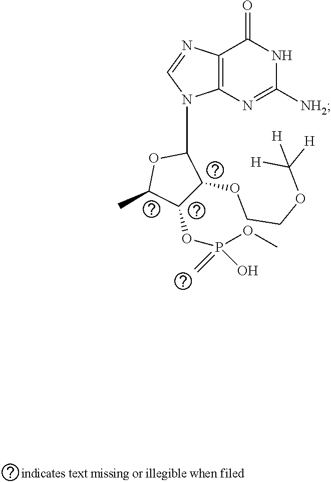 [SMILES: C=P(O)(OC)O[C@@H]1[C@@H](C)OC(n2cnc3c(=O)[nH]c(N)nc32)[C@@H]1OCCOC]
X₈ is 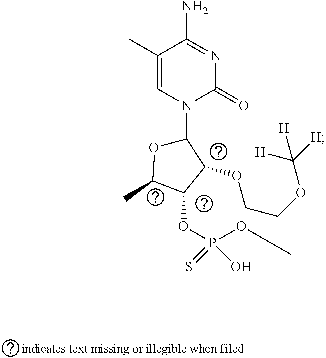
X₉ is 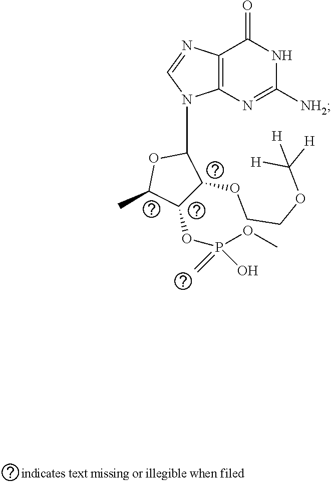 [SMILES: C=P(O)(OC)O[C@@H]1[C@@H](C)OC(n2cnc3c(=O)[nH]c(N)nc32)[C@@H]1OCCOC]
X₁₀ is 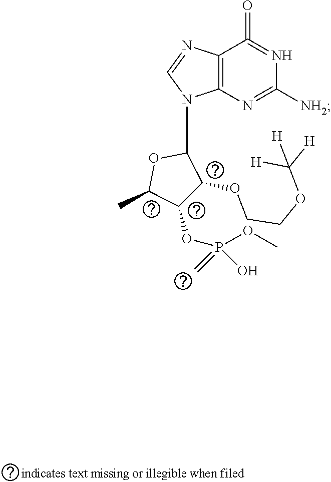
X₁₃ is 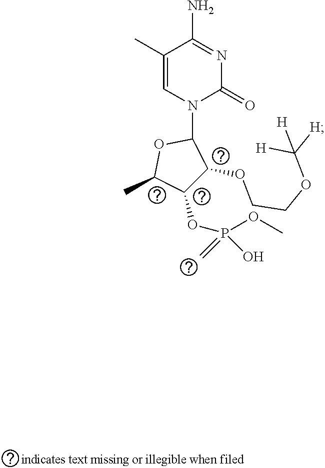
and X_{B} is

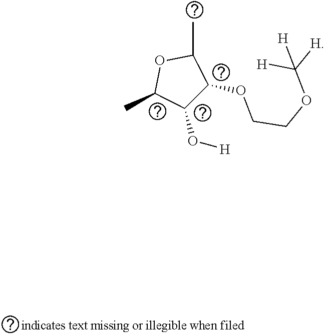

12. The compound of claim 10, wherein
X_{A} is 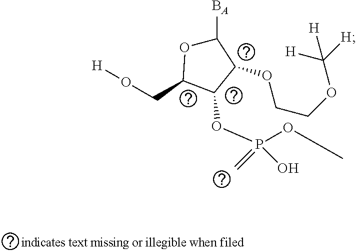
X₇ is 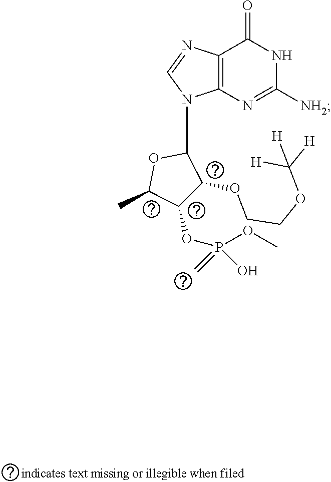 [SMILES: C=P(O)(OC)O[C@@H]1[C@@H](C)OC(n2cnc3c(=O)[nH]c(N)nc32)[C@@H]1OCCOC]
X₈ is 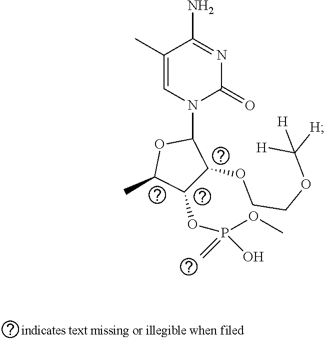
X₉ is 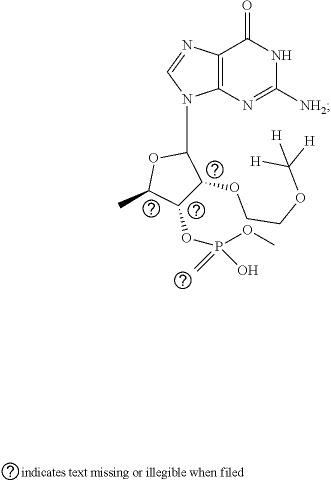 [SMILES: C=P(O)(OC)O[C@@H]1[C@@H](C)OC(n2cnc3c(=O)[nH]c(N)nc32)[C@@H]1OCCOC]
X₁₀ is 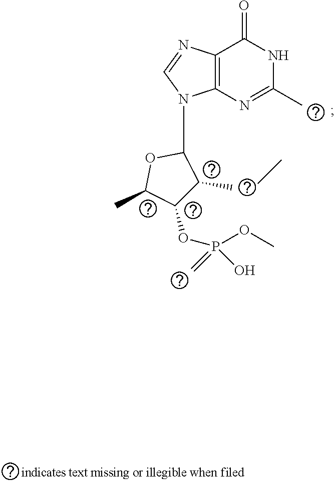
X₁₃ is 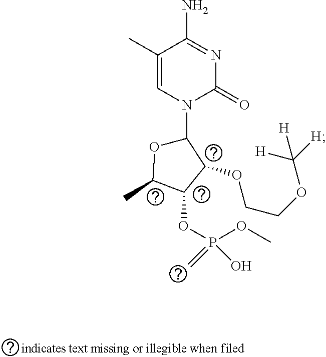
and X_{B} is

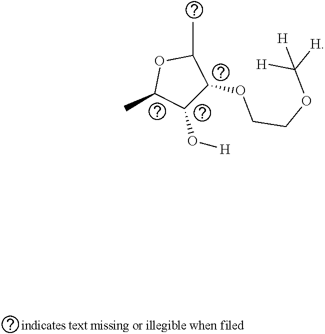

13. The compound of claim 10, wherein
X_{A} is 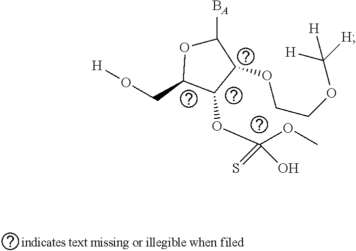
X₇ is 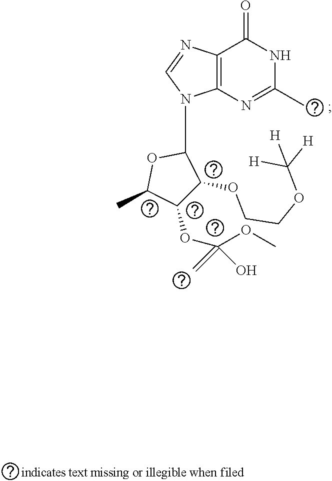
X₈ is 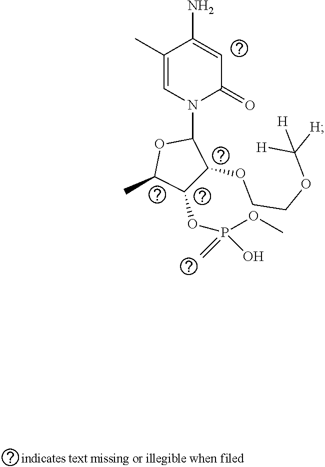
X₉ is 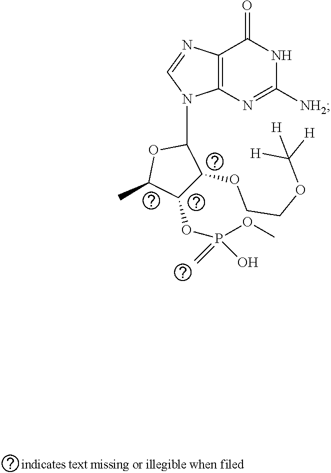 [SMILES: C=P(O)(OC)O[C@@H]1[C@@H](C)OC(n2cnc3c(=O)[nH]c(N)nc32)[C@@H]1OCCOC]
X₁₀ is 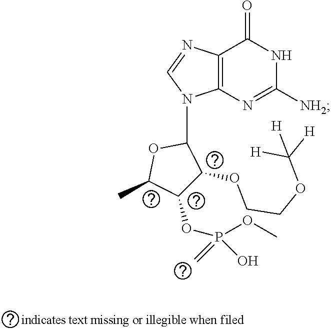 [SMILES: C=P(O)(OC)O[C@@H]1[C@@H](C)OC(n2cnc3c(=O)[nH]c(N)nc32)[C@@H]1OCCOC]
X₁₃ is 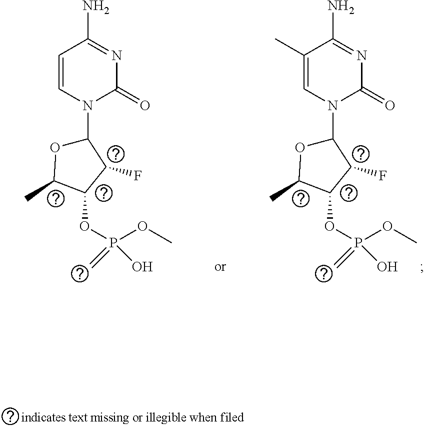
and X_{B} is

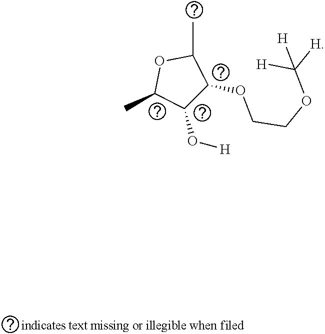

14. The compound of claim 10, wherein
X_{A} is 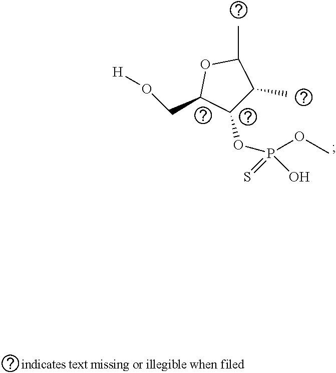
X₇ is 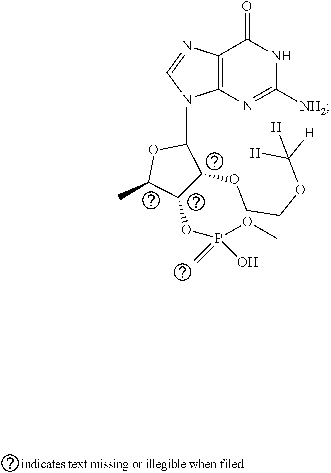 [SMILES: C=P(O)(OC)O[C@@H]1[C@@H](C)OC(n2cnc3c(=O)[nH]c(N)nc32)[C@@H]1OCCOC]
X₈ is 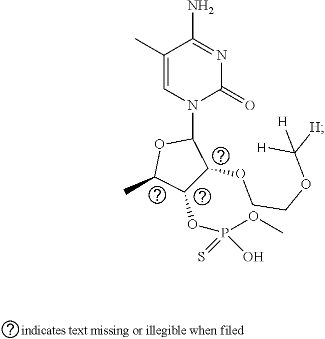
X₉ is 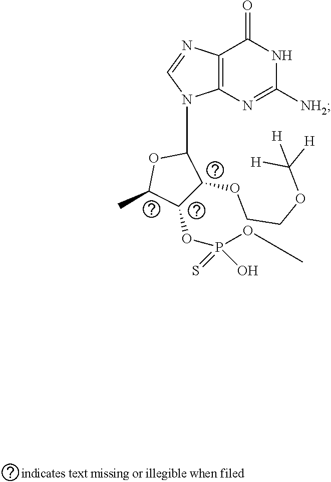 [SMILES: COCCO[C@H]1C(n2cnc3c(=O)[nH]c(N)nc32)O[C@H](C)[C@H]1OP(O)(=S)OC]
X₁₀ is 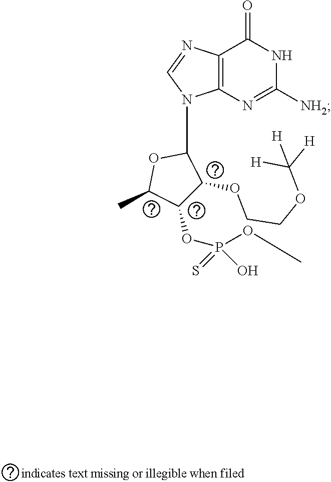 [SMILES: COCCO[C@H]1C(n2cnc3c(=O)[nH]c(N)nc32)O[C@H](C)[C@H]1OP(O)(=S)OC]
X₁₃ is 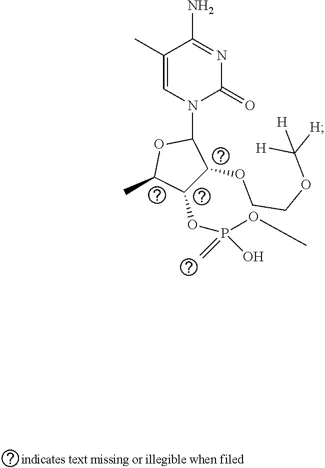
and X_{B} is

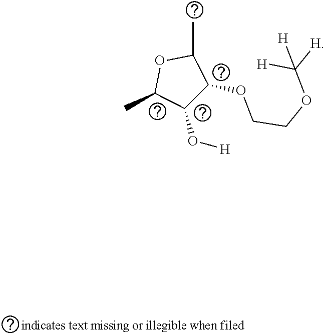

15. The compound of claim 10, wherein
X_{A} is 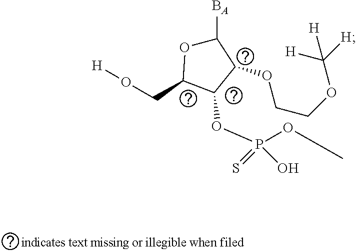
X₇ is 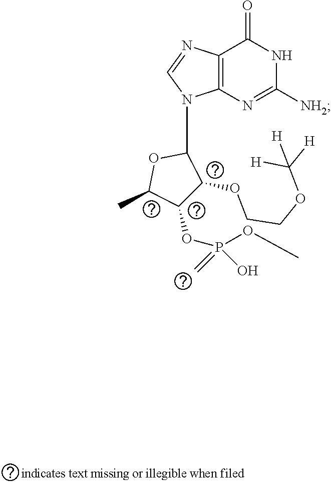 [SMILES: C=P(O)(OC)O[C@@H]1[C@@H](C)OC(n2cnc3c(=O)[nH]c(N)nc32)[C@@H]1OCCOC]
X₈ is 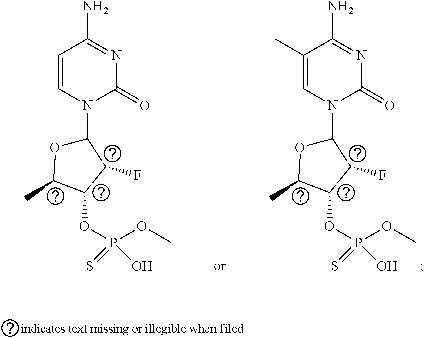
X₉ is 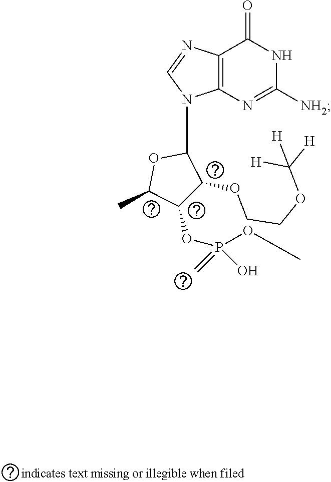 [SMILES: C=P(O)(OC)O[C@@H]1[C@@H](C)OC(n2cnc3c(=O)[nH]c(N)nc32)[C@@H]1OCCOC]
X₁₀ is 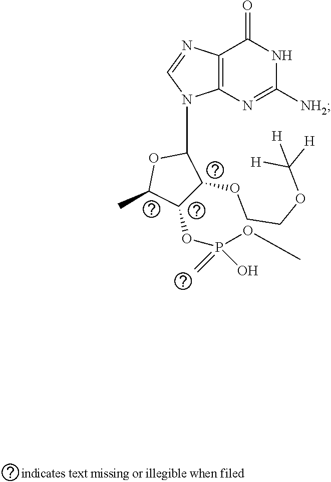 [SMILES: C=P(O)(OC)O[C@@H]1[C@@H](C)OC(n2cnc3c(=O)[nH]c(N)nc32)[C@@H]1OCCOC]
X₁₃ is 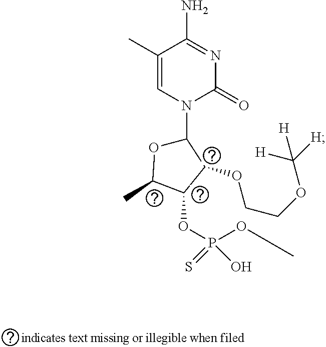
and X_{B} is

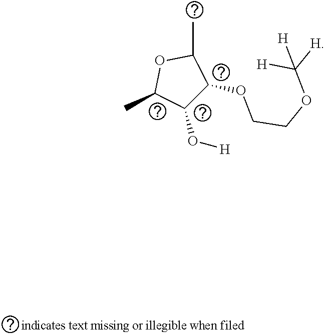

16. The compound of claim 10, wherein
X_{A} is 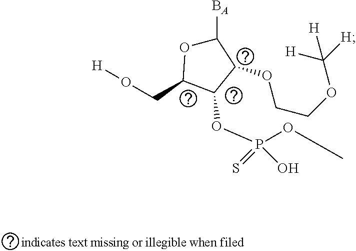
X₇ is 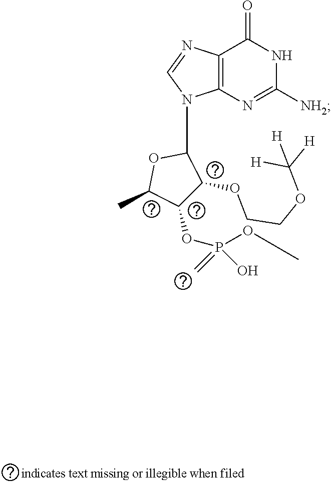 [SMILES: C=P(O)(OC)O[C@@H]1[C@@H](C)OC(n2cnc3c(=O)[nH]c(N)nc32)[C@@H]1OCCOC]
X₈ is 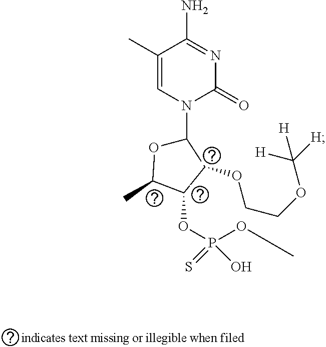
X₉ is 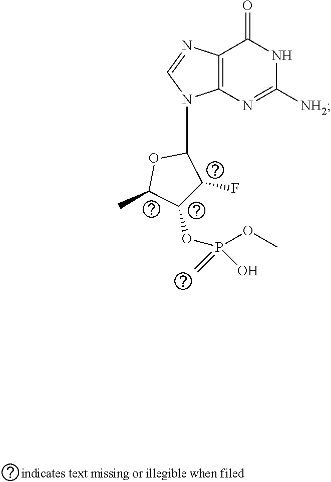 [SMILES: C=P(O)(OC)O[C@@H]1[C@@H](C)OC(n2cnc3c(=O)[nH]c(N)nc32)[C@@H]1F]
X₁₀ is 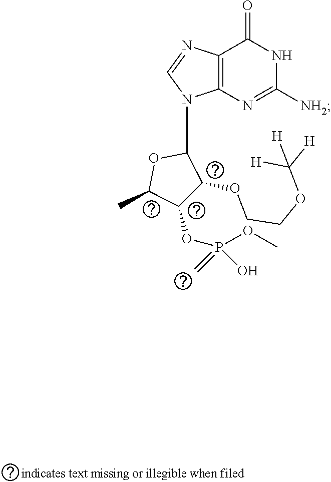 [SMILES: C=P(O)(OC)O[C@@H]1[C@@H](C)OC(n2cnc3c(=O)[nH]c(N)nc32)[C@@H]1OCCOC]
X₁₃ is 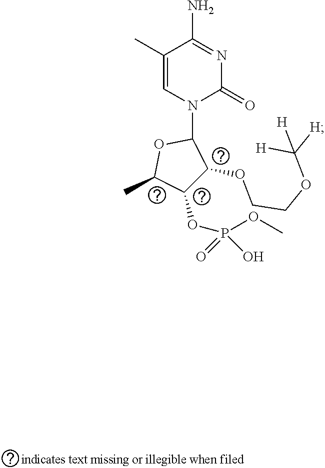
and X_{B} is

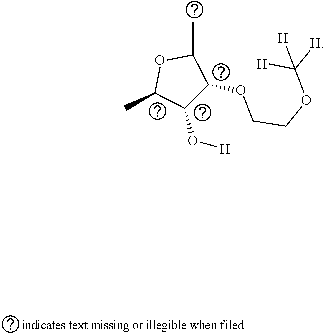

17. The compound of claim 10, wherein
X_{A} is 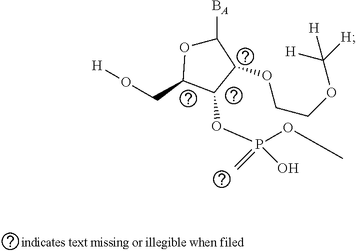
X₇ is 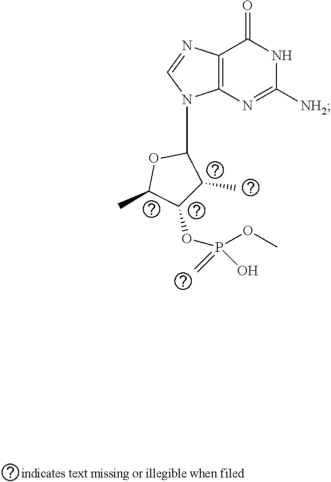 [SMILES: C=P(O)(OC)O[C@@H]1[C@@H](C)OC(n2cnc3c(=O)[nH]c(N)nc32)[C@@H]1C]
X₈ is 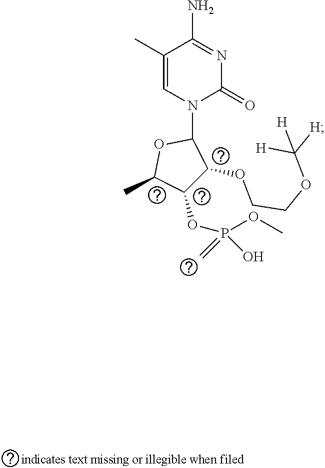
X₉ is 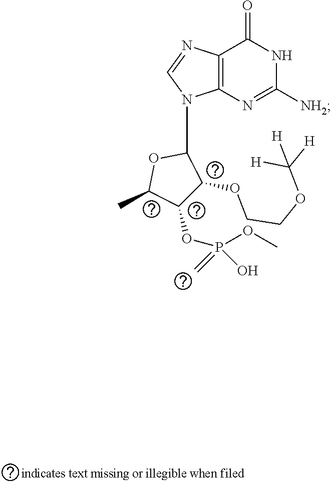 [SMILES: C=P(O)(OC)O[C@@H]1[C@@H](C)OC(n2cnc3c(=O)[nH]c(N)nc32)[C@@H]1OCCOC]
X₁₀ is 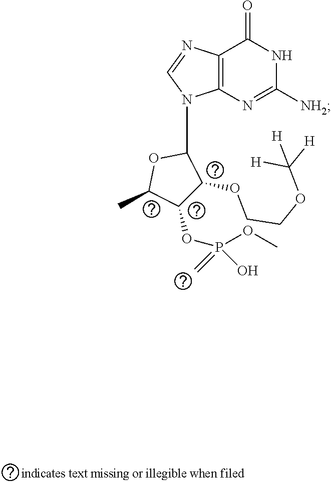 [SMILES: C=P(O)(OC)O[C@@H]1[C@@H](C)OC(n2cnc3c(=O)[nH]c(N)nc32)[C@@H]1OCCOC]
X₁₃ is 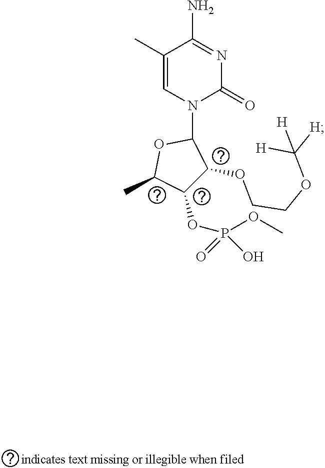
and X_{B} is

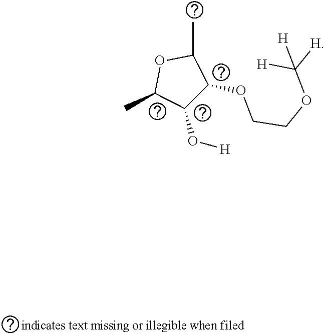

18. The compound of claim 10, wherein
X_{A} is 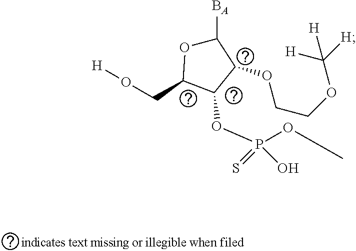
X₇ is 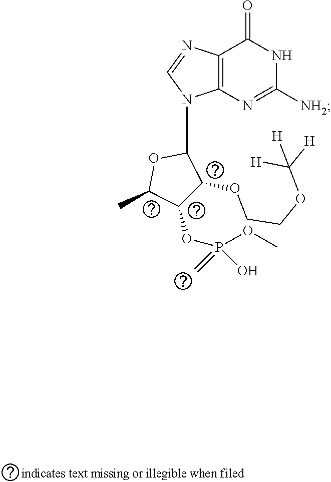 [SMILES: C=P(O)(OC)O[C@@H]1[C@@H](C)OC(n2cnc3c(=O)[nH]c(N)nc32)[C@@H]1OCCOC]
X₈ is 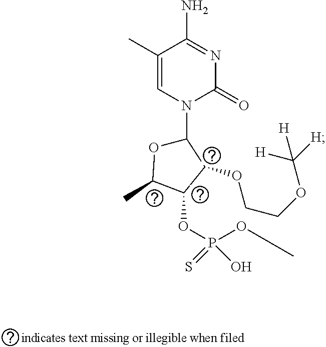
X₉ is 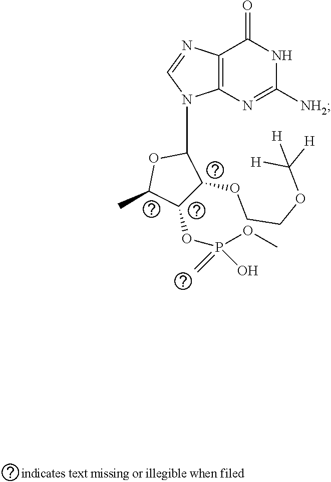 [SMILES: C=P(O)(OC)O[C@@H]1[C@@H](C)OC(n2cnc3c(=O)[nH]c(N)nc32)[C@@H]1OCCOC]
X₁₀ is 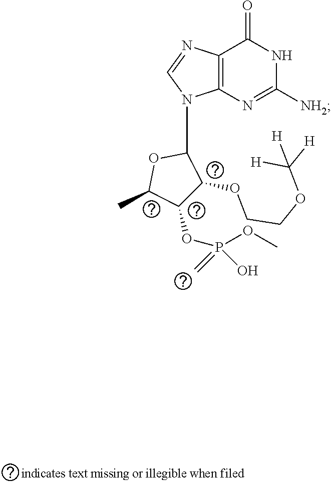 [SMILES: C=P(O)(OC)O[C@@H]1[C@@H](C)OC(n2cnc3c(=O)[nH]c(N)nc32)[C@@H]1OCCOC]
X₁₃ is 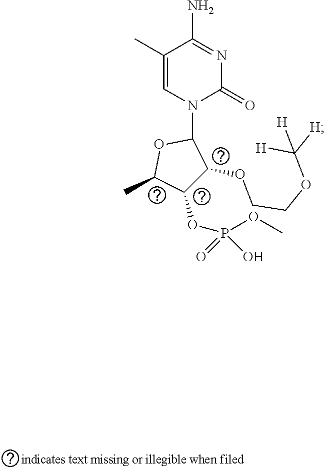
and X_{B} is

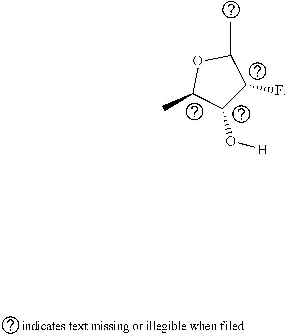

19. A compound of Formula (I):
(SEQ ID NO: 3)
X_{A}X₂X₃X₄X₅X₆X₇X₈X₉X₁₀X₁₁X₁₂X₁₃X₁₄X₁₅X_{B}
wherein
X_{A} is 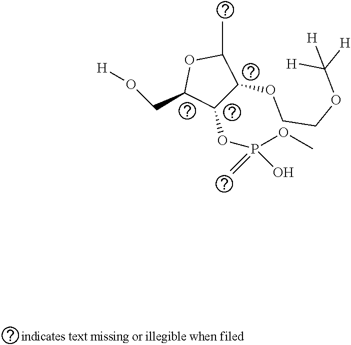 [SMILES: C=P(O)(OC)O[C@@H]1[C@@H](CO)OC(C)[C@@H]1OCCOC]
and B_{A} is 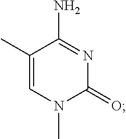
X₂ is 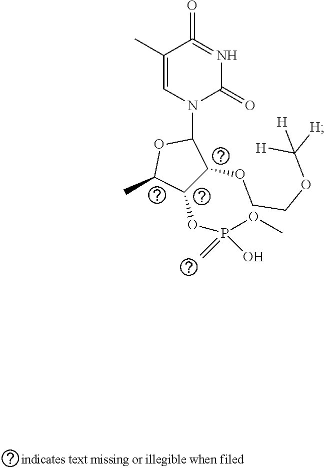
X₃ is 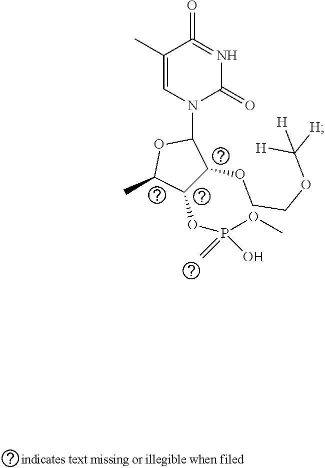
X₄ is 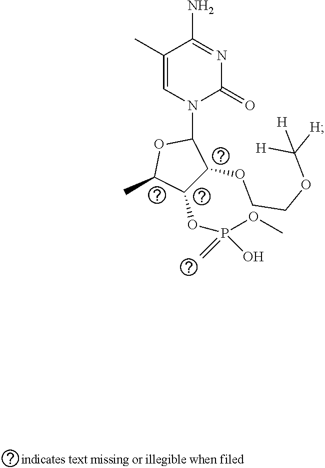
X₅ is 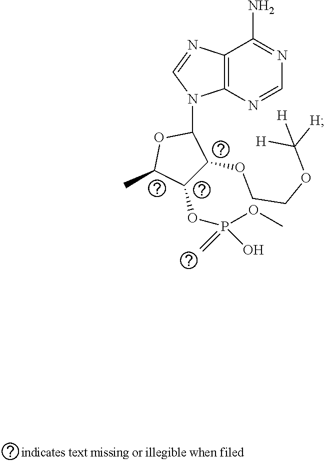
X₆ is 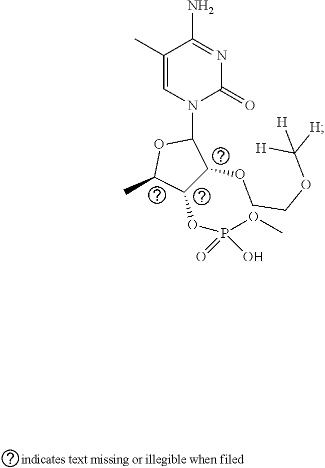
X₇ is 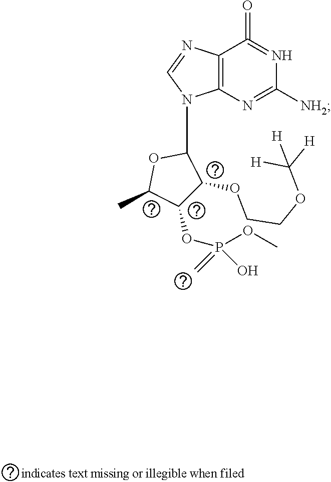
X₈ is 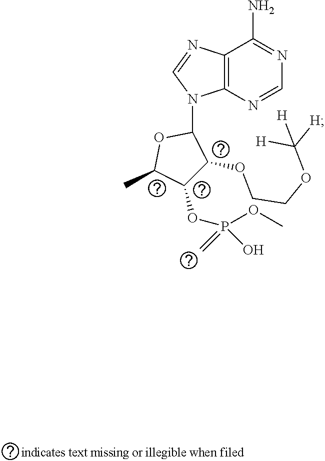
X₉ is 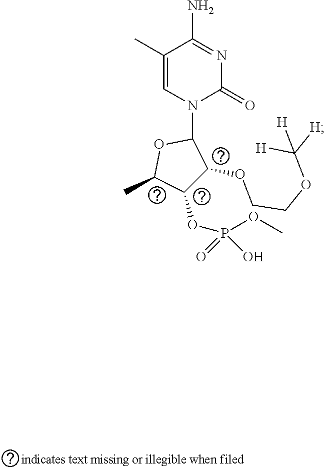
X₁₀ is 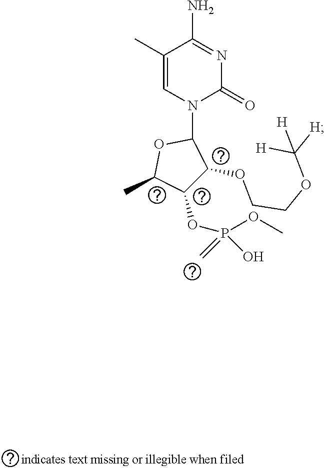
X₁₁ is 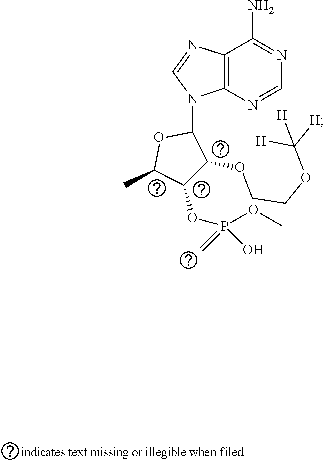
X₁₂ is 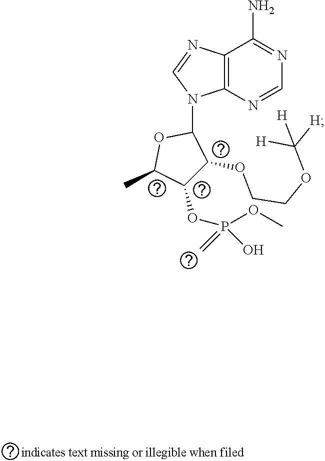
X₁₃ is 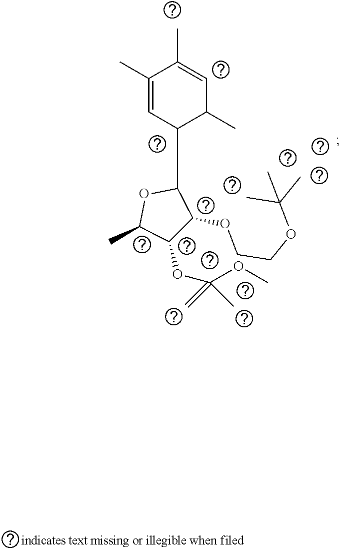
or absent; X₁₄ is 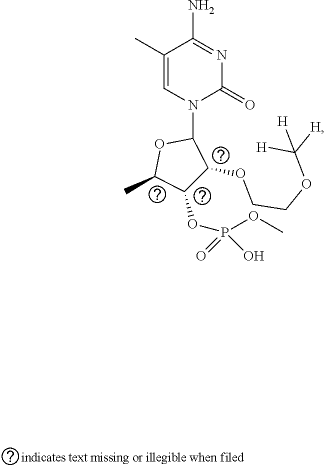
or absent;
X₁₅ is 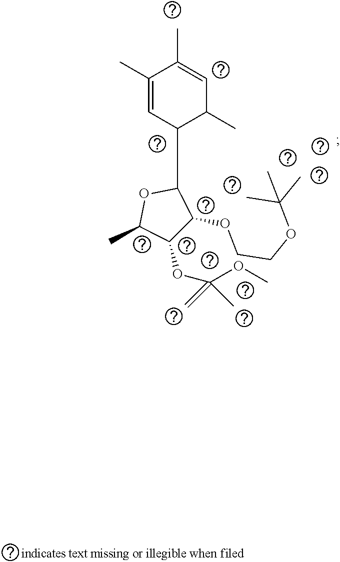
or absent; and X_{B} is 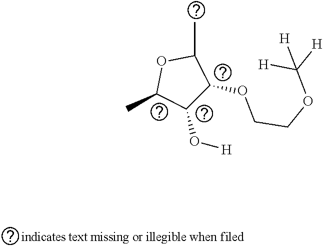
and B_{B} is 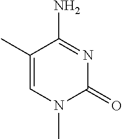
when each of X₁₃ X₁₄ and X₁₅ is absent, 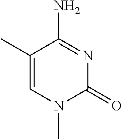
when X₁₃ is 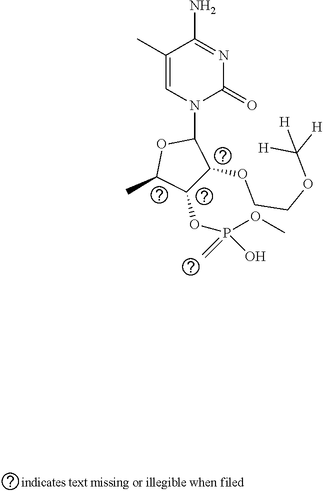 [SMILES: C=P(O)(OC)O[C@@H]1[C@@H](C)OC(n2cc(C)c(N)nc2=O)[C@@H]1OCCOC]
and each of X₁₄ and X₁₅ is absent, 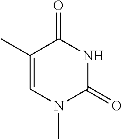
when X₁₃ is 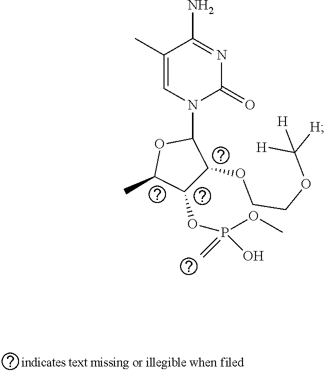
X₁₄ is 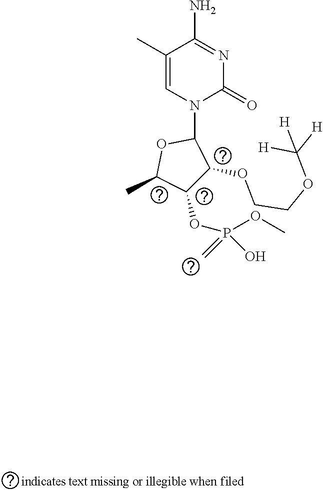 [SMILES: C=P(O)(OC)O[C@@H]1[C@@H](C)OC(n2cc(C)c(N)nc2=O)[C@@H]1OCCOC]
and X₁₅ is absent, or 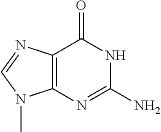
when X₁₃ is 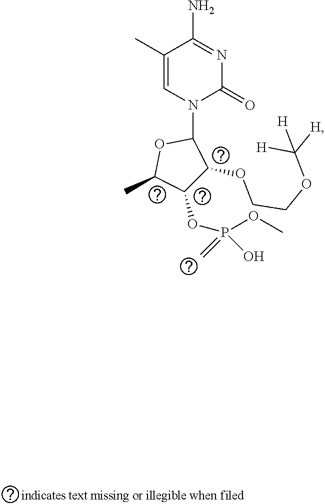
X₁₄ is 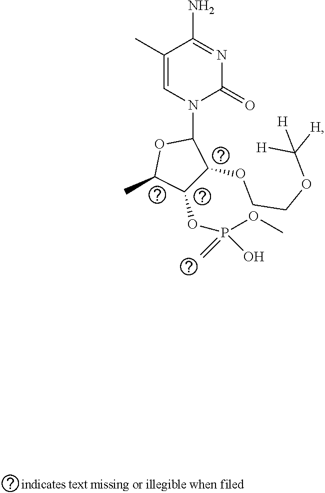
and
X₁₅ is

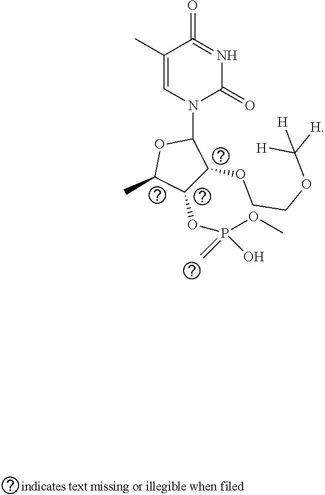

20. The compound of claim 19, wherein:
X₁₃ is 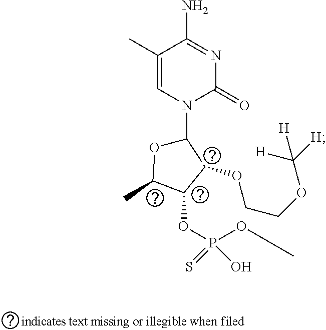
X₁₄ is 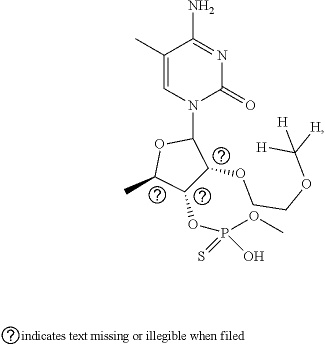
and X₁₅ is

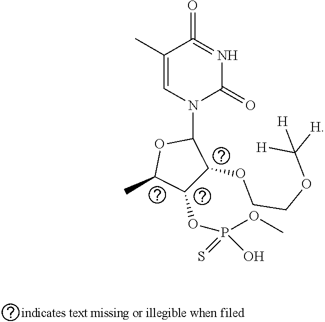

21. The compound of claim 19, wherein:
X₁₃ is 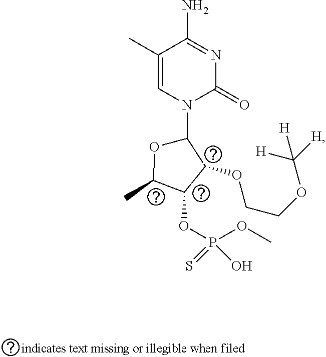
X₁₄ is 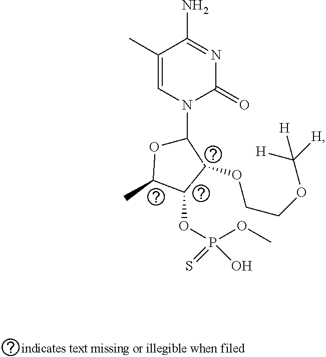
and X₁₅ is absent.

22. The compound of claim 19, wherein:
X₁₃ is 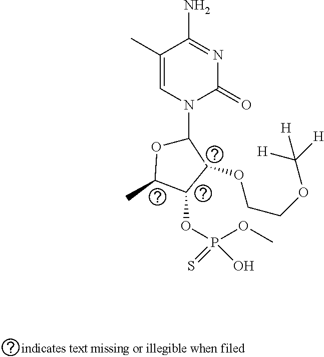 [SMILES: COCCO[C@H]1C(n2cc(C)c(N)nc2=O)O[C@H](C)[C@H]1OP(O)(=S)OC]
and each of X₁₄ and X₁₅ is absent.

23. The compound of claim 19, wherein each of X₁₃, X₁₄, and X₁₅ is absent.

24. A compound of Formula (I):
(SEQ ID NO: 4)
X_{A}X₂X₃X₄X₅X₆X₇X₈X₉X₁₀X₁₁X₁₂X₁₃X₁₄X₁₅X_{B}
wherein
X_{A} is 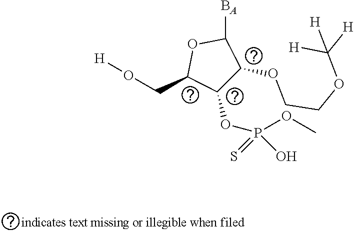
and B_{A} is 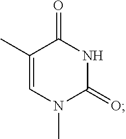
X₂ is 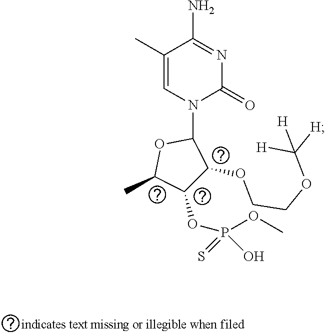
X₃ is 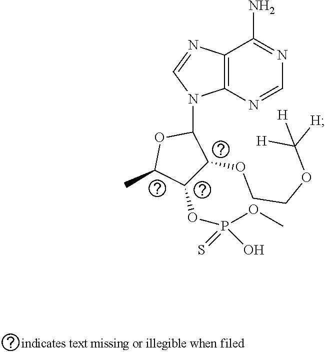
X₄ is 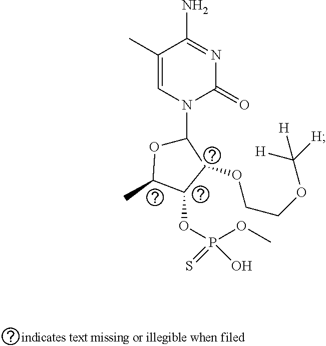
X₅ is 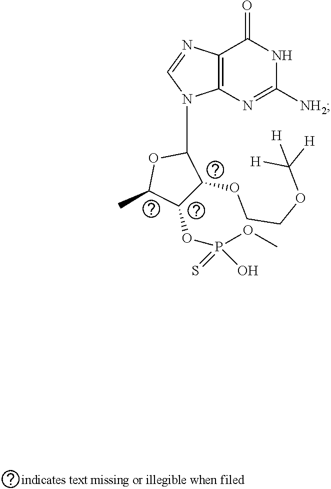 [SMILES: COCCO[C@H]1C(n2cnc3c(=O)[nH]c(N)nc32)O[C@H](C)[C@H]1OP(O)(=S)OC]
X₆ is 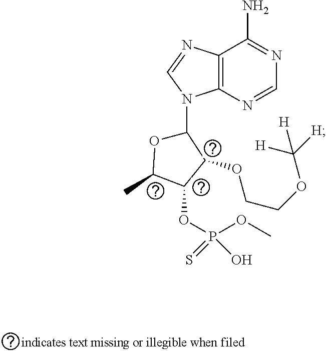
X₇ is 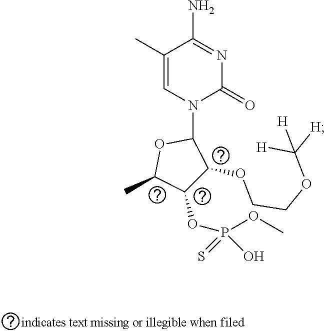
X₈ is 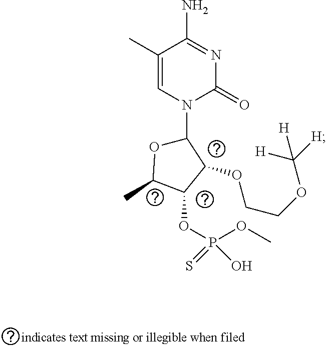
X₉ is 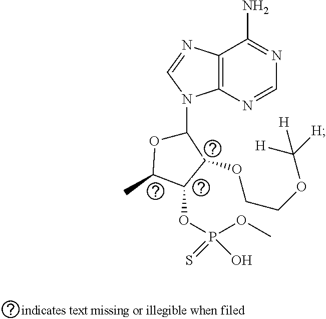
X₁₀ is 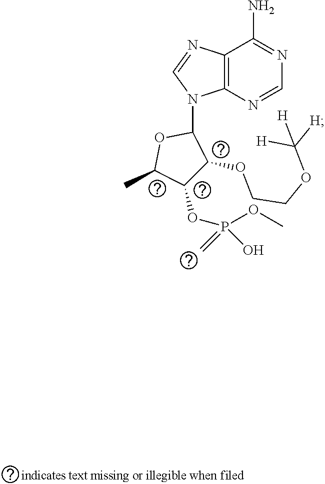
X₁₁ is 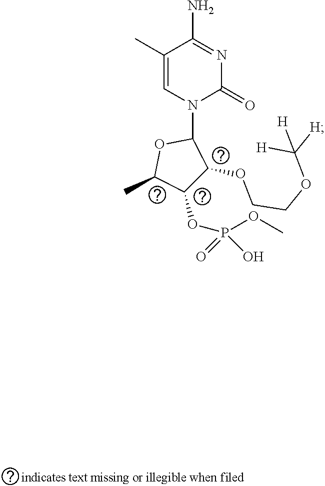
X₁₂ is 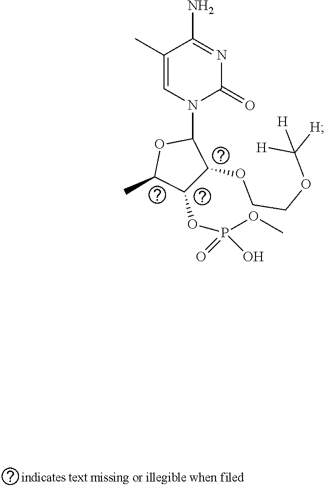
X₁₃ is 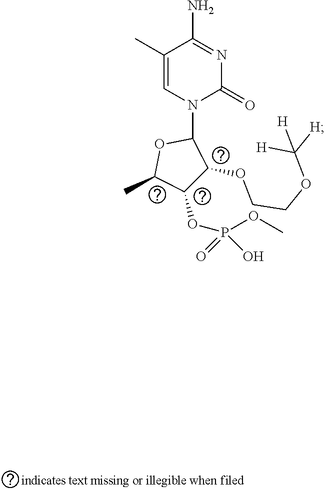
and X₁₄ is 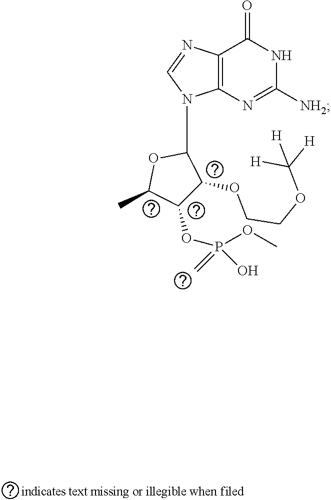 [SMILES: C=P(O)(OC)O[C@@H]1[C@@H](C)OC(n2cnc3c(=O)[nH]c(N)nc32)[C@@H]1OCCOC]
X₁₅ is 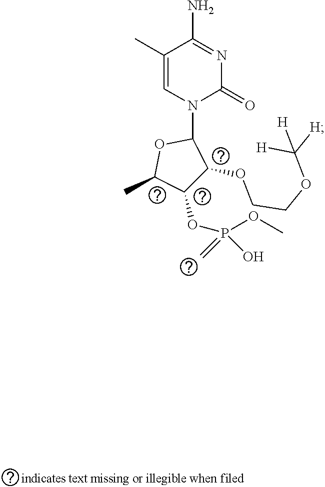
X_{B} is 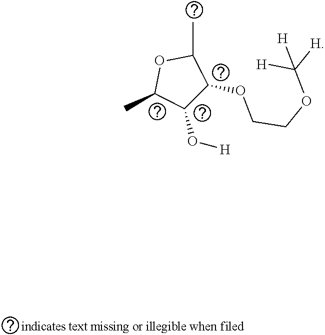
and B_{B} is

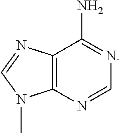

25. A compound of Formula (I):
(SEQ ID NO: 5)
X_{A}X₂X₃X₄X₅X₆X₇X₈X₉X₁₀X₁₁X₁₂X₁₃X₁₄X_{B}
wherein:
X_{A} is 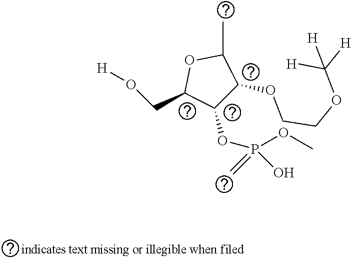 [SMILES: C=P(O)(OC)O[C@@H]1[C@@H](CO)OC(C)[C@@H]1OCCOC]
and B_{A} is 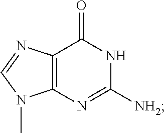
X₂ is 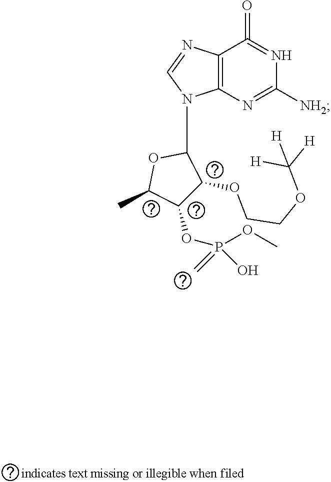
X₃ is 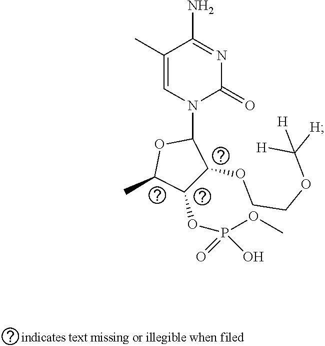
X₄ is 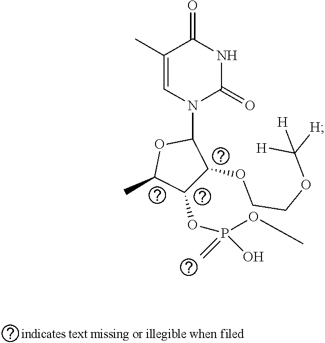
X₅ is 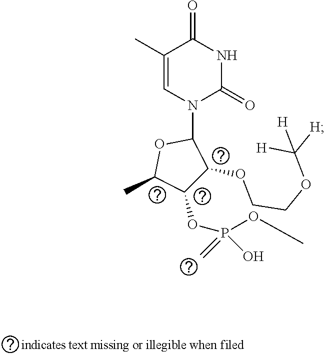
X₆ is 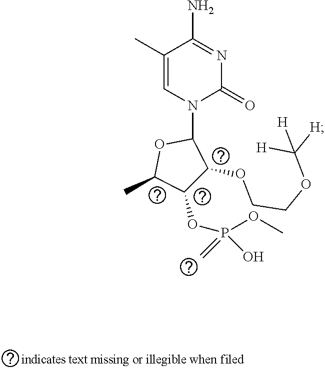
X₇ is 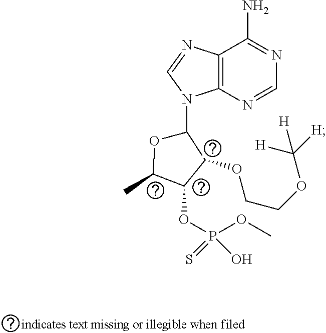
X₈ is 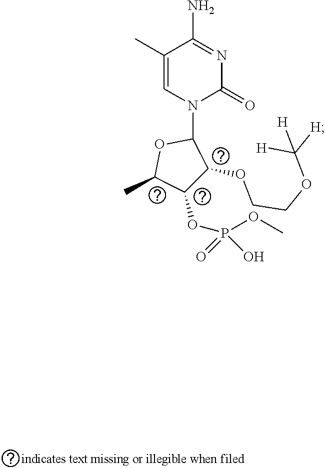
X₉ is 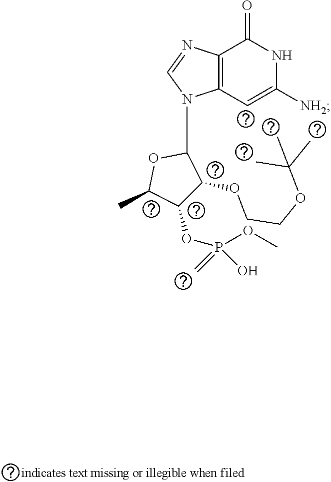 [SMILES: C=P(O)(OC)O[C@@H]1[C@@H](C)OC(n2cnc3c(=O)[nH]c(N)cc32)[C@@H]1OCCOC(C)(C)C]
X₁₀ is 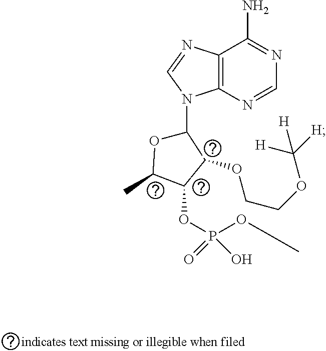
X₁₁ is 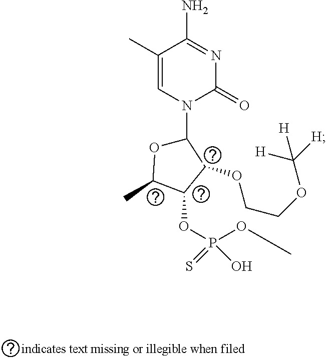
X₁₂ is 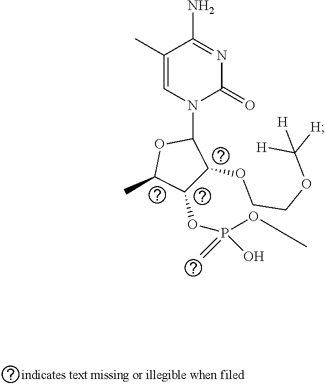
X₁₃ is 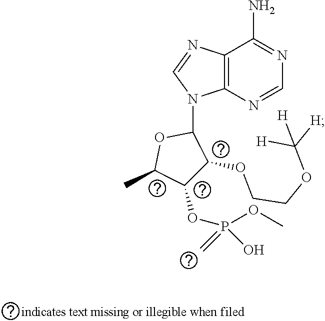
X₁₄ is 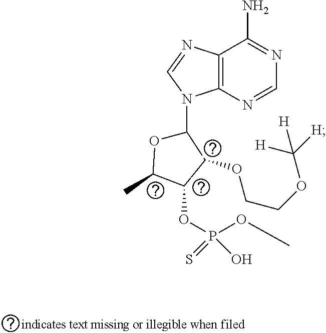
and
X_{B} is 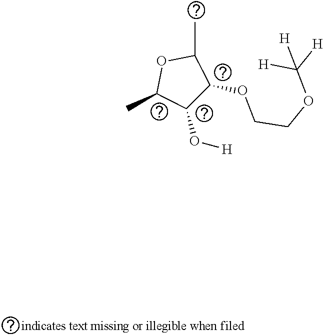
and B_{B} is

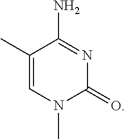

26. A compound of Formula (I):
(SEQ ID NO: 6)
X_{A}X₂X₃X₄X₅X₆X₇X₈X₉X₁₀X₁₁X₁₂X₁₃X_{B}
wherein
X_{A} is 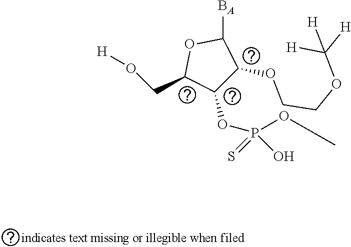
and B_{A} is 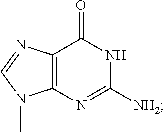
X₂ is 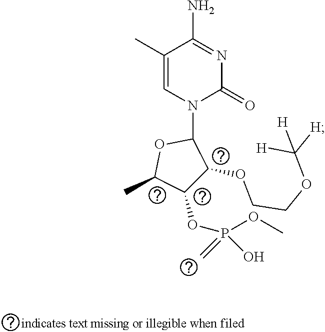
X₃ is 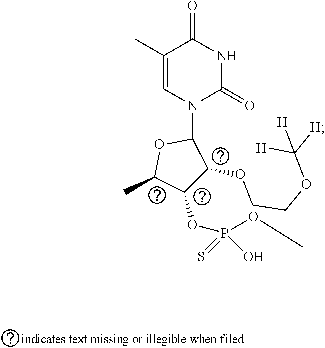
X₄ is 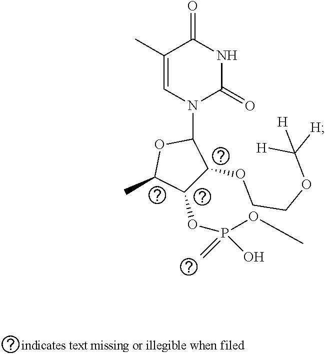
X₅ is 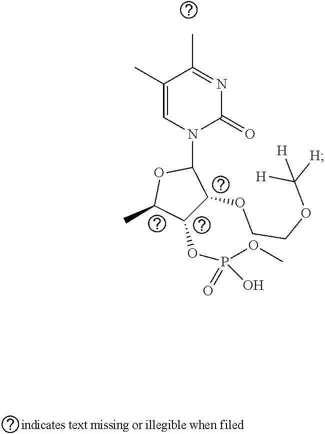
X₆ is 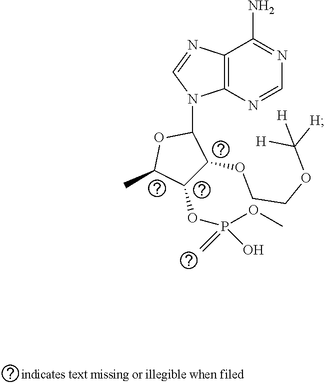
X₇ is 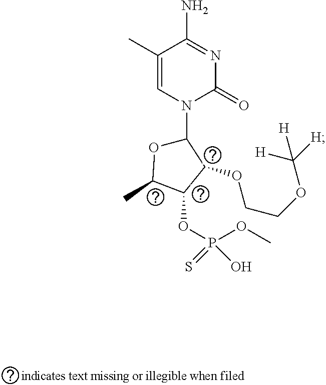
X₈ is 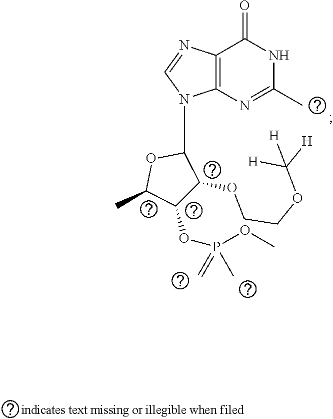 [SMILES: C=P(C)(OC)O[C@@H]1[C@@H](C)OC(n2cnc3c(=O)[nH]c(C)nc32)[C@@H]1OCCOC]
X₉ is 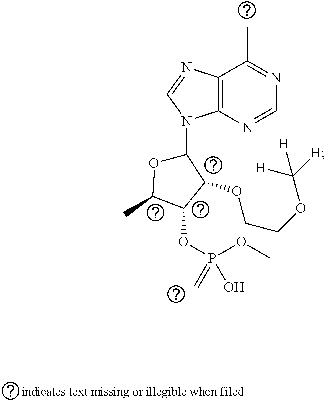
X₁₀ is 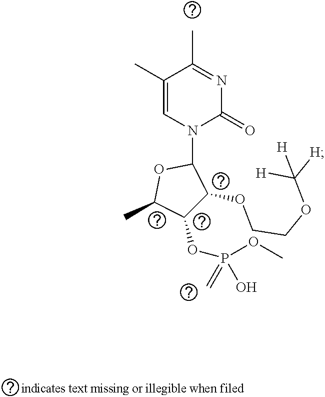
X₁₁ is 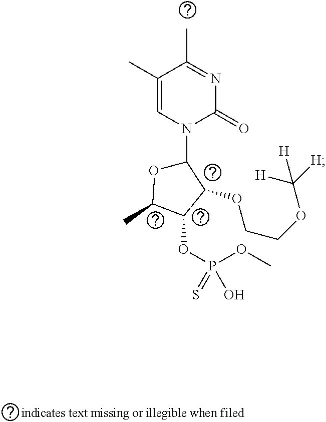
X₁₂ is 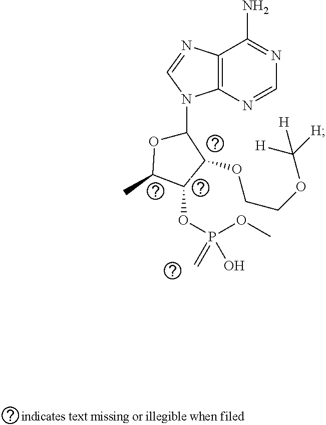
X₁₃ is 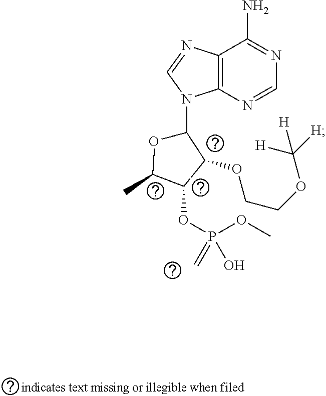
and X_{B} is 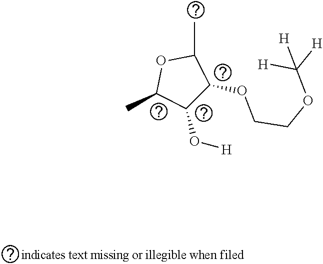
and B_{B} is

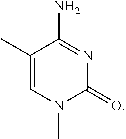

27. A compound of Formula (I): 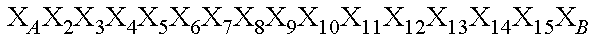
wherein
X_{A} is 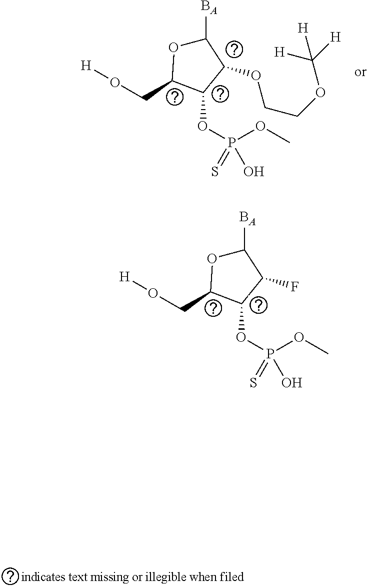
and B_{A} is 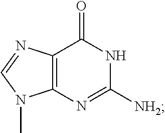
X₂ is 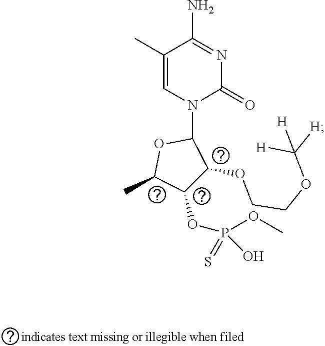
X₃ is 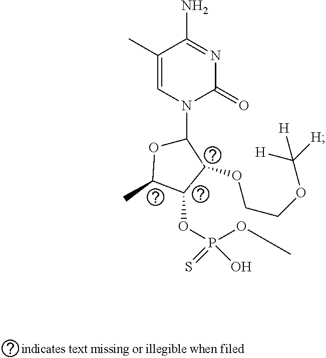
X₄ is 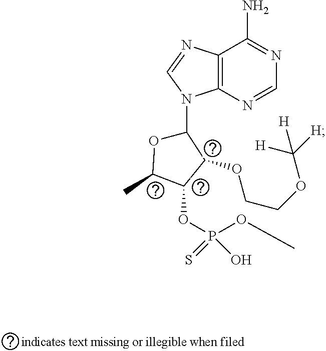
X₅ is 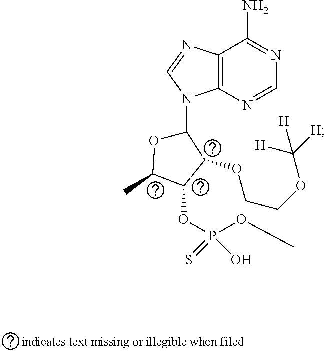
X₆ is 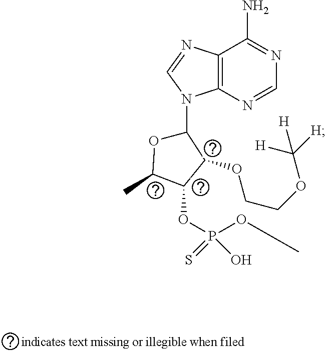
X₇ is 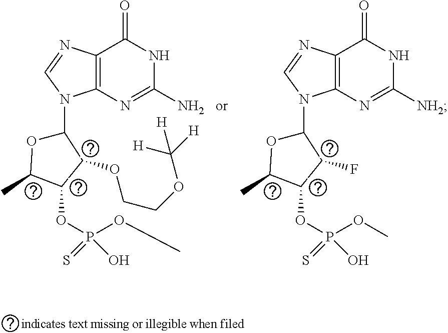 [SMILES: COCCO[C@H]1C(n2cnc3c(=O)[nH]c(N)nc32)O[C@H](C)[C@H]1OP(O)(=S)OC ; COP(O)(=S)O[C@@H]1[C@@H](C)OC(n2cnc3c(=O)[nH]c(N)nc32)[C@@H]1F]
X₈ is 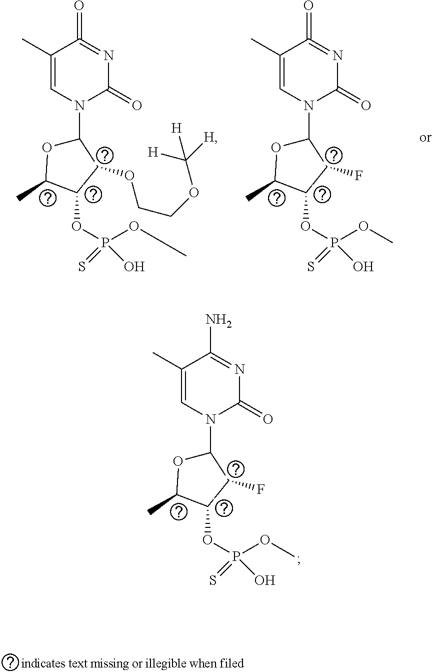
X₉ is 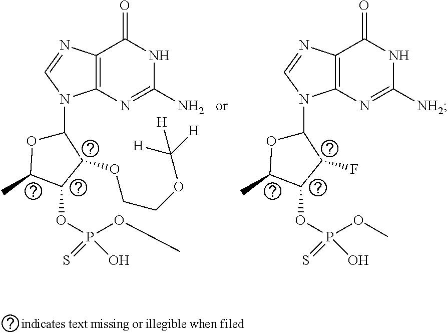 [SMILES: COCCO[C@H]1C(n2cnc3c(=O)[nH]c(N)nc32)O[C@H](C)[C@H]1OP(O)(=S)OC ; COP(O)(=S)O[C@@H]1[C@@H](C)OC(n2cnc3c(=O)[nH]c(N)nc32)[C@@H]1F]
X₁₀ is 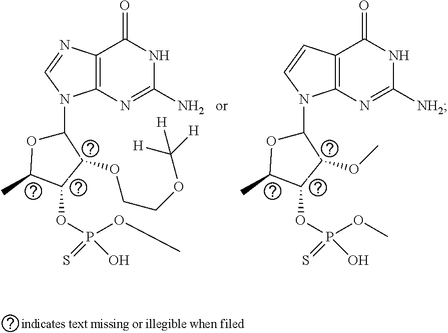 [SMILES: COCCO[C@H]1C(n2cnc3c(=O)[nH]c(N)nc32)O[C@H](C)[C@H]1OP(O)(=S)OC ; CO[C@H]1C(n2ccc3c(=O)[nH]c(N)nc32)O[C@H](C)[C@H]1OP(O)(=S)OC]
X₁₁ is 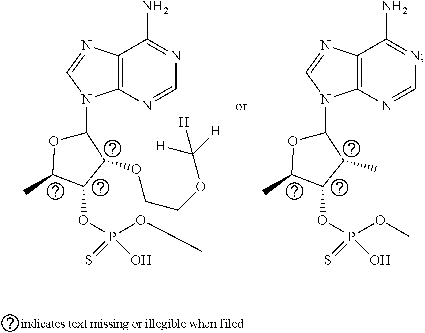 [SMILES: COCCO[C@H]1C(n2cnc3c(N)ncnc32)O[C@H](C)[C@H]1OP(O)(=S)OC ; COP(O)(=S)O[C@@H]1[C@@H](C)OC(n2cnc3c(N)ncnc32)[C@@H]1C]
X₁₂ is 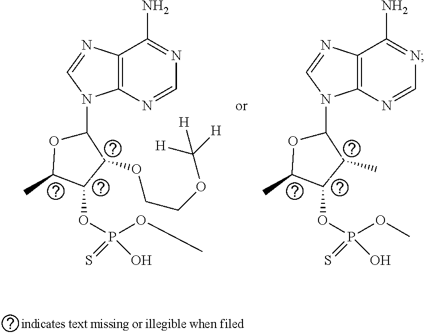 [SMILES: COCCO[C@H]1C(n2cnc3c(N)ncnc32)O[C@H](C)[C@H]1OP(O)(=S)OC ; COP(O)(=S)O[C@@H]1[C@@H](C)OC(n2cnc3c(N)ncnc32)[C@@H]1C]
X₁₃ is 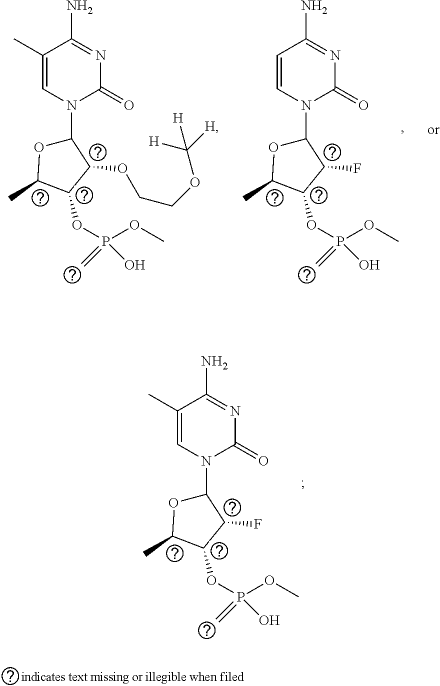
and X₁₄ is 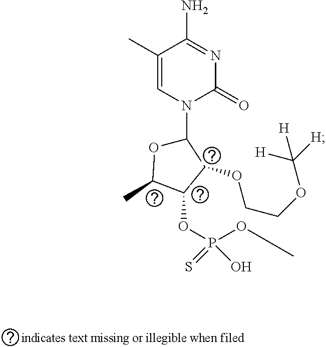
X₁₅ is 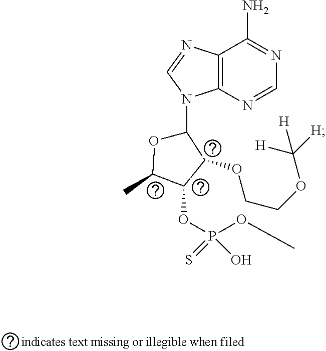
and
X_{B} is 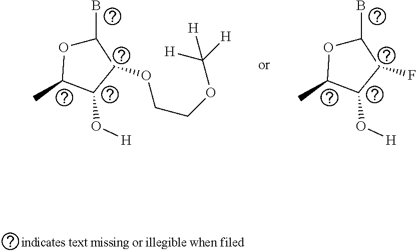
and B_{B} is

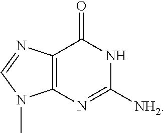

28. The compound of claim 27, wherein
X_{A} is 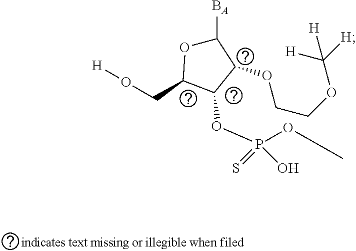
X₇ is 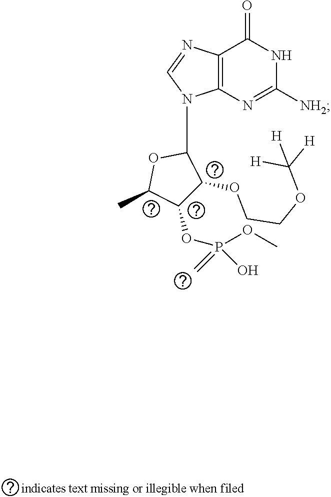 [SMILES: C=P(O)(OC)O[C@@H]1[C@@H](C)OC(n2cnc3c(=O)[nH]c(N)nc32)[C@@H]1OCCOC]
X₈ is 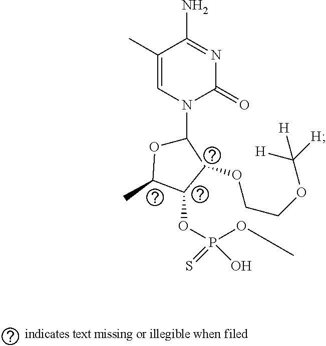
X₉ is 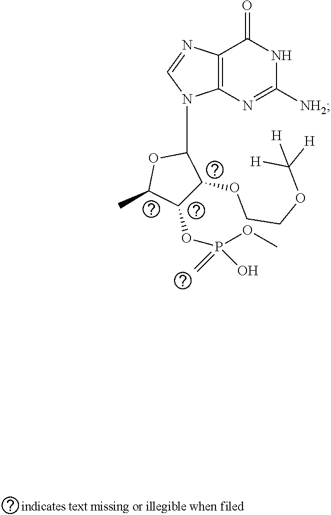 [SMILES: C=P(O)(OC)O[C@@H]1[C@@H](C)OC(n2cnc3c(=O)[nH]c(N)nc32)[C@@H]1OCCOC]
X₁₀ is 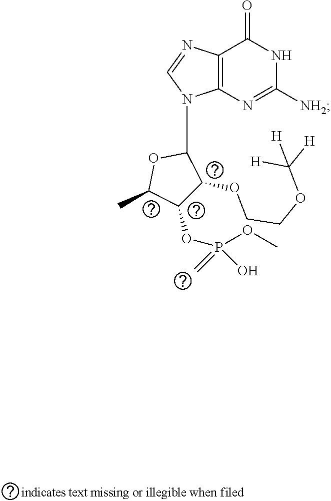 [SMILES: C=P(O)(OC)O[C@@H]1[C@@H](C)OC(n2cnc3c(=O)[nH]c(N)nc32)[C@@H]1OCCOC]
X₁₂ is 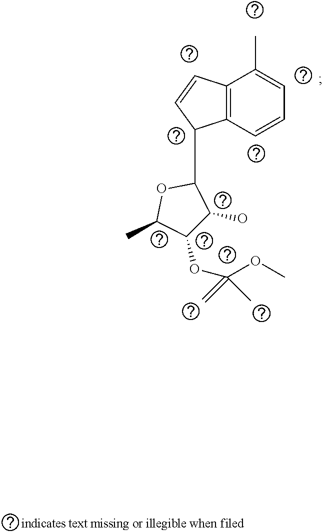
X₁₃ is 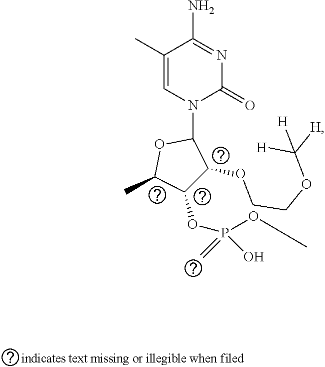
and X_{B} is

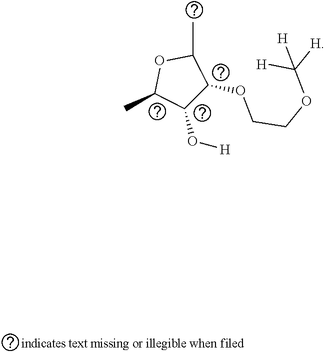

29. The compound of claim 27, wherein
X_{A} is 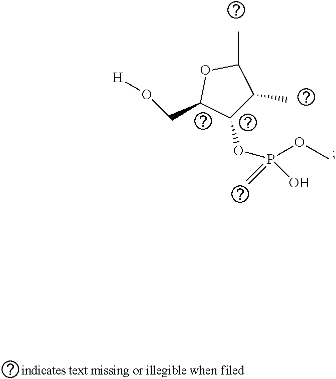
X₇ is 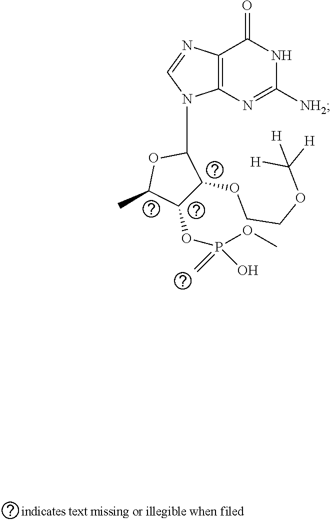 [SMILES: C=P(O)(OC)O[C@@H]1[C@@H](C)OC(n2cnc3c(=O)[nH]c(N)nc32)[C@@H]1OCCOC]
X₈ is 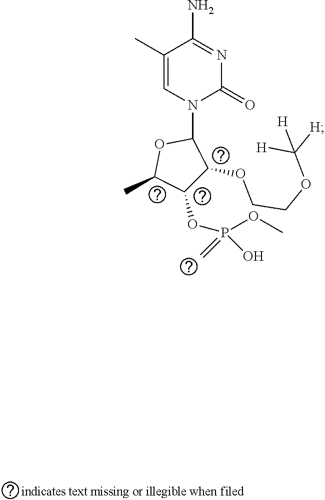
X₉ is 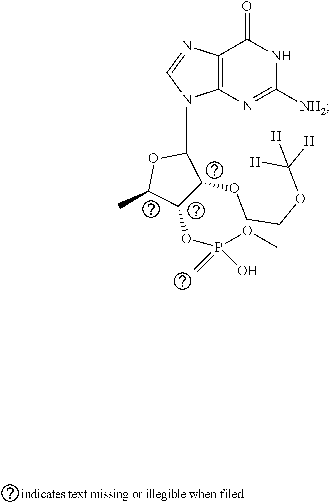 [SMILES: C=P(O)(OC)O[C@@H]1[C@@H](C)OC(n2cnc3c(=O)[nH]c(N)nc32)[C@@H]1OCCOC]
X₁₀ is 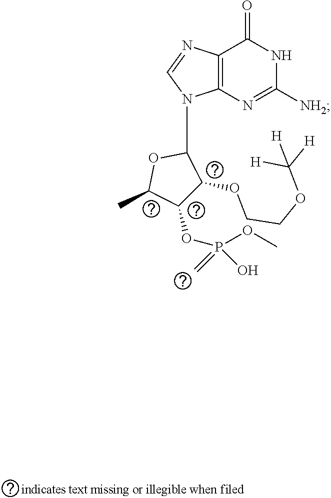 [SMILES: C=P(O)(OC)O[C@@H]1[C@@H](C)OC(n2cnc3c(=O)[nH]c(N)nc32)[C@@H]1OCCOC]
X₁₂ is 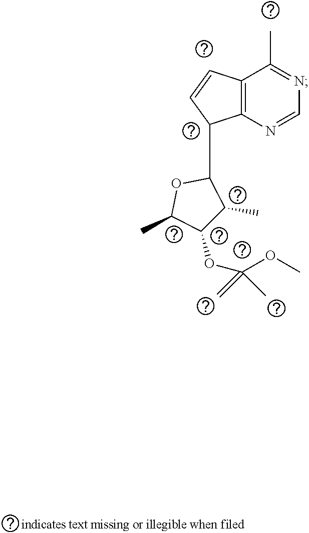
X₁₃ is 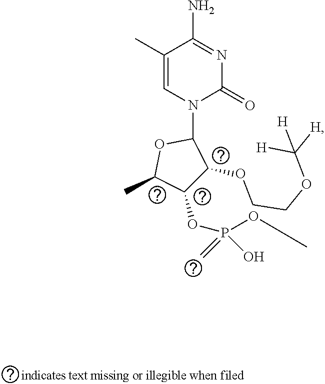
and X_{B} is

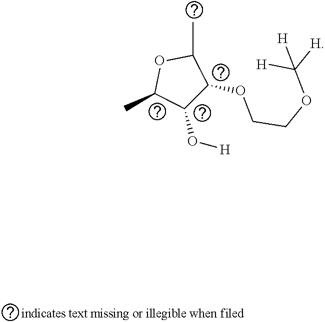

30. A compound selected from the group consisting of (SEQ ID NOs: 7-16, 1, 18-25, and 25-29 disclosed below, respectively, in order of appearance): 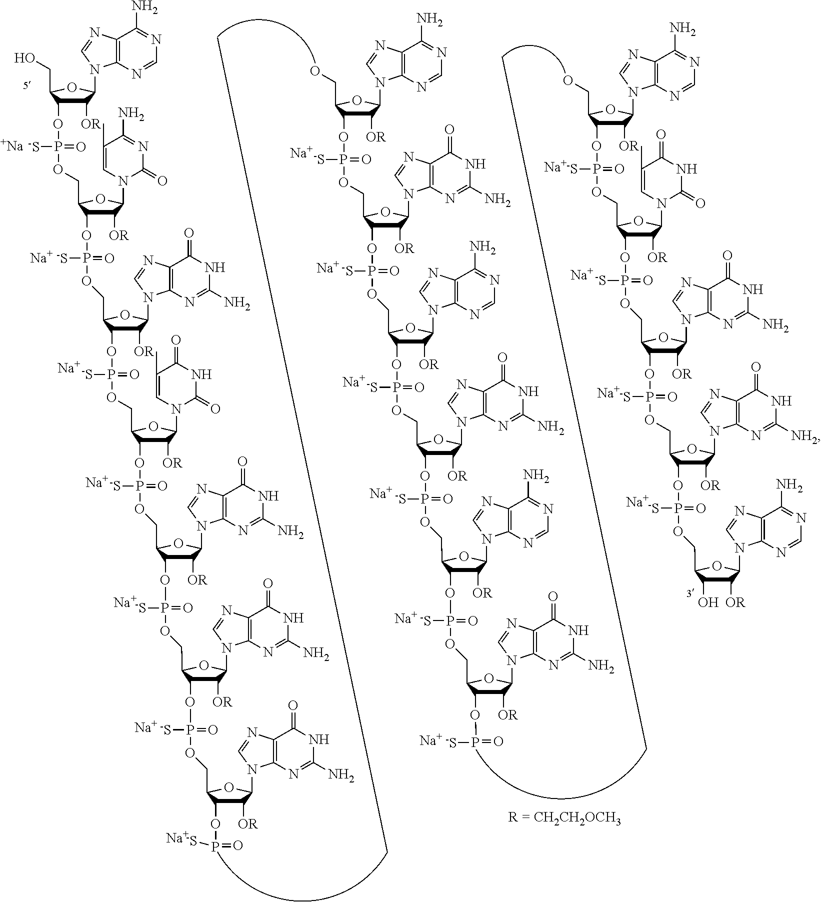 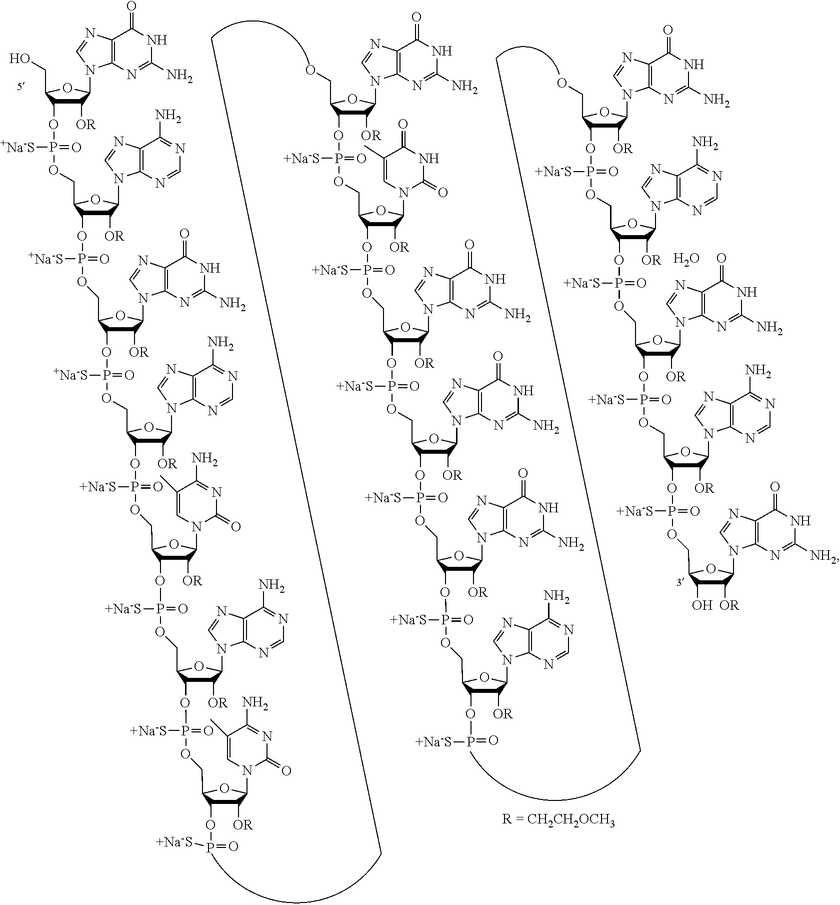 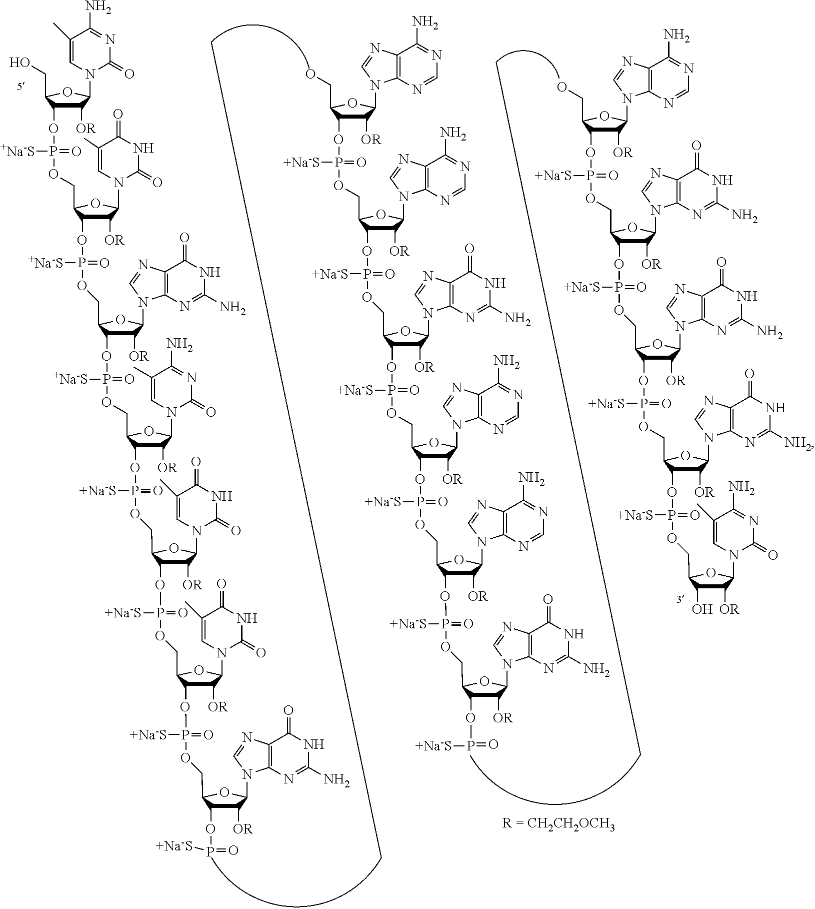 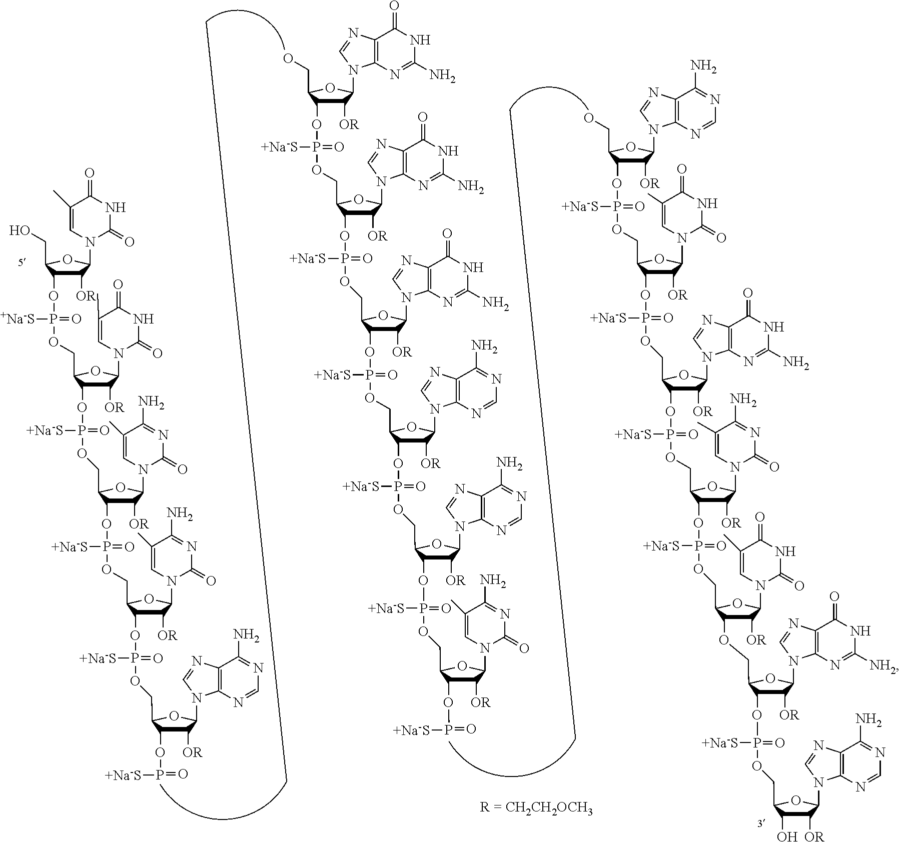 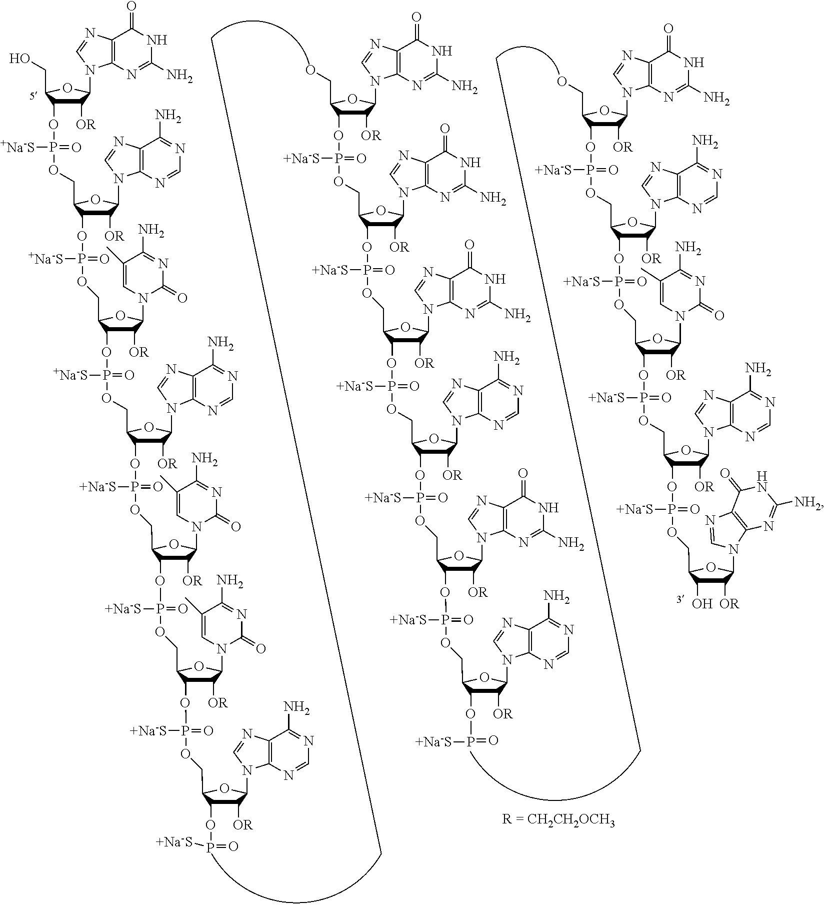 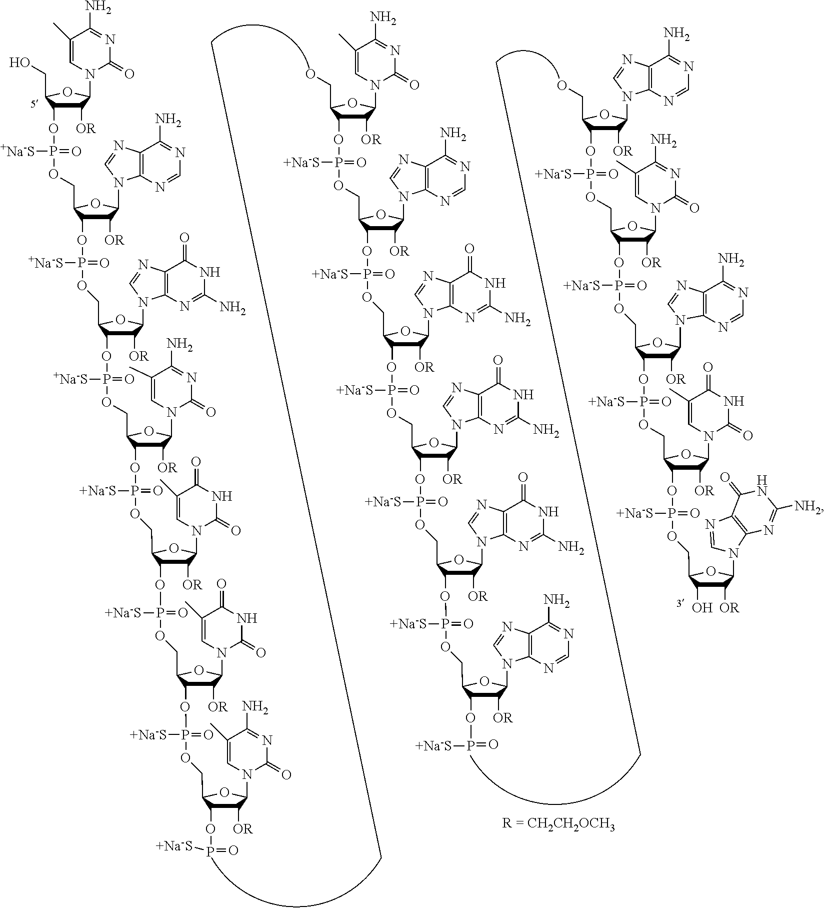 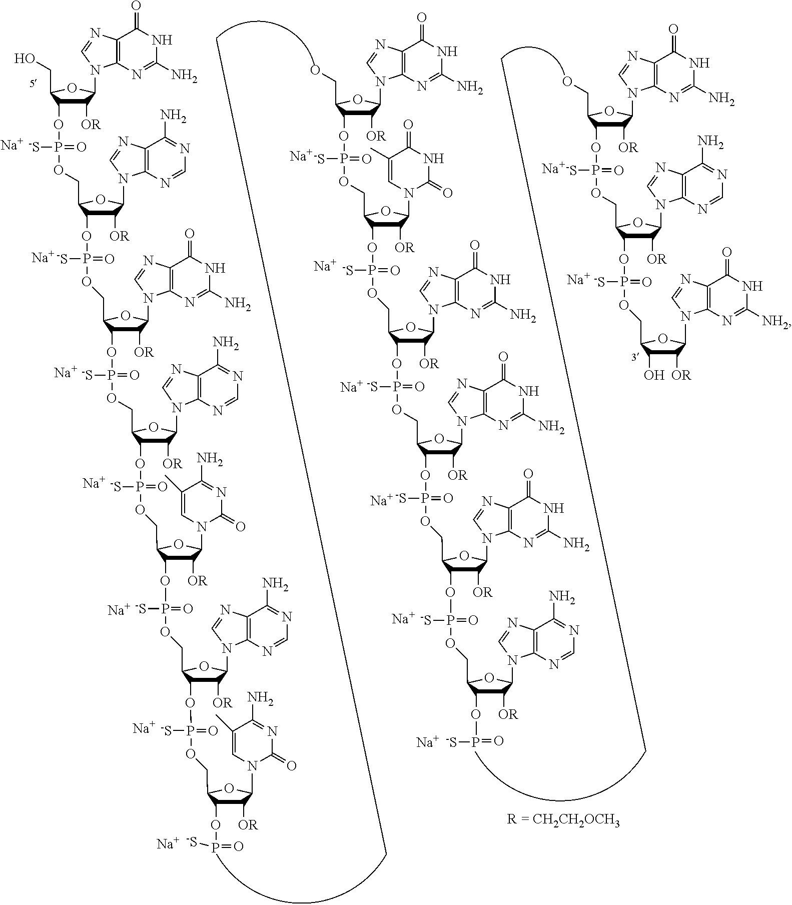 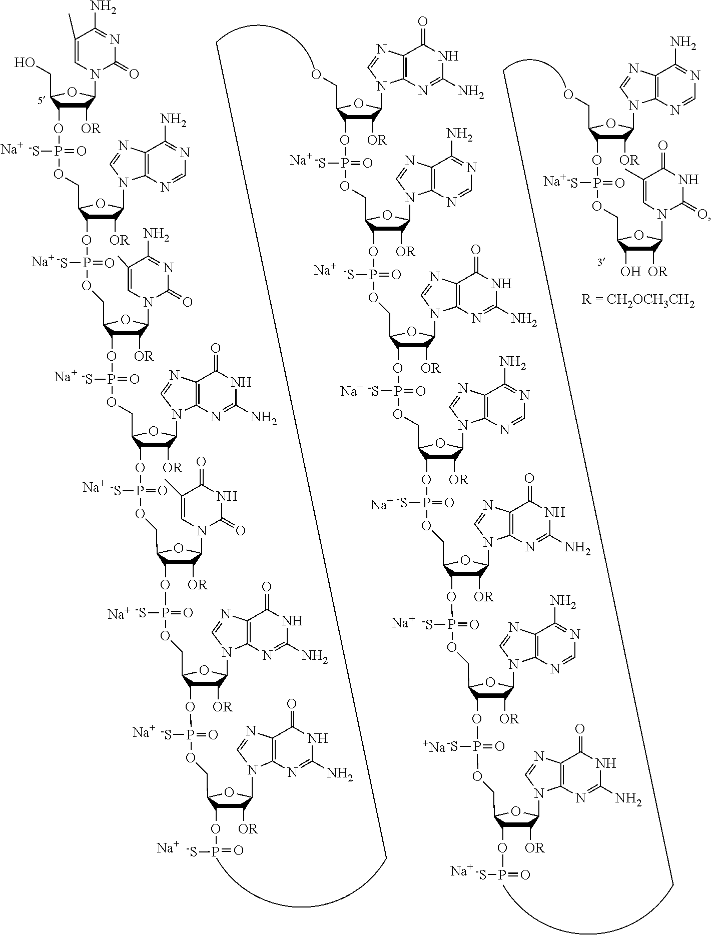 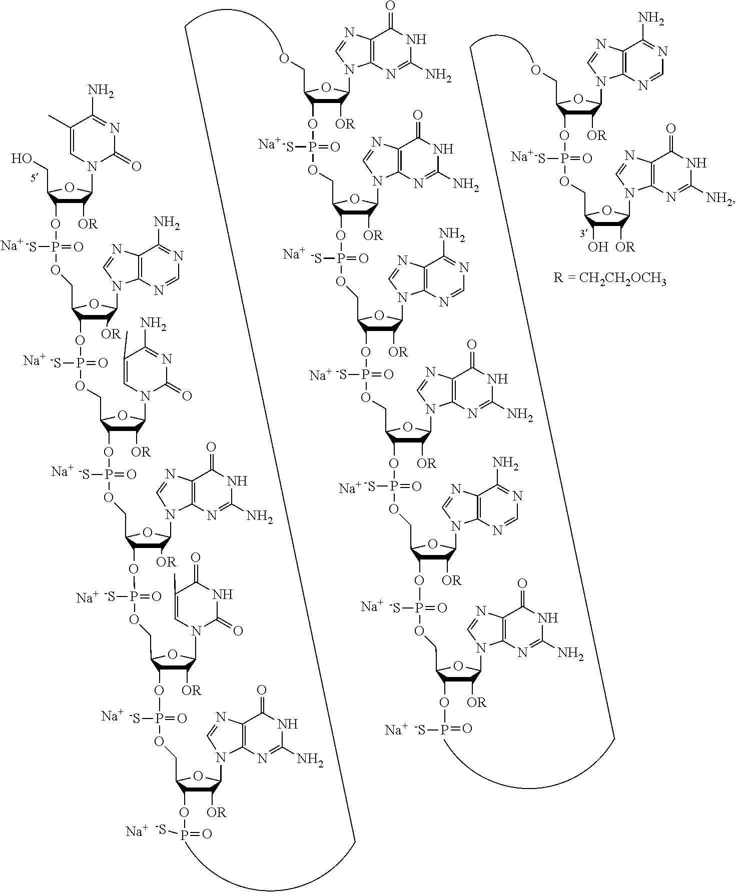 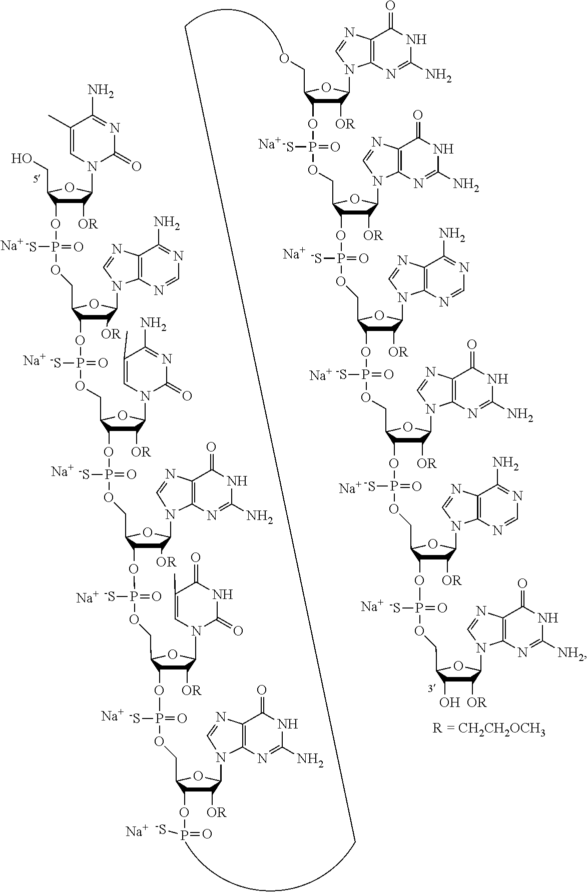 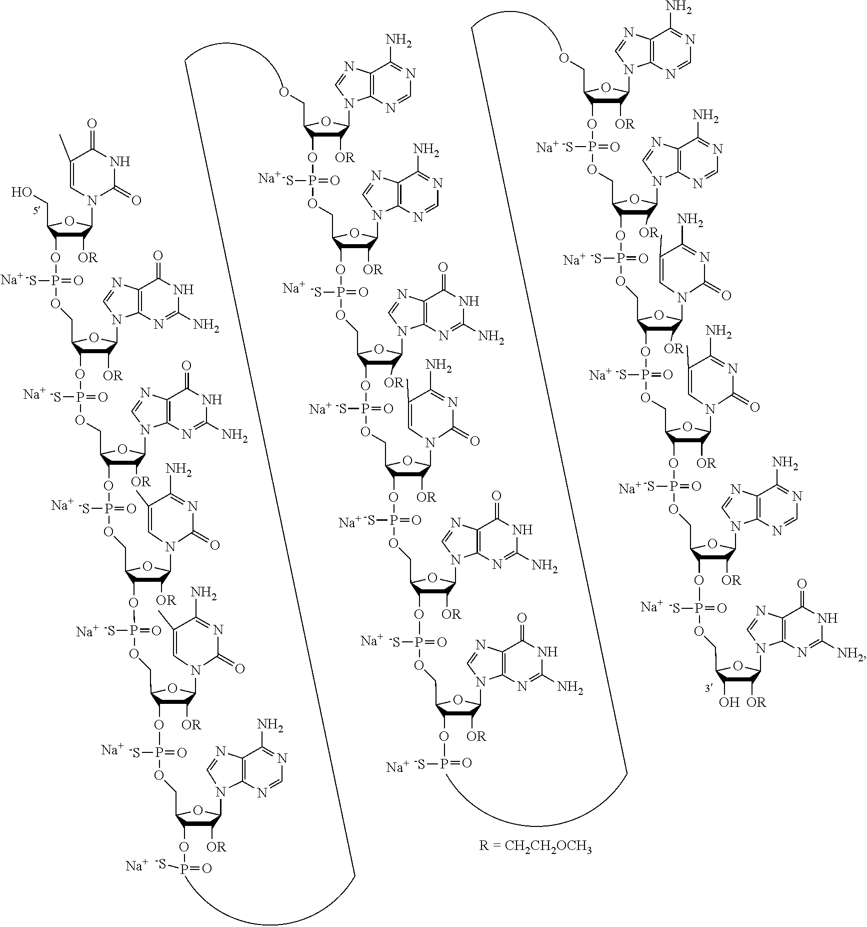 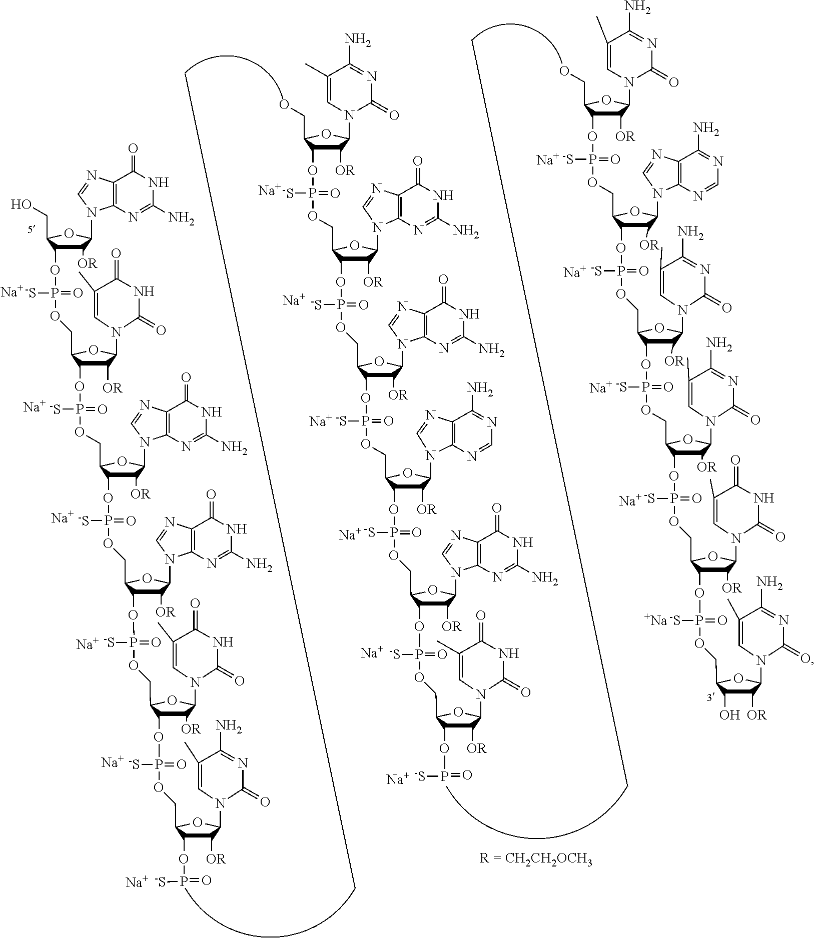 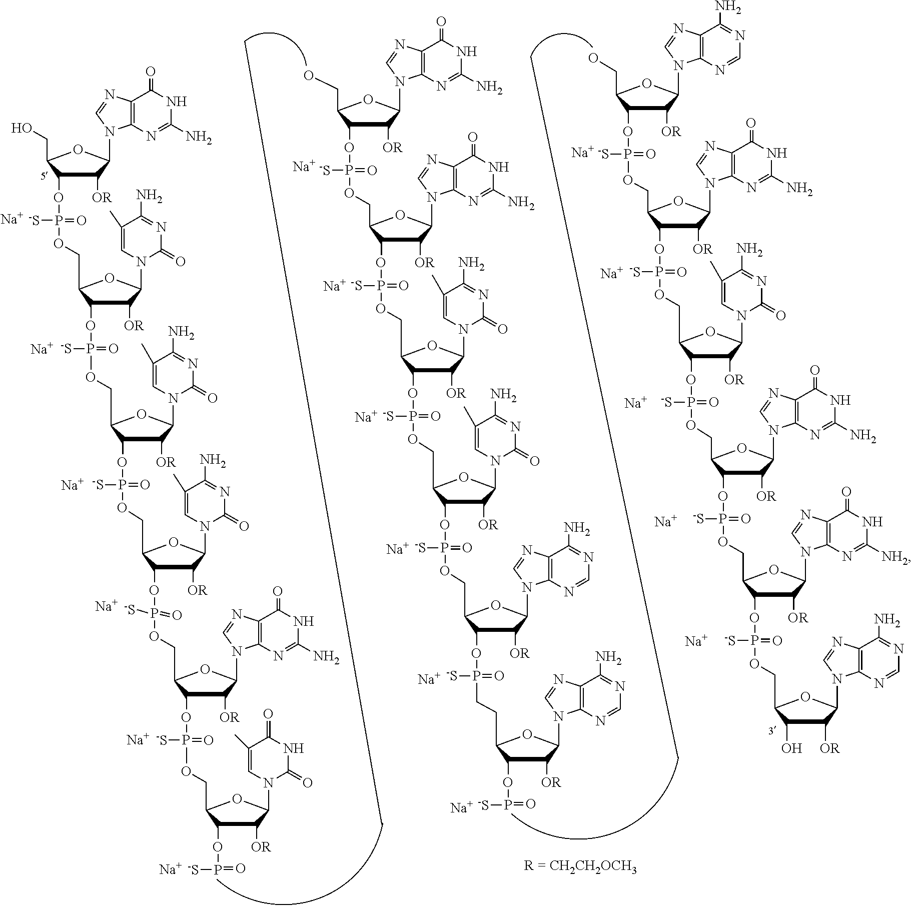 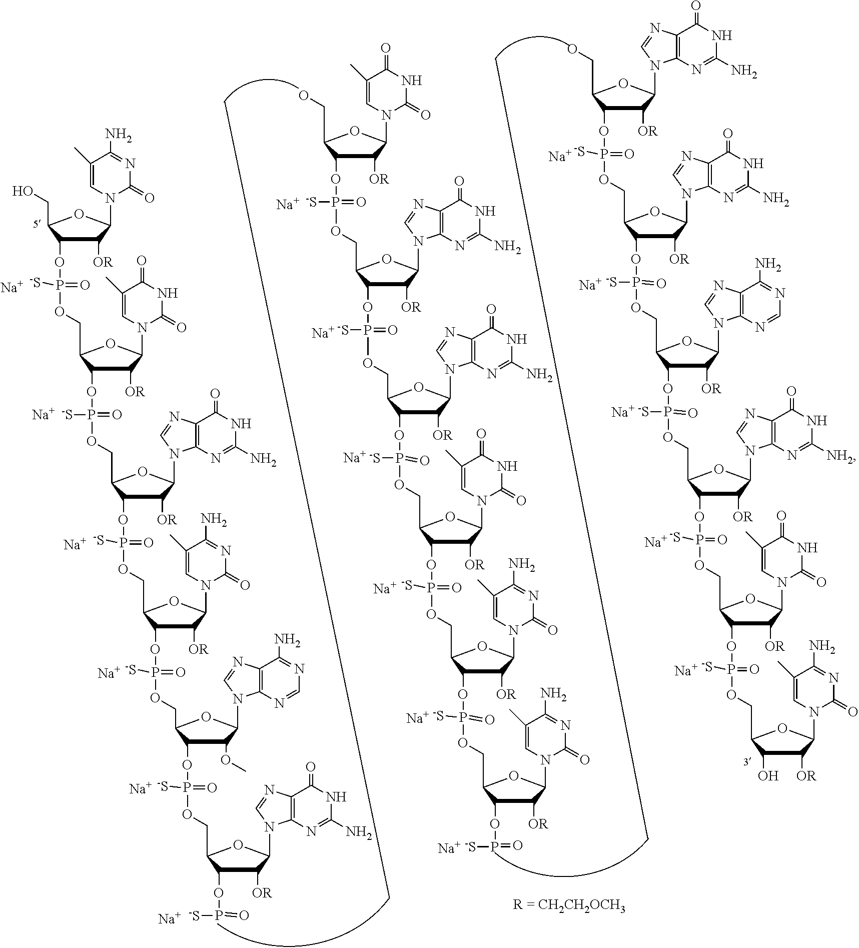 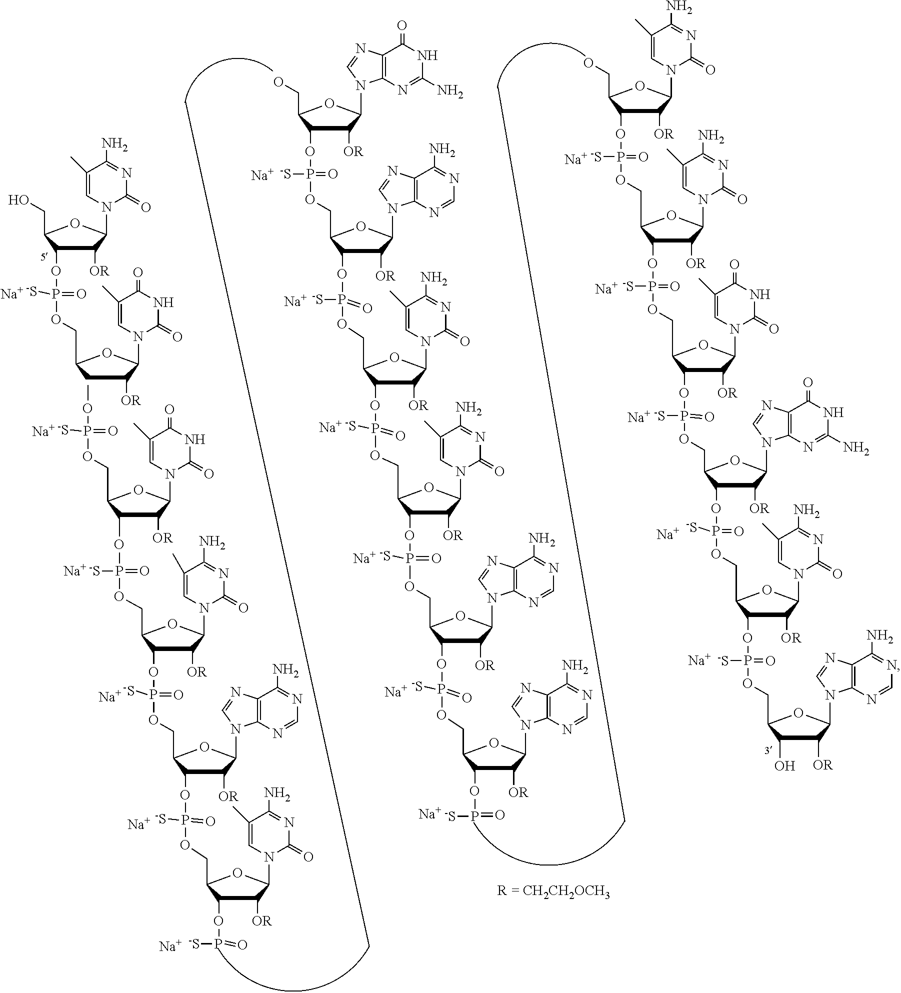 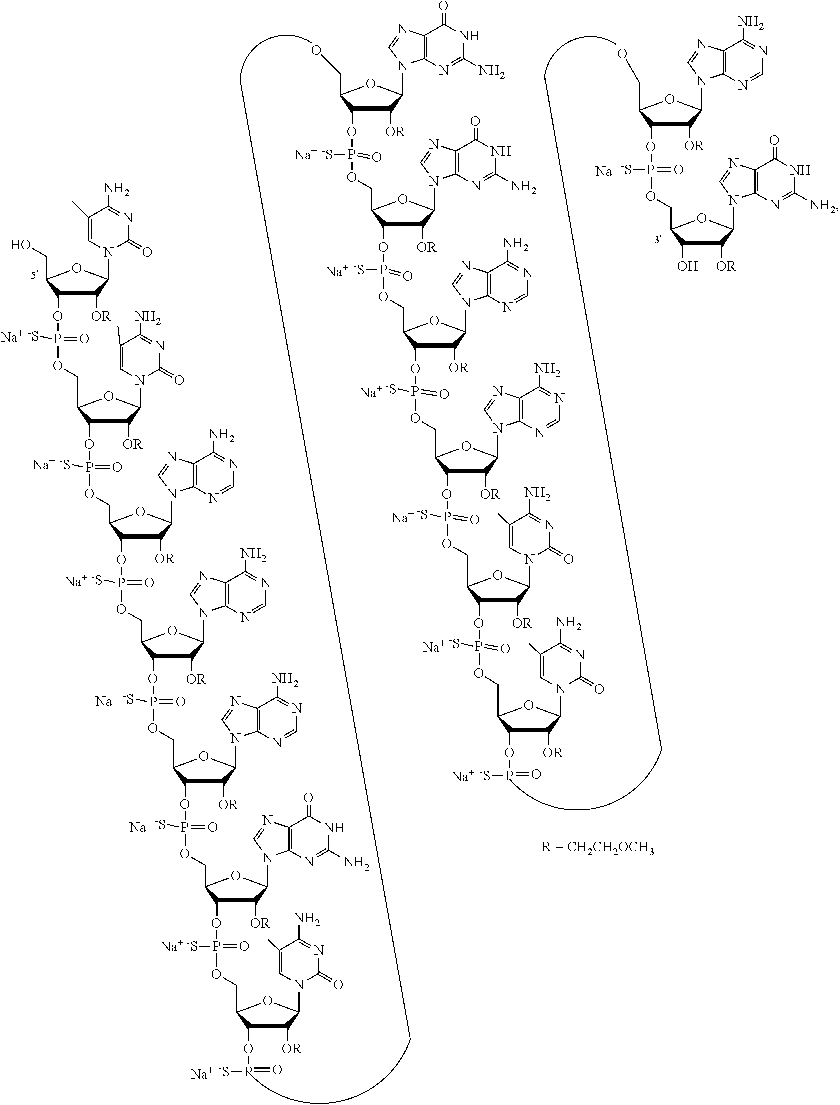 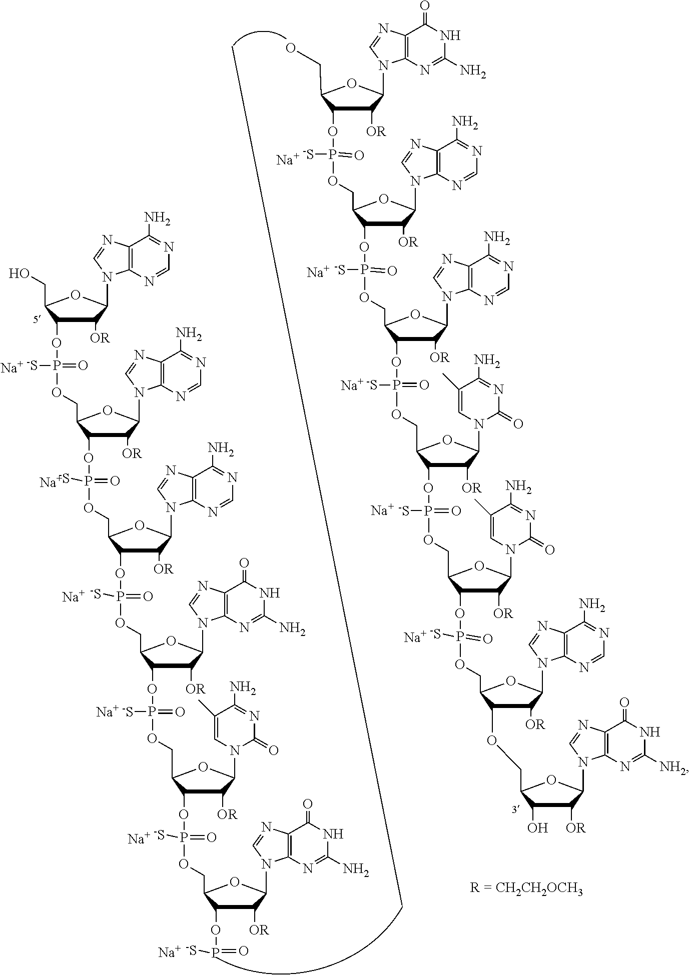 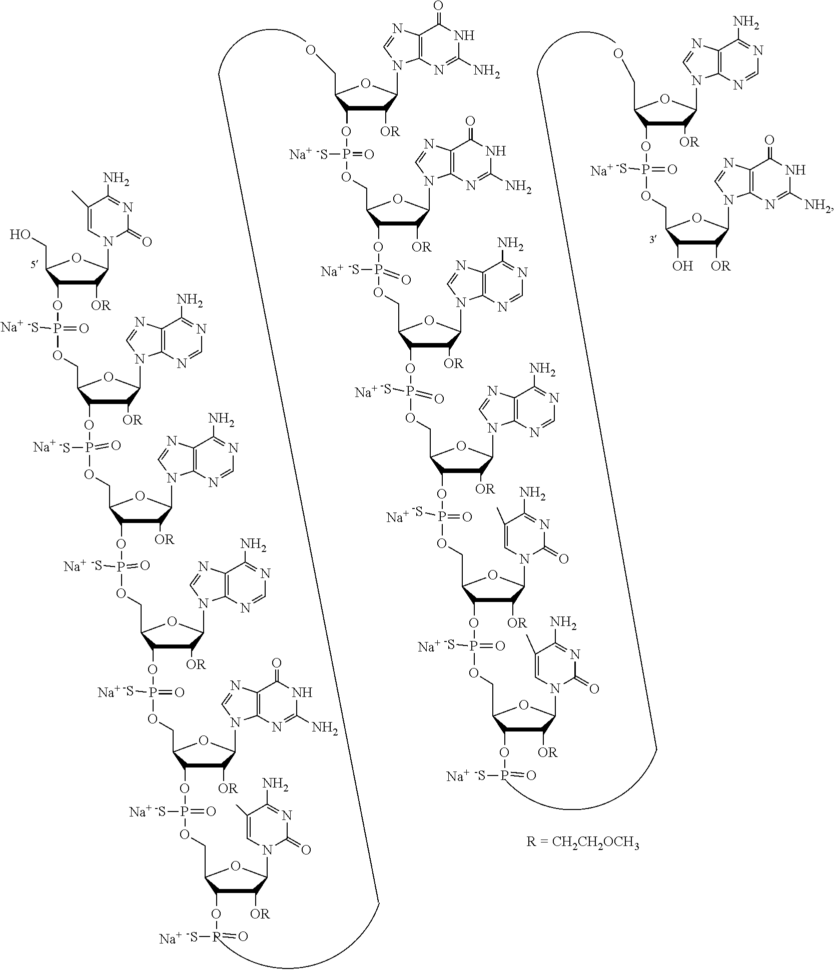 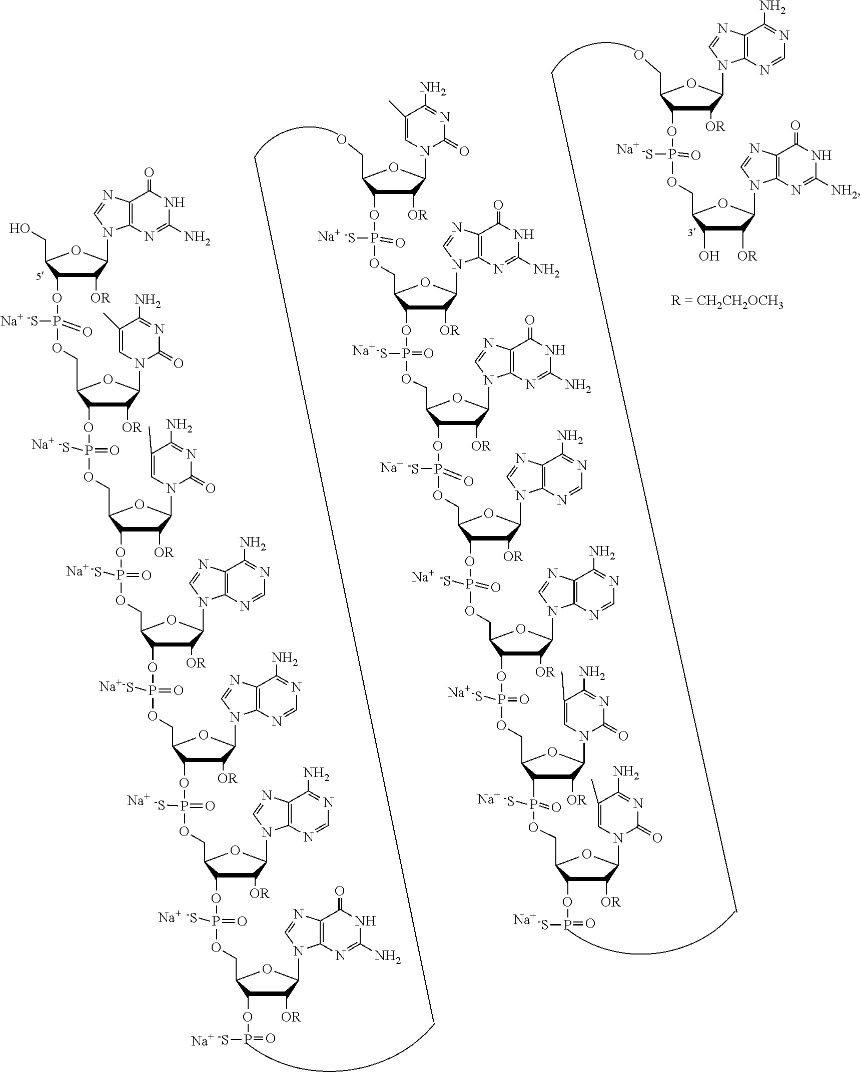 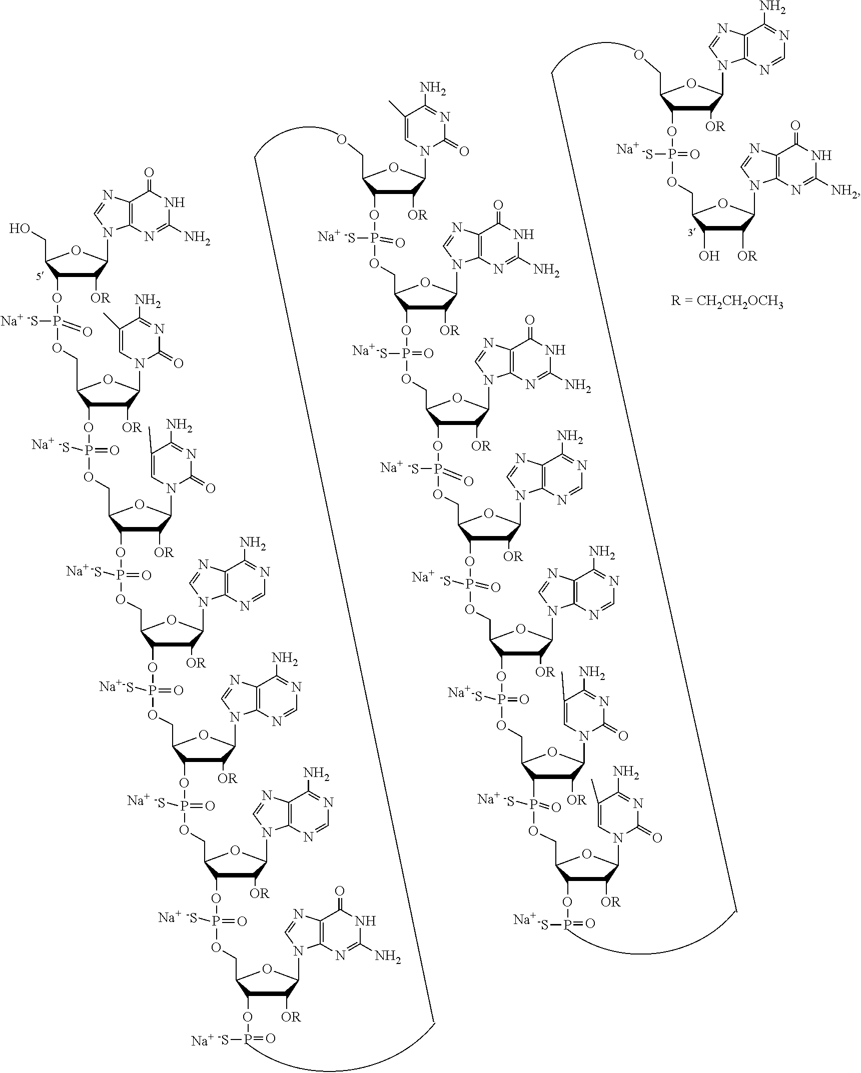 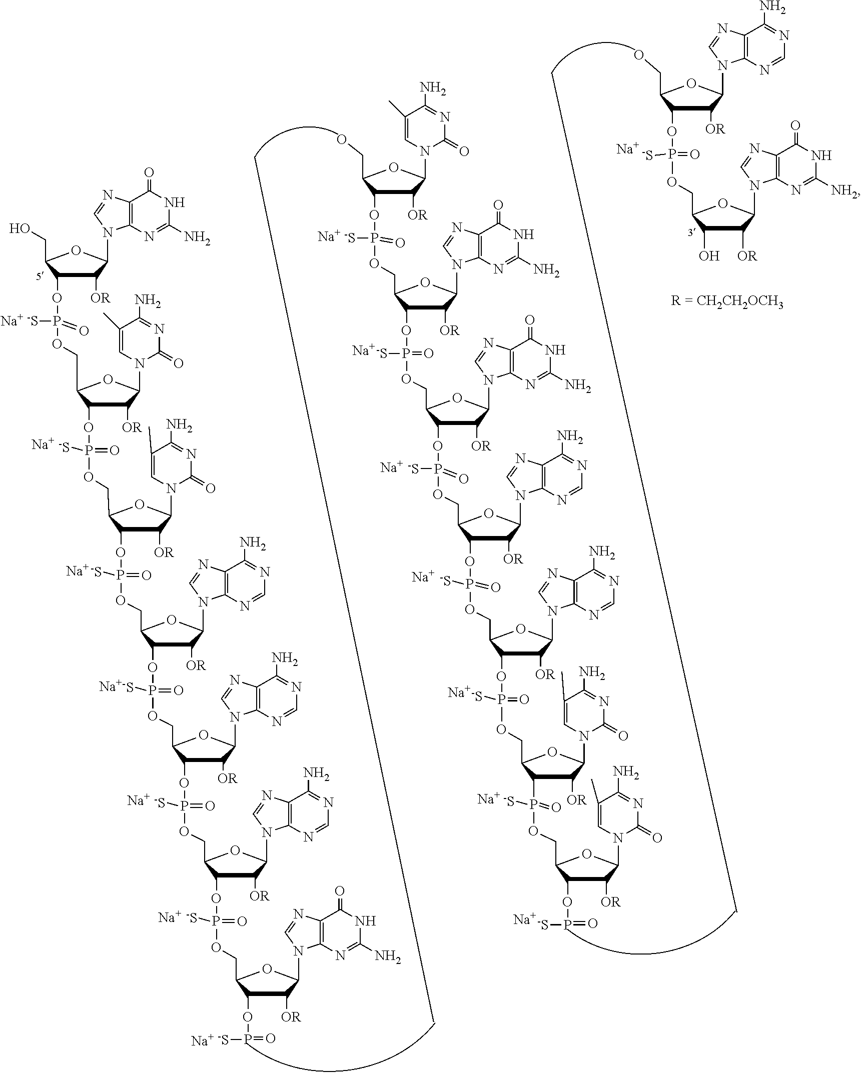 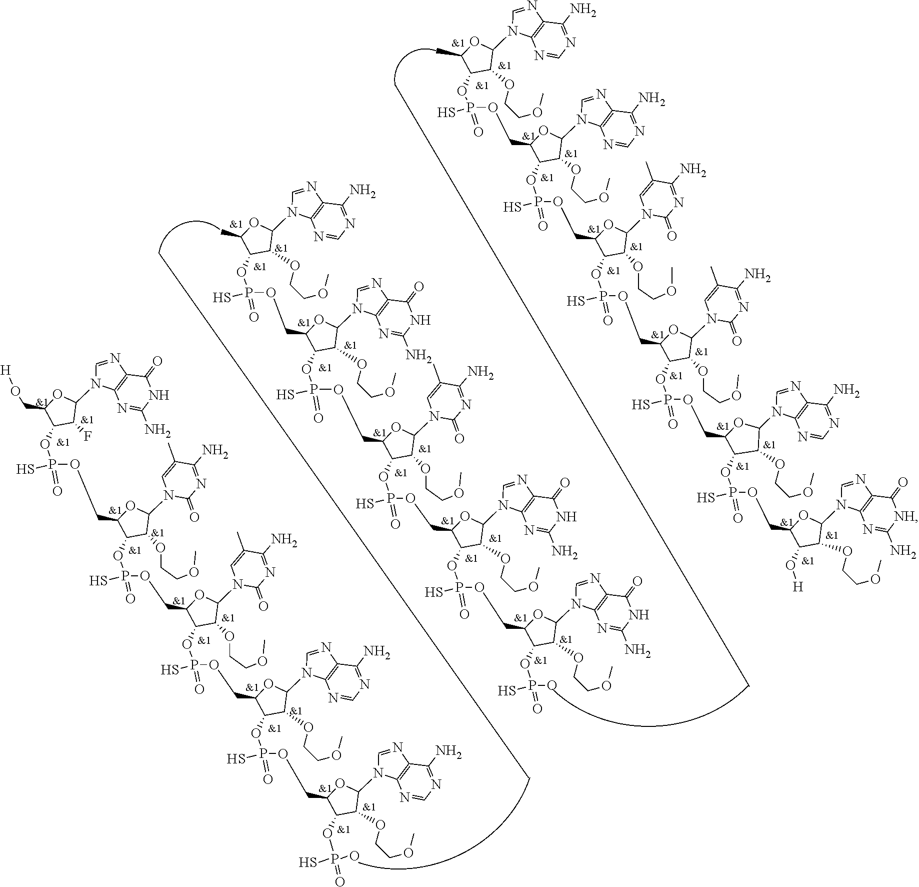 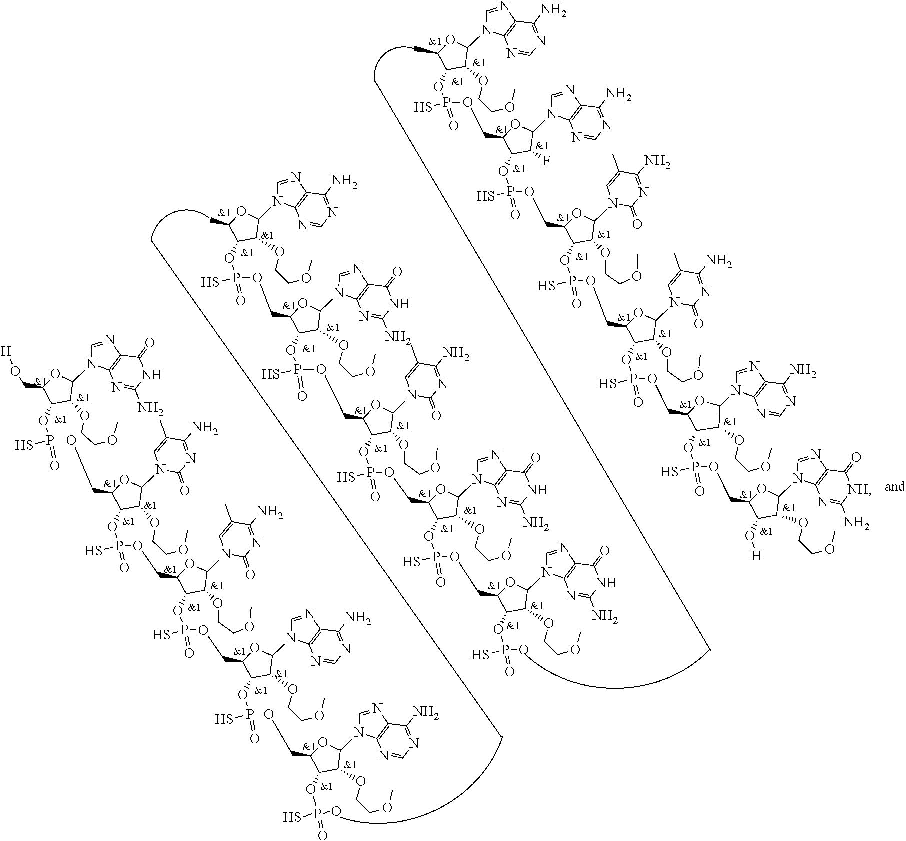 [chemical structure]
